SEQUENCE LISTING (3 sequences)

SEQ ID NO: 1 - DNA, 118119 bp, unidentified
  source 1..118119: mol_type = genomic DNA; note = Bacteriophage
gtccactggctggctagatgagaatgagtctcattaccgcccggtacttagcaggctaatagtcacggcatgttatcattacacaccgtgactatttcaactttaattattacgcaacttttaatttctcaatatattgttgcgggctgcaactatattctgcgcgttcttcctcggtcatactttcccaacataattgcaggcggtcggctaggaactgataatcattatcatcctcaagagattcatcaataaaggtttcacctatcaatgtaaccccaatcgatagaatatcgttaacggcttgcgcgtccagcgttttaaatccgtatgatgccgccgctgatagcacgcctttatgatcatcaatcatcgtgaaatcagcgttcggatataattgtttaaataactcaaagtaatggcttttataaatagcgtctttgctgtggtatatctcgctaactttatcagcctcaaaatatttgtgtagtgtatcgcgggaaaatacacggatattactatccccacgccctcgcaatccttgtttgcgcaaataataataatccgacttgctcattagtcgagcggtgcaaataatgtttaacgtgttaacgtcattcatacattggcgcatatatgtaacaaggggcaatagtgtatcttgcattattaaatcatgtttgcaggcttcatttttatacttatttaaatctaaattgccctcgctatcaaagcaaggcgctacgcgatggaaagaattaatgatagtgccgtccagatcccaaatcatgacgcgggaaatatgcgggaaattacgggtaatattcgttttaacttgattcattttgttacgttctccatttaagggattagattaattcagcattacccgctatttataagcgggtaataataaattaactattaaagaccatacgccaacgcatcagcaataaaactaatatcttcaccagatacaccggaacgaatgcccgcgcaggtatatacctttgcgctggtcaattgatcatcgaggcaagcggggcaaggcgcgacaactgcaaaggctaggccggattttacggcgtcgatgataatcgtttcttgtttcacagtgtcaacataaacaccttttgctttaatatggctagttagtttaccttgcgtataggcaaaggtagtaacctgcaaagcgtcgttaaagtccaatagctcatcaataacgtgccccgcctcgacgtggtaacgttcaccattagaataaaccagaacaatggaagattgagcgcgatcgaatgggatattttgctttttcatatttataaaactccaattaatttaataaagggtatttaatttacttgataataatctgcatttgatacggcgttaattcaccatttacgggtaaattattatcgcttagccactcataaaaagcgtcatcatcgtcaaaagggataatatcattatcacgataatcgaccgtaataatcccatccgtattagttttggcggctaggataggcccgaagctgtaaactttatcggattccatttcatccggtgaaataaatgctttttcgtccagcgttttgggtaatgtaactttcattctaatttagctcctataatagcggctatatttcaagccgctagggtattaattatttacgcaaaaaatttaccgttcttaaaatcgatcaaagtacgctgaccatttgcataggtaattaaatgcgtttgtgtccagcttgacgcgccaacgttgtaacccatgtccagactaccagccacgccagcggtatataccccgccgtaaatgctggcggtatgagtgtgccccgtgtttaatttgcccaatttcttaaactgtttcggattgccacggctgccgtttatgccattgtgaccgtgtacgccgcattcaatgcctgcaattttaaatgattggtcagtcgttaggaaaatggcgttaaactcgcaacctgcaaccttacgcagcgcataatctagcacgttgaaagtatcgtctttttctgcaatagctccgtaaatcgcggcattcaggcggtgatatagttccgcgttcgctgggtcgtctttaatgttagcattacgatcatcaagccaacgggatagcgccaaatcgtgattcgattcaacaatgattgtttgcgagaaatcgcgctccattgattccagtacgcgccccgtatcgatgagatcatccagaactttatcacgtccggcggcgtattgttttgctaggaatacacccgaagcgcggttgtgatggttgcgtgatgtaaaatcgtgaacgtcatggataaactgatattttggtttaagaatatcaagcaagctattttcgccagcccatgacgcaatggcacaatcatcatcaagtttttcggcgtgtatgtcgccatattgcaagcctaaaacatgccccgatgtttcatagcatccggcgggagtagcgcaaacattcagatcataaaacaccccgctttcgtccattgtttcaagctggcgaacaaaaaactcaccgtcttcgtcgaactcaacaatcagcgcaccgaagttatgcagtgcttccgctttctgtcctgctttctgctggatgtaatttttaagcgttgccgtgccagttgaatacatgcggcgcactacttcaccttttaaagctggcacgctttcggcggtgattttagcgtgaccaattgccagaccttccagattaagcgcggttgcagtttcagcgaatccagaaagcggataatctgcggtgggcaaaacattaatttcagccatgaaagcaaaacggcgattattcaaaaacacgtttttgctgcaaatatatttatcgaacgcggaatcatattttattccatccgcgccctcgccattttggaatccgtttttattataaatatacttgctaaccagcaaatcagcgccgatatatttagcaaattgctccagtgaggctaaaaagtttttatgcgggaacgtattattctgaatcgatgtaataataaagcgtttgccttgttttacttcccagctttccaccgtgctagaaacaatccccgccgcttctgggcgctcatcgtttttaatggcttttgctactttcgccgctttcggtttcgctggtttatttgcttccagccaatcacaaatagcggtggaatgttttggagtatcgtaaaccgcttttgatatatcgtcacaaacttccaatttagtggcgcgcaggcgtttaccgtagaaattaaagtcagcggccattttcaggataacggtttgtttttctgcggaaataatcatagtaataaaactccattaaagggattaaaaaacgtttgttattggcaaatattgccaaatataacgcctattatttgaggcgttataatttgcaacattaatttaaaggcataaggctagaactaacaccacaaacaacggcggcaataaaacaaatcagacctaaacgctttgctaacttagatttacgtttaaaataatatgaatctaaatcattttgaaaataagcgaatagtaatgaggccgccataataaataagcctagaattaaggattcagtagggtaaaagatcattttaataactccagtcaaagggaaattatagtggggaatattccccactaattaaatttattcgcctgcggcttgtttcacttcatcggcaacgcctaacagttgcgccacggcgtccagcgtttccagttttgcgctttccagtgatgccagatcgtcggcgtctttgataatgccggaatcgatagcgtgttttgcaataacgcgcacataatgcgctttgcggattgagctaccgccgccaactttgcgcggcttatcgcttttctgatacgcttttgcgctggtcaattttgagcgaacggacacgggggaggccgcgccaactgcttttgcaatctctttcaggccgtcgctatttgcgaaatctaaaccgttttcattaattaactgctggtacatggtaacagctttttgggtgttttcttcattccatgcaaattttgcggatttttcggttttaacgttagccatgattgtagctcctattagtttaatttaagggataagattaacataatgttaatcgttatatcccccgaactactagggggatataaagttaacactaaattataaaacagcttcccatttttcgttacgctggacaacctttacaacgtcgccggatttaacgcgaacgctaaaaacggttttgccgttgttactccgttccgtttcagcgtataccgcgtcgattgtatcgaacggcataagcgccgcgcctttaatgcgttcggcctttccggttaattcatccgttttagtaacgaaaggaataaaaacggattgaccaactttcggcgcggtaataacgttttgcataatataaactcctagttatttggggattgttccgcattacctgctagaaagtaacagcaggtaataagtaacaatccgctcgccaatataatcaatttaaagaactccacgcaggccgctatatcccagttagggggcggggcacattatggtaagtgccgaccgtcaaattttatcaagttatcgctcttatcgaactgggtacatcttaaagcctatcggctagggtgtcaaacaattttttgcagttttttatgattttcttttaagtcaatcataatattacgccagatcatttccagaccttgctggcggcctttcttgtactgcttaaatcgatattttaccgatctttgcgctactgtcaacaaattttttgttttgctgtccagtgccttgctaggtttaccagtgatcaataaagtttgtttcatatcgtcgattccttataatgtataagtttaccgccgataggctttaagatgtacccaatttttaaagagcgcggcggtaaacttcccgccgtgtagttcgtcattgccgccctacgagataaataataggggattcccgaaaatggcgcaaccttatttttgtaaagaaatgtaaagacaaacgccaccggacaccatgcggcgaccggggcgcggcgcattaaaattaaaagaaacgggcgcgcgaataccataaaacccagcaggttggcaagtaattttttcatttatatttgccttgccctacttgacaaaacttaaaaagctcgtggtagcgataaacgagggtaggcaatgcaatggcatggctaaaactttcatcgcgttctagggcgttttaggcgcattctagccttgttgataacttatccccaaaagcaaaaatgaaattttcctagttatccacttgacttttattttccagtggataactacttattttatgaaataaattaattgacttttatttgttagtatgcatcggaagaatagcagactaataattactagaataataatcatgctggtggattgtaacggcctaaactatcagcagactattaatcacgaaaaatctcgttagctaactaactatttcatgatgtataatatacatataaattaatgagaaataataattataataaataataactacagtaaataataactataagaaactatcggcctaagtgagaaccactatcatttaagttatacacaaagttatccacagcactttttgactttacacgataggcggcgatataaggacttttacacagacttatccacaggttattccactgtataaatatacagggtaaaaaggtagtgttaacaactgcaatcaattttaagcggcttaaatcgcattctaacgagctaaacgctgcggggtaatattaaagtaaagccttaatgcgtcaccctggtggcttcgccgtggtttgctctttattgcaccgctgcgcggttagcataaaaataggcgctaggcaaggaaaataaaaaggtaaaataatatttgcctatgctagaaatttacctttatcattcactcaccgattaacgagggaggccgaacaaatgaggctttataaacctgataatgccaccgttttaaaaggtgcattgcgcaacctgctggatggtagcaagtcaaccagcattaaacattttattaacaagattgaaactattcatcaaaacttttttgatgattatgacgcttatgatatggatagttttctacccttatatagacaaggggcaacgctagttttatataatcgggattgccatatctttacaggcaggggagcggggccggataccgattttattgttgcgcagggtaataatattgttgcgtatcatagcaattcaggccgtcaatcatttaatataaatagttttgagcttattggacttgatctgataaatggactttctagcattgagggatttttagatgttatctggaagtatttacccgctggatggcataagccggataacgacggatctcaatataccggtataatggcttttgatggcgacggtaattttttgttaccacgggagctatacccatatatttatgatggggatatagaaagcgctggtttttctttctggaaagatgaagatcagattattgacagcgtagaaaaccatgctgtaaattctaacactcaaagcgttaacactcaaactactgaaaacaaaggaaaaaacactatgaccaagattgctaacatcgttgccgctaataaatccgccgttgtaaatgctgcaaaactggaagcgggtaaaattgcactgactcaaattacgaaagtagcggctaaaaaagcaccgtttatgattaaaggttatattgatacacctatcggtcgagtagtgattgccaacctgctgggcgtagcggttgaccagtacgccacaagtaaccaaaaggcgaaagcggtggcaggtgcagctatggaagcggctatgttagaaatggtgcaaagttttaacatcgctgagatgattgatgaaatggtaaaaggtattgatatttctacttttaccactagcaccgaaagcgagtaataatattaagcggggattgttaccccgctttactattcccttgttaactaataggaggctaataaatggctgatgaaaaatatggcttctcactgggagaagtgagcacggcaaaacatgatatgatcattttggtcgattgtcatactggctattgcggggaagggaacaccgaggaatattttgttcctgctggtacggatctggacgctttcgctcaggaaatggctattgataatgcttctcgttttggtagcgatggttacgaagacgaagaaactggcgaatggtatgaaaatgaaaacgtatatgcaagcctttatcattatcaattaagtaaatccggcacatatgtaaacggcggcgatcctattaattccgtcatgagattaattattaaatatggcggcgtcgagatagttggtaacaaagcggtaatatacgcaaatcgattaaaacagcttgtttatattcctgatagtacccgatgggatgaatatgctgttttgcatgacgaattgaaacgatgtttcaatattgaatccttacaagtagtctaatgtaaagttttgtaaaggtcgccttttatggtggccttttttgttataatgcctttacaccgtttgaggacggttgaaaattttcccaaaaattggaggcttaaaatggttgcttattcttctgctagtcatctgttcaccgctcaaggctgggtatctaaccgcgttattattcacgaaatggtgcaagaatatggcattgctaaaacatacaataccattcaggcgctttatgataatgatcaaatcgatcagcaaacggcgggctttttgctggataccctgaaagcagaacattgcaccaaaaaacaagcggctaaaattgttttaatgtaattaataacggggcgttattttcgccccactaactagggggaatgaaaatggttatttttcgagctattttagcgggtatcttgggcgctattttatcgttgtttgcaatagctttcggcgttcacgcggttattattggcgcgggtatcgctgcattttcttatctgctgggctgcatgactagtaaaggagaataaaaatgatccgcaatgtttcccttgcccgttcgaaaggctttaagctggtggatgttaacacgttcgagcgtgaagattgtaaaatcgaatatgtagcacgtaataagaatgcctttcgtgttacagaaaagaaatttgacaagcgcggcaacgtgatcgctgaaacggttaaacactttgccaccttttacgctgcgtttcgtggggtgttataaaatggttatttatgagggcaaccgctttgttgctaattgccgccctgcattgctagcaaattacttaaatcagctttcaccagcttataagggcgttattaatatttatgaaggcaaggcgcattataaaatcaatgcggtcgttgcccgtgaactagcgtttcaatttttgacctttgcatcttgtgacgttcaggtcatgggtgaagcgttaattgcagaaaatgaagatgattttattaatatttttcgcaagatatgcaccgagcgcctgataatgaaaggcgcttatattcaatccactgcggatagtattgaaacagcgtttcgaaaggtggcgcaatgagcaataaaattgttgtaaccaaaatcagtacaatggttgatgttttttacgtgcccgatacgccggaaaaccggcaagcggtcgagcgtggcgagtatgataaagtgatttatgatcacgatggttattatcagcatttggtcgattcctatggggaagaggaaaagatcacgcatcaattaccggaggatggtttatgaaacgatggtttaatcactggtataagcggtttttagtacgccgtcgcaaatctagacatcaagatgagtggcgggagtgggcaaagcgtaacggttgcccgctactctgagaacgacggggattagctccccgtttttccatgcctccagttaaatacccccttgttttagtatccccaccaaaattaaacgcgttagaatgcgatctaggacgttttagcggtatattagaattattcactacataggaatactgtatatttatacagtaggataagctgtggataagtctgttaataacgcctcaaaatggctaggattctgtcaagtcaaatcgtgctgttaataagtctgtggataacttaaataatagtgtttctcacttccattcttaaatgcaaatcattatcattcgcatttcactttctaaatgagaatcatttcacttcttgaatagttctcatttcactttttaaatgcgaattattatcattctcatttcgctttttaaatgattcttattatcattcgcattcaggcgcgcagggcaattgccctgcgcttttgtgcaaatccgacattgctccacgggtgttacgtgcgggcgtgtatgtgcgaatccgacaatttttggtagtgtgcaaatccgacccgaaaaatttttatagtgtgcaaatccgacaatgaattttcccaaaaactccagcaatccgatgctgcgcatcaatcctgtggaattagctagaatatgtgcaaatccgacatgggtttatttgtgcgaatccgacaagatttttgtgactttgattggagggtgtgtgattgtatgggtttgaggagaagatgagagagcgagaaaaatttgagggaactgtgagaggggcgcaggattggtgcggagcacctgatttgcggggattttggggaagagttgagataatttgggaagatttgggataggtgcagataaagtaaaattttattagcaaaacgaggcaaaatagcttgacaagtgaaaatttctgtggctctaaaaactcaggcaatcatgggccttcggcccgaaataatgctgctattttcaggagattatatgttcctaaaaacttaggcgtttgtgcaaatccgatcataaatttctagaaatgcagtgtgcaaatccgacagtaatttctggaaatgtgttttcaaaaaatgtcttctagaaataaccgccctccctataaccgcctattgctggagtttctaggaacaatgaaaaccgcctgagttggctagagaaacagatttaaagtttaatgtaaactctagagttagtgctcaactattcctagaaactcaggagtctagaagaaagtgttgtgccatcctagcagatgagcagcaacaggtgtatcatcttgaacacagatattgcctttaaatattctcttgtaaataaaattaatagtttccccaatctcgtgagtctgctgctctaggcgttcaagatctgtatgctaatcccgtgagaactgctgcagaatacatcctatcgccggaggcgtcggtgtactcttcgagttgggatagcaataatcttgttgcctagagctaaaaacagaagttgtttttaatctatgagattattataacatgtttgggtatgagaatgcaagaaatttttgctactatttattcctttcgtctgtatttgagaacgatgcccttcgaaaccatgaagaggaaagttgctagataggaatgtgggcttttaagcgcatcataatctactgtcccgaaagaaccttcgccgaaaacttcagagttctcaaatgtaggaagtaaacgtgccaatcctacagctcctataatcttaaagaattgatcccagtcatttaacattgctttatgtgcagactgtaagattaatggcatctctaggttcttttcgagacatagatgaacctgcgcaatcgtagaggcaatccctagcaacacttcatcttttgtataagggaatccagtatcttttctttggagtctaggaagatgatcatattcacaacgtttaggtgggatgaacttttcctcgggatataattctaatgcaactctaacgtttccttctttaaatactggaagttcctgatctgcaaagacatgatatgaatctgccttgaaccctacaatcttatcaccaacagaattgatgatctttctcattgcaatataagaaggggatagagttacgaatttttcttctttattatatttaacaattccagcttctgtataagctgcgggatgaaactcagaacagaatgctactgcatgagcttcatcaggagttagttcagaaacacatttgccgtcagcattcttagccagcttaataatagtctttaaattctcactaatactcatatcaagcctcctgaactttgtctgccgggtagaaatcttctagtgaattgtagtgagcggtaaagaattcaaaacttaagtaatcagcagtattctggatgtaacgcagctcattaataaaagactctttgaagatcttgcgattaggtttgagctttataatttcttctaccaaagtctctaggatcttttttgcgtcttcaacttggtatgcagctaaggcttgagattcatagattacttcatctactccattagtaattaaagatacttctatgattattccatcctcttctggatggactctatttgcatccagctcacgaatgataacacgagtttgactagttatttgacgatttaacgcgattgtgtaatgtaacatattattctcctaatgtgcctcccccagtaaaaggtctgaggaggcgtagacctataaattaattaacgatctttattgatcctttttactctcattaagagcagcccccaacgcctcaaggatagtatcatttatgctcccaggcctaagattattaaagtgagtaacatagatgggacttttaaaaccttcagcattcgcaggtgctacgcttcgcatatcagattcaaaagcttcttgagtatgtttcagctgtacagaaggcccataatcttcaccaactcgatatgaggaactattttctatagtgccgccgcatggaacattgtgcatttccgacgttattttagaattaaatgcgataccacttttggtccaggaacctccaacagtcacaggagtactaggacgattcacctcagtcttcacgagtttataaccagcatctattaatgcgttaataaggttatcagctggaatccgagccttgccaaagatcttacgagcttcttttaatttatgctcaagatctttacaggctttaatatcagtgtacgagtttataaccagcatctattaatgcgttaataaggttatcagctggaatccgagccttgccaaagatcttacgagcttcttttaatttatgctcaagatctttacaggctttaatatcagtgtgcgcaaaggttaatttcatagatttgaactgtacagtggcagttaagtctttaaaatcataatcccatgtatttaattcatggtctggtactacttgaacaggttgttgtgcatataaagtcattacttattctccaatttgtctaacagcttatgagctgctaggttatattgtttaagttcacggttaaggatttggttctcacactttaactctaaattcttattattaagaatattacagattagtagaagtgctagtataactagcagggaacctactagcattaaaagaaaaagagtttccattatttaattcccatgtaacgtttgagatacgtgatgaagtcttctgttgtatgttctacgctaccctgaaccacaaacaactttaaagcctcacgctgtaatttaagagcagctaactcaccacgtatatcttccgctagttctctagctctgcgcataatagcaaatgatacaaatgttacaataaataacataactgctaaaataattactatgatctccatattaacatccagctcttatcgtatatttgggcatgttctactaattcatttatcgatatagaattatcgtagataccctctagtaaacgttttctgtcaaatttatcaaccaaatactctgtgccatttatcgctaccctatacagctctggaactaaagggtgtttgtttggtattggacgactacctaagtctatctcattatatccccaagttgttttaaatagtttgttactaagtgctgctagtggatttgaccatacaatatagctactcactacttatctccattcttagctttgaaaacttattataccaaactatatgattatcgtcaattacaaatttatggtaacatgctggtacggtataagttccataatccgtgacgcgctgcacaaccttgtgtgcacacagtcgacaaccatgcccttctaaatgagatctagcagtctgccagtaataaccatcatgatctggacaataaatctcgacttgctgctgcattgttttataatttacacgctcatatccgaagaaagccccgtgagctgcaacggctttcttgataaatataaggtttagtgcttcttgctggaagtcgggatgtagctgcttaatcttagttctattaatagaaccatccgatttaatgaacctatttattggaaagttgttagcttcctcgattaataattcatttactttctctctcatattaaatagtctcacagttaaggtagaaacattcgtaagcatctccagtaaactctttatcttgtagtaaacacttagtttggtcttccggtactccagcatctaataattgttgtttacttggcttttcagcgaatacttttacaaaatactcaccatcctgattatactcatttatatctctggtaaggacatatacttgcatttgaagttccttgaaaacataaaaggccagaacaattaagtcctggcctctagaattagtgaagttgtttaccacttcgtaacatagtggccgtttccataagacctagcgtagtatcacgcttattgatctgtttaccaagttcatcggtacgtttaccaatgtaaacgttaatagccatattcatagctgtagcgagatcttctttggttatctcttcatcgtctaaagctaatttagccaaagaatcaaatgtgctagagagtcctataagatcggaggtttctttggccaaaataggattaatattatctactgttacttctagcatggagttaacaatatcttctccgtctactagaccagattcaatattactctcccactccttcatctgctccaataatggatttttcttttctgtcatggtagaggtttcctgtgtcagttggtaagaaggatgattgagctttaggcggatgttgtttagcaatccagttaagaatcagtacacgactaggaacaatatcagtgttgtaccagaaatcatggacccattgacgagcatcctcaaacttaccaaatttatatgctttcttaaataattcaaacccaaaacaatctggtgtaaagtctacttcacgatattgaactactttagaaccagttaggtcttggatcgctgcattaatttctgtatgcgagaattttgtttcgtgaaacttgttaaagaatcggtagttgaagcgtccgttcgtttcgacaaataactggataaagagatcgttaaattggttatcaaaccagtaattctgtagttgttgtagtgctgtcatttacgaactccgttaattgccacgatgatgccttcgtacataaaaggaagtagcccagaagtgtagaagatattttccgttattcctagatgtcgaaaaagtatattaaataatgctgttagaaatacaatctgaggcagtaatacaaaatactcagttacaatctttggtgcctgataaacaagacgttttatcattgtttttaagtctccttattactttggcctgctccacaataataaaaactgcaagcactaatagtgctattagccccgcagtaattggtattaaatcaatcatgggagacgccatacgtaattagctgtctgtagaaccttaggtgtgtcatcatgatacattactacagactttggtgaataactatcagctagctctttacgaatcatcttacattcttcaccagtatcgatatttttaactatcactttcatattaagaggtgttaatacagaaagcggtttccaagagtcctcaatttcaattcctacctttttctcgattaccatgagaacttctagtgtatcatctggaatactaatattattattcacaaactgtaatatctcatgtaaggtatttaattcattaaaattaagcttcatctttaagaatatcctcaatcatatcacaggactgttgcaattcgtcagcaatgcgcttaacatctgtttttgttactttacctattaacatcaccagtaacatagaaagtataataagtggaactgggataacaaacagtgctaccttaacaacattctttggtgcccatgtatggtacttagtaactagaacccctaccacgattatataaaatgctagaataaacatcatcattttttaatctccgtgattatctgtgtacgcattactttagcaggtaatttattattcccaactattgcttctatattatttaatttttcttttagaatatctgagcgtctctgtgctagttcaatatcttgttcttgttctattaagcggcagtgaagtccactattagtagtaagcatactctctatattttttcgcatgcttacaataccaataatcccgccaatcgctactagtgtcataagcactagcaatccaatgagaatttcagttaacattaaatgcccctacatttttcgttatttggcgttactttgcaaatctctacgtttagctcatgaagagccttgtctacttcagttaagcgtatgttaagagaaaccatattatatattagtattcctataaggaatgacagtattgaaattgatactctttccattatttcttacctttcagttgcttaacttcacgctcaaaagatgctgcacgacgttcggcagtcgcttgatgctctttagcaaaacgtgcttcctgttttgcaaggttgaatgccatagcattcttattcagagaatcatacatctttttcattgccggaatagacatcttcatgagctgaccctcaaaaccctgcatgtcgaggagaattgccagaacgttagccatttcatttttagtcatgttttccattagaacttcctctctcattaatttatgaacttattatacagaattttaaggcttaaagcaactgaattttttaattacttaccaatcattaacaacgccggagaactcagaagcaaaaactttttcaaatttctctgtaatttcaggatgatctttgcagatcagctcaatgcatgcttctggagtcattccttcatcttcaggatcaccatagtatttatcccaaagatcatcggggatgatgtgctccgccaaatcttggaaagagattagctgataacgttctgcttcctcggggataaggatttcacaaagatctttatctatatcgctacgataatccgcaattaatagggcattgcaagcatgacactcccaaaccatcaagcgttcagaaataggattagacttatcctgaacgtaagactgaaacagggttcggaaagttgccttaacttgttcaaacttatctgctgctgcgaaaactagagctttatcaactttgttcattttgattccttctcatcaatttataaagtaattatacagaaacttaagcattttagcaaataaaacttttagttaaaatgcctaatcctgtgccaatagcaatacaaatgattataagtattgctactgtttgtatgattcgtttaataatagcctcagctatatacttagacatttatatctccacgcaatacggcagctacatcttcccctatttgtaatacttcgccggtagtacaattaactactctatcatagctagtaaacttaccagatacaatatcctccagtgggattggagttttactcatttcaaatgagacattaatataatgtggatcacgtagagaagtagtatgtacatgtccgtgaatatttagtttgccacgaagttcattagggtgcaaagggcagtgagatagccaaaactttttgccgcctccagtctgtttacctagagcctggatatcgtcataaacttccgctaaatccctgatgctcagtccttgacgttccgcatcatgattacccataataagttgattagggacattgtggataaccgactttaatttggttagaccttcacgacctactgcaatatctcctagcaaaataagttttgttcgtttaccaatatgcttagacaaagtatctagaatatactcatcgtgctctttcatgttcttaaaccacggacggaaacgtaacgctccagggtggtctagatgcaagtctgaccagaatttaactttcattactcttttccttattctgtattaaactatcaacccaagaggttaacactttggttttaaactcctcgggagtctctccttccttaagatctacctcattaaggtaggtagtaagccttgtttctatctctatttcagcaggagaattaaaacaaaaattgactgctattccagaatatattactccggtgtctcctttgacgaaagcattaactaatgccctccaacgaagggcatcaatacgcattgctcgtaattcttcttgtacttctttcattctatcattcattaaaggatcctctgtacaccgcagctatcatttttgtctgtggtaaatgtggtgattttgccagtttcatcgttaacctgcgcaaccgtcaactgcccgttgaaaacaccgccagtatctaggtaaacacggttctggtataaaatcgggtaaggtactccggtatgcccatgaaatacaaagtctactccattaaccggaggtacttctaccatgtatttctctttaaaccctgcataacgctggaaatgtgagtgttcttcaccatttttagctaagtgaaaacctatttcttggattacatctcgatcccataagtatggctcaacatggtatccaggatggtcatgtgggtcttccttagctgcttctacttgtgcaataaggctatcccatactggggtttctacttcgttaccacattctttgtacgtaaaaggtacaccaccatgaacaacaccatactttttaccacgatgcaatacagtcagaaatactggcattttttcagccatgtcttcagcaatatttttaatggtatctgcatctagttcattcatggcccacataccaccgttatacatccagttagcccaatctcccacaatcataaattggtcatgattaccacggacactgataaagcgtggattatacaggaatttggctaaaacttgtaggttctgcgtgcctcgatcaattaaatctcccacgcatacaacgcagtctttcttgccattatagccagctaattgtagtgcatcttctagtaaatcgttgcatccatgaatatcaccaacaaagaataagttaacatcatcaggtactactagtgttttatgcacattaaattcttttttcataattattcccaaataacagttaaaatagttgtgccatactcattagataactggagagtataaccctctttattgagggcttttatagcttcataactaatttcgtcacgattaagctgtacaaaggaacctccaatctctacagaagatactatcctccgcttaacccatctatacgttttctttcggctcttaaggagtgcagctctatttattctagctaaacgttttgcttgttctgctaacatttaatcctcctatcaatttatataagtattataccaaaatttttagctgaaagcaagtaaaataaaagcctgatccgctaagatcaggcttggtttattaatcctctggattatcatccaggaaatttttaatagcttcgtgcatagtttctttacgaagctgacgtccaatcaaaatatgattagcttctactcgaccattaggtaagcgatccgctagaactactacaggaacttggcgtacaccaaatttctgcattaattctgtattatcagtctctttatggacttccaaattatagtcattgactactttatcaaatactggctcgaaaagtttacacgggttacaagtagaacctttcaatagatatactaatttactcatagttattaatggcctctaagaagtttttcatagtttttacggttgcggtgctcagatcaagatttacacccaaacactcggttaatgcttcctgatagggagcctttaatctgccttccgggacttctaccttcttatattctttatttttgacctttttaatcatagtctctagtacaagaagtggtaatttagcaccttcaatatcagtacccaaaagttcagttaaaattttattaagatcaagacgagttaactttttgcctttagacataggagcacctacctttgtaccaatagatttttccgctttcttttcgagctttttgtactcaggagagtcatgatcacggagtgcaatgtttagctcgtaatgtggtatacgaaaatgttccgccatttgagtatagaaacgatcagacataatatatacctctcttaactaatttatgtatatattatataataactagggctaaaagtcaactacttttttatcttaatctactgcatgtataaatcttttcctaactagatacatagttattgggtaaattgagtacataagtatcggggagaaaagggtgaaatacgctgtaactcccccagctaacatcaaaggtattaatgctagagtaagggccataagaatcatgccgaatgaaacagccaaagctatggatataggattctctagttccatccataaccaacaatatttcgtatctctatggcaattgcatttacaaacgccgaatgaacccataattatatactctagtccgtgaataacctttattaataagctatttcgggtagttaatgcaatgatagtaaagactatcattaaaacgaatactactagacttattttccagatacctagcagcatcatagtgaatagttctgcaaacatggctttctcctatcttcaaaaattattaaagccccctttgcctaaaaaggcgggagtattcccgcctaagttattaattaaactagttcgacatgaccgccatcataggtaccacgttttatttcatcgtgatagtctcccgaagcattccaatcagcaccaaaacgaagcttaatgcctagttctttaccagcttgttcaaaagcctttttaactgcccaaaatgcttctaagtcattccaatcaattttgccattaatgtatggagcaaaatcaagagcatccccagtaatatgcttacttttactaggatcctttaaaaatgaagtaccattagcaatattctgggcactttgtgctactgtacgaataccttgtacgatcgtaaaatcatatggagataattctaaagctctacgagctactttttgtagctctggcttaacggtagctaattgtttttcgctatttttaccaaatttaaaactcatattgatctacctaaagatctctgttgtggggaacagagctttgttaaatactcgttaaatttctcagaatcggctgcatctacaggattatctttccaagcaataccaatatatccagcatagatattgtttagattgaaatacggacaagtgtatatgtaattaaaagtaacgttcttaaacgcaggtatatttaaccccatgtatttatttacttttacacttaaatccgatgcgtagttaaagccttccaagtgacgtctatatagctcagacgttttatttaccgctttatcggccaagtcagccctatctaattgcgcattgctttcccatgcaataatgttagaataatcattaatggcatcgggtttatacttgacgacaaaaacagcatccgcacccgtttgagaaaagagtaccatgctcttttctctagctacattaggaaagttatctatcctttgagtttgaacatcctgtagaaccgcagacgtggagaaagtctttaagaatgacatcacctcgctagtattactaacaaataagaaaataatgacagcaactaagatagttagcagacgtttaagaagcgatgctggatctttcgcttcttggagcagtaccgttaataattgtaagaacttttccaattgtaacctcctctaaccagtaccattatactaattttggaggaaaatttcaatgggtttttaaatgttacacttggagcacaaaattatgtcgttgtcaagaagaattttagataaaagaaaagccagggtacttaaccctggcttaaaattacttactcttcgttgtgctcttctgattgaggtaactgggctttattatattgctcagatcgatattttgccaagatttcaatacgcttacggatttccttgctatctagatagatatttttgttgtcttttaccatctcgtcgatttcatctattgagaagaacggatagtagacatctagcatcatatcttctacaaacttgttgtaagattcataggctttcttgatttcttgacctttctcacagaacccgccagagaacgtgtgtcccattagcgtagcatgtggatgtactacccatccatgacaggataggaaaattgtgcagtaagcagaagcactaggaccaatcaggtgtcctataactgttccacggcaattagcaattaagttagataactgtgcagcagtatcaacataaccacccggaccattaatcatcagattaatttcgtcgtcctcatttgcctgcataagtaccattgataaatcacggtattcatccggcgggcctaattcttcatcaaagaagaaggtatactcgttagattggcgaatggagtgaaacagattagtcttttcttttttattagccatagcgatccttgattatccaactagtggttctagctctaagtcacagaagttataaacttctagtttaaggttattgataccatcgaatttattaactaattcgtgagtaatatattttgcaggaataccactttcagatggatgatctgatttaatagtgaacttctgctcagcgggtactacatttactatcaaagaatccatatctaaatggtttgtttctctgagtaactctccttcattggggaatctaacatctgttattacggcgacatctgggtcgtctttagcgatggattgctccagaattatgagccaaatgcgttcatgcaccagctgccttcctagctctgtccctacgagctgtaacatttttctcggagaaataaataaactataaagtccgtcttctttattctctgagataagttgttgaggattaagatatttttcctcaaaaataggccaaacgtacgagaagtcttcaaacttatcgataccgtacttaaaccacacatctctggctcgctccagttgagattgtgtgaccgtaaaccattggtcaatctcttttcccctgcgctctcctaaaaactctggagttacaccgaggattacggatgcaagttcataaacgggcttagcaaaactataacgacgggataaacacgttggatacgtgtcattacaccaatcgataattaattttgcaacggtatcttttcccgaaccagcttcaccatgtaaaccgactaatactgacattaatctttcctattgttaattggtggatagtaaactaagcaacgtttttcttgtaaatcttggcgatagaagcaaaccataccagtcagtcgtgaaatgtaagagtaaactgcttcctcacattttgcttttgcattccaatctttatacggtacaacaactactacgccatcttctccatttgtagctaatttcttttcaaacttaaagtcaaagctacaaggttgaagtcctttaggccagtctacatgagcttgctttaactcatgagatggctctggttttatttcttctgcacagcctgtgattgtgattgtggctgcgagcagtactccaagtttccagttaaacacgctaatctttcctcgaactctttattctgctttttagcaattaatgttaccaatccaggctttgcagcgataacatcttggcgactaagagctttatctagctgcttaatctttttgtctagatctgcttgagatacattattcagtgtaaaatattgattcatgcgtgtctcacgacgttcactctcaacttttacattattcaaggaagtttctagagaagagatctttttagtagctaccgcaagatcacccgctaaagtctccactttatctactaagtgataagcaccgaacccgatgcctattgctagacctgctcccaggatacaccacttaccgtgccatattcctgaaagaagtttttgtatcaaatccatgcgctgctaacctgtctttatctgatactttaatcatatcaggctcaccagttcctagtacaccgaagatatactgttcgagaacttggtgtaaacggtataagttactttcaatttcctgccacggaataccggactgatggaaacgaatgttagtctgaattacgaagttacgaacatcatcgttatcgttctttttaatacctttcacatatgcaattggtacacgcagttcggataagatacgttctgctttaggttttgcttcagcgagcagatcacgcattgtttcacgaacatctccacgactcttatccattaagtaagagcgagcatgtaatcccatctcttccattttatcaaacatagtgtcgagattaaacgttttgaagctaaatgctggatgccagcagtacagctcgtgtaaccagtaaacttcacctgcaacaaacagtgggtttacaaagtattcgaagtcgccacggaaagatagtgaaccatcccgaaatacttggcgagtatgcacaatgttcttttctgcatcatcagttaccttaaccagtagctcttcagttttacgctcccaaacacgatcggggtcgaacaaactgttctgcaaatcatacacatcggcgaacttagaaataatcctcatatttaatcctcttgcctcaatctataaacatattatacaaaagtttgggcatctaagcaactaaaattttattataaaaatagcccagtcgcaaaagtgcgtgctgggctttgtcattatttgcttttgctagtaagttgacttagtgtcaatcttatgtaatattagtttctctagctttgcgtagtacccctgcatttctccataacttaatatcgtctactgtttctttataaggagctatggtaaaacctagtgggccagcactaaataaccctaagaatcctattgcacgcatatagaacaagcattcttcgcctgtcatttcttcaactgcttttctttgtttttcgttttcaaactcgtgctccttgttgtctatacgaaggtgtttaatgaaccattcagcagaatattcatctaagtgtctaagtatagctactgcatacccagcgccaattaaaaaccaagtggtaacaagaattagtattatagtcatataatctccagttaaggtcagcgaccaactcaccacttaccgcactaaggggcacgactcccttatccgtaacagttgcaggtgacgcaactcggtcctacgcgagttctttaggtattctgcatttcagctttaaattttttcatattacgagtctttctataaaagaaaaacacagatgttgggcctagaagaatatccacaataaatatcatagatctagcacatctgagtaatgttttatcatcaagaggaggcagatctaacggtcgagttttatgtataacgtataatattacatctttattcttagaaatatacagatcatcaaccaagctgtaaatagctactatagatccaatgacgaaccaaagagcaaatggagagataatcatttcagatccttttcattaattagaatattagtaaacagtggaataacccctagactcatagcaggtccaaacgtattcatagctacccacagctgcatagtgatcatcccatctttaatctctggatctttatattcttcgatacgttttttggcaaaggcaactacatctggattatttttattttcctgaatatagtcatgccaaaactgggtattctcactacgtaggaatgttaaaacatccttagttgctggaatctgcaccgtgctgttgatgtttagctttttcacgttcttctctcctacgtttgcattctttcatcatatgagctgctacaacttcatggtagtagtcattaatgttaccttctggcaagaaatgcttaatagtctctagctgacaaataactcgatgaagatggttatttggtagacacaattttatacctgcaacaaatccagagaagcctggaacactgaacatgggttgggcttccactctaccttcagagttgatattaagatcaattgagatacccattttcatgctataaagctggcaatattctccattaccagatcttgaccaaacagtgaaatcctcatcaatttgatcttggatttctgtaattgcaaatttaattctagggtctaaattattcatcttcaccttcaaagtcgcaatcataacgacaagtaaaagaactggagctattagtacgttgagtggttatttcaccgcgtttgctaatagtaacggctggagtagagaagtttttattagtatatatttctactttaccattacgttccttaacatcacgaactttaccatactcggtaataatgccgttctgttggactttaacattaccatttttcatagaaactttggtagatgccagagaaccgaaactaactgtggagatcatcagtgcaataataaacttattcatcttcatcctccccactacaatcacgacatacatcataatcgttgaaagaacgttcgtgagcttcacaccaccaagaacaagaatcgcagagaaacatttcggttttctctagtgcttgacctaaaagatcatcagcttcctcagcagtaatcttcaatccgcacacatctgccacacgttctttaattaaacgacctgcttcgtgataagacatacaggttcccacaacatcatgttcaagaacatgaacagcacagtctagacgagttttcatttcaaacctctctcatcaatttataaatatattatagcaagaaaagagaggtttagcaaatgaattatttcagaatttagtcatagcaccaccataatccacgcttacgcatgtcatgatcgcgattataatcataattttctggatctttcatcacgcggtggagttcttgacgattcccttgacgaaccattttattagaatgccacttaacattcttactaatagaattccagttataaccatcctttttctcattacgaatggttgcttccttcatttcctgttctacatcatcccaggatttagcataaataacacgaatccagtagccattttctttgatgaaagaatattcttgagagaattgttctttagacttccaccactgatcaccagattgtttacgataagtacggctcatattacctccattaagtcttttaaattaacctaatgtggatgaattttgttatacatatattatgtacctttatacagaaaaaccccagactacttattcaatagctggggtttgttattaattacagatcggggcgttcatcttcgaaacagcctgagctaattccttctccgaatcctacaaagccatcaaattcatcatttgagatgcccataatcattgcttccattttcctaacctccgaaattttcatgcctgctttactattcttataatatttgcgagggttcccgcacatccaacaagaacagatgcaaggagtagtagtgataatacctaaccgcttagggctttcttcatgtgggaagactgtccaatacttcttgcggttgttcttcacacgttgccggtgatggcgtcttaatgctcgatccacacatttctcctaaaacttgggagataaacggagtttgctcgactaccttgcgtagctgggagaaatcgtatccaaaccccattacatcgcctgagtaaatgtacgactaataacgtcgttctgttgttcacgagtcagtgcattaaatcgaacagcgtagccagagacacggatggtcaactgaggatatttttcaggatgtttctgcgcatccaaaagctgatcacggcttaacacattgacatttaggtgctgaccaccttcgatttgaggggcgacttccaccttaacttcacggaaggcttcgggatcgaattgtggtgtgataccttctagatcttcaactagaactttctgtttaagttcaccattgtcggagtatagctgaataatctggttttcatgaacaaggttaattacgccagacttaagattttgatatgctttcattagatctccctgatgttagaatttggctcagctctttattgtctatagtttaccacgatatacagaaactgtcttaccagtttggttcataacgtatgctacttcatcttcttttaagaagattgcttgatctattccagaaatagctatgcttttatttctgggattaaaccctacgcttttaccaacatgaatctgctcaccaccatcagtggacattattttaactgttaacataattgctcctatatttagctatctctagctcttttaatggtagttagtagtggatttgaaccactgtagtcgctccgtatgaaggaggtgcataaccactctgccaactaactgttatgttttactagactaagtaagtattctttatactctttagaccaattagcttttttaaactttcctggacgcatagtcttaattcctagatttggtactccgggagggatttaaacccccgatctctcggttatcagccgagtgctttagatcgctaagctaccgaagtattagaatatttacatcatacactgtctaagtaagaagtataatggaatttagcttctccagtcttatctacttctacctcagttcgtggtacttgacgaaccacaccatgaatcatcttatagcgaatgcctttagctggttcatgatcttttgggaatacttgtttaaccgttactactttatatccttcacgcggtgttaacactactacttgatcaccttctttgattttccaagaagatggaaattcataggtatatctttgaccaccagtttggaatactacagaaagataacgtttcatttggtttccctctcattaatttatatagatagtataacaagaaataagagagaaggcaaatgaatttttaaatttggtgctcccaccaggaatcgaacccggttcagatgcttacaaggcaactgcatcgccagcaatgctttaggagcgatgatcccgttatttcaacgggatttctttttcaataacaggagacttatcagtcataactgtcattgccatttgttgtagaaattcaaaggtgaaggctacacgttcttcaccaatctctacttctacacgtttcttttgtttatcgatcttgacttttaggttgtcagccatcttcgggttctcgttgctgcgctacttctagccagtctggtagaagaaactttgtacgaaattcttcgtaagtttctttgggataaaagctaaaacctcctttctcatcattcaggataacagaaccgtcagtttgttcgtatgctcggtatgtttccccaataacaataactcgtttatcagtacttttattacgaaactcaaaaacgtgcataatgttcccttaaagttggagcggctagagggaatcgaaccctactcagcgaggcttggaaggcctgcgacacaacccgtgtgcttaaccgcttattgtttggcaggggatattggatttgaaccaatgatctcgacttcaaaggccgaagctttagaccaggctaagctaatccccaactgtttggcagaaggtagtaggttcgaaccactgatcggggtttcagagaccccagccttaaccgacttggctaaccttcttcttaatactttttggatcttctaaacgtttgccttcatgatctactcgaataactccgcacccttcgcaaaccacaactacaccataaccttctttgacctcagcttctgtaatcaaaccagaaagatcaccagtatctctgccccacatatcaatggagcattgtttgcagaaatcagccatattttatttcctatttaagttaatttggggtggctagtggaattcgaatccacataggcctgattcacaatcaggatcattaaccaattatgatatagccacaattgaaaactcgcagggttagcgaaaccttcctcaattagcatgttctagaatactaatcaagttatctcctattgcacgcagctcaagacgagtacgaattttcaattgtggtggctctcagaaactcttctactctagccttagcagcttactcccgaacgcatgtcggggaagtcttactaagagcttcgaatagcaccgttacactttgagccatagatgtatcaagaagcagaaggacttgaaccttcacgagatagaccatttatctcagccgctttatgaggtatgttgcagggagtggatgttccctataaaccttttaggcagccccgtctaccgattccagcatacttcaagaatttttataaaccagcagaagcctcatttaaagcatcaacaatagcttcacgttgggtcattccatacccagtggaatctaggtgtggcaaataagccatccatcccttatctgtttcatgaaggcatgctttaaatttttttttttttttttttaccgtccttcattgttacagtaactgttttgctcataatgattccttatagttaattggaggaaggtaatggagtcgaaccatcaccgtatcactacagcgggctagttttcaagactagttgcgtaccactacgccctaccttccatttgtttggcggaagaggagagatttgaactctcaagccgttttaccagtcagccactttccaagcggttttcgtcgccaattcgatttgctcttccaataataagatgatgcctctatcacaccaggcagttacgcctcgctgggattcgaacccattaaaacagctcaaccgagatagtagatttaatttactctgaccatcaaatttggtgcgtgacaagggattcgaaccctcaaaacccgacttctaaggacggtatgtatgccaattccatcaatcacgcgtatttggtggacaaagagggactcgaaccctccctaccagattgcaaatctggtgtgctcccgctgacactattcgcccaaatttattaatccagcatgtacttctctatggcagttagagcataatagtacgcactttttaagttctgctattactacttctctgtttctacgagaagttctaaaccttgcaatggagtattccttttcttctggattagtatggtggaaatctaaggcttgtttacacctattatatccacaaatactacattctaatacaggaaatacttctttaattaattcttcatgtccagctactcgttcagctttttcacatgctttacatgttggcttccacttctttgtgccattaggagtaaatccattcgaataaaaatattcggttgttaacggatattcattcccacaggctctacatgtttttgtacacactgcgttaacccctgtaagtttactaaggctattagctcggcgtaactacagcaccttcgcttcgagcggaaggaactgcgtcatctcgcaaacccgatacactattcagttgccgctgaatctagtggactaatctagaacttctcccgaattgtagggatcgtgctttttggcagccagtcctaggtcgagctaataccttagtgaactaaattgatggataccagactaattttgctacgacgtttatcctgaggatctctttttcggtagctctgctagtgtagacgtcatacacctggtatctaggcagctattttcgcatcaatttatataaatattataacaagaaattaagcattaagcaagtacatttttagcgatttcctgtagttcctttacatcacgaacgtaaaggtaatcagagcttccagagtgcatactttgcataaccacaccttcatctggtgcattatccttcaaggtaattcctagccagaactcggcaaaactaggtgttatctcaccttgacttgccttgaatacatcatattctgtaagttcttgaccagtagataatcccaaatagacagaactaagatcaatctctttatgagactcataaaattccgatagatctttttccatatcccagccatgctgcaatgcgaactccattgtcacatgaccgaaataaccttgaatcttgtttgataattgcatttgcaattccttattttgagttaattggtactcggtgatagaatcgaactatcacaaccgccgtgtaaagacggggttctcccattaaactaaccgagcaatttggtagatcgcctgggattcgaacccagaactgacggattaaaggtccgctactcttatccagttgagctagcgatctattagtaaccattatatacctctttaatagagtgaactttcaagtaagggtgattagtattaaactctgccattacttcatcccaatttttaccataatgtgcaagtaaggatcttgaggtattattacctaaacgtttgtcacttactactaatcgaaatactttcatttcgaaatcctctattaaagtggaagcgggtgcaggaatcgaacctacctctttctagcttatgagactagcgatctctaccagagatctaacccgcaattgtttggtctccgtgggaggatttgaacccccgaccctatcgccccaaacgaaacgctctggccaaactgagctacacggagaatattatttcttcaaccacaacaaagataagcccagcatactaaagccgctaactaatccgatacatttttcttctatagtaaaagtatctagattaaatgctgttatagctattggcatacaccataatgtcataaaaacttgagctatcttgtgtttcattttgctttccttctcttcactttataaaatatattatacagataatatagcagaatagcaaatgaatttttaataaatttacttattgtgtaggccacctggaggctaccaaccccacggctgacaaagaagtttgcgagacctctcttaaatgccagaatagacccgccgatctatcggaacctacataataagtaaactttcgtttacttacgccccgttagagggactttagcgactagaaccggccggaaacttcatcgccttgtttattcagaagtccgttctctagggagcagaaatctaaatttggtggagtttgggagactacgtgttactccctctgtccgaagtaggatagtgcttaaccgtttagctgaacccctttaaatatttatacgttttatctaacaactcttgtttagagattataccatcacgatattcagtaataaaattatctatgttggagtcctttgatacaacgttatcagatagctctgtgaatttatttgctaaaagatctaattctttgtaattcacaaaacctccagaatatgccattctagacaggactcgaacctgctatcacttgtgcggacttacgagaaccattctttcactcgaatcgaacgagcttctgccctagccaagctggtttccccatcaaccacgctagaatgtttgattgttttcagtgcaagagtccgagtcttggacacgaccgcttcgcaagtacataggcagcttatcatgagtcagcacttccgaattgctagctccgtgttctgcactgagattttgttaatttaatagtaaagtacgatctacttcaccattaattgttcggcattcctctggaattgctggtgtagtcttatattcccaccattcagaaccatcatattcaccacgttctaaccaagttccatcagtaaaccatacgttaccataaagcaattgtccaccgaaaccagaatcatatttaaagtctagattatttagaaaatcttcaatatctttttctgtataacctacgggtaatacccaaacttctttgtctccataataaccgaattctatcttaatacacttaacttcagaattagtattctttagggcaagtaacaattcttctttagcattaatcatagcatatttctcatactaaattaatttggtacacccactaggacttgaacctagaaccctccgcttaagaggcggtagctctaaccattgagctatgggtgtattaaattacttttccagttcatcagctatggctttgattttttcaaagtttacatcactaataatgataccttccagtgacagagatattctagcatttttcatagcagattcttttatctgctctatttcttctggagtaaactccctacgttccatagctaacctaccttatttggcgcgaccgatgggatttgaacccacattccctcccgtgacaggagagtaccattaccaagccagccccggtcgcataattcatttactcagctttgagcttttcaatagctgcattaagttctttaagaaactcttttcgtacattaagacgattgagacttactagtatatcgtgtttaagttcttcgatgtatttcttttcaacttcaatattgttgatagtgctatcacgatgaacttctagctgcgcaattacattcttggacatgcttgtttcctcagttattaatgaaaatggtatctgtggctgaattcgatgtcagcaatgatagctggttggctaccggtttacttaactctacacagaatttggcggttcctgctggattcgaaccagcaaacctcaacttaacaagatgccgctctaaccaattagagctaaggaaccatataagtcagaactagagttaatacgttctagatatttactaagatccctagaatttatactaaaccactcatgttcttttaaatctttgaaatactggtgaattagtttttcaccttttcgtttgtcaaaccaaaacgaccaatgctctaatttataatccctatatggggaataggtctgatattgagctaatctctcttttggattttgactcatacctattttatactgatttttccatgcaggattactaatacagtatacaaaaccttcagaaataggaagtttattttcctgcttaaactttataaaggaaagtgcggcaagttttctcgaatacatagtattcgtattctttttggtattaccattagttcctgaaagatttatatattcttccataaatctttggattagttctgcggttatacagaaaggcccattagtaacaacgcctttctttaccaaactatcaaaaacttttgtatgcactacttctttcctttagtaaattggacgaccgcatgagatttgaactcacataaaaaggatttgcaatccttcgcctaagcccttcagccaacggtcggtttatcttgttctgtcatgttgcttctccagttcaatttatgtaattattatacagaacttttggaaactaagcaactacattttttaagaatttgatgcctttctttttcttcttatatttccacgttaatggattctgtcgattatgaacgcttgagatctttgtctcgtcaaagttcttcaatatcatgtaatcacaggataattgttctagtagaatgtgatcaccgtcaaaagcgtatacataaggctgttgactaacacgataatgatagactttgttaattctacgtttcagcgacttatcagcgcagtaaataaagaagttttcaggatgcatcagcagctccttcaatttggtgcaccctgttcgtatcgaacgaacctctcaagctcttcaggcaagcgctaatccatctcagctaaaggtgcaaattttggcaaacctactaggactcgaacctagaccgtacggttttggagaccgacatgctgtccagtaacactctaggaatgcagaaattggcgtcctgtgcggggatcgaacccgcaaatcctaccttgaaagggtagtgactttaccagtttgtctaacaggacttaaatttggtgttggatgatggagtcgaaccacccgagacccttccgagtcggcagatttacagtctgcttcgctacctctacgatactaatccaacatatttagaagtattcaaaaattctcatttaaacacttttaaatatgctttcaaattaggtaactattataccaagttttaaggcactgagcaagtcaaattttaaagaacttgctcagtgctatattagttagtcgaacttaggagtgacagttaaggagaacggaagttcgaaaacttcttcaccctcattactaacggtaactacacgacctttaagttcagacattaaaatatcaatatcagcttttgcatctggagccatgccagcttcaatacatttagctttaacctccggtagaagatctacgttatagagatagagccactcaatctcaccttcccagtcttcccaggatttgcagattgcactttcaataccagttgctacaacacgattttgcattcgttaactccttcatcaatttatacaactattatatcagaaatttaaagcaaaaggcaagtacatttttaaaaattctgtttggaggagctaaactccagattcttcagctaagtctaggctaataagagttatagccataattgctgcaccgtgttctttttctccggtgtttttagcagagtctataatagtcttaatctgttcgtaatagccttttacttcttgttcaatacctgcctcggctatcatgcctttagtcatgattcgttgcatagtaatttcacggttagccatgatgtttctccagcttcatttttaatagtttacaaatgaaatcaacgtcttcatctgtacctagatctctatcttcaagagttgccttgaagtttttagtcaattccatgaggataacttctctagaacgacgtgttggcggaggtggatgtctatacatcttatttccttagtttggtaggggtggtgggattcgaacccacaaaacctgaattttaagtccagtacctatgccaattcgattacacccccgtgtagtttagtcaattacttttgaggcttcaaggtagtcgatatagtgtatgttttctaccgtaatcatataatctgccactttatccatagcttgggcttcagaggtagctctggctaacctatatccttttttatattcatccgggtattctctatcttggataaacacaacataaactatatatagatccatttgtgacctctatttaatttggtggctccccgaggattcgaacctacgtaccttccggttatgagccgggtgctcataccatatgagcttgagaaccagaatttggcggaagaagcgggaatcgaacccacaaggctatatttcaaaccgacagattagcaatctgctgcaataccgttatgctattcttccgaatttataagatatattatattagaaatttagcttcatagcaagaaaatttttcaagttttcggtagacatctcatccacatctacgggagaaacaaagccgtttaattttctagcaaatttttcacccgcaggatcattatctccgacagctaccaatttaaaaggtagtaatttcagttgctttaataacgcttttcctggagaagaacccaacattgctagggctggataaccagcattatgtaacgcaactgccttaaatacagattcagcaatcaatactactttttcatctccattgagagtttctagtccccatacacactgggtagtagaataagtaaagtatttagcttcaaaggggcacttaccatgaccttttggagcagaaggattataagtttgatagcccctcaacatcccagagaagtcatacaacgggactgttagccatccttccggacttaaccagcaatgatatttatctgtatcaaatcctctagataagaggtgctgtgttagctcatcaacattcatatttggttcctctttcaaatttatagatatattatacagagaatttagcattcaagcaatcaaattttttaactttttagaggaaaatcatagggcacttaaggatagcagataagtggtgaggaccaaaatttgtgaccccactaaaaattcaggggcaatttttaaagaccccttgacattttcgctggggtagtgtaaagtatatgttatggatgagtgatcaaagagacacacattttctatatactaggaggtgtcaaaaatgacagaagaagtagctaagagtcctgagaccgtcgctagcgttgcagcagaactgcaccgtcacgaggaatcagataagaaaaagtgggagaaggtcagagcctacgaagaaaaacatcgtgaacacgatgatgaagaagatgatgatgaacattgtcataaatatggaaaacataaaatgcaagaaccagtaaacgtatttactagtggcttccctggcctactgggtggttacccagcaaacgcaggtgctttcggtggtgaaggtatgggactattcggagctatcctaatcggtgctctattaacaggcggattcggtggtttcggtggattcggtggacgtggtgtagttggtgatggtaccgcagttgatgcagtacttcagaaccaagataccgctagtattctagcagctattaacactactcgtacagaagctaaccagggtactggagctgttttaagcgcagttaacaatgggtttagccagacatcccaaaaccagagccgtgatactcagtctgtactaactacgatctatggtacaggtgctaatctgtctaatcagatctgtcaggaaactcgcaacgttattcagggtcagttcgatctttctcgccagattgatcaatctttcgcagctaatcagatagctcgtgcacaagataagtttgattctgcacagatcgctttccagcagactttacaaactaataaccagtttgccgctgttcaggctcagatgtgtgctaaccagaactctctggaacgtcagatcgctgaatgttgctgtgaacttcagactgcgatcgctggtgtaactactgcgcgtattcaggatgagttagatgcatgtcgcctacgtgaagcaatcgctgcgggtaacggtgctaacggtcaggtaattaatcaaatcgctgtaagtcttggtgctatcactcagactctggctggtctgcaggctaaactgccggcttaatattaaggggaggagcccattatgagtaaatatggttttgtaaataacggacttaatcaaagtatgggctcccagcagcccattccgggtctggataacccctacttacaacaattgcaacagcgtttagcagaagcgcaacagatgcagcaacaactccagcaaaaccctggaagtgtgagtcctatgcaaatgatgcagcaaatgagcgggcagtcgcaaatgcctcaacaaatgcagcaacaggtgccgcagcagcaagctcagcagcaaacccaacaaccgcaggtttctgcggaaggacaggcagtactagcccttttcgaagacttcgcaaagacagaggatggtaagcagcttgtatcacttatgggtaagtttaatagcttctgccaaagccaggttgcaaaagctcaaaatagtggtaataactcctaaggaggaattatgtgttgcagaaaatctgtttcgtgctgcccaatgcctgtagcacgttgttgctctccagcaatgtttacgcaaataccggcgttcaatccgtttgcctttaagcctactcttatcctgccccctagagtagattttcagtcaagattggcctcccgtatgggtggttgctgtaataagagtatatggttctgaagaaccaaacaataaagcccctagtcgaaagacttaggggcttttcttttatcttaaatacagaaaagccctgcgctctccaggaaacgcagggcttatttcagatgttacatgcgggtattacttattacgcatatccataatcatttggccatcatagccagtgccaaccacagtctgcggtacaccaccttggtatttctgagcacgaatcatttcaacttccagttgcttccagcgaatcatctcaggagtaatggtacgttgcaacgcggcgttagcttcagcttctttcttagctgcgtacagtttggcatctgcgtcacgctcattggcaatagcttggttattacgagcttcgcgatctgcttctgcttgtttaaccttctgctgtgcttcttgttcaacacgagccagttcagctttcgcagcattaacttgctcttcacgaactttggtattctgtacctgttccatgattaccggtggcaaagtaatatcctgaaggaatacctgcttaactgtataaccatatgggcgtgcatactcttcaacttcctgttgaattgcagtttgcaattgagcctgaattttagcatcaaacaaatcttgtgctttaggtacagacttaccaaactcacgaatagtagacagtaatttttcagttacatatttgtctagcgcttgatcctgagtacctgcgttaatacggttaatcggtgccttagaaccatcaaactgcaacataacagtcaggtcaacagtggatttaaacttatcctgactaggaacctgaagtttatctaattttagagcaatatctttagtactaaaagtatcgaaagaagcaaaaggatttacaatatgaaagccgggtagtactgggctagggtcaactttacccaggaaagtctgggttttaacagtaccgtcttgaacaacggtataagagttaaaggctagaatcaagcctgctaaaccaactatagcaccaataccccaacgtttaatagtacgaattactttctgctcagaagtcagttctttcatatttttagtattaaacatatatttcctttgttttagttacagttaataaaattggccgaggtgacaggattcgaacctgcataaaggaatttagaagactcctgcctctccattagactacaccccgtttaagtatttttaagaactctcgtaaaaactcttaaaaatacagacgataccgggagagtcataagtctcactcattaagtatttcaagacaaaatacagtaacgtctattattttccagtttaagtcaaggtgacttagtggttatgtcctgcggtatactgaggactcggttaggattggggtcatgacttcccgccttttgctgctactcaacaggagttatcgcgaattacctgtgctattgtgcaccattagtgacatctctcatgtcagcatttcccactttgtggatagcttgaaaaactactccgatatggagaaagaacttcactatcctagaataccttctaaagttcttctttagcctggacatagttaaatcgtaataaaactattcagcagactagttggttagtctggaaccaactaccgcgtggtttaggcagtgcgcaactaacctcggaagcttttacgttaaaagcatcagttatccagttcttaccgaccgtaggattcgaacctacatcttccaccagttacggcttgataataccaattatactaggacggcagtgttactacataagtgatttccacaccccactatttataaattagggaagagtttaggggagaggtttcagacgcctcattttatacgttctagccactaaccgggattgccgccccctgtgtactagttttacaactaacccaccgactctattcaatgcgctccggttgggattctgcactcagcgtaatcggctcgctgttccctaatttatgaatatattatacagaatttataagcatttagcaaatgaatttttagaaaaattcactagcgatcaaagcagttgttttaccgtcataacgtccaggaaattctgccttaatagttttcattaatagagctttggtaatacctggatttaatttaaccagttctgcaaaatactcacgaatctgaacttcagttaactgctccagagcatattcatgctctagacctgctaacaagtcgagatattcagcatctggagtatattcgatatttgcttttgcagcagaagtcattgtattctcaattgctttgaactgtgatttaacccagttagtaaactctagtctagatacgcaatctttatcaattcgttgtaagtcgcctaaaatcgtactaaggtgcttgactgacacagtatcaccgtgtttgcgagctttttcaattaattctcgcagcacacgaatcatattcgtacccattatttcatccccaatgcttggttaaaagattgttctgtcataactgctgagactttggcgaaatcaaactttttagggttcgtagtgatttcagcaacgttagaagttttaacagttttcgtagtacgaataccacgagtgttaacaccaataattacaactgatttcatatttcgcttcctctcattaatttatgaaagtattatacagaaatttaagtagttaagcaagtaaatttttaagttttagtgacaacaggtttataaaacgtatgtcccttaatcttagttgttttcttaaacttctttgtccagtaaggtttatctttaccactatggaagtatagtgctccatgtgtaggatcttgtggtaaatcaacgtagtatatcactctagctaaattcttagcggtttgccaagcctcatcagttttatcaacttttggtctcttagatacccaagagaactggttagactgatatactactttacagtaggaatctgggaatttaccggagtttacacggtttttagtaacatatgcaatcgccgtcattccctttatgccctcaccacgactttcgaaatagatatttttagcaatgcaatctatttcttttgcatcgtgcgatgcctgactactaaagctgaaggtaagggcagctactaaaagcaaagctgccttcattctttttactcctgccaatcatattcaacacgattgggattttctttataccctaagtcaagcaaatcattacgcatattttgcttagctactgggttcgaggttactagtaccactttctttggtctctgtccaaattctaagagtaaacgaagaatttgataaccttccataccatctacagccaagtcattatccatataaacgaaatggcctgtggtatcagttttattcagaaactctacagcggcttctggggttcgaatgataatgtccattccgtgaagattgcgcacatcatcaatcaaaatatgaaaagtcataatagcctcgtttcatcaatttaaaaatatattatacacataatttagtaaattagcaaatacatttttaaattgtgaaactagtaaaccgataaacagcctagaggcgccacaggccatatcttatgagccatgtacctttgatctagggtttgaatcatcaacgaatactttgcatcttttagctttagaagtttagaagtttgagttctttaaacttttcagctttcatacttaaatgttgatctttgaagttgtgtcggtaatatcaacgacgctttgtttttacgtgtcagattaaatgtctgatgtaataaacataatctccaatagattatactttatagtatggtcagcggtcttatttaacctcgtatttcaaccgctgctgcgaggtaagatgcttgggctttcgtctaccgatttactagcttttaaattattactctacttcaatgaaggtagaggcgttagactcggacagaacaaagtcaacatttgttgcaaagtcctgatattcctcgtcatgctgcttaatcagcttctccagttccagcggatcaatcaagaatggagtattcttgagttccagcaggttgagctgcattttaacctcatcttcggaagttttcttatcgcgggtagccatgctatcctgcaaacgctcatatttagtatcaaactgtgctttctgcgtattaaacttaaccgtagcggcgtggaactgcttacgcatgttagccagcagttgagctttaagttccatagaacgcttagcttcaattgcttccgcaacagtcatcttacgagaaccaatctcaactactgtttccgcattgctcttaatcagagcagctttgatacgatcacgctgggacatcatatccaggagagattggaaatctgccttaatacgagcagatagatcgtctacttcgattgcagcgccaactacaaccttatttttatccttaccttcaccaacagcgatcagaagctgttgttcagtagctttacggattttagcttccagagatttgatagttgccagagcgcgagttttagagatacgagtagtcattatttgcttccttatatgaagtttgagatttgaatttgtttaaatttggtctattacagaccaattgccagcagagtgcgcagaacagtatcgttactgttatcaattttaggattagcacgatttagcagatcaaagattttctggatacctgattgacgttcagccatacgacggatacccagattacgagtagctttcttaccatttttacgaacatttttagatactggcatatttgttttccttttattgttttctcaaatttatgaatatattatacagaattttggagccggaagcaaatcaaattttcattttttcgatcatagctgcaacacaatagctctcaactaaggcatcaaatgctgggtcgtgcttaaccggatagatcccataacgaatggtgtccatcttctccattttagcttttgcccagctaataccttcagcttctagatctttgtaactataaccagcttgcatccagagattacgaagagatcgaacattgcccaaattccagaatttccacggaactacctcttcattggttccagcatggaacgtgtttgccgagtagatagtcatatcaaactctgggccattgccgtaatacaaagcattttcgcctagagccaactccataatattttgagccatagagaaggcagcatagttagagccaagtttattagtgcaatgatgtttaccattttggaaggcaatcagctttggattacgcccgttcatagcttccataatgtggatagcagacgggctatttttagcttgatccatccagaatgctagagtagatgctgtaactttggcaccagaattaagttgatcttgtacatccaaagtaacaaatactagatcagggtctttatcaattccatgcatagccacaaaagcgaatgaaggcatcgcgatatgagtagtaccacaatctcctggagttcctagagattcaatatctagcataccaaatggttttactttcataacttatcctttattgtgtagaaaactattaaagagctgtctctttaatagactcccacttcttggggtctttaaaaccaggaagcatatcctcactgacatcccagttctcttccatagaaaagtcaggccattcattccagctaccttccagctcttcataacaggtatccattatctcatgactagaaagtcgtgtataatccctagcagacttatgagtgctagcacggttaaaatcattctttgctacaaaattacgcatagtttctccaaataagaggggctttcgccccctgatttaaatcacttgcccagcagtttaaggatattttccataatagtttgggcagacttttcactcttgttggtttcagccaactttaaagcgagttcttcctgctgtttctgaatatccgcagcaattttttgatcagcctcaatacgagcttgaagctcatcagtgatagtggtaaaggaagcaacgatttcagtaacggagcgtggtttcttagagaacataaaaaatatccttgattagttagagaattagtagattgtaggggcttctgcccctatttatctattacttcgctttacgcttgcgagctggtttagcagatttacgagcagaatcacccgtgaacttaatgcctacaccgtaacacagagcacgaacgttatctttgctatactctacaccgaagatatttggcttaacagaaccaaactgtccacgagcatcacgcagtacatctgccgttggtttgcgtttctggttaactgatttctcgtaatctttcatggccatgccattacgagggaacaattctgctccagcattgaaactcagtgttactggaacagatttgtgaccataaccttgtacataggtcacaaggcgggctacagaacgagtagcgttcttagccttcggcttttgacgaatcggggccggacgattcagtgcctgtgcagcttgagcatcagtagggtgtttgcgcaggtggcgttccagacgagctttgcggttagcctcagtctgagggaaagttttaactgccggagctgcttgagtttttgcgtttttctgcttagccattatttgttttcctttttgtttatcaatttatgaagtatattatacggtaatttgaagcgtttagcaaatgaatttttaatttattaactgtaagcttcttttaatttcagaccttacctgcatcaaaagaactcctaaagtattgtccccgatgcctcggcaaacaccccaatatgtatctccccaggtatttccttcgatcaaagttgcattttttgttgctaacagtttgtctctcaaatcagggattgcaaactttaaccgtaaaagctctagcattacatcatcttttaccttatcccagtcagagcgtagagttaatttacgtcctctacgtttggcaactcctgcatttagggagcgcagtacatgagctacatcatctttttggtcagatttcatagcctgaaaagcatgttctacagtatcagcatgccatatttcatttagatattcgaaagtaattggtgataaataaaagttagataagaaactataagtacctttaaattcagtaatcttcattttttacgccccgcatctttagcctcctcggctgtatagatatactgataagcccctttggagtaggctggagcagtacacattttcttacgctcaatctcttcttgagcttttctttcgcgttcagctaattcttcgtccagataaactaaacgttgttctggaggaggtggaagacgtgtttctggctcaccaatattagcctttcttatttgacgaggtgattgtgcacgttttctcaatggtttaaatcctagtgaatttgagttataaatccgcataaagccctcctcactttcaatacgaatattatactaaattagagagctttaagcaagagaatttttaatttatttattacatctcagccatttcagcttcagtaataactgtgttacacagtgcatctacagcttcattaccactaataccagaatgtcctttaaccttaataaaggtaggattagtattatgaaagttgatatactgctgtgttaatttaaatgcttcctgccaaagttctagattaagaggaacttcaccatctgctttcttccagcctttcctttgccaagaaaacatccaactttccataccatttttgcaataagcagaatcagtatagataacaattggtctattatcttttttaactgaccaacgtagagcctctacaatagccgtgagttccatctcattatttgtagttttaggactatatccagatttagaacctaaacgatcatcgttatcatcatagacaataaatccccaagcacctggacctgggttagacttacaagcaccatctgtgtaaatgtggaaaaccgacattatttgctccagtcaatttcaagccaaaaaccttcacgctgctcatacttagcttctacgacatagcctagtttagtgagtcgttgcataacgctgtttctggcgttttctgaatattcagttaatccctgaatatggcagttaaattctttttctttgattgctttatcaatctctacggcaataagtgactctagattaagtacttcctgtttaataacagcaatagtattttgttttgcttctaaagcagataaaagaatagacattagttttcgccttctataattaccctagtcgctggaggaattggcggcataggaggaatacatggttgacctctctgtgcatcatttagtactcttggcggttcaggtctaattgcctttaattccttttctaggaattgttctaacgcccgttctacaaaatgatcgaaaggtaagtgataatactgagacagagctaagaacctacgttcttgactttgtgttaaataaactttctttttaaatagtcccatagttgtctccgatttataaaatatattatatctaaatttcactcagtaagcaaatgaaatttaaatataatagccccgaagggctatttgagttaagaagacatcttagctttaagagctgggtgaggatcgtacccctctaaatggaatgaccaagctgcagtattattctccagtaaatcgtagagatctacaaattctggcattactagagttggtaaagaacgaggtttacgttttacctgttcttctacctgttccatatggtcattatagatatgagtatcaccgccactaaagataaggtagcgaggttttaatccagtcagttttgccaggatatgtgttaataaagcataactagcaatattgaaaggtaaaccgaggaaacaatccacagagcgttgtgtccatttaagatctaggtatccatcgttacttacatagcactggaagccgtagtgacaaggtggtaatgccattttatcaagttcagctacgttccacgcagaaactagatgtctgcggccatgaggattattcttgagtccttcaataagttctagaatctgatctcgcccaccaaagttacgccactgtttaccatataccggtcctagattacccttatcataccccatacttatagcttggtcttcataatttggagtccagatggtccactgtccttcgtcacgaccaaatgtgaatacacgtagatcatggagatttgtggaaccagacaagaaccataagagttctcctactacggatttccaagctagacgtttagtggttacagctgggaatccctcacgtagatcgaatttagcgtaagaatcaaaaattgaaactgtgcctgttcctgtacggtcattacggggttcacctagtaaaattacatctgtcaaaatttttaagtattgttgcattaataagtaaccaattcctgtacaaatcgcaggctgttatcactgtttgcgccaactgcatagactaatcgagtggattcatagttataatccagtatcatactatctaggtgtgtatctgcctctagcttctgttcgctaaagattgtagatacaaaagcgctttcaatatgcccatgttggtacatttctagaagaagattagccccacctagaactactgtttgttgatcttttagaaaagtaggtaatgtatcacctatcattgatatgggagtgtaaatatcatgtgtaatatcgtctggcagtggacgatcagcacgaataaataggtcagaagctccaatcatacgttctctaacggcgtatggaagagctaaataagtacccgcaccaatgataatattatctggattcaacacgtccagttgagaatagaaagcatctagttcttctttgaaggaaccccatggaagtttaccgcggaggccgaactccccattcggcccaactgcatacattgctgtaatcataagaaatctccaaattcattacccagatctttcagagtatcagtagaggtctcagttacctgacctacacggtaggctgcaccaatctggatttcttgcggagcaggctgtagagaagtagtatccaaccagttcatgatccacggaatagggtttttggtgattactggtaagtgactaggccattctacaccaatgtgcataaatgcattacgccccacaaagtacagatattcttctagaagttctgcgttcaagccaatcagactacgacctttgaaaatgaaatgaccccattcaatttcagttttcagagttttcaggagctgtgctggtgctttagccactgcttcatctaccagatctttatcaaactgctggaacataatctggatgattgctttagacatctgggtgtgcagagcctcatctttagcgattaattgcagatttttagcaataccttgcagaatatcgttctctgctaatgcaaaggtacatgcaaaggatgcgtaaaactgcattgcttccagaccatagatagcaaagtaagcgtctaatagtttagcctgagtttcacgtttaacttcagggaactccgtttctggatatgtattgtcgccacgatgatcgcgtactgcgatgaaatactgacctaactggtacagttcatcgaatagttcaacagaatcagcgatgcgtgcgaatgcttcttggtttttggttacagaatcaataaattctgctgggtcagtcagcacattacgaataatattgctataagcacgactatgcaggtcttcaaaatagctccactgtttgagcataccttccaattctggacgacttacaagtggcataatagctgcttcaggggcacgactgataaaggaatcagtttgagtctgccatgctaggttcaggagtgtaatttcctgaatttcccgtgggaggttaggccattgtttcttatctgcttccagactgatttcagtttcagtccaaaattgagagcgttgcagaagtgccagacgttctagttctggatgtgctacacgtacataatctgcgataccgagagaatctcccaggaaaaggtcagcattagtatgctcccaattcaggtttagtaaagtagtcattgattttcctctttctcactatttcaggaatattatagcaaatctggtagcgattagcaagtaaaatttcaaagaaaaaggagacccgaagatctccttatttttattaaatagtacaaccaccggatgcacaaccggctgcttcttctccagcacctccaccatttgcggtattgaagttagcataataccaagttttccacccatacttaacagcagtcatgaaatcacgaactactactggaccaggaatgatttcattctcaaacttggtgtagtcatagtacatattcgtactgatagactgactgaagaatttctgcatagttgctacccatttgatccactcgatacgatctacatcataagccagtttatagctcattaacgtttcccaatcggttgcacccggagcaattgcaattactttattcactgcactacccttgatagatacaatcttacgaggcggttcaatagagttagtaacacctaacagaacagaagaactttctcccggcatctgagcagtcagaacagagttacgcataccgtatttcagaatatctgcacgcagagactcccaatccatttctagacctacggataccagttcatcaacagttttcttgtacgtgtctattaccagaattcctttagatggtttagtacgatcaaaccactcacatgcaccctgttctttagctaggcgtacagaggctttatgcaggaagtaagacagtttttcagcctcacggtgaatccagttacgggcttcaataccttcataagccagaccttctgctgccattgcacctgctgcgttcatcaggccaataccaacattacgacgtttcttagccgtatattccatagttgggaacggataatcctgaatttcaataattgtatcaacgaatttcagaaggatgtagcaagttttttcccactcagccagagattccatgcgccccagtacaacaccacctaagttacacagagatacttcaccaatgtcctccggcttcatttgatcaagctgctcttttgtcttatacagctctgtaatgtgatggaatgggcgtgtaggctgagtaatctcaacacaaaggtttgtcatacggatcggatcaaggaaattaccgtgacgattagattccccgatatgatgagcatacatacgtcctgtttccattcggatacgtagccaagtatctaggatttcttttgcagagactttaggagctgcaggaactgtttttccttcgtggtcgatctttgtaagagatgccacacgtttctccgctgccacgtagatttcttcaaatttagcctcgtcatcactataaaacgcttcatgaacttctggagcatagaagtatgacattagcgtaatatcttcatttttcagataacgttttaacaaaagattgttgaaactcagagaataatccatcttatcgattttattctcatctgtagcacgttgttgcttaacctgcatcaattgaatgatttcagggtcgaagtacggataagacactgtagcagaaccaccacgagtttgctgagtattggcctttactgagcggtcaatgtgtcgataatatggcagttttccggaatgcgggaatgccccatttcgcaccggatcagcaattgatcggctttcaagatgatacccgattcccgctctggctgcgaccattttgaagacgatgtgctcggcggtgtcgattgaatccaacgtgtcagtggaatccacgaggcagcaactagcaaatccacggtcactagagcgcagaccaaccagtggcggcgtaggaacgttgattttgtggagtgacattgcgttgtagaggtcgattgcatctagaattgtccagttaggttgagatagcatcgccattgccatacccatgtaggcaaactgcggggtttcataaatttcaccagtggctatattacgacgggaatatttatcaaagaactgtttcagcccaccactagtaaaaagacggtcacgatcatggtcgataacctgattcagagcttcaaattgctcatcggtaatccatgcactcatgtcttcccatgcacctacgctaaccatgtggtcatggaagaaacgtagagaaggtggttcgaaagagtcataaaggcgcttacgcatttgcgcaagacgtaattcctttgctggcacatcgtatgctgggttatctttgataagactttcagcagctttaattaatgcgtccatcagagtgtcggaatctacaacccctttaggtagagttttttgagcagccatagtaatggctgaccaacttacatcaactgttttgcaaccatactccgcccaaccattgagtttttcaggagcaaagtcttctacagtaccgtcacgtttaattactttttcaatgcgatggctcatttggctttcctttttatatttatcaggatttaaggaatattctactttattattggtcattaggcaaataaaattttctgatagaaaaggcccgggtattttcatacccaggccctgtatattaccagtctagctttttagcttattcaattactttagctctagccacttcttttaatagaatatctatggaggtgctcagctcgttataattgaatggtttatttggatttgaggttacaattttccacgctagggttgtcttaggactttcttccccttccttacgtgtaatcatattggcagcactcagtgaagtacgatgtattttagttttacggctcagatgaacgacgtttcccatattacaccttccccatgtctttaaagatgtcatagaatgctttagactcaacagaacgaacgttatcaatcaattcaatcattgagaatggggcattttcgtatttctcatagacttccataaggagtgctagacctgagtcacctttaatgtctttctgagctatatctccgcagataattaaacggcagtcttgccctacacgagttagaagacaaatcattgcctcaacggaaatattctgagcttcatcgacaattacataggagttgttgaaagttctgccacgagcatgttcgacagccaaaaatttaattttctgacgttcgacatatcccttataggcgtgatcgccgatggcccatttcataccatcagctattggctctaaccaaggctctagcttttccgctagatcaccaggaagcataccaagagacttacctagaggttcattaggacgtacgagaatcacttgctcaatatctgagtgaatgtctacgagttcctgagctgcaagaacggacggaataaaggttttaccagtacctggctcaccgatacccacagttactgtgttgttcttgatcatattgatatatgatttttgctcgcggtttttacctactagggatttagcaactgggttagcgtaatcattagaaaaatcagcctgaattacgttattctcatatttgttgccacgctttcttgaaccattgcggttctctcttttttgacgtgcttttcccataaataacactcctatagtgtacttcgctaacagaggatcaacaaaaagatcttgtttatcaacttacaaatatattataccaatatatacagtaaaaagcaactacaattttaaataagcctatgatattgcatttaaataaaaaatttgcttgcttttaacgtgagatgtgtgatactagataatcattttatatcgcgtgcacgagtgcgtgattatacaatttttaatagaaattggcaaataaaattttttgactcagatcatgttgcagaactttatagtaaaatattgttgacatccgccctcgaatgagttataatagtagtcttagttagagaaggagagtgtaatgaatcgtaatgaagaactaaaatataaagaaatgcaggcgttaattagaaactgcaaagagattatcgaagggtcagcggatgaagagctgctgtttaataatgccaataaaccctcagaacgtttccctacacagagggatatgcttgcaggtgaattgagtaagtatctgattctagaagaaatgccaattcagattcgaaatgctcatatgattggggatattcatttccatgatgctgattatgctgcattaggcatgactaattgttgtctggtagctctggaagatatgttgaagaatggtatgaaggttggtaatgctgaaattagcactccaaactctatcactactgcagcggctattactgctcagattatcactcaagttagctcccatcagtatgggggcacttccgtggatcgccttgatgaagtattagctccttatgttcgtaaatcctatgacaaaaatcatgcgtttgcaaaacgttggactaaggatgaagccaaagcatcagtaatggctacggagatgactgaaaaagaagtctatgacgcttgtcagggtttagaatatgaaatcaataccatgttcaactctaatggtcagagtccatttattacctttggttttggtctaggaacttcttgggaagcacgtctagtacagaaagcaatattgaaagtacgcatggatggtttaggtatcaataaacgtacagctatcttcccgaaactggtatttgtacttcgtgatggcgtaaatatgaaaccaggggatgttagttatgacatcaagaagttagctatgaaatgtactgctgaacgtatgtatccagattatatcagctatgacaaggttgtagaagttactggagactataaagtttctatgggctgccgttcattcctagctgctacagaagatggtgaaactgctggtcgtaataacttaggtgttgtttctgtaaacttgcctagaattgctatcgaagctgaaggaaatattgatctattctttgatttgcttgagctacgtgtggatacagcgttaaaagcattggagtggagagttgatcgcttaaaatacatccaagctaaagctgcacccatcctctatatgtcaggagcctttgggttacgcttggagcctgatgaatatgtattcgatcatttctataaccgtgcttctgtatctctaggatatattggttgccatgaaatgctacagttcatgtttggcaaagatgtagatacaatgtccgagacctgcatcaagtttgttcagcgtgtgctgcagtacatgcgagatagagtcgatcagaagaaggaggagactaagttaggttatagcctgtatgctacaccgtcggaatccctatgcgatcgtttttgtcgtctagaccgtgaatatttcccggagcatgaagatattctgtccaaaggatactacactaactctcaccaccttgatgtggaacgtaaagttgctcccaacgtgaaatttgattatgaatctaactttactccgattgcttctggtgggtgtatttcctatgtagaactaccggatatgaaacgattcccagatgcactagaatgggttattaattatgctgctagtaaagttcattacttcggtattaatactccggtggattcttgtggtaaatgtggattcttaggtgaaacagtagcatccgaagatgggtttaagtgccctatctgtggtaatcatgaccccgaaactcttgaagtaactcgtcgagtttgtgggtatcttggaaatccaggtgctcgtccatttaacccaggcaaacagcatgaggttatgggcagagtgaaacatcaggatattcgagctaaataagctaatcagccgggttgctagtcgacctggcttttattttatttgctttcagccaaaaatttatatataatagatttataaatttgagaggagaaagatatggaattaaagcactatatagttgaatctacatgtggtagtttccatctgggtgtagatgatgttgctgaggctgtctttgctgccacaggtgattactcgcatatagagggtgttggataccctaccattattggcgattgggtgggaacaaatctagatgtatcaccttataacccatattgcttgtttgatgaagacagagatgaagtaaaatgtaatcttgaatttgtgtataatagagtgttaaaagtataccaacgtagccccaatgggtacgaagatattacttggagtattaaacagtatcttaaagatgtgtactctagtttttctgacgaagacctagaaacagttgcagtggagtttaataatgactaagaatgtgaaacaccttaaatatcgcttaatttacaacggacgtatggaaacagaagatctggcccagttagctattaattctaatgaggttacggtgcgtgcgctggcaattgaaaagttgcgtgcttcttatgaaaatcgtgttatggaactagaggagaaactttatggcattctcgaagccgatgctaccagataagggtttaaactccgctttggaaaacgcattaatgcagcactgggaaactggctataaggatggtgttgaatcctgcgctactatgctagaagttattgctaataccttaatggagaaaggcaataaagaattagctgagctattagacagtattgggcaaaacttcagagaacaggtgaaaaatgcgtagattaattattattagcggtgcaggtttgagtgttgaaagtggtgtacgagcttttcgtactgatactgctagtggtaaagcactgtgggatgattatgatctagaagaagtatgtaatattcatgcctttcgtggtaatttttatcataaaacccatatgttttacaacaagcgtcgtgaagagctaaaaactgttgaacctaaccttgcgcatcttcgtattggtgagtggtataaacaatacccaggtcaagtagtaaacttgactaccaatgttgatgaccttattgaacgtgctggcgttccacatagtgacattctgcacattcacggatatttaaaagaggttgttgtagctgacagctacaatagtagtaataaacgaattattgatgttgggtataactctattgacccagatgattataaatgggttaaacctaatgtaatcttctttggtgagcatgctccagcatatacagaaatgtacaatgtttttgatggtattaccagtcaagatatgatcattgtggttggttgctctaaccaagtaattaacttctactgggaattatttcctgttcttaatttgactgctgccaaattaatggtagttaactactatgatagtgccctagcgacagagccgggctaccaaggtatgactcgtagtgagatgtaccagctggaagaacgcggtatcccttactggagtaaaggggctgtagatgccttcagtgaccctgattttattgctcgtgtagaagctcatctagagggtagaccttatatgccggggaaacaaatatgtccataattaaatctgtgtatacacaccctgatgatatatttgtatgggcggacggtagttggtgttatcgctgtgagttgtgggaaatggactataaatccgatgactatggtgttatatatgtggatacggttgagtatgatacgttcttagaaaggaacaaaaatggaaataagtaatactccaattatttttctagacattgatggggtgctcaattcgagcatctcccaccatcatgcacctgatgatgaaaagattttctttggaagtgactgggttttcaaaccacttctcaaggcttttcaggactttattcgtccttctccgataatgattgttggagtatcctcatggttctctgtcaggaatgagatggagaacgttcagattatgaccggattaggtcttatcgatcgttttcttggtacaacggattttaccgggggtggattatctaggggtaattctgttcttcgcttcgttgaaaagcataagctcaaacattggtgtgtgctggatgatgccggtgctatgatgtatcagtatccaacggttatagtcaatggtagaacaggaataaatcttcaggatttaaaagctgttagctatatgctagaatttagcccagatcttgaaatgtgtaaatccttacaacaatttaaggtgtaaatatgttcaataatgtattctctaaagaagctaacccaattttggttaacttttggcgtactctcccggctagcctgtataatgaggctattaactccttgaaaatatggtgtgagaataacgaaatctcctttgcttttaaagatgatattgatgaggccccctgtattggtctagttgtgcaggtagaggagggtcttgaagagattgtgggctggaaagagttagacgaaatgggcttagtttttgcgttaaattataagctgtttatgcctgctaagcatcgcctagttgttaactataaaactagtgaatctcctggttttcaggttaatgaacgttatggttggtcttactcaccggaagaagtaaatgatggtatccaaaaacttcgtcgttttggttatcaaattccgggtttaaccgcctaagggggcataatggaacaagtatattggcgtcagattgatcctagtttggtgaagcgtgcggagaagctgctacaacgttggttggtggtaagacaagtctccttcacttttccggagcagcaaacggatgatccatgcattggcgttttcctgtctggatatgccggggaatgggaattggattggaatgaattgtctgctatgggcttgattgttgccctaaacttctctatattccatccacgtggtttggccatatgtcgtgtgcctgatgacggttgttcgccccatcttttacaggtggaagatgatatttgggagtacactccagatattctcgcagaagctaaagaacaattaaatcgtgttgggatttatgttccgggcttaaatgatggatgatttttattagctaaactctgattaccgagtcattttggctcggttttatccgtacttaagaccaatgatagcaccatccggaaaattttacttgcatgatgcccaaaatcttgatataatatttatattgaattgagagacattagtaaaaattgattttgctaatttttcttgtttacaatccgcgatacggaacagttaggctgccccattaagggttgttttaactattactagtgtttatattgcattagttaatcttcaattaagtgatctgtgtaccctgaattcggaaaggtaaatccaaaccgtgcatatttagctcgttatataggcagagttttatatgggggtgcgaacgaaaagagcagcgaatggcggcacgtacagtagtcacctaccaataggatagtgaggacaaatagaatcggcttagcggccaatgacctaggttctattctgtatatgagcgaagagagcacagcactgagatatgataacttattcatattgcttgatgataattaatgtttacgtaaatagtagtaatagttgttctattctttctgaatagtcctaaagaaaagatttcgattaaccaacacaggagtattgttgtgttttctattctccaaggtcacgccggattttctcgtgatttagcaacaggtatttggcgagaaatcaaagcggaagattatacctttgccaagcgattctcgaaaaagcatccagaaggtaaaccagcttctatgcccttcaaatttgatgtaatagaagagcatgacccgcaaagccttgccgaaatgttaccacttatgagacgtttaacgtctgaccctcatatcgttgcggtacgaggtcgatgtcttgcacctaaaaataacgtgcgacgtaagaaaggtaactttaatgtatctaatcctagtaatattattgctatggacgtggatggtatcctagataccggtgggtatgacaagtttaaccttgttggtatggcccgccacattattaagatgttgaatagtattagtgaggacatgttccctcttgatgcaggctttatagctcatgcatcgtcttcggctgggcttaaaccaggtatccgaatgcacttaatgctagaatctaacgtcaaggtaactcagggtcagttaaagttcttatttacatctatcaacgatagtagtaagcaaaaatttggttttgatattgccgacttagcttattattcatctgtccagctccactattttgccgaccccttatttagtgatggtattgttgatccgtttaaagcggagagtaagccacgtctggtgtatgttaaaggttcgaaagtaaatttgcccaataatctggttgactatgaaacaactagaggggagtttaaggaagagttttattctttacttgatcaaattaaaggcaaaaagatcgcgtctgataaagtagaagaaaccatcagcgaattggaagaagctgaggatggggtgtacctgcgtattatccccaaactataccatagagcattagaggatggtgttgattttgcatggctagaacgtgagattaaacccgctttatctgaatatattgcaactaaagataacagtcgtaatattcaagattactttaataacggtcgtaagcaggctctcaaagcgtttgttaataattctaaacgtgagattccattaaatctgaaaggtgttccgctcaagaagttagaagtggattctccaccagaagttccgtacctgaagattaacattgtgcctccaaaaggccatataacatttgttaaggcaagtcttggtactggtaaaactacggcagtaactaaatggctggacgcaggtgttcttcctggcaactttttagcagttacaaatactagagcactggtatcttcaaacgctaagaaatttagcgcaggacaatatgataagtctgtagatatgctcaactttaaacgtggagctattgatcgtatgtccacaactattcactctttgcataagttcaaaagttttattggtcaaatagataccatttttattgatgaatgtgatgctgtaatgaatgatctattattcgcacctgttgttaagcaacgtcgcgaatgtattcaagttctacgtgacatcctcatgacagctaaaactgtaattctatcagatggtgatattagtgccgaaactattgaagcatatggttctctaattgattttgataaaccagtagcattttataatcatcaccgtaagatgctgtcaaaagctcatgcttatgagttcccggatgagtccagtatttgggttgcacttcagacttctctagagatgggtgagaaatctatcctagtatctgattgtgggccggatgaactgaatgagaagggcatggcgttgcgtcgtaatacgggtgcattagttaaggaaatccactcaaactctacgtctgatgtagatattagacgtattctggattacacaactaatgagctaattgaccaacaaattgattgcttattatgtagtccatctgtaacgagtggtgttgactttaactattttgataacgtattcgttatcactagaactagcaaccaagcgccaaacatgcgtttccaagcaatcaggcgcgaccgtggtgctcagaatatctattattttattgataaatccactagtgggttctccgcaggttctgaacaatataatattgatgaaggttggctggagttagcacagcaattatacgcacgtcgcagagaactagaatctaggaactatactagtactttacgttattacttgcttgatcagggtgcaactattgatattttcagcgaaagctggggaactattgaaggtgctggtaaagaatacacagaagagcgaatcaaagctatcctacattcaactccggattactgtgcaccacgtcatgcggatgcgtatgaggctaaactacttcttgttcgctattatcatcttgagtctattaaagatgtaacagttgagcatgttgaacaatatatcaaagataaaccgaatgatcgagctgcattcttccataagatgcacgagatgttctgggaagatattaagaagtgttcaaatgtgaccattaaaccattcatagaggctctgaaaggtaagaagaaagacttcttccttaaaacaggtcagagtgctaacccaaaatatgctagaatgtatcttggtatgatgggtattggtaaggatatgaacactgaaaatattgtagactggtacagaacctattgtaaaatcgagtgtatgccaatcccatataagtttatgactgatgaggagaaacatatgcatgatgaagcaatggcagagctaggtgctagaaatgacagtgaagaataaaaaatgggaaacaaggaagttcccagtaaaagaacgtaaagttcctgtttatgatacttcggaaaagctatgtagggtggtaccacgtagattagctggtatccctaacatatccgagtggttacttaaacaacggaaaacaaacgtgtctcttcgcatcggctttgaattagataaagtctatgctgaacttgcttctttgctcaaggaatcgtaaaaatttatttgctttcaggaaaaattttctgtataatagattcataaatttgagagaggagtttaaatatggccggttctcgcagaaagaaacatatccatgaaatcccggatgaagtctttaaaaaagttatagagcatctggagaacggtggtactaagaaagcagcttgtgagatgctcggcgtatcatccaatccaactatggaaaggatgattgaagagtggcaagaccgccagattcaagttgccgaaatgaagaaaaagaaacgtggcacactcattgaaggtattgagttggccaacgttattgaacagtatttatctggtgattcttttgaagagattgccgatcgtaattatcgctctgtagcaatggttaaatctgttctggaacgctatggtgctctgcttcgtttgaacgatattgtagacccgttgaatcctcctattattcctgatgatgctgtagcagaagaatttgaagttggtgagcttgtttgggttcctggataccagtgtatcggtgaaatcaaaaaagcaatggataatcctgttggttgctaccgtgtatggcttctatcggaaggcaaacaacagaacgttcattatatgaattatgagctggcttctgttaaacacctggaaaagttaggagttgatgtaaaatctctggggtataaatggactcgggaagaagtcattacgttgattaacgaagctgttaaggccgccttgaaacttgataaagaaaaaggaaaacgccgtgagtagactaactgatttacttaaagatggggaagtaaaaacactatatcgtagaagctggggtaacgataataacggcttagtattactaactttagatgctgactcggatgtagctactcagcatacctgggtagccggtaactatccatatagcgaggttgtttcattaacagtatgcgaagctattgcagacgattgggaggttgccagcatcgaggatatgctcgctgttggcctggtggagtaactacttaaaaatttcagttgcttaatcctataattcttgatataatattctcatagtttgaaagaactattctgtttaattcttaattaaggaaatataaaatatgactactccaactcagtggactgatgaactgttcgaaaaaatgtcttccgaatacgttgctcgtatggagcaattcccagaggatgaacgtcctggtgttagcatggaaattgttagcgaaattgcccaggaaaatggtgtaactccgaacggttttcgtatgaagctaactaaagcaggtttgtacatcaagaaagctgctggctctgcttctaaatctagtgcatctactggagaaaaagcttctggtggttctcgcacttctaaagctaaagctcatgccgaccttcgttccgcattctctgatgctggcctagcaccagattttctggatgatgcaattattgacaaactgaccggtaaagcggctgctcatctggcagaagcaatccgagctatcactaagtaatttttagataaatccaccaacaaaaggaactatagctatgactaaagctgaaattattgcacagtgcgaaaaatttggtgagttttatcttcattacgagaaacttcgtcagaaaggaaccacgtaccttcagggtacgatggaatttgatccagcccaagataaatatctggctgagcgtattaaacgtgaacgtattcgtaaagcgaaagatgatgaaattcttgtcttctctcgtactaatgatagtttccgttttattcctgtcgagaaagtccgtcgtgtgactagcctccaatcagagttagatcgtgcttctccagtaggccgataagaaaccaattagcccctatatggggctttttgtgtatttgggatatgtaaatgagcgaacaggtaaatcaaaactatgaaggacacgttgatgaccagtccatcatactttgggagaaggagggggaacaagtaagattaacagtatctgaatttcgtgggaatctatacatgggtattcgttactggcttcttgatattaatgatgaatggtttccaacgaaatctggcttctcttttccttacaccctagaaacaacatcacagcttttctatgcgtttacacagattcttagcgaatctgaggttttacatgaagtacagaaacgagctgaagaactcaaagccaagaatgcctagttcttggctttttctttataaatgtatttgctttcaactcagttttgttatataatatttatataaattgataagaggatcaattatgaaagtaacacttccaaaaattcaagcaaagttaaatctagtatcgaatgctagaaataactatctagataactttggtggaatcaacgataaagcgtacaaagatgcgtggttgaaagaagcacaagagttagccgatctcattgctcagtataataaagaactacaggtagaaatgaatgactaatgtaaaagactttattaaacgttgccaagaagcatattatcaaggtatgtctttaatctctgatgaggagtatgattgcctggtaaaacgcttcccactagaagaagagatcgggcctaagggcgatattccacatctgtatcgtatgtattctttacagaaagtttattataatcgtggagataaacccccatttaacccattaggtcaggtagaaactgacaagttagatggttgtgcgatttctctactatatattaatggcgaatttgttcaagcactaactcgtggtaatggtattctagggaatgatgtcacaagcaacgtaagattgctgaacattcctaaaaagatttcccaaaaagtacctactcaaatcactggtgaagttctaattacaaaagaagtagaaaataaacgtaactttgcctctggtgctattaacctaaaagatagtgatgctttcgtacaacgtatcggggaaggtggtttaatcttcgttgcatacggtatccagtgttctgctgaatctgtaggtattacagaatcatatttaaaagatatgctttggctagagaatgaagacttcttaacagttgttaatgtgcgttccttctttagatggattccgacggacggtaaagttgttcgtattaatgacaacaacaaattcttccgagaaggctggactaataaattccctcgcggggcgttcgctattaaagaggatgaagaaggagagattacaactcttactaaagttgaatggcaggtaggggcttctggtaaagtaactccagttggttatttcgaaccggtaattattgatgatgctacaattgttaaagcgactcttaataacgttgattatattaactctctcgatctagagatcggttgccagattcgagtgattcgtgctggtggtgtaatcccgtgcatagtagagcgagtatactattaatacataaccctttgctacccatctaccatataggaattataaagtggttattgacattttcgccgcttaggtatatactattatcattcagttgagggatagaaagttatggcgagggtaagcaaagttagttaaaattgtagttgctaaatgcttaaatacttgctataatatttatataaattgataaggaagaaatttgatgaaaatcgaaattccaacacaatgtccctcttgtggttctaagctagatcttgtcaacggacaattattctgtcgtaataagtccaactgtccagcgcaatcaagcaagttaattgagaacttctgttcaaaaatgaagttaaagggctttggcccaaaaactattgagaagctggagctgacgaagatttcagaactattttacctaaccgaagaagatttggttagagccgtgggtagcaaggttgccgctaagttaattagcgaattaaatactaaagttcgcggagacattgactttggttcagttctcggttctttaggaatccctctaattggagaggttgcagcaaagaaattatcccaaaattgcactagctttcacgatgtgaaggctgacggcaaggctggagaaaattacaaagcctggctaaattccccacaaggcaaggatgttattaaactaccgtggaaattttcaactggtattaagggcactaaagctgacgtcatcattacagatgaattagttgcccagccaaatggaatcgcggtatgtattaccggatctttgcaagattttgcaaatcgaacggatgcaacaaactatttagaaggtctaggatatacggttaagaaatccgttaccaaagacgtcaaatacctaatctgtgaggatgaatcaaaacgttcttcctcatcttataagaaagccgaaacgaatgggatagaaatcctgtcgattaaagaactattggagaaaaataataatgtctaaactgaactggaacgtagaaggtgtaaccgagtctctgaaagcaaaagccactgctctgggtgtttctgtaatctctcaagaacaggtagctgctatcgctgctgaactggctgctgaaactggcaaagatgttactgctcgctctgttggctctaaactgcgtaaagaaggttttgaagtacagaaagctaacgaagtacagaaatccccgtggactcctgagcaggaagctgaactggttgatttcctgaacgctcatgctggtcagtatacctatgctgaaatcgctgctgctgtagctggtggtcagttcggtgctaagcaggtacagggtaagatcctgagtctggaaatgaccgcttctgttaaaccaactgaaaaagctgctgctgttcgttcctttactccggatgaagaaactgattttgttaatcaggttgttgctggtgctactattgaagctatcgctgctcacttcggccgcaatatcaagcagatccgtggtaaagctctgagcctgcttcgtgaaggtcgtattgctgctatgccggtacaggaaacttcaagtgctaaaactcgtgaagatctgttagaaggtctggatctggttaacatgactgttgctgagatcgctgagaaaaccggtaagtctgagcgtggtgttaaatctatgctgtctcgtcgtggtctggttgctaaggactatgatggtgccgctaagcgtgctaaactggatgcaaaagctgcggctgctgaataatctggtaaattaaacctaaggggagggggctatgctccttcccctttttgcatttagaaggcacgaaagcgtgcaaaggagaatgaccttgtttaacgtgcaagctgtagtgttgaagatgcttctagcttccgaacagaagcaaatagcattggaaacattctcaaaactgcataaagatcattttaatgacgctttctcctcaatttaccaagccgtccagaattattataaaaaatataacacaatgccgtccattgacgcacttatgcttgaagctaataggaacgcccgcctttctcaagctctagtcgtcttagctaacactcaaattccagaagtgagcatggaacaggcacttgaagtcctagaagccgaatatacacaggatttattcctgaagcttctagaaacagacgtgcttcaagatttaacaatgctggatcagggagaaattctcaaccgagttgcctcccttcacttaaaattagaagagaaagtaacgaatactggaaaagtattcaacgcagataacatgcgtattttccagagagaagaagataccaaactgaacttaatcgctcttggtatttgtaatgagttcgacgctcaaattggtttagctcgcacagaaacgttactgcttggtggttggcgtggtactggtaaatccattatctgttcaaacatacaggttcagcaatatttgaatggagatattgctccatacttctcaattgagatgaaagaacacgaagtatttagacgtaatctagccatgttagctggtgtatcagcactagcaatgcgtaataatactctagaaggtgcagctctgttgagattagccagaactagagccagaatgtttaatggtggtgaagagctttttgataacttcgttaaacagtacacgatggctaagatgagtgatttctacgatatggagagtaagctaatagagggatatgaactacatactcctatgattatcgtttatgaccctgaactgtccattacaacagttgacgtagaattaaataaattagttgctagatatggtgataaagttacagtagctttactggactatattaaccaaacccgacttccagattctaaaaccattgatatgtatgactggaaagaacagatggttgttagctcgtctttcaaatctatctgccagaaacataacgtggctggtgtagctccttaccagatcgatcaagatggtagaacacgtatgtcaaaggggattcttgattccgcagacatggctgctaatcttaatgctgcaaaagctgataatggtcaaggtgctattatgtttgacttcgttaagactcgttcttctgatagcgtgaagtttatgcctaagatgaactgggaaactctgcgaatggataatacaactaacctagcaatggaagatatatctcaaatggaagctgagttcgttatccctattgagaaggataagccggctcaacctaaacgtgctaagaaagacaaaactgaaaatttaacaggtgaacaggccagcgatatatgagtagaataacagaactacttgatctgaaaggcattgaatataaagacacaggtggggacatcctcatctgttgtctcaatccagatcatgacgataaacacccaagtttgcgtattgatcctgaaacaggtattatgcactgcttgagttgtggcttcggtaaaggtataccgagcatttaccattattttaatgagactcagtaccgacagtctccaagactatcacaggtacgtaaaaagatttcagagattaggaatggatcaacgaatcttgcaattcctgaatctgcttaccttttcgaaggtgatttccgaggtataagttctaagaccttgaagaaatattttgctttccaacaccaagcagactgggaaggcagaattgtattcccaatcacagatgctgttggacgcaatatcctatttttgggtcgttctataaacagttctgcccctcctaaatacttagtaaaaccaaaacaagtttcaccgccaatttttcctgtacgatataatactccagttcttattctagttgaaggcatctttgatatgctgaacctagaggataatggtatagactatgcttcttgctgctttggtacacatcagtttacttcggataacattgcagataagttcagtccttatattattgctggggtaaaagttgttgttatcttactagataatgatgcttctggtaataaagctgcacaagcattggctaagctaattcgtacaaaaacgcgtttaacacctgtagttgctaacttcctcctgcctgaaggcaaagatccaggtgatttaaacaaagatgaaattgatatgttagcaaaacgtattgaaattttagttgctgaatcgcttaaagatttggtataatatattggtaagttagagaagaaacactgaagttatacttacttaccaagaggagattaaatttgaaaatcgcagtagttgataaagctctaaacaacactcgttatgataaacatttccagctatacggcgaggaagttgatgtattccatatgtgtaacgagaagttatctggtcgtttgcttaaaaagcacattactattggaacaccagaaaacccatttgatccgaatgattatgattttgttatactagtaggtgctgaacctttcctgtacttcgcaggtaagaaaggcattggtgactatactggtaaacgtgtagagtataatggatatgctaactggattgcgagtatcagcccagcccagttacactttaagcctgaaatgaaaccagtctttgatgcaacagtagagaatatccacgatattatcaatggtcgtgagaagattgcaaaagctggtgattaccgtcctattactaaccctgacgaggcggaagaatatatcaagatggtgtataatatggttatcggacctgtagcattcgactccgaaacctcagcactatactgtcgagatggttatctgcttggcgtttctatgtctcaccaagagtatcagggtgtatatatcgattctgattgtctcacagaggttgcagtatattatctccagaaaattctggatagtgaaaaccacactattgtttttcacaacttgaagtttgatatgcacttttataagtaccatctgggacttacttttgataaagcacataaagaacgcaggctccatgatactatgttgcagcattatgttctagatgagcgtcgtggtactcatggcttgaaatctctagcaatgaagtataccgatatgggcgactatgactttgaactagataagttcaaggatgattactgcaaagcacataaaatcaagaaagaagatttcacatatgatttaattccgtttgatattatgtggccttacgctgcaaaagatacagatgctaccatacgtttgcacaacttcttcttaccaaaaattgagaagaatgaaaaactttgcagtctgtattatgatgttctaatgcctggttgtgtatttctccagcgtgttgaggatcgtggtgtacctatttctattgatcgtttaaaagaagctcagtaccagttaactcataatctgaataaagctcgtgagaagctgtatacttatccagaagtcgttcagctagagaaagatcagaacgaagcgtttaacccaaactctgttaagcagctacgtgttcttctgtttgattatgttggcttaaccccaacaggtaaattgacggatactggagcagactctacagatgcagaagctctgaacgaattgtctacgcagcatccaattgctaagactctactagagattcgtaagctgactaagctgatctctacttacgttgagaagattcttctgagcattgatgctgatggttgcattcgtactggcttccacgaacacatgactacttctggtcgtctgagttcttctggtaaactgaacctgcaacagttacctcgtgatgaatctattatcaagggttgtgtggttgcaccacctggataccgtgtaatcgcatgggacttaacaactgcggaagtttattatgctgctgttctatctggtgatagaaatatgcaacaggtatttatcaacatgagaaatgaacctgataaataccctgacttccactctaacatcgcacacatggtatttaagctccaatgcgaacctcgtgatgttaaaaagttattcccagctctgcgtcaagcagctaaagcaatcacctttggtattctgtatggttctggcccagctaaagtagctcattctgttaacgaagctcttctggaacaggctgctaagacaggcgaaccgtttgtagagtgtactgttgcagatgctaaagagtacattgaaacttacttcggtcagttcccgcagcttaagcgttggattgataagtgccacgatcagatcaagaatcatggatttatctatagtcactttggtcgtaaacgtcgtctgcataatatccactccgaagaccgtggtgttcagggtgaagaaatccgttctggatttaacgcaatcattcagtctgcttcttctgatagcctccttttaggtgcggtagatgcagataatgagatcatttctcttggcctagaacaagagatgaagattgttatgctggttcatgactccgtagttgctattgttcgtgaggatttgatcgaccaatacaatgaaatcctgattcgtaatattcagaaagaccgtggtatcagtatccctggctgtccgattggtattgattctgattctgaggctggaggttctcgtgactactcctgtggtaagatgaagaaacagcatccatcaatcgcttgtattgatgatgatgaatatactcgttatgtcaagggtgtattacttgatgcagagttcgagtataagaaactagctgcaatggataaagagcatccagatcatagcaagtacaaggatgataagtttattgctgtatgtaaagatctggataacgtgaaaaggattctcggtgcttaatttcaaattgcccgtctatgcgttacgggcatttgtatcaattgagcaagaaggtgattattccgtaattacaacaagatataataaatatgtgctagataacaggaagttgcctggcacgttttctcaacgtaggcttattctatttgagaaacggaaagaattaccatacaagctctatcctattaggggtagaatatcaatgttgtctcaattagtggggtcaaagcgtaaccaatttatagattctgatggaaatcttattaattggaagaaaactacgttctatgatgttgtcacctctaaagttttgcattcagctagaatttataatggaaaataccaatgctatgtggctaaagttccatatccatttgtattatcctatgttccagcttatataagctatattctggtaaataatagccctgttatttatcaagtccatcaagaggagcctgaaattcctagacttagaataaaattatgaaagttgttatatctaataaagcctatttcaagcccgatgatgaactttgggattattgtagtaagcaaaccacttatcacatagagacaatgactagtaaataccctattatgtacaagaatagtggtgttgttgccaaagagattaagtggattcctattacgcgtctggacttactagatgctaaaggagtaaaatacgaattagtagataaacgcacattagctccggtagctataccagaacctaagttcaagctgcgtgaagaagatcagcttccaatatatgaagaatgtgacgatacctgtattattaatggtaaacctggatttggtaaaactatcctagcccttgcacttgcgtataaatttggccagaaaactttggtaatctgtacaaatacatctattcgtgagatgtgggcagcagaagttcgtaaatggtttgggtttgaaccaggcataataggttctgggaaatataacattgatccaccaattgtggtcagtaatattcaaacagtgaataaacatgcgaataatctttctaaagtattcggtactgttatagttgatgaagttcaccattgtgtagctacaactttcactaacttcctagaaatatcctgtgctcgctataaaattggactatccggcacattaaaacggaaagacggtctacaagttatgttcaaagatttctttggatataagatatttagtccgccggttaataatactgttgcacctacaatccataggtactccgtacctgttgagttatcaggaaaccaaaacgtaccatgggcattacgtgctaacgatgtgtataatcatcctgaatatcgagaaactattataaacctagcacatttatatgtgaatatggggcataaagtactcattgtaagtgatagaacagagttaatccaaacagtactagaagctcttgcacagcgtggtgttacaacatatgaaattataggggcaacccacttggatgaccgattaaaaattcaagaagatatagcaaaaggtggtccttgtgtacttgcagcagctcaaagtatcttctctgaaggtatttcccttaatgagctatcttgtttaataatgggaagcctcattaataatgaatctctcattgaacagcttgcaggccgtgttcaacgtattgtcgatggtaaactcgaccctattgttgtagatctaattatgaaaggtggtactgggctaagacaggcttctgggcgtatggcagtatatcgtaataacgggtggaaaactattaccatgactcctgagaaagcagttcaattagctaaaattgcatttggcaatagctcataaatgatgtataatatatacataaatttgagagagaaagtttcggattgataagaaagtccgaagcagaaaaataaaaattttagttgctaaattctctcgaaatctagtataatatatacataaattcgaggagaaaacaaaaattaaattcttcgattatgaaaagctatacttactagctagaggaaattccgacctaattattaagctattcaaaagaatgcttacagagcctgatgctcatcaattattggtcggttcctcattcattttgaatgaatcaacaatagttgataatccaaataaattgtctaatagacaactggcagaatatctaggaattttaagtctacgaaattatgccgaatacaagtttacaaacgatcctagtttggacatacaatatgttccagtatggataccacgtttagtaatcgacactaacccactaatcgcaattaacaaatcgaaattaatctttaaagaggaaataaaatatggctaagtcttggggcgaaactactggcggttctaacgataaaatcgaattcctgaagttcaacaacggtatcactcgtgttcgtatcgtttctggtgttcttccacgttatgtctattggctgactaataaagagggtagcgtagctcctttcgaatgtcttcgtttcaaccgtgacaaagagagctttgttcgtggtaaagccgatccggttcatgagatgggcttctttgagaaagagctggataaagacggtaatcgtgttccgctgaaaccgaagaaaaactatatcgcttttgttattgaccgttctgataacaagctgaaagtaatggaagtcaaggctactattctgaaaggcatccagtctatcatgaagcagttgaacctggcaactccgtttgatattgatatttctatcgagaaaaaaggtaaaggtttcgatactgagtatgatgtacagcagattgctgctatgcagttccagattaagctgcaagatcctaacagtgcggaatctaagcaatatgctgcggatgtagatctgatcggtgaagctatgtgtgatgaagatggtgacatcattaagttcgaaaaagttccttctctggaacaaacctatccggttcctacctatgaagagcagaaagaagcaattcaagccttcatggaaggtcgtgagaataaagatgatgccaaatctggtaacagcaatgctggttcccagaaaggtattgaccaagaagctgcaagcgatctggacgactaataaatagaggggcttcggcccctttctttttaaccaatgagaatattatttagtgctgatcatcatatcaaactaggacaagataaagttccaaaggaatggcagaagcgtcgcttcctgatgctaggagaacggttaaatgatatattccataatcataactgtgatcttcatattgctggtggtgatatacttgatgttgccgacccgtcgtcagaagaaatagaactgcttgaacagttcatgtcaagacttgaccatccaggcaaaatctttactggaaatcatgaaatgttaactaaaaccatttcatgtctatatcattatgcgggggttattaataaagtaacgagtggtaagtgggaagtaattaccaaaccatatcgttcccctgaatttgatattgttccatatgatgagatccataaatccaaatggaaaccatctgaatcaaaactatgtttcacacatgttcgtggtgaaatccctccacatgtaaaaccagaaattgatctaactaagtacaactgttatgatactgtaattgctggcgatttacattcttatactaatagccagactatcggatctactagacttctctacccaggatccccattaactacatcgttccatagagaacgcacaaaaggtacaaatggttgctttatcattgataccgacacattaaaagtagaatggattgaactaggtgatttaccacaactgattcgtaaaacaatcggagctggtgaagagatggaacctagtgattatgatcgtgtagtctatgaagttactggtgacgttgttcaattaaagtctatcaaagactctgatttattagataagaagattaatcatcgagtcactaaagacgctaagttaaatcttgttgatcttgatatgttaggtgaacttgaactttacttccgtgaagtcgagaagctatctcaaggcgacattgatagaatcttggctagagctgcgaaatatgtcaaagattacaattaagacactaaaatttagtaacgttatgtcttatggtaaggacatcgtaattcatttcgataagaatccagttactcaactaattggtggcaatggactagggaaatccaccatcgctacggttattgaagaactattttataataagaactcacgcggcattaagaaggatgctctgttctcttggtcttctccgaagaaagagtatgatatgcacgcttacttctcgaaagatgaagatgagtacgaactgcataaagtagttaagtcaactgctaaagtaacccttattaagaatggagaagatattagtggacacacggcaacccaaacatacaagatgattgaagaaattatgggtggtgacttccaaacattcaccaaactgatttatcaatcggtgggttccaatctagattttctaaaagcaacggatgcaacacgtaaggcttttctggttaacttgttcaatcaagaacagtataaagaaatgtcagaaactattaaggctgaccgtaaagaaatagcaaataccttaaataatttgcagggccagatggctgtaattacgaaaatccttaatgggaaaaataatctaggtactttgcaggaacctgtagaagtaccggagtttgatgaagagccattagcacaagaacttactgaatcgaaaattaaggcggcattagccaagtctcaagaggctaatattactaaattgcgtaatttggacaaagctgtacaagttgctgaacaatctttcgagcctttcaaaaatttacccgcgcccactgaccaaaatgaagagatctcaagtgttacgcgtgacctaacgattgtgacctcacgtgcgggcgaagttaagaaacgttatcagaagttcaagcaagaggcttcaaatactgaatgtcctacctgtggtactcatctggatacaactgctgcgcaaaaagcaatggatatggctagaatagaatatgaccctctgttcaaagagaagcaatctcttgaagctaagctagaacagttgaagaaagaacaacttgagtacgttgcatatactagagcaaaggatgctttggataaggcagtagcagctagagacgaattcaaaaattcaatgagtgatacttcttttgaagaactcaatgtgcaaaacctacaggtgcaaatccgacaattagaacaggaaatagctgatggccgttctaaggttgcaattgccaaagagcataatgcaaatgttgaattagccaatgcaaaatataaagcgaaactagaacagattgagaaagctgaggcagaaatgtctgagattacttctaaactggatgaggtatcagaagctgttgctgatctcgatattctgatcgctgcattaaagaatctagtaggatataaactagagcatagtgtgaaagtatttgaagagcttatcaataaatatctttccattatgactggcggtaagttcgcacttggatttgaacttgatgaaacaaaattacaagtagtaattttcaatgatggaaaccgtaccagtatggagaactgctctactggtcagcaaagtcgaatcaacctagcaaccctgttagctattcgaatgctactaacatctattagtaaagttaatattaatcttctattccttgatgaagtaattagctttattgatactaaaggacttgacactcttgttgaactattaaatgaggaagaaagtctaaactctatcattgtttctcatgggcatacgcatcctttagctcataagattactgtcaaaaaagatgcagaaggattttcctacttagaataaaacatggctgtagatagtagagagaagggtaaacgtggagaatatcaagtaagagatattttacgcgaacgtacaggtcttgagtgggaacgtgttccaggctctggtgcttttggccagagccatggactgaagggcgacatctaccttccaccacaaagcggacacattagtaaatactgcttcgaggttaagtggtataaagatgataatatatcaagtaatttatttaatgttggtgaatccactctagagaagtggtggcagcagtgctcacgtgaaggtgagcagatgaactccaagcctgcattaatattcaaaaaagacagaggacagtggttaatagctttggatagctcagacccgatggttgacaacttaatgagtcgtacccatatggtgttaaataagaaagacatggaaatcgtaattggtttatttgagccgtggctacatcatgcatctgttgaggacttaattaaataatgagtaaatcctggggaaaatttattgaagaagaggaagctgaaatggcttcccgtcgtaacctaatgattgtcgatggaactaacttaggctttcgcttcaaacataataatagtaaaaaaccatttgcctcaagttatgtttccactattcaatctcttgcaaaatcttattctgctagaactacaatagttttaggagataaggggaagtctgtattccgcttagaacatctccccgagtataaagggaaccgagatgagaaatactcgcagcgtacggaagaggagaaagcgctagatgagcagttcttcgagtatttgagagatgctttcgaattatgtgaaactacattcccaactttcaccattcgtggtgtagaagcagatgatatggcggcttatattgttaaactcattgggcatctatatgatcatgtttggttaatctctacagacggtgactgggatactttactaacagataaagtttctcgcttctctttcacaacacgtcgtgaatatcatcttcgtgatatgtatgagcatcataatgttgacgatgtagagcagttcatctctctaaaggcaattatgggtgatttaggggataatatccgaggtgttgaaggtattggggcaaaacgcggatataatattattcgtgaatttggtaacgttctggatattattgatcagcttccactgcctggaaaacagaaatatatacagaatttaaatgcttcggaagaattgctttatcgaaacctaattctggttgatttacctacctactgtgtggatgctattgctgctgtaggtcaagacgtattagataagtttacaaaagatattttggagattgcagaacaatgattaaaattaaattaactcatccagattgtatgcctaagatcggttccgaagatgccgcaggtatggatctgcgagcattctttggtactaaccctgctgcagatttacgtgctattgcaccaggcaaatccttaatgattgacaccggtgtcgcggtggaaattccgcgaggctggttcggtttggtagttccacgtagttctttaggtaaacgccatctgatgattgcaaataccgcaggggtgatcgactcagactatcgtgggactattaagatgaacctgttcaactacggttctgaaattcaaactctggagaattttgaaagactttgtcagctagtagtagtaccacactactcaactcataattttgaaatcgttgacgaattaggggagactgatcgtggggaaggcggatttggaagttcgggaagcaaataattttactgatcctaatatgatcggtgttgatactagtatgtcaacttatcagattaaaactgctgaaccagaaacgttggtgctaaaaacagataaagaaggtaatgttactttctgggaccctgaaggttttgtagagctttgtactaatacggataatgagcagttaaaattactactaactatttataatcttggtgttatagaaggttgtagaagcaagtaatagaaaaaccccggtggacaaaatccaccggggtttctttgtactatgtatatgtactatgagttatctggcttaataagctcgctaacaagtttcttcagttcggttaattcttgctcaagtttgttaactttatcagcttgataacgaaatgcgcaagctagatctgccataatgacgttgttatcaagcgctaatgtgtcatccgctttgtcaacaacaatttgacgatctacatactcttcaccctcatcgttaacacctataacatctgcaaagatgtccccgccaggaattaattttacatattccggatctatttttagcaggtcttgggctattaagccgaaccgctgaatgtcgctgccgttgtatttaaacattgttggcagccactgcataactcggtcatagctttcttttccgtccgtatagatgatatcatgttttaagtctctgtcagaggaagcagccttttgaaaaacgtagttaccagtggaggaccatgttgttatatcatttgacgctggttgaaactggaagttgcagaaaaactcaccatcgccacgaagccttaacgagcaaaaaggccaattattattaccttgtgcaataagaccccatgttgcgcgaagtggatatccaccagtggattgtgtaccccatgaaaggccaggtgcccatccaccatcattgttaggaataatctcgccccaaacaagagcattaccccaatagttaggatcatcccatcccatttgtttggttttgccagtgtattgagcagaatgcgttctgccgtcagggtaaaacttgtgcatataggactgtatccctgcttgatacaactctattccaaaagattcaacgtgtgtgtcgcctcttctgattaaaccagcgatagcgtaatcgtcataccagttgtaacgacttacaccagtccatgtgtttattggtggattcatgattgaaaatagttgtgacgcattgcgttcattcccatctgcataaataccagccccaggagctaaggaaatacttttcgctcctgaaaaagtaccattttcattaaactgataatactgtgatgccccattatgaaatacgcctattgtatgctttcctacgcctacttgagtctcaaaataagactgagaagaaccaatatcttcgccttgtgtattctttaattttgaatgatatatacctgcatttgcagtagttgcattttccgtttcgttttcaacattaagacttaggaactttggtgcatgtttctcaccaagccctatattatttctgataccattcgcatccttagcacctgtaccaccaaattgaatacctagtggtatccattgattggttgtagcatcataaacgccccaatgatccccctcatctggaatggttagatacttgtttttatttggagaatataaaactgtttccccagaattctgaaacagcctatcaactcctaaattatatctagcaccttcaggagtatctgctcctgtaccaccagccgcagttgataatggaagagtttcccatgatattccgtcaccttttgtatctctctgaagtacaataagaccatcatctctaataacaaatctatataatctattatgtgagtatagaaaagtatgaaccccatcttgtacaagtgagtctataaggagtgtctgacgagaagcattgatatttgacagatctgagagattattttcttttagtaacgatttactaatgataccttcagcttttccagcagccacttctgcgcgtccggcatctgtaaatgcactttgtgcgctaccagcagcattgccagcttgttgtcctgcttcgatagcgctattacttgcttgtgcagctgaagcagcagcagcatcttttagcactgtagtttctgttactgcgctatctttaatactctgtgtatctatttttaattgctcaacatcagattttaaagccgttgtttcacttatagtgctgtctttaattagttgagtatctgtttttaattgctcaacatctgattttaagccttgaatagtagttactgattggctagctgaggctgcggaatgggctgcattagtctcactagtttttgcattagtctcactagttttagcatttgtttcactagttttagcatttgtttccgaggtttttgcagcagatgctgatgcgccggcttgtgcagagaatatctccgcctcatctctataacctttagaatccgaagcagaagcagcagcattagtttcactagattttgccttatcttcactagcttttgctgcaatagcagaagcagcagcattagtttcactagattttgccttatcttcactagcttttgcattagtctcactagtttttgcattagtctcactagttttagcatttgtttcactagcctttgccttatcttcactagtttttgcgttagtttctgatactttagctgctgcggctgattctgcagatctatcagctgatgcagcagattgagtagcagatgtagcggactgagcggcagaggcttcagaagctccagcagaaatagcagcttcctgttcagagttcttagcgttaagctccgaggttttagccgctatctcagaatcttttgcagcagcagcagacttagctgctgcctcaatagaagatactaattcacttgcagtaatagagctaatagaattacctagagttactgtatatctaggaaacttactagcagtagctttagtatcatcaatatttaatatttgttgtacaataattttagtagttactgccattatttagttaccccatcactgataaatactttaccctccattatacgaaaggagctaattgcagatcctataacatctctagtcataattatatcatagtatcctgcgaatcgctgacgagggttatatttatctctatctttagatgctttagcccctatattttccacagattctactggcaaacttatagctacagccccttgagcagcatcaataattttagtagtaaaagatgccaatatctccgcattctcatctagactattttttacagtacctcttagagaatatccagttaagttaactggggtatctgtagacgggtaagtactatcatcaacgtctaaaaactgcatcattaaaccatatggtacattttcgtctattactaagtctataactctattttcagtactcattatttaccttatatgaatatacccgttttgcaggtctatcttcattctgtaataggcttccggattatcagataatgcagcgtttctatcataaataacgttaacaccgatctgagataagaaggcattatttgctacaatcttactggcagttaccgtaccgttaactatcatatcaccatgcagtaccattgctggattaatccaaccggaaccattccattgacgagtaaacgcagattgcggagcattactattaaactcagtcaatacgtcatatctaactggtgggctaccaaagttattctggaagaaggtattagcttgcccatcatcccaaccaccaagacctggaatacctaaggaatacatgccaggacctctttgcccatctgcaccgatagtaccttcagtaccactccattgaactacatcagaccagctagtacccgcaagtagcttattagttactggatcaagcgtgcctgtagtcacatagatattttgagtcatatctgtgcgattaggtggggttttagaccaaccaggaggtggataatcttgttctagtggttttgctggaagtgcagttgctagcttataaacaaatactgtttgcttgccctgtatacctgtttggatatctacatcgccatttggtgttccaattactacaccactagaaatacgcagagtagtaccatcataacgaatccattgtgtagcgttaccaatgtctaatttagctttggcagaggtattatccatacccatccaaataccagagttattatcaccccaagatttaccttgagtgtaaattgctgggttatccttaccagataagttggtcattataaaattcgcagcgttaatatctctagtaattactttaccatcaacgttaacaacacggtttataggatcaaaagaaataggtgcttttccctcttcatctagcagaccaatgttaatagccccagtagctgcgtcgattaagaatgtctgtctccatatttctccatctttcatgctgcccttaataaaggcatagttaacatcaatacccgtctcattgacaaagctgttgtctagaggggtatcttcatttaatataaatgttacaggagctgattctgttatatcttgtttatttggtccccaggcaattgacgaaaccttaaaggtatgttcaactttccacgggaatgatataatcgttgcagctctagcagcaccaacgtttatcttttgagctttagtccagccagtcttagcgtattcttcagaatttatataagtaaccaagaactcacgaacattagcaccagctccgcgttcccaatcccactcaattctaacatcatagcgttctttaccatcagcaattcgtgcagctttaaaaacaatattgataggtgcagtaggaggaacaaaattataagctactgttaaaatattagggaattcatagtacccagaggaatcaactgtaacaccatcaggcatagttacttgaccggatattctgatcttataatctccgataggaactccaccaaatttaatagtagaacctaaagcccccatgtaatacttagtccaaggactgtcttcagtagctgtactttttaactcaatagtacaatagctcgcttcaccagttgtttctacgacaactatcggtgcacctaccccaacatccacaggttctgattcagatcttgctgcaacaattgttggcttctcttttgtcttaaagttggtttcattagatagattgataccaattttagcctctagtaattcagaatctataatagaatcataaaaagccccttgaatctcgtaagaggttgaaggggttagattattaatcattacgaaaaaagtatctataccggtataatcacgtctatctatactttctcccctctttaaccaaaaagatctaccaataacatcatagtcagtatatatagagtgctggacatacgccattgtatatccagtcattatactatttaagaccattttggctggtgcattattcgaaatcattaaatggatgcccactccacagactgttctccatcatctccttctgctctaatacgaaaatatagttttctattgattcctagagcatcactattatgaagagcaaaatctgccttattatacattaataaatagtcataagtatactgattttcaatacgtacacttcttagcattctattctgggaatcatagatttccagagtatagaatatactttctattatatcttcttcgggtatcctatcccaagctagttttacatctgggcctacaaattcagttacatccccagaagccgtattagttaccctaaaattagatactacgcttaggtttttagcagagttaagttctatagataatgttactggagagcttcttctaccgtttatatccacagcccttatttcaaatattgctaaacctgcgggctctccaattatctcttgtatcatgcgttcattgggatttgtttctagttgctgcacaatataaggttcagcatgaccggaatgcacgatagagtaataaactacgttattagttaagcttggaagccaagataactcaccattttttcctatagaaccaactaaaccacctggtgtcggggtatatttaaaatctctaggaggaagaacgttgttactaatatctggtatatcattaccactattatctacttgatcagagttgataaatacatcttccccatattcctgtagagtaacatttatctttccttctctagagttttctacttcatctactaaaaagtacttcttatcccatccgtaacggtcgtatgtaaatgcaatagcatcgttaggctcaataccaatgaattgatatggtaaagaaaatgagagggttctagaatacctagatttcttaagttctctgtctgcaaaacttctagcagtatagtagttagttatatttgcaaaagataattgtaattttttgtctaaatttttatcttgttctttatatttagagttgtaaaaggtaatagagttagttttccagctaagagctgggtctacgatggatgcttgaactgagttgaatttatttctaccagtagtatctgatagctctaaatcaccataagtatccagaaaattaatctctaatggagtattagaatatttttctacagtaatcctatattgaccagataagttgttaatagccccaccgtaggactctagcaaaccttgcacgtttttaaatactgattcagacgtatctagaatagtattcatttgtactatttgtctattttctgctaatggatcagtccacccaacatatctccagtatggctgccaggatacttggtaagattcatctataatatctaaaatagctgcttcctgtattaattgctgaagggggaactgatcaatggtaatattagcgccatatctatcagaggttaagtaatcaagtgtttgccaaataccatttaaactagtactattagcagttactctaccatcagaatgatagacttttacttttttaccttgaatttcagcactaacttcaggaatctcagtcctattttcattaatagtgaaacgcaccactgcgtatgcagtatctagtagtttatacctagcatcccagtactccggtccattgccattcatattctgaaggtagaacccacgttctttagctatatctactagtacttcagaagccgtttggtcaggttttccatgatacgtccaaatccttatatcaccattaccatcattatacttatattcctgaccatgcacagaaggactactagaagatattcctgatgctattctttgcatggtgtctccggctacttttttaacaccgaaacaggttctagcagagctatcattagagtccatacaaatcatagggttatccccaaaggaaaagtcaagaaaaccgtctatctctccttcagcaaaagcatatactacgtaaactatattaggattgtgtaattccgtatccgcaaaaataggtattccaggtattttctgtacaccataaactactggtagatacttagcagcaaggttaaagtctatatctacttctttagtaacagtctcgtaatactttttaagactataacttctagatagtccaaatagctttttcttagactttagcttgtatctttcttcctgtacctgatactttgctagaatagagatacttttattggcatgaaagaaaccgtagtcttcttggtactcaagtcttttagccccgttagatggaagtaattgtcctgctacaacttcaagccctctatgagaagcatcatcagtaaatctaccattaaccctatcaaaatcatagaattggttagaacaattccaagtaattgtggaggttccaactcccgaagtgctaatattatccttaatacccccaccagtaattctacctctaaagtataataaaggcccatttgtgtctgggtctacaggtaaaatagaaccatcttctgtaataattgcttggtgaattgatactaatctatctaaaaaagacacaccattctgcactaatttcagtacttcatcatgcgctgtacctgtaatagtaaaagataggctgccaatagataaatctctattttgtttatgtgtgctaatagacttaactttaccggcctggtacaaaataccattatatagtacatctctaaaatagtcagttaagtaaataaaagcagtactagtaccagtagagccgggtaattctagagaaattagactagcagtttttattctgctattattttttaagtagtttctagcactatctaatatttttttcatagactttcccgtaaatttaatgagatgttggagtatgttccgttattatttagtgtagatccaaaagcatcaccattcatgagttttgttctaaatagtattccattaaatactggcttctccgctccagtagtagttataaataaatcagggtataagtttatagaccatgtattacctgatttgttaaatgatgttattttatatacttttgggtggttagacagcttaaatagatcccccggttttggagtacctgtaagaagtccttttgtatccatagttatattagaacccttctgtccagcaggtatatttactagactagtattacctctaactctaaaagcttcgtattggggtaatataacatctatataaccaccagtccttttgtattctagaataaaagcatctagaacactatattcatctggaaataattctggataagaaatatttataccccagtattgagcagataccttaacttcgttaactttaccatttggtaactcatcacgaattactgggtcattatcaatcaggttaacactttcgaatcctaagcctgaaagttctggattcgtgtatgggtctggtaatctcatagttaatttctcctctttaataacaatattataatagtattagaaaattttatcaagaaatttttatttttccataaagaaaaggaggactcaaagtcctcctccagttttaagaatttcctaatgttttcagactagcaccgttctcattcagagctaattctactgcgtctcttagagcaccactattactagaagcaaactctctaaaacttgcagcatccatagcactaatattcaagatgataggtcttcctgaagttgagttagatgaggtttttagctcatcattaggcgtagctttcataggaaccataggggtaactacttctgtaccatgctcacccatttgatagctaaccccagggtacatattaccaccctcagcacgaggaacgaaagagttagcaccgcctatgcctttatcaccacggatataagataattcaccagcattagcagacatagacacatctatattcttctgacgctctcctaaggttaggtaactagttgtatccgctccagaatcagcaatacttgacataccagatgcagaagatgcttgtgctaatgccaatgcacccgctaaacctgccgcaaccattagtggaatagagaacggatatggaacagcggtggctgcctgcattacagctactgcagtttggatgataatctgcttcttagctgcgtcttgttgaatcttcagcttttcagcttccaacttcttcaacttagctttagatgcttctgatttaccatcacgtttctgctctgctgcaatagcctgatcaattgcactaacctgctggttggtactatactgtatcatagaagatacggtttgcataccagaagcaatcagggaagtagtatctaaagacccctgggagaactggatcatggcattagttagattacccatactttgagccacagcggttgcttcagagtttagctcagataatttagagattgcctggtcataagaagccattctattttgcatatcagcaaaatctttatcttctccagatagtccagttgtaggagtatatacagcacctacagaagatccaacagaagcttgcatctccgcatttctctgtgcccttaaacctgctagttgtgccctagtttcagctatctttttattatactccgcttgtgccgcagcattacctaatgttttctgctttaagtcttcatataattttagctgttcctgtaagcgctccgcatttcccattaatggtttagtagtaaattgtctttgctgttcttcctctctaccagcagccattgaagcaatctgatcttggcggaatttccagatttcacgttcaatttgggcttgagcacgtctagactgttccgcctccttctgtttatcagcttgttttgtataccattcgtacttctctttttcaatggtcaattcaagatttaactgtgctagacgatactgagtatctgtcatagtacggttatttaagagagctatctcacgatctacacccaggattttatctgtataatccttaaccttcttctgggcctccataccctgattggtaagtttaagtttctccagctgaattttttcaatttctttctcaacagctttatttccctggtttgtttgaagaacacggcgtaaagcagcttcttgctcttctaactgttggagttctaaacgcctaccggcttcctgtgctttttctttatccttagtttggttatatacatcagctatcttctgttgaactgctaactgatttttagtttcattaccagttagtttaacatactcagatagagcctgagatgcagttttcatttcagacagggtgttataacccagattaaggtttttaacatattcatcagcactcttaccagtacctagagatagagattctatgttttttatagcatctgctaaagtagcactagttttagcagcagtatctgtattctgagcaacgttctgtatgtcattagctaagtcactagccgttttcttagtctgattgtacgctttctgctgctcagttagtccttttaatacagtagagttaaaatcgtatacagcatcaggatcagtagtatctctctgtagattaatttgcttctgaaactctggactagcctttgcagcagcggccccagcagcttgatacgctgatttccaagcattgagattctctgtagaatcctctacttccttaccaagtctagttacttgtagacccatagctgcagtttctgctacaaattgcttattggcctccgaagcacctgctgctgcaagaccaacaggaataccttcaccaacttgacgtatagctaattttgtatagaaatctgcttgttccattcgtttatcgaactcagattttagcttattacgcttatcaagagattccttatacatatctagagctttattatacccttcttcagaagcggctaaacttaaagcaacagcaccaacgttggtaggatctatctgtctattagttttttcttgagctctagctgcttcagcagatacagaagcctgagaagtataaaatactgtgttgatagcatcaattacgggccctaagtatttagcagcagcctgctgaatctttctcagtgcggcatcagcattagcggcaaattgctcccacggagtagcacgtaaaacttcatctaagtagccaaaacgtttagtagactctgctataacagcattagcatacgcctgctgtttctggaaagtagtaaggctgttaatattatatgttatacctgtgtttgcagcatttaactgcttaacgtaatcagcataagcatcattaagacgaatagtaacaccaagttcatccagaagttcgatttcttgtttagatacacccttaattacacggttaagtgcatcagtcatatcaacgccaagaacagcagcagcacgacgagctactaaaccaaatttattaagttgttcggcgtcaaatccataagcggacgctgaggatgcttgcctcatagcttcttcaaaagagattgcatagccagctgcctcttgtagtgatctagcaagggactgaacaggagtacctgtctgagtacctactataacaccaaatttttctagacgatttagctgatcacctagtttaagttgttcaaatgctgattgcaaaacgaagatgttggaagcaagagctgcgtacataataggtagactaccaccgatcttagccattgccgcaaaatcacgagttgcaccacgtgctgaaccagaagtattaccaatagctctagaagctctaccagcggcaccagcagtatcattaaaacccctagctgtgccacctaatgctctattagtatcatataatctatcctggactttttctgttgcagctgcaacatcattacccattgttttaacagatctagaaactccatcgaatccaatttctagtttatctgttacttcaataagctggatagccaggtagtctagcttatcaccaataccatcaatggatttagtgataccagccatacccctactagcttgcattttagtaagacttttcgctgctctatctgctgccctctcaatggaataaagagttctgggcattttacctaactgctcatttgtcagttcggaagcagcagcagcattttccaacgcatcagatacgttttcaatagactttgcagtacgagttgcccccttctgtttaacgtctattagtaattctcgtattagcttatcagtcatgttttatttttcccaataaaaaaggctctgggcaaatcacccagagccaagcctcatcgtggaacgcccattggaggaaacttttaccgtgcacgagatctagcatgtggcttaacgttaggaatccttccgttttgcggtttatgcttctcagcacgtttacgtgcagcatctacagctttagcatcaaatatattgataatgtgtagaacaaattctttctctataggatcagtaatcccataaatatcaaataaaacacctagagcagccttatctttacctataaagataggaaagtcacctggtatgaagcagtcaattagactgttataaatattcatactagtagctacaataggtggaaagtcttcaagttcaacaggcatggccttaggatcaggctctatacccattgattcacaaagtattaaatattgttgtttagtcattttagatgctgagcttttaatacacttatctgcaaaatcagcaatagcatctaggagttctttacgcttttgggctacgaaaagtgtctaagtggaacactgtctggttgatccaagaatcaaacgcagcagagttctgcatgagcattacagcattatcgcggctaaaaggaacttcagtagcaggatcagcatctgtttcaatcagcatcagtttttcaacgtcacctacagtcaaaccggtccaacctttaatagctgcgtcaacgaaggcttcaatgaacttatcatcatcctgtacctgaatcaaagtaccattaacccattcatcgcgtttagcagaagaaataacacgtttagaggtagcacgagacatatagttaagttctaatttaaagttcggcatacctgggtaggaaagagtaatagtacgagtatcaagagtaatatcttttaaattaatcatttttaatttctccaaagaaatctattctcactgggtctgaagaagcagtaggtataatatcccactcaactctatacacatctgagaaatctaggcgttttgtaatacgtgctgaaggaaaatctatagaaatattattatttcttatatttaaaggtacatctgtctccggttcgatattgtaaaccatatctcctgcaaaacgttttaagtaatatagagaaatggttgccgaagcattcatttcatttacataagctcttttattattataaattttatttatatcaaaaacactcttatcctctctccaggagcactgctgttggaaagataaagaggcagaaataagaccgggtaagatgctgccattagtagaagctattactgggctgtaagacattacttcaccctgtataatagaagctgcttctctataggtagatacttctgagaatttcccagactcaataccaacattaagaattggtatgttcttatctaaagaaaaatctaccgtagatacataacagttttcaaaatatgcacagttattatctttatttactatatagatattgaacataataggttcaatattactactatatagtgggagtaagaaagtattaccttttctatcaaaacctaaccattcaaagaagttagcctctgtaagagtattactgaaatttattgctagagagatagaagagggggtttgagcatttataatagagtctgcatagttagttctacgatgaacagtcctacgaagagtcttgaattcctcgtatgaagtctgtatatcatagtttgatagggcatcaaaatgaaatgccctaccatcatactcaactataacttttgactctcgcattagagagtagtacatttatttaaccaccagcactaactgtgatagttttagtaccttctacaccagaaccatctttagctacagccttcacagtaactgcaccagttttagaggcatcagctgttaataacccagattctgcatcaattgtagctgcgttaccactagaaatagaccaagttacagccttattagtagcttctgaaggtgttacctcaacagacatctgtaaagtattactatttgttacagaaacagcctcacctgccgacttaactgtaatggatttaacgggaactggaggagttttagcaccatcccccgtagcaatcagattagccacagtagtttttgtatacttgttagaaaatccaagataaccttcgtcaccagtatccaaatcagtaggaatagctttaaactctactgaagtaccaagtacatcatcagtctcaatagtagggatattaacgtgcgcctgtttagcaactaacacagctgccggacgttcctcgtcatattctccacccaaaataagtgcgatttcaaaacggttaactaccttaagggttttaatcagatctttgtacagttccatagaaccaagagacttatcattaaggtaagctgtcaaagaacccgttaactcaaacgcacccgtaaatgagccgattggaatatttacacgtgacataatattcggggttaggtacgtgatgttgttattaatggtaaaagtaccaccagtaataggaatatcatatgccttatcagaatccatatccttaatttttaggatagttaacttgttcttaatgtaagaactctgaatagtcatataagtttcatcatcaatgcctaatgcatcagggtcaaatggttggctatctaacggaatgagctgattaccgttaccagaccaagttacacgaccaatatcttcaatatcaacgttaacttctgcctggttgatctggcaagagtcaatgtaactccacgcattgtcagttagaatgtaaatatgcagcatagccagttcgtgatatgcgttatctttgaagttaaccatgaagttagtagcattattatgtgctccagtatctccatcaaggttaatagctttaccactagaaagtgcatgccataacatatagtcaggaacaatttgtttgctagtagttttatccatgtatggcaggatataggtggagaagctccactcagctgcattcaaggaatcgttaaaacgtttagaaccacgagtaggacgcggaccagcttcgttaacagtaatatctgttgagttactgtcctgaccccaagaaatatcatcctgaactagaatctcttgcgtgttggtcttatcgtgaccagtcttaaccgtagacacgaagattcgagtattacgtaatagttgtaaagacatttaattattttctcctaatctgtaacttatctgcgaagtgacctacgtggaggctgatacctcactgttacatttatttctgctaaaccatatggggccagtagaccctcatctgtactaactgatgtaatgctcatatcagtcgcctcacatgggaaggtcgatccattaggtttactaacagtatattctaaatttccacctgtgtcaataacggtttttatatccgctacgagtttttcaagttgctcttgaatgtctgttttctctttatcatacaccaggataggaagttctaaaaacatccactgttgacctgagggaagatactgcccagtttcagttccgatatgaactgcaacataaggaaactctcgtatctcctcaaacttataagtttgacgtgagacgtttccatacaagttattaaaatattcatctggttgagagccatccatttgtttggctattcgatcaaccagagcttgtgctatacttgttctgtgatccatttattaggttccttgattaaccttaattttgtatctggagtgaattaagtctcttgcagctttagctatcgcctctccaattagtctttgagggtttctagcacctgggtagggtcgtagtgacaatcttcgatatgtagatactgcaggattaaatactgaatatggacgtaccatataattatatgtaacgtttaattccggagcacctttgctagttccagcatctctaagcataacatctttaacttttagagagtttgcaaaacgacctgttctaaatttaagtggagctcctgcttttttcatatcctttattaagtattctttagcaaccaactccagaataaccttcatattactatttgatacaaaacgaccagaagcacctgttactgcaccagaatatccatcttctacgtctctaggatccccaaagtttacttgaactctaccagtagaacgttttttacctacagcagattttttaccagcggttgcttgtattaaattctctagagcacctaaatctcctgttagatctaagctagaagaaactgcttttagaatagaagcggtcctaacattaaggagatcttgggatacaggttttgagaatttaaaagatacctgagcaacagtaccatctctagtaggatctttcagtcctgtttcaacagaatatacaacagtattcttattgttctcggaacgcccaacattatctagctgctccttaataatttgtgtagctaaatcagaaagagacattatactctcctgtaaacttcaataatagtacgaatatgctctggaataccagacttagtattattaaaagtaactgtttctccgccaattgttttagcttgacggtaatcctgtttatgccaatgatctactagcatacatgctgcaagtttgagatcctcaggtattggattaaatccaccttgtgtgtattctacatccatataaccttctgggggattaaatttaagaagtataaccccatcagaatacaacttatattgttctggatctatctccttgtcgttaatagtcattttggttactgaagtagccgagggagaacttaagaagtattttttacgagtaggttttgtattgattaactggtctaccgcatcagcatcatccatacctaaaaggctagtaatcagcgcattggcagcagtaatcatcatctctactccagattctagttcaggtcgttttaacccaccatataatctatagtcttcagctgtgattatttgcatttatttttcccaataaaaaaggagagccgaagctctccttatcggctgaaaagcctgtattaagatgcagcgtaagcaccagatacaacgccatttgcaaagtaacgttgcaggttaacacgctgagtaacgtagtatgcgtcacgctgtttaccagcttggcgctcacgttcaacagtaacagcacgctggcgtggcattacgaagttatctttataaacaataactgcgaactctgcggaagtagctttagctgggaagtactcagaaactacaaccggcagaccataaatacgaccaacctgaccctgcagtttaacagcatcattaccaacttgggcaacatcctgccattcttcatcttccagcagatcatagtaagcatccatagatacaatcagtaccagtttgctcagtttcagaccatgacggcccagtttacgacgcaatttagagatagttttagcagttaccagtacggaaccatcagctttagcttcagtaataactttagcactatcttcactagcaagagtcagcagacctttcggtttaccggaaccatcaccagtcatgaaggcttcttcaatagaaacagcgtgtgcttcgatcagacgcttacgaagcagtggcagcagggagaagattgcatcttcttcagtttcatcagtaatgaaggacttagcagccagtttgtaagtactgaagtggatttctttaagagcacctttaacttcatcaccagtagttgcatcagtaccataagtagaagcagcaacccaagtagctttaccagcatccggttcaaccagcatagtaagaatcttactggacatcggcagttcttcgaacagtgcgccaactacaagctctttctgcaggtcacggataatacgctgagagaaaatagtttcatagctttcactagaaacttctacagaagaagactgattcactgctttcaggtgtttttgaccgtgttcggtctcgaatacgcccttttccattacataagataacagaaccagtttttcaacttcatcttcaaagttttcctgagtaccgtatagtgctttagcaacactatcaccaacgaaggaacgaccttcacgagctgtcagcagagatttaatttcatcctgaagaccaacaatagtttcctgttgtttttctacagtttgtgcaaacagttctgcgctcttcttagatttctcatccagagacttaaccagatccagagcctcttccagacgcttacgatcttcgccaactgctttagaaaccaggtcattcatacgagccagttctttttcttcctgctctttacgcatacgttcagcttcctgagctttctgagcagcggtcagaccttccagagatttagccagatcacccagaccaagttcttctttcagcttattaatatcaatagtcatttttaaataattctccgttattctttaacgtagcctaaagctatcgcaagtttttctagttcagaaagattacgttcttgagctgggacagctttagaacttattgcagtaaaagatttacgccattcagtataatcatgaccattcatgctcttagcgagattgaacgttgagtcctgattacaaggtacagaaactaccgaaacttcgtatagttctaaatctttaataataaatatatcagtagcttcatcccactctgcatctaggcagcggaatccgatactaaaagttttcagtacaccgtttttaattagtgaaaagatagccgggtcagaactttcatagatctcgcattcgatttcgagacccatttcagtagggttaagatcaatacacttaccgattggacgacgatgatcgtgtccgaaaagaataatcgggtttttcatatagttagtaagtgcattagatgttttccacgcagaagccggaattacatcaccagcgcgatctttactaattgtattagcgaatccacggatttttacaacgccctctttggattcgctatcaatagatttaatataagcatctaaatgaacgggtgctgattttaacttgttatagtcaatagcagcttgtgtcatttattagcctccaggaacagtaggattaactgtcacagcacatgcagtagaatccttacttacttgcttactatctgttacagttactttataagagccggcatcctctgctgctgctgtaggcttagtataggtagctcctgaagcttccggaataggagaaccatctttagtccatgcataagtatatggactagtaccaccagttgcagtaacagaaagtgttaatgctgctccctcttcaactgacatgctcgcagttaaatctttagaaagagttaatggtggagccggtggagcaggaggaactgctttaccataagcctctacaaatttcttccatactttacggttagtatgcgcagatgaaagccctagttctctacgcagaaaagcatagctaggaacatatttatgcgcagcaaccgtagcaaagaagatatgagattctggtagtttttcaccaaaaatggtctttagaccattataatcaatcattctttatctccctcggtggaaccttgaggtctaccaccttcttgaccagatacacctgttgcagaaccagcaacgttagcaggaatacgaatcctatccatctgctcatcatctaaaggttcaaggttcagctctaaacgagcttcgttaccagtcataataccattattaaccaatgaggttaaatgtttagcctcagcttctttatctggtgttaatgcagctacctccttagtattaggagtaatcttataaccaaaaaagaaagtaagagaactagtcagtttgttcagcataggaatgatagtcatataatagaacaattcgatgtttggtcgaatattcgcgttattaccaccatcaagcagtacttgcggaactccaaaggctagacaaatagatttattaaatccttcgatgtcttccttaaagtctagatctttaaaagaggatatttgggagtacggtttagctttcataccaccatctagaatcaggacagaagactgaccagtactaggattataatcgagttgtaattcttcttgtttacgctcacgcaatttcttgttcaggatttcatccgtttcaagaataagaccaatcacggttccgttatcgaggaatttctctttaaagttaagcatcttagaacgcttctcaagagaatcaataacagtagcaacacgagattgtccagaaatttgagaatttgtgccacacacgtaactgttatcctttataaagataatctcatctacgcgatagtctatctgattattaaatataaattttttgataaacttattggcatctgcctcgacctgcataagagcagccgggacgtggtaaagcgatgtgccatcccaatagatgtatgcacaaccttcaaaaagtaggtcagtgactacaagtctacggaatgtgcttatatccatgaatggattaggtcgtacatttaagagagtgtctagagtctttgttttgacgccattagcgtacgtaacaatattatatttatctccgacagtataagaacactccgccgcactatctataaccatattagcagttctattgagaatctcaattttactgtaggcttgtccagtggtaaaaggcttacggttagtgcgatgactaactggttccatgtctcttataatacgttgacccggatttagcttttcagtaatccagcttttaaaacccattataagaaccttgcgaatcctgattttgtttcagtcttgacttgtgtcttttcttgaggattctgtaatttgtctttctgcttttgaacccacactttttgcttattagcagaaaataaaggaggttcttttgtgtaaaccttatgtaataattggtgatggtgtacacagagggtaacagtgtcttctactagctcatgcctatatcgatcatagaatgccgtcctatttgaaaggactgtttcttcgtcagtgaaatccaattggagttctttagcaaattttttaactagtagagacaccgtatggtaatgatgaagctctaattcttcagcacaaccgcagatagcacattgagagtcttttttatattgactttttattccgtctctcataagggatatagcatcgcgtttatattttatattgcttgccatagtgttctcctcaactgtatgaatatagtatacataatcgtatcgaatttgtaaaggggattttaaaatacaggaatgggataccaactgagtagtatcccatcctatacttatttatttaccacgggaaatagaataaattccatagcggagcgcatcacaaaggtgagagttagcatcatgacgtggtttttcacgtgataatttctcttcaccttcttggaagtcccacttgtaattctgcaatgcatgaattaaggaggaacatgaggcatttacaataatcttgccctgttggaacagtgcttgcaggcatgctaggccatctaggacagattttttagctggagctgaggcaatttcatgctcataagctaagtcctggcggaactgagctgctgcagagtcaacaaaaatacgatcaactttataacgatctatacagtgctgaatataagcagcatgctgagctgtagttttctccgcctgctggtactcttctaatacatagtaagtatccgtatcgtaatgatatttaatagtaagaactgctgtaggatcacgataaccaacgtcaataccaagcaacgtttcgaatgcttcatcatctttaaagaagtgacgcatacctttgagatctttaacatgatcgatagcattaaaggtatcaaatatctgtccttcgaatacagagaagtcagcttcgtattcttggcggaagtagtttttactaaccgtacggcgtgcctcctcaatgtcattcaaatcagcacgtgggttatcacgatatgtaccatgaatagacacccagttaggtaacgtatcatcgaatccgtaggcgtaaaactctttaaaccagttacctccgcgaggggtggaaataaatagagccttagaattaggcttatctagggtaggacgcagctgaaccctgaaggcatcaccacccacatcggaaattgccgcctcgtcaaagatgataaaatcatatgaacgtccaaccgcggagtcagcctgagccgcggaagctagtttaaatagagaaccattagctaactcaatctctttatctttagcgttttcacgttcggtttgtaggccgtacttcttaataagaccacgaatctgagaccatccgatattagctagtgaatagttaggggcaactactagtaccttcacattaggttccagcaattttaggaacccaagtgtatacgctataaaagatttacctacacggcgtgatacacatgccgttacaaaacgatgtctagggtcttctagggcattgataattgctatttgagggccattaggtgtgataccctcttgtgctagtattccggaaacgggtaaacgaaagaaacgcttatctattccgaaatcaataacatctactgtattaacatagggtcttgatacttccattacttcttacctccagtagctagggaagtaattaaatccatgtagttttgatccccggctccagcaataatgttattctggatattagtctgattagcaggagctcggacggcagctttagctttttctaactccaccatcatcttcatttcttccatcttcattttgtgtgctttccagagaatatccataatatcctggtcagaacccatgccagtttcttctagttcctccagcttacgcttaataacttcatccagaacaccaaataaacgatccctattacggaaaccactctccataaaaatatcatttaagtagttcttaacctcactacgattcataatatctcggaaagcaatttcggacattccaagactacgtgcggcagaaggcacatcgctgccgcactgtaaataagcttcaattacatccatcccttcaggggacataagatctggtactaatacatcatttgccatatttcctcctttaggtaaggcgttgaattataagtttaatatttggtaagcatgctttcatgccaattatattgctcatagaggattctaaccaaatagagatattattatttccatcatttctcatatagtatgttagagtatagtaacccgtttgacctacggatcctggcaacccagatacccagtacggagttggagtatacccagcagcctgtaatttacaagcatttactacagtacctttaatagtaaaaaatggtacatattctcctgcaggtagggacaatatttggtgaagttttttatcaatagtattgccacccattctaaacctactatcgccatattcagcgcctttagatagcatttccgtaggattaaagcagccaatacctcctgcagacaaccatgtgtcatctaggaactccatagtacatacaattgcaccatttctactactattaagatctagtcccacggtaccaacatgcacggaaccattaccctgaaatactatgttaccagatagtacagtacttccgctaccacctagaagtcttaaaggtagaagattttcactccaaacgtccccataattactccctatttttggaggattactagaaacataccaagatgctgaaggtaaaaagttataaatactaactacattagtatttaaaccaccaacgttaccacccttaatacttggtgcgttattgctttctgtggttggtagtccaaatgttgctatgccagagatcccagtagaggggccacccatactcaagttacacccaacatcacgaaaatcagctctgctccctacattaaagtttttctcaacaaagttgataaacttccaagatgtattattaacgttaatacctttaataataaattcagtggggcttaattttatatcattacctgtaaataatccagcatcaatactataacctgaaccattatacactaagttagtaacataccaggtaattttagatggataaataccgaggtgctcactatatatacctatgggcccattagcagcatcattcggatcacccatactttcccaatacgcatagttagtgggatcagcacgtgcgaaacctacagagtacttaggagccgagggattgctaggattagacataaagttaagagcccctgaactcgacgccccatgtacagctccagcagtggtgtagttgggcccttccataccataaagaactctattacaagacctaccacgaacatatatggcgttaaatccaaagtttctagctccgggatgcctaacgtagtcgctagggatagttagagttcctgaatccggcactacatatcctgaaccatcccaggcagacttataaagagcggcaccccctaaatacattgcactagatgcaaaccctctacgcccactaaaggcgccaaacatatgctgacatccagtaccgtctcttaatttctgggggatcgtaccgctagtaccactattaaaatagcctacatcaagagatagatcagcactagctgttatttgtgtttttaaaccaccttgagacatgacctgcggatttataattccatacgctttagctcctacccccatagacatgccagatagcatatgtctagagccatctgagaactcaatctctgtcagcacaaccctattaggatcattattatgtaactgggtaattctgctaggcatatcagaccagtagtaatctccagcaccttgatctagcctcaaccctgtggagtgagtttctgttatgataacatggggcatatctgaatggaatatggtctgagagtcaggattaatatgttgattagtatctccgccactacccctgcgtagagagagtatagatttgttatttagcttgccagcgaaaaaactcataagttctccaaaaatttaattgcttatgttgctataattctgtataatagtattataaattaaacagaggagaatgtaaatgaaaaaattatttttagctatggcagtggtacttctttcggcttgctctacttttgggcctaaagatattaaatgtgaggcctactatgtacaagatcatgtgaaatataaggctaatgtttttgatagaaaaggggatatgttcctggtggcaccaatcatggcctatggatctttctgggctccagtaagctactttaccgaggggaatacatgtgaaggagttttctgaggagaatccctcccataaacccatgccacttagggtggatcctaggtcaaaactagttcacaatgcaattgaggtcattcagaacaccatataagataaaatgttatgacttgtgcaattgctagaatattccaaataccacatttacgggagtatgaaagacatgtcaaacggcaacaaaagaaagaacagacgtggagaagtgtacgatagctggattaatagctttgggttcggaactattctattctttattttcgtaggtttttggttagcagctatggcggggtggatctaatggactgggtaattattgcactactggggattgtgattattgggcaatgtatcttagataatcacttagctagaatcgaaaccttactatcggagaaacgtaaatgatggaagtggcaattacactaatggtaatattagtctgggcagttttcattatttattacaccgtgtacgttcgcctaaaaacgctagaagctcaagtgaagcaacagcaatttgctatagaaaaactatcagaattgcaacgttgtgatagtacccgtattctgcaaattgggagggaattagatgtttagtattgtaattgcttttattatcgggatactcgcgggggttttcgggacaactgcggcaatcaacaagcatcgtaaatatattgcgggagtgacgcgggagattgcggaaagggagcggaagtttgacgagaaacgtgcggagtttgagcgagaatggtcggatggatctcgggcgtcgcggaagagatttcgtgagtttagacctgaacatctgtttgatgacccttctatgcgctaatttgacttttgccaaatttcataagattacacatggaggtgtgtccactggctggctagatgagaatgagtctcattaccgcccggtacttagcaggctaatagtcacggcatgttatcattacacaccgtgactatttcaactttaattattacgcaacttttaatttctcaatatattgttgcgggctgcaactatattctgcgcgttcttcctcggtcatactttcccaacataattgcaggcggtcggctaggaactgataatcattatcatcctcaagagattcatcaataaaggtttcacctatcaatgtaaccccaatcgatagaatatcgttaacggcttgcgcgtccagcgttttaaatccgtatgatgccgccgctgatagcacgcctttatgatcatcaatcatcgtgaaatcagcgttcggatataattgtttaaataactcaaagtaatggcttttataaatagcgtctttgctgtggtatatctcgctaactttatcagcctcaaaatatttgtgtagtgtatcgcgggaaaatacacggatattactatccccacgccctcgcaatccttgtttgcgcaaataataataatccgacttgctcattagtcgagcggtgcaaataatgtttaacgtgttaacgtcattcatacattggcgcatatatgtaacaaggggcaatagtgtatcttgcattattaaatcatgtttgcaggcttcatttttatacttatttaaatctaaattgccctcgctatcaaagcaaggcgctacgcgatggaaagaattaatgatagtgccgtccagatcccaaatcatgacgcgggaaatatgcgggaaattacgggtaatattcgttttaacttgattcattttgttacgttctccatttaagggattagattaattcagcattacccgctatttataagcgggtaataataaattaactattaaagaccatacgccaacgcatcagcaataaaactaatatcttcaccagatacaccggaacgaatgcccgcgcaggtatatacctttgcgctggtcaattgatcatcgaggcaagcggggcaaggcgcgacaactgcaaaggctaggccggattttacggcgtcgatgataatcgtttcttgtttcacagtgtcaacataaacaccttttgctttaatatggctagttagtttaccttgcgtataggcaaaggtagtaacctgcaaagcgtcgttaaagtccaatagctcatcaataacgtgccccgcctcgacgtggtaacgttcaccattagaataaaccagaacaatggaagattgagcgcgatcgaatgggatattttgctttttcatatttataaaactccaattaatttaataaagggtatttaatttacttgataataatctgcatttgatacggcgttaattcaccatttacgggtaaattattatcgcttagccactcataaaaagcgtcatcatcgtcaaaagggataatatcattatcacgataatcgaccgtaataatcccatccgtattagttttggcggctaggataggcccgaagctgtaaactttatcggattccatttcatccggtgaaataaatgctttttcgtccagcgttttgggtaatgtaactttcattctaatttagctcctataatagcggctatatttcaagccgctagggtattaattatttacgcaaaaaatttaccgttcttaaaatcgatcaaagtacgctgaccatttgcataggtaattaaatgcgtttgtgtccagcttgacgcgccaacgttgtaacccatgtccagactaccagccacgccagcggtatataccccgccgtaaatgctggcggtatgagtgtgccccgtgtttaatttgcccaatttcttaaactgtttcggattgccacggctgccgtttatgccattgtgaccgtgtacgccgcattcaatgcctgcaattttaaatgattggtcagtcgttaggaaaatggcgttaaactcgcaacctgcaaccttacgcagcgcataatctagcacgttgaaagtatcgtctttttctgcaatagctccgtaaatcgcggcattcaggcggtgatatagttccgcgttcgctgggtcgtctttaatgttagcattacgatcatcaagccaacgggatagcgccaaatcgtgattcgattcaacaatgattgtttgcgagaaatcgcgctccattgattccagtacgcgccccgtatcgatgagatcatccagaactttatcacgtccggcggcgtattgttttgctaggaatacacccgaagcgcggttgtgatggttgcgtgatgtaaaatcgtgaacgtcatggataaactgatattttggtttaagaatatcaagcaagctattttcgccagcccatgacgcaatggcacaatcatcatcaagtttttcggcgtgtatgtcgccatattgcaagcctaaaacatgccccgatgtttcatagcatccggcgggagtagcgcaaacattcagatcataaaacaccccgctttcgtccattgtttcaagctggcgaacaaaaaactcaccgtcttcgtcgaactcaacaatcagcgcaccgaagttatgcagtgcttccgctttctgtcctgctttctgctggatgtaatttttaagcgttgccgtgccagttgaatacatgcggcgcactacttcaccttttaaagctggcacgctttcggcggtgattttagcgtgaccaattgccagaccttccagattaagcgcggttgcagtttcagcgaatccagaaagcggataatctgcggtgggcaaaacattaatttcagccatgaaagcaaaacggcgattattcaaaaacacgtttttgctgcaaatatatttatcgaacgcggaatcatattttattccatccgcgccctcgccattttggaatccgtttttattataaatatacttgctaaccagcaaatcagcgccgatatatttagcaaattgctccagtgaggctaaaaagtttttatgcgggaacgtattattctgaatcgatgtaataataaagcgtttgccttgttttacttcccagctttccaccgtgctagaaacaatccccgccgcttctgggcgctcatcgtttttaatggcttttgctactttcgccgctttcggtttcgctggtttatttgcttccagccaatcacaaatagcggtggaatgttttggagtatcgtaaaccgcttttgatatatcgtcacaaacttccaatttagtggcgcgcaggcgtttaccgtagaaattaaagtcagcggccattttcaggataacggtttgtttttctgcggaaataatcatagtaataaaactccattaaagggattaaaaaacgtttgttattggcaaatattgccaaatataacgcctattatttgaggcgttataatttgcaacattaatttaaaggcataaggctagaactaacaccacaaacaacggcggcaataaaacaaatcagacctaaacgctttgctaacttagatttacgtttaaaataatatgaatctaaatcattttgaaaataagcgaatagtaatgaggccgccataataaataagcctagaattaaggattcagtagggtaaaagatcattttaataactccagtcaaagggaaattatagtggggaatattccccactaattaaatttattcgcctgcggcttgtttcacttcatcggcaacgcctaacagttgcgccacggcgtccagcgtttccagttttgcgctttccagtgatgccagatcgtcggcgtctttgataatgccggaatcgatagcgtgttttgcaataacgcgcacataatgcgctttgcggattgagctaccgccgccaactttgcgcggcttatcgcttttctgatacgcttttgcgctggtcaattttgagcgaacggacacgggggaggccgcgccaactgcttttgcaatctctttcaggccgtcgctatttgcgaaatctaaaccgttttcattaattaactgctggtacatggtaacagctttttgggtgttttcttcattccatgcaaattttgcggatttttcggttttaacgttagccatgattgtagctcctattagtttaatttaagggataagattaacataatgttaatcgttatatcccccgaactactagggggatataaagttaacactaaattataaaacagcttcccatttttcgttacgctggacaacctttacaacgtcgccggatttaacgcgaacgctaaaaacggttttgccgttgttactccgttccgtttcagcgtataccgcgtcgattgtatcgaacggcataagcgccgcgcctttaatgcgttcggcctttccggttaattcatccgttttagtaacgaaaggaataaaaacggattgaccaactttcggcgcggtaataacgttttgcataatataaactcctagttatttggggattgttccgcattacctgctagaaagtaacagcaggtaataagtaacaatccgctcgccaatataatcaatttaaagaactccacgcaggccgctatatcccagttagggggcggggcacattatggtaagtgccgaccgtcaaattttatcaagttatcgctcttatcgaactgggtacatcttaaagcctatcggctagggtgtcaaacaattttttgcagttttttatgattttcttttaagtcaatcataatattacgccagatcatttccagaccttgctggcggcctttcttgtactgcttaaatcgatattttaccgatctttgcgctactgtcaacaaattttttgttttgctgtccagtgccttgctaggtttaccagtgatcaataaagtttgtttcatatcgtcgattccttataatgtataagtttaccgccgataggctttaagatgtacccaatttttaaagagcgcggcggtaaacttcccgccgtgtagttcgtcattgccgccctacgagataaataataggggattcccgaaaatggcgcaaccttatttttgtaaagaaatgtaaagacaaacgccaccggacaccatgcggcgaccggggcgcggcgcattaaaattaaaagaaacgggcgcgcgaataccataaaacccagcaggttggcaagtaattttttcatttatatttgccttgccctacttgacaaaacttaaaaagctcgtggtagcgataaacgagggtaggcaatgcaatggcatggctaaaactttcatcgcgttctagggcgttttaggcgcattctagccttgttgataacttatccccaaaagcaaaaatgaaattttcctagttatccacttgacttttattttccagtggataactacttattttatgaaataaattaattgacttttatttgttagtatgcatcggaagaatagcagactaataattactagaataataatcatgctggtggattgtaacggcctaaactatcagcagactattaatcacgaaaaatctcgttagctaactaactatttcatgatgtataatatacatataaattaatgagaaataataattataataaataataactacagtaaataataactataagaaactatcggcctaagtgagaaccactatcatttaagttatacacaaagttatccacagcactttttgactttacacgataggcggcgatataaggacttttacacagacttatccacaggttattccactgtataaatatacagggtaaaaaggtagtgttaacaactgcaatcaattttaagcggcttaaatcgcattctaacgagctaaacgctgcggggtaatattaaagtaaagccttaatgcgtcaccctggtggcttcgccgtggtttgctctttattgcaccgctgcgcggttagcataaaaataggcgctaggcaaggaaaataaaaaggtaaaataatatttgcctatgctagaaatttacctttatcattcactcaccgattaacgagggaggccgaacaaatgaggctttataaacctgataatgccaccgttttaaaaggtgcattgcgcaacctgctggatggtagcaagtcaaccagcattaaacattttattaacaagattgaaactattcatcaaaacttttttgatgattatgacgcttatgatatggatagttttctacccttatatagacaaggggcaacgctagttttatataatcgggattgccatatctttacaggcaggggagcggggccggataccgattttattgttgcgcagggtaataatattgttgcgtatcatagcaattcaggccgtcaatcatttaatataaatagttttgagcttattggacttgatctgataaatggactttctagcattgagggatttttagatgttatctggaagtatttacccgctggatggcataagccggataacgacggatctcaatataccggtataatggcttttgatggcgacggtaattttttgttaccacgggagctatacccatatatttatgatggggatatagaaagcgctggtttttctttctggaaagatgaagatcagattattgacagcgtagaaaaccatgctgtaaattctaacactcaaagcgttaacactcaaactactgaaaacaaaggaaaaaacactatgaccaagattgctaacatcgttgccgctaataaatccgccgttgtaaatgctgcaaaactggaagcgggtaaaattgcactgactcaaattacgaaagtagcggctaaaaaagcaccgtttatgattaaaggttatattgatacacctatcggtcgagtagtgattgccaacctgctgggcgtagcggttgaccagtacgccacaagtaaccaaaaggcgaaagcggtggcaggtgcagctatggaagcggctatgttagaaatggtgcaaagttttaacatcgctgagatgattgatgaaatggtaaaaggtattgatatttctacttttaccactagcaccgaaagcgagtaataatattaagcggggattgttaccccgctttactattcccttgttaactaataggaggctaataaatggctgatgaaaaatatggcttctcactgggagaagtgagcacggcaaaacatgatatgatcattttggtcgattgtcatactggctattgcggggaagggaacaccgaggaatattttgttcctgctggtacggatctggacgctttcgctcaggaaatggctattgataatgcttctcgttttggtagcgatggttacgaagacgaagaaactggcgaatggtatgaaaatgaaaacgtatatgcaagcctttatcattatcaattaagtaaatccggcacatatgtaaacggcggcgatcctattaattccgtcatgagattaattattaaatatggcggcgtcgagatagttggtaacaaagcggtaatatacgcaaatcgattaaaacagcttgtttatattcctgatagtacccgatgggatgaatatgctgttttgcatgacgaattgaaacgatgtttcaatattgaatccttacaagtagtctaatgtaaagttttgtaaaggtcgccttttatggtggccttttttgttataatgcctttacaccgtttgaggacggttgaaaattttcccaaaaattggaggcttaaaatggttgcttattcttctgctagtcatctgttcaccgctcaaggctgggtatctaaccgcgttattattcacgaaatggtgcaagaatatggcattgctaaaacatacaataccattcaggcgctttatgataatgatcaaatcgatcagcaaacggcgggctttttgctggataccctgaaagcagaacattgcaccaaaaaacaagcggctaaaattgttttaatgtaattaataacggggcgttattttcgccccactaactagggggaatgaaaatggttatttttcgagctattttagcgggtatcttgggcgctattttatcgttgtttgcaatagctttcggcgttcacgcggttattattggcgcgggtatcgctgcattttcttatctgctgggctgcatgactagtaaaggagaataaaaatgatccgcaatgtttcccttgcccgttcgaaaggctttaagctggtggatgttaacacgttcgagcgtgaagattgtaaaatcgaatatgtagcacgtaataagaatgcctttcgtgttacagaaaagaaatttgacaagcgcggcaacgtgatcgctgaaacggttaaacactttgccaccttttacgctgcgtttcgtggggtgttataaaatggttatttatgagggcaaccgctttgttgctaattgccgccctgcattgctagcaaattacttaaatcagctttcaccagcttataagggcgttattaatatttatgaaggcaaggcgcattataaaatcaatgcggtcgttgcccgtgaactagcgtttcaatttttgacctttgcatcttgtgacgttcaggtcatgggtgaagcgttaattgcagaaaatgaagatgattttattaatatttttcgcaagatatgcaccgagcgcctgataatgaaaggcgcttatattcaatccactgcggatagtattgaaacagcgtttcgaaaggtggcgcaatgagcaataaaattgttgtaaccaaaatcagtacaatggttgatgttttttacgtgcccgatacgccggaaaaccggcaagcggtcgagcgtggcgagtatgataaagtgatttatgatcacgatggttattatcagcatttggtcgattcctatggggaagaggaaaagatcacgcatcaattaccggaggatggtttatgaaacgatggtttaatcactggtataagcggtttttagtacgccgtcgcaaatctagacatcaagatgagtggcgggagtgggcaaagcgtaacggttgcccgctactctgagaacgacggggattagctccccgtttttccatgcctccagttaaatacccccttgttttagtatccccaccaaaattaaacgcgttagaatgcgatctaggacgttttagcggtatattagaattattcactacataggaatactgtatatttatacagtaggataagctgtggataagtctgttaataacgcctcaaaatggctaggattctgtcaagtcaaatcgtgctgttaataagtctgtggataacttaaataatagtgtttctcacttccattcttaaatgcaaatcattatcattcgcatttcactttctaaatgagaatcatttcacttcttgaatagttctcatttcactttttaaatgcgaattattatcattctcatttcgctttttaaatgattcttattatcattcgcattcaggcgcgcagggcaattgccctgcgcttttgtgcaaatccgacattgctccacgggtgttacgtgcgggcgtgtatgtgcgaatccgacaatttttggtagtgtgcaaatccgacccgaaaaatttttatagtgtgcaaatccgacaatgaattttcccaaaaactccagcaatccgatgctgcgcatcaatcctgtggaattagctagaatatgtgcaaatccgacatgggtttatttgtgcgaatccgacaagatttttgtgactttgattggagggtgtgtgattgtatgggtttgaggagaagatgagagagcgagaaaaatttgagggaactgtgagaggggcgcaggattggtgcggagcacctgatttgcggggattttggggaagagttgagataatttgggaagatttgggataggtgcagataaagtaaaattttattagcaaaacgaggcaaaatagcttgacaagtgaaaatttctgtggctc

SEQ ID NO: 2 - DNA, 167170 bp, unidentified
  source 1..167170: mol_type = genomic DNA; note = Bacteriophage
atgaccaaagatatgacgttagaagaggcaaaggctaaagcaaatgaagctttggatttgcttcttaaaattggcagtaaaatgatggaagaaaatgagaaatatatccaggaaaacaaaattcctgatggtccattagtaggcaaaaggaaatcacatgattgaagtagcaaaattatattcaatagaatttatgtccaaagaaggtaaatcagtaaatacacttgataaaaagtgctcattaattattcctttagcagaaaatccggatattttaattaaagatataaaagaaagaaaatatccagaaaatgttattctaattataaagcatactgaagatattttgcagaatactgattcaccgttttcttcttctgaagctttaactattaaaggctataaaagagctcatgaatatggtctttttgacatgtttgaagacgataaagttaaattagcgagtcaaccttctaaaagtaaaacattcattattgaagatattaaagatataaatgcatttgttaagatggtctgggctcattttgatgttggactacgctggagaatgtccgaagaagaaagaaagattattgaagctaatcgtcaatttggtttttatcgctaggaattagtatggatttatttgagatgttagaagataatcattctatgaatatccagaatgattctagtgattataagaaagagtaccgtatagtattacagaattatggaattgaagccccagatgctcttctagaagaactagcttcataccatcttgaccctccgccttgggctccctgggcaaaataattcaaaaagttgtttactttcctttctaacgatgatatgatagcttctgaagtatacggaggctatcatgattattaatcctgcagatgttgaacagttatctataaaagctgaaagcgttgattttcaatatgatatgtataaaaaggtctgtgaaaaatttactgattttgagcagtctgttctttggcaatgtatggaagcaaaaaagaatgaagctcttcacagacagttgaataaaatcattaaaaagcatttaactaaatcgccttatcaattatatcgtggtatatcaaaatcgacaaaagaacttattaaagatttgcaagttggagaagtgttttcaacgaacagggtagattcatttactactagtttgcatacagcgtgttctttttcttatgctgaatatttcactgaaatagtacttcgcttaaaaactgataaagcttttaattattctgaccatatcagcgatattatactttcttctcctaatactgagtttaagtacacgtatgaagatactgatgggttagattcagaaagaactgataacttaatgatgattgtacatgaacaagaatggatgattccaattggaaagtatagaataacttctatttcaaaagaaaaattacacgattcatttggaacatttaaagtttatgatattgaggtagttgaatgaaatactcagtaatgcaactaaaagattttaaaataaaatcaatggatgcatcggtgcgtgcttctattcgtgaagaattactttctgaagggtttaatttatctgaaattgaacttttaattcattgtattaccaacaaaccagatgaccatacttggttaaatgaaataatcaaatctcgtttggaaccaaacgataaacctctttggagaggtgttccagtcgagactaagcaagtgttaaatcaaggaattgatattattacatttgataaagtagtatcagcttcatatgataaaaatatagctctacattttgcttccggtttagagtataacacacaggttatttttgaattcaaagctcctatggtattcaatttccaggagtatgctataaaagctctacgctgtaaagaatacaatccaaactttaagtttccggatagccatcgttatcgtaatatggaattagtttcagatgaacaagaagtaatgataccagctggaagtgtatttagaattgcagatagatatgagtataaaaagtattcaacatacactatctatactcttgattttgaaggatttaatctataatggaaggacttagattcattataccacgaaagttttaaagcattttacataaagttgtttacaagctgaagtaaaaatgatatagtataagtaagttaaccgtccgtgagaaaaatatgaaactgtctaataaccaaattcgtaaaattaaacgtcgtttagagcatactcaggcatctgcaaaaagacgttctaaagattttaacttagacttcaattacattaagaacattttagatcaaaaagtttgtgcctattcaggagaaccttttgataatcgtattgaaggagagaaattatcattagaacgttttgataataacattggatacattaaagggaatgttattgcagtaaagaaaaagtataatacatttcgttctgattatactttagaagagttgattgaaaagcgtgatttatttgctttgcgaattggtcgttcatctgcgaaaaaagttcataaactaaatttagatgaaaagaaatgggctaaaatcaaaaagacttataatcaaattaaagctatacagaaaaaacgtgaaaatcgaattgaacacatttctcagctttctaaatcaaaacagacctctgatattaagctaagaattatagcacttaaagctcgtattgatggttctcgcatagcagaaggcgctgaagttgttaaattgaacgttcttcttaaaggctcggattggaaaattgtgaaaaagttgtcagaagcagaaatgcaatatgatatgtgtgataaaattattcaaggtgtagagcggtatcaaaacttgtcttttattgataaacttaaactgaaaagaggatacccgctaaattgttcaatttttaaacttatccgaggataatatggtttatgtatatgcgatagtttaccgagacaaagacggatttacggcgccagttccacttgatgaacatcgtcctgctgtgttttttgaaagagagattgctgatagagtttttactactctcaaagaccaatataaactagctttaggtatgggaattccaagactagttgagactccacgcaagttttggtttaataaaatagaagttaaacatgttaagcctgatgtagatacacaaagattatatcaacgaattctagatactgggcgtattgttagtattccaattgcagggactttacgatgacatttgatgatttgaccgaaggccagaaaaatgcctttaacatcgttatgagggctattaaagaaaagaaacatcatgtaactattaatggacctgctggtactggtaagactactcttactaagttcatcattgaagctttaatatctacgggtgaaactggtattattttagcagctcctactcatgcagctaaaaagattctttcaaaactgtcggggaaagaagcgagtactattcacagtattcttaaaattaacccagttacgtatgaagaaaacgttctttttgaacagaaggaagtaccagatttagctaaatgcagggtattaatctgcgacgaagtgtcaatgtatgatagaaagctatttaaaattctgctttcaactattccaccctggtgtactataattggaataggtgataataagcaaattagacctgttgacccaggagaaaatactgcttatatcagtccattctttacgcacaaagatttttatcagtgtgaactcactgaagttaagcgcagtaatgctcctattattgatgtagctactgatgttcgcaacggtaagtggatttatgataaagttgttgacgggcatggagtacgtggatttactggtgataccgctttacgcgattttatggtaaattatttttcaatcgtcaaatctctagatgatttgtttgaaaatcgcgtaatggcatttacgaataaatctgttgacaagttaaatagcattattcgtaaaaagatttttgaaactgataaagattttattgtcggtgaaattattgtaatgcaggaaccattaattaaaacatataaaattgatggaaagcctgtgtcagaaattatttttaataacggacaattagttcgtattatagaagcagagtatacatcaacgtttgttaaagctcgtggtgttcctggagaatacttaattcgtcattgggatttaacagtagaaacttacggcgatgatgaatattatcgtgaaaagattaaaataatttcatctgatgaagagctatataagtttaacctgtttttaggtaaaacggcagaaacttataaaaattggaataaaggcggaaaagctccgtggagtgatttttgggatgctaaatcacagtttagtaaagtgaaagcgcttcctgcatcaacattccataaagctcagggtatgtctgtagaccgtgctttcatttatacgccttgtattcattatgcagatgctgaactagctcaacaacttctttatgttggtgtcacccgcggtcgttatgatgtgttttatgtgtgaggatatatgattaacatcaattcaaaatatttaaatcgtctaatagatggtataaggaagcatactaataagcaagataatctcgatgttatggtaacaggagctgagctccttcataagctttatcttataagtgatactatattagcaattaaacgaattgaaaaacaatcatatcacagtaatacagatacggtaattacactagatgaaagtgtctgtaaattactaattaaatttgaggaagctattcgtggaaataactaaagatcagttttatcttcttcaggataaagttagcgaaatttatgaaattgctcatggtaaaaatcgtgaaactgtaaaaattgaatctagtaagttgatgcttcaattagaagaaattgaacgagatttaattgcgttagaattcttttgtggcgaagtgaaaactgttacaatcagtgattatgttttaggtgaaattagccatctttataaggcggttattaatgattgaattaagtttgcatcagtttaaatctcttatgacaaatgttaaagctgtcattgaagaaaatccgggtcctgaaaatattactattcgcgaaaaagcttcaaagatagtatacagtcttgaagagatacaaaaagacattgaatctattgcaaaatttattgatgagcctattaataaagtttatattcaagactatactgtaggtcaaattcgcgatttagcaaggaaaatttaatgtttgattttattatagattttgaaacaatgggaagcggcgaaaaggcagctgttattgatttagctgtaattgcttttgaccctaatccagaagtcgttgaaacattcgatgaattagtttcacgtggcattaaaatcaaatttgatttaaaaagccaaaaaggacatcgtctttttactaaaagcactatcgaatggtggaagaatcaatcccctgaagctcgaaaaaatattgcaccgtcagatgaagatgtaagcactattgatggtattgcaaaattcaatgattacatcaatgcgcataatatcgatccttggaaatctcagggttggtgccgtggaatgtcgtttgattttccaattttagtcgatctcattcgtgatattcaacgtcttaatggcgtttccgagaatgagcttgatacatttaagttagaaccgtgtaaattctggaatcagcgtgatattcgtaccagaattgaagcacttctacttgttcgtgatatgaccacttgtcctcttccaaagggaactttagatggattcgttgcacatgattctattcatgactgtgcgaaagacatcctgatgatgaagtatgctttgcggtatgctatgggtcttgaagacgctccatcagaggaagaatgcgatcctctatctcttccaacaaaacgataaaaagttgtttacttcctcggttagttgtggtattataacaccatagctactgaggataataaaatgaaaatttatcgtgttgaatcatcgtttagtattcttgattatgaagatgctataacaatacgtcgaaatctttgtgttcaaataacgccgtacaggagtataatagattcatggagcgaagagtggctattacacgtaggttatgacagacctaattttatgcatcatagcgataataataaaagaattcctttaccacacgaagataaactattagttaaaaatgctaatatcgtaattaatactaagttcaagaaagattatgttggagtagaatatcatattcccggatggtttatagctctttatcattttgcttttgctagcgaatatgatatgatgagatggttcacacgagaagagcgtgaagaattatcttcgaaaggattttatcttgctgtatacgaagtaccagatgaccaagttattattggtgggcatcaagtaatgttccgtaaatcccatgctgaacttgtagattttattgaaatgagataattatgaaatttaattataatcctgaatacataccaaatccggtagctaaactgattgattttgatgttgtaagcacttatgtatgccctgttaaaccactggaaattaaggaacctactatgactaccgctattgaaatcggcaaaacctacaaactagttgaaactgaaattaaaacaactgaactaaatttgggttataaagcattgactagtgtttttggtgaaggtgagtttgtggttaaaagcttttcaaaaagtaactggtatgataaaaataaagcttacgtgattcgagcacatcgcttagataatgatgaagtaattaaaactatagtctttgaagcagagttcatcctctttcaagaatgcgatcctactgaccttttaactcgtgcggtagctatcaagactccatttatttcggtctgtggttgggtaacagaccaatggattgaagatggtgttgaacttctgaacgttgttcatgcaggtgattttagcgtagtacctcgtagtgcagtggtagctattttgaattaatagtttacaaactcttgggaccagagtataatggtcctgtggagtataaaatctttttaacaagtgagagataactatgattattaatattggtgaaattgctcgtgtatctgataaatcccgttctaaagcagcaggaaaattggtcgaaattgtaagcattcagcttaaacacggcgttaaagatgaagattctgaagtaaaagtgcgtatcattcctaaagatggaaagtctaaacctcagtttggctatgttcgtgcgaaatttcttgaatctgcgtttttgaaagctgttcctgctaaaggaattgaaacgattgatacttcgcatgtaggtgtatactttaagtggaaactcggtcaggctattaagttcgttgctccttatgaattcgaatttactgaagatgaggaaggggttgttcaaaaacaccgtactcgtgctatgtgcgcatacattaccgatcagtgggtagaagacggtgttaagctgtacaacgcggtatttttaggaacatacaaagtcattcctgaaagctggattaaacactatagcaaagctcgctgtgcataaagtttaaattttttcataaaactatatacatcagtagttgattatggtactatatcaatatcaactactgatacagaaaataacttggagaataaaatggaaaacttaattatcatcgagcaatctttcaacgattatggtatggcttacggttatcgtgcaataatggaagattctcgtggatgtgttatcgatattgctgaatgtaaagatttactgcagcttttgaagattgttcgcaaaaattgggattgtgaaaacattaaagctcgtgttgtaacggaagaagaaactgtttttcatgatgtaaaattcgctaaaggtgctgctactcttctgaaacgcatcgctccactgttcaattaatgaggaaattgtaatgaaacgtaaaattgttcagaattgcactaatgacgaatttgaagatgtattatttgatccaaatcttgtagtagttcaaaaggaacatactaataagtttactcacttgacttcggtttatgtgtacgaaaaagttggtgataaacaaccaatttatggtgtgtttcgcgaaattactgaagatggtacaacttattggaaggaaacttattaatggctattaaatttgaagttaataaatggtatcaatttaaaaataaacaagctcaagaagattttatcaaggaccatactgaaaacggaatttatgcacgacgtcttggtatggaaccttttagagttacagatattgactatcttggtcgtcctactaaacttgtgacatctagtggaaaattggcgttatcttctggtaaagatattttggatgaagattttatctggctttcaacgagtgaagctgtgttctttgatgaagtggaaaatccataccaggcagctgaagagcaagagcaggaagagaaagagcaagaacaaatagaagatttcacagaattcccggtcatgaaagttactattgaaaataatgaacaggcatggtctttgtatcagatgttgaaagcacactttaaggaataattatgccaatgtatgattataaatgccaatccgaagattgcgggcatgaatatgaaaaaattaaaaagatttctgaacgagaaaatgatgtttgccctaaatgtcatcgtttgtctactcgtcggccttctgctcctaagcatgtgaatggcggtttttacgatttacttaaagggtgattatgtttaaaatcggtaagaaatatcgtgttcgcgaaggtgaagaaaagaaatatctactttctgctagtaataggaatggttctattaatgccgtaatatctacaagcgaatttatcgttgaagatatgaaaggtcataatgttacaatgattagtacagcaactagcagaaatgctggaaaaattcttcacagtctgcggagtgaagttctaatttatgatgaagaatttgacttcttcgaagaagttcctgaagattttgattttgaatgcactatcactatgaaatctggcgaccctctttcttttacagttaaagatgaaagaagccgtttgagaattattagtctccttcaagccattaaatttaagtgaaaactatgaaatatattaatcgttctattgcagcattagtattagcagtatctttagtaggatgtactgatgctgataatgcaactaaaattttatcatcgagtggttttacgaatattgaaatcactggatataattggttcggttgctctgaaaatgatttccaacatactggatttcgtgctattggacctactgggcagaaagtagaaggaacagtatgttctgggttgttctttaaggattcaactattcgttttaaataaaaggccttcgggcctttagctttatgattgccggagtataatattcccgaaaccaaacgaggataagtgatgattaagaatgaaattaaaattctgagcgatattgaacatatcaaaaagcgtagcggcatgtatattggctcttctgctaatgaaatgcatgagcgctttctgtttggtaaatgggaaagtgttcagtatgtacctggtcttgttaagcttattgatgaaattatcgataactcagtagatgaaggtattcgtactaagtttaaattcgcgaataaaattaatgttaccattaaaaataatcaggtaacagttgaagataacggtcgcggtattccacaagcgatggttaaaacacctaccggtgaagaaattcctggtccagttgctgcttggactattccaaaagcgggtggtaactttggtgatgataaagaacgtgtcaccggtggtatgaatggtgttggttctagtttgacaaacattttttctgtgatgtttgtcggtgaaactggcgatggtcaaaataatattgtagttcgttgttcaaatggcatggaaaataaatcatgggaagatattcctggaaaatggaaaggaactcgtgttactttcattcctgattttatgtcatttgaaactaatgagctgtcccaagtttatcttgacattacactagatcgtctccagacacttgctgttgtttatcctgatattcaatttacctttaatggtaaaaaggttcagggcaattttaagaaatatgcacgacagtatgatgaacatgctatcgttcaagaacaagaaaattgttctattgcggttggtcgttcacctgatggttttcgccaattaacttacgtgaataacattcacactaagaatggtggccatcacattgactgtgtcatggatgatatttgtgaagaccttattccacaaatcaaacgtaaattcaaaattgatgtaactaaagcacgcgttaaagaatgtttgactatcgttatgtttgttcgtgatatgaaaaacatgcgatttgattctcaaactaaagagcgtttgacttctccatttggcgaaattcgtagtcatattcagcttgatgctaaaaagatttcacgcgctattctaaataatgaagcaattctaatgccgattattgaagctgcgttggctcgtaaattggcggcagaaaaagcagcagaaactaaagcagctaaaaaggcttctaaagctaaggttcataaacatatcaaagcgaatctttgtggtaaagatgctgacactactcttttcttgactgagggtgattcggctatcggatatcttattgatgttcgtgataaagaacttcatggtggttatccattgcgcggtaaagttctcaacagttggggtatgtcatatgctgatatgcttaaaaacaaagaactatttgatatttgcgcaatcactggattagttcttggtgaaaaagctgaaaacttgaattatcataatattgctattatgactgatgctgaccatgatggtctaggaagcatttatccttctctgctcggattttttagtaattggccagaattgtttgagcaaggacgaattcgctttgtcaaaactcctgtaatcatcgctcaggtcggtaaaaaacaagaatggttttatacagtcgctgaatatgagagtgctaaagatgctctacctaaacatagcatccgttatattaagggacttggctctttggaaaaatctgaatatcgtgagatgattcaaaatccagtatatgatgttgttaaacttcctgagaattggaaagagctttttgaaatgctcatgggagataatgctgatcttcgtaaagaatggatgagccagtagtttactttaccacaaggatgtggtataattaattgggcaaatgaggatattgaaatgaaatcatatagagtaaatttagaactttttgataaagcagttcatcgagaatatagaatcattcaacgctttttcgatatgggagaagccgaagaatttaaaaaccgctttaaagatattagagataaaattcaatccgacaccgcaactaaagacgaactactagaagttgctgaagttattaagcgtaatatgaattaatgaggaaattatgattatcaccactgaaaaagaaacaattcttggtaatggttctaaatcaaaagcatttagcatcacagcatctcctaaagtatttaaaattctgtcgtctgatttgtatacaaacaagattcgcgcagtagtccgtgaattgattaccaacatgattgatgcccatgctctcaatggaaatcctgaaaaatttatcattcaagttcctggacgtttagatccacgatttgtttgccgagattttggtccgggtatgagtgattttgatattcagggtgatgataattctcctggtttgtataattcatacttcagttcatctaaggctgaatctaacgatttcattggcggatttggtttaggttctaaatctccgtttagttatactgatacgtttagtattacttcgtatcataaaggtgaaattcgtggttatgtagcttacatggatggtgatggcccacagattaaacctacattcgtaaaagaaatgggtccagatgataaaactggtattgaaatcgtagttccagttgaagaaaaagactttagaaactttgcttatgaagtttcttatatcatgcgaccgttcaaagatttggctattattaatggtcttgaccgcgaaattgattattttccggattttgatgattattacggtgtaaatccagaaagatactggcctgatcgtggtggattatatgctatctatggcggtattgtttatcctatcgatggtgttattagagaccgtaactggctaagcattcgcaatgaagtgaattacattaagtttccaatgggttcacttgatattgctccatcacgtgaagctctttcattggatgatcgtactcgtaaaaatattattgaacgagttaaagagcttagtgagaaagcatttaatgaagatgtaaaacgatttaaagaatctacatctccccgtcacacatatcgtgaattgatgaagatggggtattctgctcgagattatatgattagtaattcagtcaaattcacgactaaaaatctgtcatataaaaagatgcagagcatgtttgaacctgacagtaagttatgtaatgcaggcgtggtgtatgaagtaaatcttgaccctcgactgaaacgcattaagcaaagtcatgaaacttcagccgttgcatcaagttatcgtctgtttggtattaatacaacaaaaattaatatcgttattgataatattaaaaatcgtgttaatattgtccgtggattagcacatgcgctagatgatagtgaatttaataacactttgaatattcatcacaatgagcgtcttctgtttattaatccggaagtagaatcgcagattgatttactacctgatattatggcaatgtttgaaagtgatgaagttaacattcattatctgtcagaaattgaaactttagttaaaagctatattccaaaggtagttaaaagtaaagctcctcgtcctaaagctgctacagcatttaaatttgaaattaaagatgggcgatgggaaaaagaggaactatttacacttacgtcagaagcagatgaaattactggttatgtagcgtatatgcatcgttctgatattttctctatggatggtactacatcactttgtcatccatctatgaatattttgattcgtatggctaatcttattggcattaatgaattttatgttattcgtccgcttttacagaaaaaggtaaaagaactcggtcagtgccaatgtatttttgaagctttacgtgatttatatgtagatgcttttgatgatgtagattatgataagtatgtaggttattcaagttcagctcaacgatatattgataaaattatcaagtatcctgaactagattttatgatgaagtacttcagtacagatgaagtttctgaagaatatacacgacttggtaatatggttagttcattacgcggtgtgtatttcaatggtggaaaagataccatcggtcatgatatctggacagtaactaatctctttgatgaattatcacgtaatgcttcaaaaaatagtgataaaatggttgctgagtttaccaagaaattccgtattgtttccgacttcatcggttatcgcaactctttaagtgatgatgaagtttcccaaatcgctaaaactatgaaggcccttgcggcctaataaggaaaattatgtacaatattaaatgcctgaccaaaaacgaacaagctgaaattgttaaattgtattcaagcggtaattacacccaacaggaattggctgattggcaaggtgtatcggttgacacaattcgtcgtgttttgaaaaatgctgaagaagctaaacgctctaaagttactattagcggtgatattacaattaaagttaatagcgatgcagttattgctccagttgctaaatctgacattatttggaatgcatctaaaaaattcatttcaattactgttgatggtgtaacttataacgcaactcctaatactcattcaaacttccaggaaattcttaatctgcttgtagcagataagttggaagaagcggcacaaaaaattaatgttcgtcgcgctgttgaaaaatatatttccggcgatgttcgaattgaaggcggaagcttgttctatcaaaatatcgaattacggtctggtttagttgatcgtattctcgattcgatggaaaaaggtgaaaactttgaattttattttccgttcttggaaaatctgctggaaaacccaagccagaaagcggtatctcgactctttgatttcttggtagcaaacgatattgaaattacagaagatggttacttctatgcttggaaagtagttcgtgacaactactttgactgtcactcaaacacctttgataacagtcctggtaaagtagttaaaatgccgcgtactcgtgtgaatgacgatgatacacaaacttgttctcgtggtctgcatgtgtgttctaaatcttatattcgtcactttggcagttcaaccagccgagttgtaaaagttaaagtacatccacgtgatgtagtatcaattccgattgattacaacgatgctaaaatgcgtacctgccaatacgaagtagttgaagacgttactgaacaatttaaataagggcttcggcccttatcatattaaggaaaattatgttaggttatcaagcacgagtaaaagaagaatacgatcaattaatgctcaaaattaatgcactgagcaaatttttagaaagcacaaagtttctaacggttagtgcagttgagcaagaactgctgctttcacagttcatctcaatgaaatcttatgctgagtgtctagagaaaagaattgcacaattcaaataaaataagggcttcggcccttttgttttaaggtgaattatgatttattgtatgaatatcggtgattccgacataaaagaaatttctttacatggtaatcatcacgctaatattgtgtactgtaataaatttgagtttggacacgaaaaacttgggtttctttactgtaatgatgttatagtcatggataaaaaggagctcgatgagcttgacctcgagtctttagatgaaaatgaccgaatttattatggcactcttaaagtatataacgcctatctcagcggtaaaaaggaaagtctaaatgcagaaaacgaatccgggtttacagagactatttcagattccgacatttaccctatcgaacagtgacttgactagtgaaatgaaggtcaaaattgctgatacagcaagatactctttaaaacaaaacccgaaccaggataaagcagaagttattgaaagatgccgtatcgcggtgtacgcagagttttttgtggcagattggctaagcggatatgtcaataaaggccaagaaaatgtcgatgatccgtacacgtatgcatgggatgttttagcgcatccaaaatattgcgggcttcgtgtagaagtcaaaacacatcaaactgactcgcgttggatttcggtaacaacgggatgcagcggagagtatccatatggttctggaataaatttagggcccattctaaatcatcaagtcgctgactgtataattatattcaacactaaagaaattcatccaggtgtcatccagtacactccgaagttcatcggtgacagagaagaccttcgtaaggttgtaagaaaaagcaactacaacggatggtatctttccatttaaaaattttcacaaaacggtttacataccacaaggactgtggtaatatacaactatcaactgatacggatttggagaataaaatgtttactacagctgaattaaaacgagcaaaagctaagaaagggcaaggaaaatataaagctgaattagttaaagaacttcagtttgctgaggctgaattgaattcaatgattattcaaaatgctccagaaactgaaattgctcttaaacgtattgcgaataagtgtcttcgtgatgcaatcgtcgatcttttagcggattattgagtaaaatgaaaatcgttgagattgaactatgagttcattatggtggtgttttgtttggttaattagtattccattaatttgtttaacatttacttttgtgatgaggttattatgaaaatttttaattctgtacttattgcttgcgcgtggtgggttgcacaggtttcagcggtagtagttggtattcacatttattacgaatatttttaaaaaagttgtttacaagactgttcctctgtggtactatacaactatcaactacggaggaacacaaaatgaaaaagattgttaaagctatatggaatgtagttataatactaatagttttgagtatattcccaatcgttttaatgattgatatgttaaacgcttattttggatttatgtgaggaaaatatgaagcgtaaacgcggtgcttttacatttattgaatggtttttcgataatatttttccggctttatttatttttatgctgattttcgccttaggttcagttgtagttggaatctatttgatggcagtagtcggaatggatatttatcaaaatggtttaaaatccgtagttgaaacaatttggaatggtgtaaaatgatgaatttgttgagcggttggttttatattcttatgttttacattggtgcaaattttccatattggatgggatggtcaacaactgcatttggattttatactccttgaggtgaattatgaaaagttttaaagatgtaaaagttggtgaaattttctgtttagataacggtgatcagttaattcgtatttcacctcttaagagcactagcgagaaaccgacagttaacgctactttagcaaataacagtaatgaacgtttctgtattgaaaatgatactgaaacttataccgtagaagagttttgggaattgagcgtcgactgcgacgattaatttaatggccgtgtgtattcatgcggccttggagtagaaaataatttagaggaaattaatatgaaatatatgactgttactgatctgaatgatgcaggtgctactgttattggtacaatcaagggcggtgagtggtttttgggaactccgcataaagatattttatctaaacctggattttactttttagtaagtaaattagatggtcgtccatttagtaatccatgtgtgtcagcacgattttatgtaggtaatcagcgttctaagcaaggattcagtgcagttctaagtcatattcgtcaacgtcggtctcagcttgcacgtactattgcaaataacaatgttccatacacagtgttttatctgcctgcttctaagatgaaacctctgacgacgggatttggaaaaggacagttagctttagcgtttactcgtaatcatcattctgagcatcaaacactcgaagaaatgaaccgtatgttggctgataactttaaattcgttttgcaggcatattaatgagtaatttccataacgaacatgtgatgcagttctatcgtaataattttaaaactaaaggcgtcttcggacgccagtgaggaaaatatgaaatttgtttatggtatagcctttttctgctggtcctttttattcgccggaacagtaataactggagaaacttcagtatttttacaagcaacagctcaaggcgtattagcaattttatcattgttttggctattaaatgaaaatgattaagaaaatcttgggctattcattagcccttgctactttattagtagcactatattatggagtaatgttcggattaattcaagtcgtgcttttcatttctgatgttattatggcactacattcactagtatggtaaatttatgcaactgaataatcgcgatttaaaaagtatcattgataatgaagcattggcttatgctatgtacacggttgaaaatcgtgccattccaaatatgattgacggatttaagccagttcaacgatttgttattgctcgagctcttgatttggcacgaggaaataaagataagtttcacaaactcgcttctattgcaggtggtgtagcggaccttggatatcatcatggtgaaaactctgcgcaagacgcaggtgctttgatggctaacacttggaataataattttcctctgttagatggtcaaggaaactttggttctcgtaccgtccaaaaagcagcagcaagtcgttatatttttgctcgtgtaagtaaaaatttctataacgtatataaagatactgaatatgctccggtacatcaagataaagaacacattccgcctgctttctatttgcctattattcctactgttcttcttaatggtgtttccggtattgcaactggttatgcaacttacattcttcctcatagtgtttcttctgttaagaaagctgtattacaagctcttcaaggaaagaaagtaactaaaccgaaggtagaattcccagaatttcgtggtgaagtcgttgagattgatggccaatatgaaattcgtggaacatataagtttacttcacgaactcaaatgcatatcactgagattccgtataagtatgatcgtgaaacttatgtgagtaaaatcttagacccacttgaagataaaggcttcattacatgggatgatgcttgtggtgagcatggttttggattcaaagttaaattccgtaaagaatattctttgagcgataacgaagaagaacgccatgcaaaaattatgaaagacttcgggttgattgagcgtcgttctcagaatattacggttattaatgagaaaggaaaactacaagtttacgataacgtagttgatttaattaaagactttgttgaagttcgtaaaacttatgtccaaaaacgaattgataacaaaatcaaagaaactgagtcagcttttcgtttagcctttgccaaggcacatttcattaagaaagtaatttcaggtgaaatcgttgtgcagggtaaaactcgcaaagaactgaccgaagaactttcgaaaatcgatatgtattcttcttatgttgataaactagttggaatgaatatttttcatatgacttccgacgaagcaaagaaacttgctgaagaagctaaagctaagaaagaagaaaacgaatattggaaaactactaatgtagttaccgaatacactaaagatttagaggaaatcaaatgagtccattcataggtattacaagcgctgcattagtatccggtagcattttactggcgggtttaggtgttgttccagccgtagcaggaggtcttcttgcgttcggcattcaacgtgttatcatgccagttatcacagtcatgcagtaatttttagggagagccgaggctctcccttttttatttcaaaaatttttttcacaaaacggtttacaaccaaagcatactgtggtactatacaactatcaactactgatacagaattacggagattagaaaatgtctaaagtaatttacatcatcaaagcttctaacgatgttctgaatgaaaaaactgctgcgattttaattactattgctaagaaagatttcattacagctgcagaagttcgtgaggtgcatccagatttaggtaacgcagtagttaatagtaatattggggtgttgattaaaaaaggcctggtggagaaatcaggtgatggattaatcattactggtgaagctcaggatattatttcaaatgcagcaactttatacgcacaggaaaatgctccggaactactgaaaaaacgagcaactcgtaaagctcgtgagattacttctgatatggaagaagataaagacctcatgttaaaacttttagatgaaaatggatttgttcttaaaaaggtcgaaacttatcgtagtaactatctcgctattttagaaaaacgcactcacggaattcgtaattttgaaattaacaacaatggaaatatgcgaatttttggatacaaaatgatggaacatcatattcagaaatttactgatatcggaatgtcatgtaaaatcgctaaaaacggtaatgtgtatcttgacattaaacgctcggaagaaaacattgaagcagtaatcactgtagcatctgaactgtgaggaataaataatgaacaagttagaaattatcaatgaacttcgtcgttgtgcagaacctactcaagagggttgggatatttggtaccatggagcttatcttggaactattgtaaagattaagactggtaaatacatgattattcgtgaaagtaaagatgctccagtaggtattcgcaataatttcatggcagcgataagttcctttacagatgcagcttacgaaatttaccttgccgattataaagaattccaggaatctcaaccagttattcgctcaattggtgttaacaaagctcaacagaaaactttgtggcaacgtattaaaggatggtttaaatgaacccatttatcaatcgtttaaaaatgctgaatgttcctttatctcgtgaaactccagaaagtcttgctgaaaaatttaaagcgcatggttataaatgcacagaagaagatattctaaaagaagttcctgaaatatgttggcagactgcatattgggatgaaaaccaaaagtatcaacgacgaattgtctgcgcggccaaccgttttaaattaaaagatggacgaactcttattattccaggtgctcgtcattattctaaagatatggccgaagttttagatgtagttaaacctcaattagttactcaacaagtttgtgatgatgaccaagggtttattgaccaatatagtaattattggacacgtgaagaagcaatgattattgcaacttacgctggacaagtacgtattgaacggggtggtagtgaaaaagaactttactctgaggacctttactaatgaatattaaaaagtttcaaatcgatggaattatgaatcaaattcaggcactggaatatgccaataaaatgatgtcaactaattggggaatttatgcgaatgagccagcatttaaattttgtgatatggaatttaccaaaaagcttgtaggaaaagattatgtatgcccatttagttctccggtaaatggaatgctaaaacctgctttacgcgatctttatattgcgatgaacgaagaaatgataaaagaattaaaacgtcaactgaaggtgattcaatttggccagggaaattaattcaaaatctgattattttaattctctcaatgataaagataaaaatctaataaggcattttattgttgagatgggatatactgatacacgtgatttaagagaacatatatttgaatgtggtgtagctaaaaagttttcattcacatgcaaatgtttaagagaggtaattcagcactatgaacaatttagtcgcaaaacataattttaataaagcttctgttcataaggataagaagaaagtgtttaaagaatctaatcgcaaacagaaacataaggggaaggtctatgattattgattctcagtctgtggttcaatacacaatcaaaattgatattctagaaaagctatataagtttctaccaaatttataccactcaattgttaatgaattagttgaagaactacatctcgagaataatgatttcttgattggaacttataaagacctctcaaaagcaggatatttttacgtaattccagctccaggaaaaagtattgatgatgtattaaaaactataatgatttatgtccatgattatgaaattgaagattatttcgaatgagtcataatcttgaaaaagtaatcgagcataatgtagctcaggaacgtgagtcgttcaaggaattcgtagaaaaaatttttcaagaaaataacacagaccagtttacaaatcaaacgtctgatgatattataacaaagtcaactaattgagtggtatagttaatgaataaaaatattgatacagttcgtgaaattattactgttgcgtctattttgattaaattttccagagaagatattgttgaaaatcgtgctaattttattgcatttctgaatgagattggagtaacgcatgaaggtagaaaattaaatcagaattcattccgtaaaattgtttctgaattaactcaagaagataagaaaaccctcatcgacgaattcaacgagggttttgagggtgtatatcgatatctagagatgtatacgaacaaataattatttagcccttcctaatattctggccgcctgagaacatattgattcaaggcggtcattacttatatgatcatttctataccagtacatggttatcgttccagcatagatattatccaaattaaaatatggacaactgtacatgtagtttatttcgggagtaggctttttagttggtaaaaaagcaaattttgagttggaataataatgacgtccatttaaatgaactgcatattcatccatagttttatcaacaggatatcctccaagtgatttttcacttattgttgaaggtaattttccttcatatgctataatatcgacaaaatagtttaagtttttagggcggaaagaatacaccgcactaaagtctgcctcagatgatatgtgaactatctggagttgttccagggcgacagattcaaagcgtgcatttctttccttttcaataatttcactgtatgtttcatactttgattgcttatagtactcaaagaaactatctcccctataccaaacaatcgccatcataaacaaaagaattacgacagctacccgggaagcaagaactttcccggtagcgttatctttgaacaagcgatctagaacaccaaatagaatatcagagggcgaaaatgatattctaggtgctgccatagaccctccttttaaggatatttattcaaaaattatactcttgaaccgtatacggctccaacgttttgccatgttggagccgatcctgctacagcagcaccaggatttgtcggaggtgttgaataaccattacctggattagcatattgacctggttgaccaacttcacctccaccgccagcccaatattggccagtattaccaccagcaccaggtgctgttaagctagcatttccaccaggccatcgagcaccattatttccgcctaaaccaaatggacgaccaccgccgcctccacatacatattttcctgcccagttattggcttgcgaatatccaccgccaccgccaccgcctccagcaatagctcccccattattaattcgtaggcgtccacctatatcattttggatacatacaccaccgtcagacgctccaatcgctccagcagcccaaccattcccgccgcgaccatacatcgttacgccgtgtatattcaattgaacatattcattcggtgtatctccgtacatgaagaataaaggaacgtctctagaatatgatactaaatctccagtgatattaaacactataggtgcactacctgcctcaaaacatctatctcggaaccattgaccattgaagttatggtccgctccaagtgtgtgaattacttcaacagatcgacctatcatattactcatccagaacggaatgcccattcttaatcgttgggccgcgccacccatccaattttgtcctgtattaattactgcagacgatcctacccacggtcctgctactgccatataaacctccaaggggccgaagcccctcattttattttaacaatgatttaactaattttttaagctcttcaatctctgacttaagttctgcaatttctgcagtatgctcattgattgcagctgtatttaaaccaattacaccgttatagttcaaacgaagtaaagcttcgccatcagggtcaccttcaactaattcaggcaaaatagcttgaacttcttgagctatcaaaccggcgttaggttcccatttctgattgccttcttcatctaggcctcgcttctgcatataagtgtaaccgttaatttttgaaagtgtttgtgaagcattttcaaatttaacaaggtcttttttaacacgaatatcagaacggacataaacatcgcggacatatgtggaataactgtcattcgcctggacaagatggccgtgagaaataattgctttgccaacttctgtccaaaaagctccatttgaatcccaaaggccaggatgagaatgataagatgaagaccacgaagcaatatgcgctccatttgcaaccatattgattcggccgtcaccaccatgaactaatccagtatcattatcaccaataacaagacacgggccggtccatacagggtcaccaccgccagctgataatcttgctccagcttgaaccatattaggagcatacagaactccattgtggtgaaattcatagatagcttgtgggtcagaaccattttcctggttaccaacacggataatagcctgagcccattggttagtagtgcgtcgcataccaaaatcaacacccgatatgtatccttcattagtaataccagattttcctttgataatcgggtaataactatcattaccgacattgccaaaatctacaaatattggcgcttcttgtcgccattggtttccccaatcaccagcttggtgcgcaacccattgtgtaccgtttatatgaaggcggtctctattaaaattaaatgttccttgaccttgaggagaaagcgatatttcgccgttatttgttatgtatacaccgccttgtgtaatgtaactataaaaacctagaccattgtcgccgccgcctttgcctatataccagttacctgtaccacctacatcactacggatatgtgatgaagcaccttcgcccgcagttaaaatgagagcatcattgcctgccgataaagtcaatttaccagtcatcgtgtcgccttctttagaaacacgagtagctattgcatcgcgaatatttgtggtttctcccgcagaagtaaacgatttccaaatatctgagaaaatattaccattacgagcaatagtagcttcaccgccgccaacttgaacgttatcggggaaattaactgtaccatcagcattaaatcttgatgtatggccttgtgaaccatcttctttcaaatgatgtaaaacaaatgtaccagaattaatttcagtacctaaagaccaaacggctttaccgtttaaaaacttctgtttaacaataggataatatttgcttacagcagcatcatcgatttcttggaaaataggagcagcttgtgaatactgaatatcataaccaccggcaccagccccatgaccagcaaatttaatataaccatttgcagcgaacgtattataacttgactgtaaatctggaatagtaactttaccggaatttaaagctacgctgagaggacgtaaaggcccaatatcaccggtttctccttcgccaaatgctgtaggaataatatgaagactttcttctgagcgtctaaaaataactccgtaatcattgttccaaatacgaagaccattaacagcatttgtagtgatttcgccaatggtatgaactgctcggtcaacccaaatatcctttttcgcaataatattttcattaattgtttgttgaccatttaacacggcttcaaccgtattatctttattacgttgaatataatgcatccatccaataccatcagaaatttccatgacatttttacgggtgccttcacttggattagtaaaagtatttccccaaacagctatatacatgttatcaacggagctttgacccatggtatagcctttaaacattaaagttgaattattatttctaccatcgatattaacagaaccaccaataacgtcaatattgcctggagtaactaacaaaccgccatgagtattaatatttgtagtgcccttgccgcggaaataatgatgatagttaccgccttgataatatcctaaaagagtctgaccatccccaaacgcgttattatcatggtatgtcactacagtagcaagagcataattttcagttggaggagtagttaaatcggttccgttagtagaatatccagctataaattttcttaggctagttgtttcgcttggactaaataaaaacgtttttgtgccattgttaacactgaaataatatccgtcttgatgccatttaaatccagtatcattatctcctaaagcgatactagacggtcctaatattgagtaatccttaggtccgggtgttaaaccgatggaaattgagtgacttgatttaattccgccgctccaagtaaatgaccaaagataagcgtctaatccatctccactataccatgttattccatcattatctttaacttcatggaaaataccagaaccaactgcattacgcataacacgaagatgattaatagcagtagtgctttcagataatggaacattatctgccatcgaatagatgtcataatcatctttaacttttttattggtgataactttattggtataaattaccggagttgatacagatttccacccaaaaacttctggtgaagaaaacaaaccatttccattaaaaacgaaagtgctttctgaaccagctttgtaatcttgaactctgatgttaactacctgtgtagttaaaccgtcattagcaggagaaaaaataataccgcgttcacggtcagctgaatcatgaaaacgaacgtgtgccgttcctgaactttttgtaataagttctccgccttcagtcataatctgcccagcagcaataatatcacgagttactcgagcaataccggttaaattaaaattaccagtctgggtgaatgtaccgtttaaagtataatcgccagtttggttataattgcctttatgaataacattcccgtcaatactaccgcctttagcaaaacctaaatcaataatatttccctggtcatctttagtaaaaagtacacggtcttttaagtttatagccaattcaccttcggctaatactgaagcagctggacgttgacctgccgttttgcttcttttaaattgtatttgttttaaagtagccataagtcctcttaataatagccgaaatcttgaacagaatccttaattacgatttggtcaaatcgcggaacatgcgagggttgagatgcaggattctgtgaaaaaaggtttggcgcagttaaatttcctatcatagtgtctccagagcgtaatactctagagtttgcatttgttgtaacaatatttattgctccatcaacataatcttttctcgtaaggtcataattatttactggtgataacgaagttgcgcgtatttgtgatgcttcaattacaccctttgagctaatatctccgtttcgtgtgttaattactacagttctacctgctgccccttgtgatgatttaaagccgataccataccatgaaacaatatccatatttgtagtatcatatgtagcattatcaccaggaccggcaaaaataccgttgtcacctgacccagcaccaggaacagtcattcctttattaaaaattactttatttaaatatgtaccaccattagctttagaaacgaaatcgttatcagcagcttgtggtttattatattctgaatatattttaaatgatttatagagtacatcgtcaccggctggattcaatggaaaatttccttgatgccaaatgacagagcctccagttgttgaacctacttttaaatcagccattgtatgcccctttattttaatagtatttataaagaaaaagggaacccgaaggctccctcaatttaaacttctctaaattcctgtccaaacactttaccagttttagacgatgcttgcgtaggaagtaccattatatccggaggtgaagcagattcacagatataattaactcgaataccattgacgccaaactcagcaggttttgaaatgccaccatttcttgacacttcagaaaagcttaaatttctcataccgccttgacccgcttgtgcagttctctgtgcatatatcgtaaatccaactgcattttctggaacaactacatagtcttcctttaattcccaagacccagcctgtccagtaaactcagcttgtgtcgaggaaatatatccattcgacgcattataaaaacgaattgaaatattagtagttcccaacgctaataaatcagcgtcagcatatagttgtgctttaagataaagaacatctccaggaattaaattatagtcagaaagtttacttattgcagatgctgtaggtagacgtgcaatttcgttattagttccaccaacagctgacataaattgctcaacgctttcatatgttcttttaggaaatcctgtcgctccaacatcttctaaactatcaaatactacatctaatatagtttgatagtcatctgtgctttttctgttactcagtttaacatgctctagtgcgatagctcttttagaagaagtgtaaaaggcagcatatgatacgtcaaatcttgacaataaagaatcagatggaaaagcagatgttcctgccgttcttaaccaagatactacttcaggaggaaaattaacctttccgctagttaatatagcaacaagtctattgcttgaaaaagaattcatgaaactaacaaaggcagcagatgttgtatcatttgaagtagaaaaaacatatgacttactatcaaccaatgctcctgtagaagggtcaaaaactcttaaatgaagacctgcactaaatgtttgacttcctacaggattatcttgaaatttaacgtatggtcctgcggtagaaagcgggcaagaacccgctatacttattttatatcttactgaattgctttccgataaaaatggcgtttggacatatccttgtccaaactctgccataaatttttccataatacctcttattcaacccattcaaatttaacagttttattcactgggtcaggaataatgcgaacattaccaattcgtaagaaatcacgaatagtaagattacccattgtagcattatcagatggtaaagcaccaatatcagatggttgaggaggattacctccatcaaatacctgaacaaaacttgaccaagaatttttggttttctgccatgtacgtgtccagcgagtggtacgcgcttctggcgttgttggataagtaatccaatcttggtaaagcgaatcaagtgtgttaccaaactgagtcagtgtaccaggagatttaacttcttcgccacgttctaaatatggaagaccagtcacttcattagttttttcaaccattttaaaataacctgggaactggttataagtagctgaatcgttaatatcaattgaccagaatcctacagtatcagatgttggcgcacgtgtatataagtcagatgttttagtaccttgagaacgaattctcgagttaacagttaaaccaccagaatttatagtagcacctttggcgatgattaagctttcaccaatcgttacttgaccagatgcattattaatagctaatgggcgtaatccgttaaatccaccagtctgatcacccgatgcagtaagcataaaattcgttactgatccatcattgcgaacaatgaaaccataatttccgttaatagcacggaaagcatttgcgcttcgagaaataatttcaccagctgcagtgactgaattaccaaatgttgctacgccatttgcattcaatgttcctgaagcattaacatttatcggtgttactgtaccgttgatattaaatgttatatttccggctttattgcgctgagaataaaaatgactagatgtttcatcactaacttcaaatacggtagaacgtgttgtatctaattccccactaaattgatttccccacactctaacagtcatcgtttgggctgggtttgttccagtttgaggtcctttctcaaaaatcaatcgagttgctgttccagtattagaaatagttaatgtactatttgctgaaactgatccaccgaacgtagcagtactagatgatacaagaggggcactcagattcgtttgttgagttaaggttagtgaaccattaaccgtctgcgcaatatccctacgaatgaactgagatgaatctagaccatccaataaattactatctgcagcttttgcttttaatggcaaataatttgctaatacacggtttaattcgtatggtgatactgcatagctatttttctcgtatagctctaagtcctgggtagaaccggctgtatcattaccaacgaatgtaattgaaccagatgaagttttaacaaagcctcttactgcagtagtagctgcccaagtaggttcactctgcacaatccattttaaattttttggagatacagcagtatttgccgatgttccagcaacagtttcggtttgtgttgcaactttaataacaccttcttgtgcttcagtagatttagtacctaaaagctttttaggagttattaaaacattatctaattttccagcagcagcttcaacttgtgtagccacacgaagtgtaccacgttgtgtctcatttgcttcaagaatattaagggtataatggtcccagagagttcctgattcaactaatccagatagagcaacaacagaagtacgatcagtactattaaatctagttttaatttttaatggtgtagagatacgagtatcgtcgacgcctgcgtcgaattcaacttgtgtagcaatttcagctataccacttaaactttcagttgctctacggtcatttaaagttttaggagtgactgcacgagtataatcagttcctgtattaacttcactttgcgtagcaatttcaattaaaccaattcttccatcagttgatgtctttttatgtaacgtttctggcgttacaacagcatttgcccatccttcctgactttgtccagcaattacttcactttcaactgctaaaattactgcaccttgttgcgttggagtagctttatactgatccaaggctttaggtgaaacaactaagttattagtgtttttattataaacatttgtaccatttaattcacggctagaagccggagtagcacctgcagtagatacaaaagttacaataccagataatgattcagaaccttgacgagcttgaagctttttaggagtaatgatggttgtatcatctgttcctgtgtttgtttcttgctgcgtagcaatttcagcgacacccctacgagtttctgtagcagttctttcattcaactttttaggagtgatgataagatcatctgcaaaagagaatgtggtatcctggtttacttgcgcagttgttgctattcttgcaatacctctacgagtttctgtagcagtacgattagctaacgtttctggagtaattgccagttctttttgtggagaattttctagatcagcattagcttgagcctgtgtagctaaagcaattacgcctaatcttgctctagtagaattatttaaagagtctactctttctacagttggaacgttttgctgtacaacccaatattttccatcggaatcttcgatataagcaagctgtaaaactggaacataattggtttcgccattaaaaactaattcttgaacagtcacccatttagtttcaggtgggtattctgaacgttttggaaattgcagcaattgaactgaagaagcaattttatcttcaccggcagctttgattttaactgtttgtccttttctcatataattcatggaaatcttaacagtatcaccgatagaaatatcagttggaagctgaagttcaattgtttgggttgttccattattcgcgccaaataccatgacttcttcatttggacgaatattagaattagtcgttataatgcgtaaacgtgctttactatccccgtcaaacaatctccacaatttctcattatcatcaaacattaagaaaccatcaatagatgtacggccttcaatggaatgagttccaacttcttgtactgaagttgtttcatcatacgtggtaacaattgtgtgataaagtggatttaatttatctaaatcaacaaaattaataatatcgccatgattagcaaatcttggaagtttaacattaattggtgcagcagaagtaaatctacgtacgataaaatcgtttgattgcgcttgataaagacttgccggtgttacaactgccgcttctctgctataatcagcaacatacatttgccacagacgattactaaaaattaaaactatctgtgattttggatgagtcattagtactgagcgtacttgttgacctctaaagtttacaatgctttgtaccggagctgtaattaaaacctggttaactccaggttttcctccaatatcttggagaacgatagtatcaccatcaattggagaagatggcaaagtaaatgtaatgtcattacccgctgcggtgttaactgaaatcgattcgcctgattttaattgataaggtcctgacgaaacagttgtccagtttgcatcagtgcgtaacgctctccatcgtacgctattaaaagctcctgctggttttggaatatcatttatagcagcccaaaaacggttatcataaatgattacaaaatcttttaaatatccacgagttggatcatattgttgaactgtgttttcttgaattaagtaatcaacgtttacaccgtcagttcctacggtacgatcagctaaagctacgttgattattttatcaccacctgcgtccagaccgtcttctgctctgaactttcttttaatctcggccattctcccgggctcctattgtgttttcaataataagtatttatacttgtttactttaagatttggatggtatataatagaaatctcactaattgaacgaggttcatatggatttagaaatgatgttggatgaagattacaaagaaggaatttgctttattgactttagtcaaattgcgctttcaactgctttagtaaacttcccagataaagaaaaaattaatttatcaatggttcgtcatttgatattgaactcaattaagtttaatgtcaaaaaagcaaaaactcttggatacactaaaattgtactgtgtattgataatgcgaaatctggatactggcgtcgtgatttcgcgtattattataagaaaaaccgtggaaaagcacgagaagaatctacttgggactgggaaggttattttgaatccagtcataaagttatagatgaattgaaagcttatatgccatacattgttatggatatcgataagtatgaagcagatgaccatattgctgttcttgttaaaaagttctctttagaaggacacaagattttaatcatttcatctgatggtgactttactcaacttcacaaatatccaaatgttaagcaatggtcaccgatgcacaagaaatgggttaaaattaaaagcggttctgctgaaattgactgtatgactaaaatccttaaaggcgataaaaaggataacgttgcttcagttaaagtacgatctgacttttggtttaccaaagttgaaggtgaacgaactccttcaatgaaaacttcaatcgttgaagctattgctaatgaccgtgaacaagctaaggtgcttctcacagaatctgaatataatcgttataaagaaaatttagttctaattgattttgattatattcctgataatattgcttcaaacattgtgaattactataattcatataaattaccaccgcgtggcaaaatttattcatattttgtaaaagcgggtctttctaaattaactaatagcattaatgaattttgaggtgaataatggctaaaaaagaaatggttgaatttgatgaagctatccatggcgaagacttggctaaatttattaaagaagcatctgatcataaactgaaaatttccggttataatgaactgattaaagatattcgaattcgtgccaaagatgaacttggcgttgatggtaagatgtttaatcgtctattagctttgtatcataaagataaccgtgatgtgtttgaagctgaaactgaagaggtagttgaactttatgacacagttttctctaaatgatattcgtccggtcgatgagaccggtctttcagaaaaagaactttcaattaagaaagaaaaggatgaaatagcaaagcttcttgatcgtcaagaaaatggatttattattgaaaaaatggtagaagagtttggaatgagttatcttgaagctacaacagcattcttagaagaaaattctattcctgaaactcaatttgctaaatttattccttcgggtataattgaaaaaattcaatcagaagctattgacgaaaatcttttacgcccttctgttgttagatgtgaaaaaacgaatacgttagactttctgctatgattaaactccgcatgcctgctggtggtgaaagatatattgatggtaaatcagtttataaattatacttaatgataaaacaacatatgaatggaaaatatgatgttattaagtacaattggtgcatgcgggtgtctgatgccgcttatcaaaaacgaagagataagtatttttttcagaagttatcagaaaaatataaattaaaggaacttgctttaatctttataagtaatttggttgctaaccaagatgcttggattggtgacatctctgacgctgatgcacttgtgttttatcgtgaatatatcggacgcttaaagcaaattaaatttaagtttgaagaagatattcgcaatatttactattttagtaaaaaagttgaagtttctgcttttaaagaaatctttgagtataatccaaaagttcaatcaagttatatttttaaacttctgcaatcgaacataatttcgtttgagacgtttatcttgcttgattcatttttaaatataattgataaacatgatgaacagactgataatttagtctggaataattattctataaagttaaaggcttatagaaaaattttaaatattgattcacagaaagctaaaagtgttttcattgaaactgtgaaatcttgtaagtattgatatgaatatagtatattggtttacatttgaagaccgtgtcaaaaataagactccaccatactactatatcggtagtaaattaaattgctcatttgagaacggaataatatatgactcttccggaaaggaatactggagctcatgtaaacaaaaaagatttttgaatgcgctaatgcttcaaaaaccgagcgttaaaataattcaaattgatgatgacttggatgttattgaagcagaacgaaaataccaacttgaagtaaatgccagagataatccagactattttaatctggtatatgctggtggtggatttggtgtgagtggtgaaactcatccagccaaagacccggaagttagagagcatatgagattggctaattatatgaaccgtgacgattttagaccttggaaaacatcacgagctaatatagagtcttggaaattatctcatattgcttatgagaattatgtgttattattatcctctaatctgtacggtaaaactcctggatggcgaagagttaaaggtaatataaatataactgatacaactgctaaatcgatggtaaagtatttcaactcaggttggatacctctcgaagacccagagtattgcaaattatgccagctatgaggtaaagtgtcatagcaccaactgttaattaaattaagttaaaaaggaaataaaatgtttaaacgtaaatctactgctgaacttgctgcacaaatggctaaactgaatggcaataaaggtttttcttctgaagataaaggcgagtggaaactgaaactcgataatgcgggtaacggtcaagcagtaattcgttttcttccgtctaaaaatgatgaacaagcaccatttgcaattcttgtaaatcatggtttcaagaaaaatggcaaatggtatattgaaacgtgttcatctacccacggtgattacgattcttgcccagtatgtcaatacatcagtaaaaatgatctgtacaacactgacaataaagagtacggtcttgttaaacgtaaaacttcttactgggctaacattcttgtagtaaaagacccagctgctccagaaaatgaaggtaaagtatttaaataccgtttcggtaagaaaatctgggataaaatcaatgcaatgattgcggttgatgttgaaatgggtgaaactccggttgatgtaacttgtccgtgggaaggtgctaactttgtactgaaagttaaacaagtttctggttttagtaactacgacgaatctaaattcctgaatcaatctgcgattccaaacattgacgatgaatctttccagaaagaactgttcgaacaaatggttgacctttctgaaatgacttctaaagacaaattcaaatcgtttgaagaactgagcactaagttcagtcaagttatgggaactgctgtgatgggtggtgccgctgcaactgctgctaagaaagctgataaagttgctgatgatttggatgcattcaatgttgatgacttcaatacaaaaactgaagatgattttatgagctcaagctctggcagttcatctagtgctgatgacacggacctggatgaccttttgaatgacctttaatagattataattactaattaattggggaccctagaggtccccttttttatttcaaaaattttttcacaaaactgtttacatccctgttcttctatggtactatacaactatcggcaatactgctgatgatcaaagaggaaaataatatggctaaagttgatattgacatcgttgattttgaatatattgaagagattattcgtaatcgttatcctgaacttagtatcacaagtattcatgatgatcccaattattgcaatttttctattgtcattgaaggtcctcttgaagaccttacccgctttatggctaatgaatattgcgatggcatggattctgaagacgcagaattttacatgggactgattgaacaataattatcaaggggctatttgatagccccgttaaaatgaggaaatcaaagtggaagttggaaaaacttatatcattaaccctttatttaaaaatgactttgctaacgagtgccgttgcaatgtaatgatgctaaatttaatagagaagcacggacctagttttgttgtagatgaaatggaaagtgatgattgtgctggtgactgtgtggtacgtgtaactatggaagacggcactgaatacatggcgggtggcccacttgagacgtattttgagatttactctgaagaattcaaatactttactgaggtagatttagtaaagcctcaatctgctaatgaaggtatagtttctggcgtaacaaaaattcactgcattgttgatgaaaacaatgtagatgaaatcattgaacttttgcgaaaaacttttaaggcctaatatgactaaagatgatttcctggatgcggtagaaaaagctagtgagaaatttgaagtgatgggtgtacgggtccataaagacagaaattacggactcgatattcaaacgtattcgttatggcttgtgggtgaacacagtcttgtaaccagtttagttatgggtgtcgatacctatcctctattggttaagttcagaagtatggaaaaggctactaaagaaatgattaagcttggtaagtggctcgataaaattcaaaaataattgtttacaacagggtagtagtgtgatactattaccctatcaacttgaggagaataaaatgagattacaacgccagagcatcaaagattcagaagttagaggtaaatggtattttaatatcatcggtaaagattctgaactcattgaaaaagctgaacatcttttacgtgatatgggatgggaagatgaatgcgatggatgtcctctttatgaagacggagaaagcgcaggattctggatttaccattctgacgttgatcagtttaaagctgattggaaaattgtgaaaaagtctatttgaggaaaataatatgtccgacattcatgtaaccggaattacccaagtaagaattcgttgccaacttaaaactgcacccgggatggcttatattaatttatcacatgacctagattctcgcgaaagtctgttaactggtgtaattacgttttacactggaatcggcggaaacgaatttactgtaggcgagattgttggtcataaattaaaagttcagaagaacgtgttagaactttttaatgacctttatgatgatagcgatgaagtttttgatgaaatcgcacaagcagttaacaaaggaatggtaactttagttaaaatgattaactctatcggatatgctcctgacccttttcgatgggagataatataatttttgtatttgagtttatgaatgatgaattctattatacaatgctttaaaaattgcatagtcttgatttaaatgaatttgaggaaaatataatgcttactgaatacacttgctttgctaaagatgatgcacatcctgaactgattcttgaagttaaggttaatacgcttgaacaaaaatttgattggagcctttacatctatcctaaaaatattattgatggtccacagaaatatacgcttaatgacaagtgcattagtggtatcattggatatggtacaagtgaaagcaagtattcattagaagatattcgcaattctcgttgtgcggtatcatattttgatacagatataattaaacatctttataaattggttcttgatattcgcaaagattttactgccatcaaaaaggcaactaaggaattaaaatgattaaattagtatttgcttatgctccaactaaaacagtcgaaggctttaatgaattagcattcggtttaggtgatgggttaccatggggacgagttaaaaaggacctccagaattttaaagctcgtaccgaaggaacaattatgattatgggtgctaaaacgttccagtcattatctacattacttcccggtcgtagccatattgtggtgtgtgaccttacgcgtgattatcctgtaactaaagacggcgatttagcacatttctatattacatgggaacagtacataacttacatttctggcggcgaaattcaagtgtcaagccctaatgcaccattcgagactatgcttgatcagaattccaaagtaagtgtaattggaggacccgctctactatatgctgcgttaccttatgcagatgaagtagttgtttctcgcatcgttaaaaggcatcgtgttaattcaacggttcaattggatgcaagttttcttgatgatataagcaagcgtgaaatggttgaaacgcattggtataaaatagatgaagtaacaacccttacggaatcagtatataaatgaaataacgcgtggctgaaaatatgaattttaattattatcctattctattagaaaaagatgagaaacagccaaagtggcagggtcctcagtttattaaaggtgtctatcaattagtagttcctaaagacaagatttatagtagctgtttcactgaatccgcttgtagtatttttggtaatagttctccgtattggaattttgacataaagctggatagaaatatcgatatttggttgaaagctatggatattggtaatattacgtttgatgagaataattatcatattattggtcgcttttccaaacgcggtaaagaattatatttcacacctgaaattgagagaaaatttgatgctaaaccgtattgattatttagcttcataaaatgtattataatttatataaggtactaaatgaaacaataccaagatttaattaaagacatttttgaaaatggctatgaaaccgatgaccgaacaggcacaggaacaattgctttgttcggtactaaattacgctgggatttaactaaaggttttcctgcggtaacaactaagaagctcgcttggaaagcttgcattgctgagctactttggtttttatcaggaagcacaaatgtcaatgatttgcgattaattcaacatgattcattaattcaaggcaaaacagtctgggatgaaaattacgaaaatcaagcaaaagatttaggataccatagcggtgaacttggtccaatttatggaaaacagtggcgtgattttggcggtgtagatcaaattatagaagttattgatcgtattaaaaaactgccaaatgataggcgtcaaattgtttctgcgtggaatccagctgaacttaaatatatgacattaccgccttgtcatatgttctatcagtttaatgtgcgtaatggctatttggatttgcagtggtatcaacgatcagtagatgtttttctcggccttccatttaatattgcatcatatgctgcgttagttcatattgtagctaagatgtgtaatcttactccaggagatttgatattttctggtggtaatactcatatctatatgaatcatgtagaacaatgcaaagaaattttgcgtcgtgaacctaaagagctttgtgaactggtaataggcggattgccttataaattccgttatctttctactaaagaacaattggaatatgttcttaaactcaggcctaaagatttcgttctcaaagattatcagtcccacggtgtcttgaaaggaaaaatggcggtataattttaatttaattgcgaggatatatgattttacgatttaaagatacttctggtgtagttctttttacacttcctaatccaagtgagttagaagttccaggaccaaatcagcctattatcatttatggcaaaaaatattatactcataaaatgactcgtgagtattttgataataaaatttccacagttaaaacttcttctgactgttactacgatattactgttttaacggaagaacaatatgaacacttacaaccgcgcaggccgtatatgccgagtagtgaataaatataaatccgactttgatgttaatattcaccgtggtacattttggggaaattacgtcggtaaagatgctggcagccgggaggctgccattgaattattcaaaaaagattttatacgtcgaattaaatccggagaaataactaaagcacatttagagcctttacgtggaatgaggctaggatgcacatgtaaaccaaagccgtgtcatggtgatataatagctcatatagttaaccgattgtttaaagacgattttcaagttgaggacttatgcaattaattaatgttatcaaaagtagtggtgtttctcagagctttgacccacaaaaaattattaaagttttatcttgggcagctgaaggaacatcagtagatccttatgaattatatgaaaatattaaatcttatctccgtgatggaatgacaactgatgatattcagactattgtcattaaggccgctgcgaattctatttcggttgaagagcctgattatcaatatgtagctgcacgctgtttaatgtttgctcttcgtaagcatgtttatgggcagtatgaaccacgttcatttattgaccatatttcttactgtgtaaatgaaggtaaatacgaccctgaattgttgtcaaaatattctgcagaagaaattacatttttagaatcaaaaattaagcacgagcgggatatggaatttacttattccggggcgatgcaattaaaagaaaaatatctagttaaagataaaaccactggtcaaatttatgaaactccacagtttgcatttatgactattggaatggcattgcatcaagatgaacctgttgatagattaaaacacgttatccgtttttatgaagcagtatctactcgacagatttcattgccaactcctattatggctggttgccgtactccaacccggcagtttagttcatgcgttgttattgaggcaggtgattcattaaagtctatcaataaggcttccgcttcaattgttgaatatatctctaaacgcgctggaattggtattaacgttggtatgattcgtgctgaaggttctaagattggcatgggtgaagtacgccatactggtgttattcctttttggaaacattttcagactgctgttaaatcatgctcacagggtggaattcgtggcggcgctgctactgcttattatcctatttggcatttggaagttgaaaatcttcttgttttgaaaaataacaaaggcgtagaagaaaaccgcattcgtcatatggattatggtgttcaactgaatgatttgatgatggaacgttttggaaagaacgattacattactttgttcagtccccatgaaatgggcggagagctttattattcttattttaaagaccaagaccgtttccgtgaattgtatgaagccgcagaaaaagaccctaatattcgtaaaaagcgtattaaagctcgtgaactatttgaattgctcatgactgaacgctcaggaacagcaaggatttatgtacagttcattgataatacgaataactatactccgtttattcgtgaaaaggcacctattcgtcagagtaacttgtgctgtgaaattgctattccaacaaatgatgtgaatagtcctgatgctgaaattggattgtgtactctctctgcattcgtactggataattttgactggcaagaccaagataaaattaatgaattggcagaagtccaggttcgtgctcttgataatcttttggattaccaaggatatccagttcctgaagcagaaaaagctaaaaagcgtcgtaacctcggtgtaggtgttactaactatgcagcttggttggcaagtaactttgcttcttatgaagatgctaacgatttaacacatgaactatttgagagattacagtatggactcattaaagcatccattaaactcgccaaagaaaaaggaccttgtgaatattattcagacactcgttggtctcgaggcgaattacctatcgactggtacaataaaaagattgaccaaatcgcagctccaaaatacgtttgtgactggtcgtcgctgcgggaagaccttaagctctttggcatccgtaatagcacactatcagcacttatgccatgtgagtcatcttcccaagtttctaacagtacaaacggtatcgagcctccacgtggaccagtctctgttaaagaatcaaaagagggctcctttaatcaagtcgtgcccaatattgagcataacatagacctttatgattatacatggaaattagctaagaaaggtaataaaccttatcttacgcaggtagctattatgctgaaatgggtttgtcaatcagcttcggcgaatacatactatgacccccaaatcttccctaaaggcaaggttccaatgtcaataatgattgatgaccttttgtatttctggtattttggcggaaaaaatttctattatcataatacccgtgacggttctggtactgatgattatgaaatagaaactccaaaagctgaagattgttcatcctgtaaattatgatataatttgactcacggacgagtcaccaactattaactaagcggaaaatttatgagcacagtttttaatacaaatccagttgatgttttaaaagaacctatgttttttggttcaggtcttggtattgcgcgttatgatattcaacgtcataaagtttttgaagatttaaccgaaaagcagttgtcatttttctggcgtcctgaagaagtaaacttaatgatggatgctgcacagtttaataagcttcctcaatatcagcagaatatttttactaataatctgaagtatcaatcacttctggatagcattcagggtcgtgcaccgtctgctgtacttatgtcattgatttcagacccaagccttgatacatgggttgctacatggacttttagtgaaactattcacagtcgttcatacactcatattatgcgaaatctttatactgatccgtcaaaggtatttgatgagattgtattagataaagctatcatgaaacgtgctgaatctattggtcgttattatgatgatgttattgagaaaactcgtgaatgggaaaacgcaaaagaaatttttaggtgtttttcagagaatgaatatgatttggacgcatattcaaatagccttatgcatgtagaagaatgcaaatacgaactaatgcgttctctttacctttgtctgcatgtaatcaacgcattggaagctattcgtttttatgtatcctttgcatgtactttcaacttccataagaacatggaaatcatggaaggtaatgcgaagattatgaaatttattgcacgtgatgagcagcttcatcttaaaggtacacaatatattattcgtcaacttcaatctggtactgatggcgatgaatgggttaaaattgccaaagagtgtgaacaagaagcagttgatattttcatggaagttaaccgccaagaaaaagattgggcagttcatttatttaaagatggcgatgttcctggattaaatacaaatagcatgtggagctttattgattacttaactgtatctcgtatgaaacagtgtggtcttccatgcccgattaccgatgctccggttaaacatccatatccttggattcgtgaataccttaattctgataatgttcaatccgcgccgcaagaagtagaactgtcatcttatcttgttgcacagattgataatgatgttgatgataaagttatgatgagttttaaaaaatacttttaaggagtgggccgcaaggcccattttattatgaaagaaattgcaacagaatattcatttattaaatatactgagttagaattagacgacaacggaagtataaaacaactatctattccaaataagtataacgtaatttatgctattgctataaatgatgagcttgtttatattggaaaaactaaaaatttacgtaaaagaataaattattatagaactgctattaatcgtaaagacaaaacgtctgattctactaaatctgcattaattcatgctgcgctaaaggaaggaagcaaagttgaattttacgcccgccaatgttttaatctttctatgacaaatgagttaggtacaatgacaatcgcgacgattgacctagaggagccgctattcattaaactgtttaacccgccttggaatattcaacacaagaaaaaatgatgcttccacatggagtgtggtactatattcaaaacacaaaagaggatacacaatgcaagaactttttaacaatttaatggaactatgtaaggattcacagcgtaagtttttttactcggatgatgtaagtgcatctggaagaacttacagaattttctcatataattatgcatcttattctgattggttacttccagacgcattggaatgtcgtggaatcatgtttgaaatggatggagaaaaaccagtaagaattgcttctcgtcctatggaaaagttttttaacttaaatgaaaatccattcacgatgaatatcgatttaaatgatgttgattacattctgacaaaggaagatgggtctttggtatcaacttatttagatggtgatgaaattctgttcaaatcaaagggttcaatcaaatccgaacaggctttaatggctaatggtattttgatgaatattaatcaccatcggttgcgtgatagacttaaagaattagctgaagatggatttactgctaacttcgaattcgtcgctccaactaatagaatcgttcttgcttatcaagagatgaaaatcattttattgaatattcgtgaaaatgaaacaggcgaatacatttcatatgatgatatttataaagatgctgctcttcgtccatatctagttgaacgatacgaaatcgatagccctaaatgggtagaagaagctaaaaatgcggaaaacatcgaaggctatgttgctgtgatgaaagatggttctcattttaaaattaagtctgactggtatgtgtctcttcatagcacaaaaagttcattagataatccagaaaaattgtttaagactattattgatggtgcatcagatgatcttaaagcaatgtatgctgacgatgaatattcatacagaaaaattgaagcatttgaaacgacttatctgaagtacttagaccgagctctgtttttagttcttgactgtcataataagcattgtggtaaggatagaaagacttatgcgatggaagcgcaaggtgttgctaaaggtgctggaatggatcacctgttcggtatcattatgagcctatatcaggggtacgatagtcaagaaaaggtcatgtgcgaaatcgaacagaattttttgaaaaattataaaaaatttatcccagaaggatactaagctgtttacaagtccctcgtgttgtgttatagtagtcttactgacataacatgaggactttatgatggatttgcagcttattactactgaaatggtcgttgaagcatacggtgatactacagatgggatttctgtattcaaaggaaatcgtcgagttggatatatcaccgatcttaagaaagatttagctaagcaagtcaagtggaaaacgaccattaaagaatatcgaaatcgtcgtcttgagcaagcccgtgatatgcttcctgatgcggttgaggagatgaaagtctttttagaaaatcaactcgctaagtatgattgtgaagtgtttattaatcagactcaacctaatgttcatatcaatagctgcaaatgctatatcatcgttaatcctttgacaggaaagcatcgtcttggaattagtaatccaaatcgtagcgcatcggatatggcagaagatgttgcggcgtgctttaaaatttctaaatctccggctgaacatcatattttaattaacggtctttctcaagacgatattatagaggttattaaaactttatgcatgtaagtaattttacagctggattgctattacttgtaatagcatttggcggaacatctattattttaaaaaataaggtagaaagattagaaacgtctgttgtagaaattacaaaaacggccaatgaaaacgccttagcattaaataatttgcgaattcagtataattatattgatgcgatgaataataaaaatcgtgaggcgattgctgctattgagcgtgaaaatgaaaaactgcgcaaagatgcaaagaaggcggatgtggtggctcgtaagccaggattggttgaaaaacaaatcaacaactccttcaacaagttcgcagaagacatccaggacctttctaaatgattaaattatctgcagtaatattatctattggtcttctagttggttgttcgacaaagcctctagaagtaaagaaagaaacagttcatcctaattggcctgtgcaaataaagtcatatgacgaagctaaactatcttggcaagttaaagttattgatggtaaagcttgggtaggtatgccatttgaagattctcaggaatttcgtatttggcttaatgatgtaaaacgatatgtacatgaccagaaaactatgatatgttattatcgtcaagagctaaaagaggataaatgtaaatgatttcatggtatcaatttgaacatctaaaaggattaatttatgaatccgagatggctgcaatgatttatggacgacagattcaacgattagaatctttacctccaactaatgatgttttattagctcaatcacgcgctaatctcaaaaatgaatatcaaaataagtggggtaaagcatctaaagacctgcatgattatattcaatcattggttgagaaaaataaatgaaaactctgttagaacgttatattgaatgctcagaccgttacattgacgtatgccgtgaaaatgtatcaagcattagcgaagacattgaacatgctaaagctttagatgatgctggtaaagccctgcgaaaagaagcgaaagctcgtgggcttgatatgtatcagcttaaaaatcacatgataaaatttatttcatccaatgttcagagtaaatcagcgaatcaatcaacagctgaattgtacaaaggacggcgtgagcataatattcgtattcttgaagttttcttaggaattaaatgatgaaaaagattattttgactattggctgtcctggttctggtaagagtacttgggctcgtgaatttattgctaaaaatcctggattttataatattaatcgtgatgattatcgtcaatccatcatgggtcatgaagaacgtgacgagtacaagtataccaaaaagaaagaaggcatcgtaacttacatgcagcatgatgttgctaacatgattctatgccaagacgtaacgaagggtgtaattgtttcagatactaatctaaatcctgaacgacgtaaggtctgggaagagtttgccaaagagcttgggcatcaaattgaatataaagtgtttgatgttccttggactgaattggttaaacgtaactcaaaacgtggaactaaagcagtaccaattgatgttttacgttcaatgtataaaagcatgcgggaatatctcggacttccggtatataaagggactcctggtaaaccaaaagcagttatttttgatgttgatggcacattagcaaaaatgaatggtcgtggtccttatgaccttgaaaaatgtgataccgatattattaatccaatggtcgttgaactagctaagatgtacgataagcaaggatattacatcgtagtcgtttcgggccgtgaaagtggaaccgaagaagacccaacaaaatattatcgtatgacccgtaaatgggttgaggacattgctggtgttccattagttatgcaatgtcaacgcgaacaaggcgatacccgtaaagatgatgtagttaaagaagaaattttctggaaacacatcgcaccacattttgatgtgaaattagctattgatgaccgaactcaagtagttgaaatgtggcgccgcattggtgttgaatgctggcaagttgcctcaggagatttttaatggctttgcatcatgaaatttgggctattattattgtaaatagtggtttagtcggtactagtaatgggcaattttgtgtattcactagtgaaatcagagcttgggaggaatgccttaaattaagagaaaagaatcctgatgttgaactaacagtaaagaaaactaaactgcctttaccgtggaaaacttatgaataatatagaaaaaatttatcatctttgtgataaaattgaaaaagaaaagaaatacctgttttgtttatggcctattgttaatggaagagtaggtttagatgttcttgattatgaaacagaagacaaagtagatggttcaacttttgataatgcgttggatgttattgattggctcgaagaaaattatgtgaggtaaatatgtttccgacttattctaaaattgtagaagtagtgtttagtcaaattatcgctaataatatgtttgaaaagcttgataacgcagccgagcttcgaattcatgctcaagtgactcatgtattgaacactttgcttccagaccaggtggattctgttgccatcacgttgtatccaggttccgcgcatatcattgtcgtattcggtcttgatgctgagttagttatcaaaggcgatattcgttttgaatcgcagacagcagaattcaaagcaatttaatagtttactttacggtagagttgtgatattatagctctaccaaacaaatgaggaaaacaaaatgctgctaagtgaaaaaccgattactgttaaagaattccaagaaaaagttaagctatttgcacaggaattggtaaataaggtttctgaacgatttcctgaaacatcggttcgtgttattactgaaactcctcgttcagtattagtaattgtgaatccaggtgatggcgatcaaatatcgcatcttaaattagattttgatggattagttgaagcacaaagggtgtatggcgtactatgatgaatttaactgatataattgataattgtcttgaaaatgatactggcgatcatagagcgcttgactctgaaacagcacagttcattagaataactttaatgaatgacactctagtaaatagtattcatccttccgtatatgatgctattattgtgacgaagtatccagttgagcttcacaaaaagatggttggcgcaatttttattgataagaaaaaccgctttaaagatgggcagaatataattagttctgttattaaaagtataactaaacttcgtcacgaaatttatcgtgttgaaactgctaaatctgcttatctggtgattatgaaatgaaagcgagtacagtacttcaaattgcatatttagtatcgcaggaatcaaaatgttgctcctggaaggtaggagcagtaattgaaaagaatggacgtattatttctaccggatataatggttcacctgctggtggtgtgaactgttgtgattatgctgctgagcaaggttggttgctgaataagcctaaacatactatcattcaaggccataagcctgaatgcgtatcatttggttcaactgatcgttttgtcttggcaaaagaacaccggagcgctcactctgaatggtcgtctaaaaatgaaattcatgctgaactaaatgcaattttgtttgctgcacgaaatggttcttctattgaaggtgctactatgtatgtaacactttctccttgtccagattgtgcaaaagcgatagctcaatctggtattaaaaaactggtttattgcgaaacatacgataaaaataaacctggctgggatgatattctgcgaaatgcaggtgttgaagtgtttaatgttcctaagaaaaacttgaataagttaaactgggaaaatatcaacgaattctgtggtgaataatgaaatttcgtttggtaaagctcacagcaattagttcttattctaatgagaacatctcatttgctgtagagtataagaaatattttttctctaaatggaagcagtattataaaactgattggactagtatagatagaccatatagttggaaatctgatttagaaaaatgccaaaaattgctttccactcttaaagaacgtggaacaactcatattaaaactgtgataggtaaataaatgaaactgacgactgaacaaaaagtagcaattcgtgaaattttgaaaactaaactgtccatgggtgtttcaaacgtagtttttgaaaagtctgatggtactattcgtactatgaaaggtactcgtgacgcagactttatgccaaccatgcaaactggtaaattgactgaatcgactcggaaagaatctactgacatgattccagtatttgatgttgaacttggtgcatggcgaggtttttctattgacaaattgatttctgttaatggtatgaaagttgagcatttactccaatttattggtaaataaatgctttaagaattatttgttattattaattcatctgttaacaaaaaggaaaaacgatgtctgaagtacaacagctaccaattcgtgctgtcggtgaatatgttattttagtttctgaacctgcacaagccggtgatgaagaagttacagaatcaggacttattatcggtaaacgtgttcaaggtgaagttcctgaactatgtgtagttcactctgtcggtcctgatgttcctgaaggtttctgcgaagttggtgatttgacttctcttccagttggtcaaattcgaaatgttccgcatccttttgtagctctgggtcttaagcagccaaaagaaattaaacaaaaattcgttacctgtcactataaagctattccgtgtctttataagtgatataaataataatatgaattgggtgtcggaataataagttaaccgaacaattctatgtggtagtctacaactgagagatctgtcgaaagaagatgaaattcagaagaacgtgactaccgagttttaatctctaacgagaatttttaaatgattaaacaattacaacacgctcttgaactgcaacgaaacgcatggaataatggtcacgaaaactatggcgcatctattgatgttgaagccgaagctcttgaaatcctgcgttatttcaaacatctgaatcctgctcaaactgcattagctgctgagcttcaggaaaaagatgaacttaagtatgctaagcctctggcttctgctgcacgaaaagcagttcgtcactttgtggtaacactgaagtaatttattggagattcactaccttagtgtgagctaaaatcgaggagccgtcgaactgtctgattaatgatttgcgaatcattatagttttaagaccccggcagttttacggtgtacctcttgaatgttactatcatgcgtcagaaaagtgttatctgtgtaagtcttggtggtatgttgacgaggtttatggttatcctgtcggtaaatatacaaaacctatgttcccccttgagggcttgcgcaggcaatgccaataagtcctgcattttcatttaaaagagaatttataatggcaaaacaagttaaagcaaagaaagcagttgaaaagaaagctggtgattctaaacgcgctggttacaagcgtgggtcgaactctcgtatcaatcaaactgttgagaagatcatgcgccgagcacgtgcggttcttcgagatgatgcttctcgttttggtaagcagaaagcataagttgaggactccttcgggagtccttttttattttccaaagattgcacaaagttgtttacagtatggttcctttgtgatagtattatcttacacaaacaaaggagaataaaatgaaaacgattaatctgaacgctgcagttaagactaaatgcttcaatggtaaatatgatgaaactatgtggttcttaatggcagttgaaggtgatattattgaagtagaaacaacagaaggtatgggaacagatttcacctttacaattcaagttcataatttctttactggttggatttatgaattgaatacagtaatcgttggaaaaattgaacaaaatgaattaggcgaatggtattatgttacagctcgccaacgtgtcaaacgtttgattgagaagatgaaaaaagttggtaaacttgacatgaagcactggaaagtagtaaaataattgtttactttggtacaggatatgatattatatacctgtaccgcaattaaacatctcggagaataaaaatgaactacatcaactttgaacgtaaatatgtttctaatagtattgcaggttctattgatactatctgcctttggaaacatcaaaatggatcagtatgcgaaattgaacagtatatgactcctaactatgtttatatgcgatttgaaaatggcatcacggtttcaatcacaatggaaggttccaactttaaaatcgctttggatgatgattttcgtcaacgcgatttagggactcatccttgctggaatggtgataatcgcaagcttttggttaaaacttggattcgtcatattctgagtaacagagctaaacctgagcatcttgaagcaatctttgatgtagttcttaacgaatttgatatttaaaataaaatgaggggcttcggccccttactgaggaaaatattatgtttatgactacttattttgatacccgcaaaaatttttgtgaagtggttttttctaaggcgcctaaagaccttcctgctcatttgcaacctactagtgaatcgattaaaaattatgttgatgtggtttgtcctttagaattccgtacagtaaatgggcgtgatactttagctatcgctaaacttaatcgtgaaattgacattgacccctcaattgcgtgtgaaattaatatttctgatattggcggcggtaatgttaaatcacacggctttcagatgaggttctgatgaaattctttttaggtcaaactgttgaattaaagggagttggtatacctggattaatttctaaggttctacctccatttaaatggagcggtgttcaaataaaagaggcttatattgtttcctgggtagatggaaatgaagaccttcgtatgggtgatgaattatctcctatttacggattaaaggaattagtatgaatataattaataagatttttggaattcagtacattaaggttacgtataaagtaacagataaaaatccatatactgacgagcatgaagaaccgcaagtcgagtctattatactagaaaagggtagtgactggccagttgagtttcgtcttccttgctatggtcattgggctgatgttgaaattataagtattgaaaatgtctgagttagagattagaagcaattttaaatggccatcatgcgcattaagtaatttcgcccaatggcctttcatcatggatggtattcaatttggaggtctcgaaggattcctccaaggatgcaaggtgaaaaatgttgagcaacaaagacgtatatttgggttatccgggcttgccgcccaacaagctggaaggtcttatgcaagagcccaggaccgtgggaccctcttctggattggagttccgttttcaagatactccccggcgtggaaagagttgtacacaaatgcatattttgaagcagcgattcaaaacaagggctttcgtgatgcattactagcctcgaaaggaaaagttttgaagcacagcatggctagtggtctaacaaaagatgatacaatactaaccgaagctgaatttattgatgtgttaaacctattaagagactctctatgaagcctactattttgactgatattgacggggtgtgtttaagctggcaatcgggccttccttattttgcccaaaaatataatcttccgttggaacatattttaaaaatgattcaggacgaaaaatttatttctcctggtaagctctttaattgcgatgaagaacttggtgttaaattaatcgaaaagtataatcgttcagattttattcgttatttgtctccatataaagatgctctatgtgtaattaacaaattaaaagaagattataattttgtagctgttacagcattgggtgattctattgacgctctgctgaatcgtcaatttaatttgaatgctctttttcctggtgccttctcagaagtattgatgtgtggtcatgattcttcaaaagaagagttgttcaaaaaggcaaaagagaaatataacgtaatttgttatattgacgatctcgctcaccactgcgatcacgcgagtgaaatattaaatgttcctgtttattggatggctcgaggagagcgtgacaatattccaaaaactgcgcagcgagtttatacatggaatgatgtagagaataagctttttccatcgaaggaaaataaagaaagttttgatagtgaaaaagctataaaagatgtaattgaaaagatgattaaaaacgattcttttcgttggaatactacctggagaactcctggattaaatccataccatccatattatacacatccatatcatgtgcatccatttcatacatggaactatattaaacctggtggcatagagtatttgtataatagacctactagtggtgataatattttacaaggagcattctaatgtttgttgttcatactatttatgaaaatgaaggtaatactacacgtgattacggtcacgtaaatcaattttttagatgcaatccagaattccgagctcaaaaagacgaacgaatttttaaaaaatgtgtagagcaaggtttcatttacgtcaagcactggatgcaaggaaataaagttagaaccacgtaccacaggtctttgactgagcttaatgatgaattgatttataatagagctgtaaaccaaactctaaaggatgaacaatgattcttaaaattctgaatgaaatagcatctattggttcaactaagcaaaagcaagcaattcttgaaaagaataaagataatgaattgctcaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcggcaattgaggaattaactggatatattactgacggtaaaaaagatgatgttgaagttttgcgtcgggtgatgatgcgagaccttgaatgcggtgcatcagtatctattgcaaacaaagtttggccaggtttaattcctgaacaacctcaaatgctggcaagttcttatgatgaaaaaggcattaataagaatatcaaatttccagcctttgcccaattaaaagctgatggagctcggtgctttgctgaagtcagaggtgatgaattagatgatgttcgtcttttatcacgagctggtaatgaatatctaggattagatcttctcaaggaagagttaatcaaaatgactgcagaagctcgccagattcatccagaaggtgtgttaattgatggcgaattggtataccatgagcaagttgaaaaggagccagaaggcctagattttctttttgatgcttatcctgaaattagtaaagctaaagaattcgccgaagtagctgaatcacgtactacttctaatggaatcgctaataaatctttaaagggaactatttcagaaaaagaagctcaatgcatgaagtttcaggtctgggattatgttccgttggtagaaatatacggtcttccggcgtttcgtttaaaatatgatgtacgtttttctaaactagaacaaatgacatcaggctatgataaagtaattttaattgaaaaccagatagtaaataacctagatgaagctaaggtaatttataaaaagtatattgaccaaggtcttgaaggtattattcttaaaaatatcgatggattatgggaaaatgctcgttcaaaaaatctttataaatttaaagaagtaattgatgttgatttaaaaattgtaggaatttatcctcaccgtaaagaccctactaaagcaggtggtttcattcttgaatcagagtgtggaaaaattaaagtaaatgctggttcaggcttaaaagataaagccggcgtaaaatcacatgaacttgaccgtactcgcattatggaaaaccaaaattattatattggaaaaattctagagtgcgaatgcaacggctgggtaaaattagatggacgtactgattacgttaagctatttcttccaattgcaattcgtttacgtgaagataaaactaaagctaatacgttcgaagatgtatttggcgattttcatgaggtaactggtttatgaaagcttacttagaaacaattgtcgtggctcaaaaagaaggtggagatgtttccactgcggtatcgcaggttattcttgaattcccagataaactagcatatgatgaattcacggaaagatttgatccatatgaaaaaggtccaaagtttgaagtatatcgtactcttttagctctctattaattaaaggccttcgggcctttaattttataaatagaataaacactagagaggataacatggaacttattacagaattatttgacgaagatactactcttccgattacaaacttaaatccaaagaagaaaataccacaaattttttcagttcatgttgatgatgcaattgaacaaccaggctttcgtttatgtacctatacatctggaggtgatactaatcgcgatttaaagatgggcgataaaatgatgcatattgttccttttacattaactgctaaaggttcaattgctaaattaaaaggtcttggtccaagcccaattaattatatcaattcagtttttactgttgcaatgcaaacgatgcgtcaatataaaattgatgcctgcatgcttcgtattcttaagtctaaaaccgccggtcaagctcgacaaattcaacttattgctgatagacttatccgtagtcgttcaggtggcagatacgtccttcttaaggaactctgggattacgataaaaagtatgcatatattcttatacatcgcaaaaatgtatcactagaagacattccaggagttccggaaattagtacagagctctttactaaagttgaatcgaaggtcggtgatgtttatatcaataaagatactggagctcaagtaaccaaaaatgaggcaattgctgcatctattgcacaagaaaatgataaacgttctgaccaagctgtaatcgttaaagttaaaatttcccgtagagcaattgcgcaaagtcaatcattggaatcttctagatttgaaagtgaattattccagaaatatgaatctaccgcagctaatttcaataaacctgctactgcacctttaattcccgaagcagaagaaatgaagcttggaattaattcattagcttctaaaacaaaggcagcaaaaattattgctgaaggaacggcagatgaacttcactatgattataaattctttccaatgagtcaagtcggtgaagtttcagaaaaaattaaagaagtaatttttaatgcaattaaaaatgaaccaactacttcaataaaatgtttagagaaatacgcagcagctgtaaatcaattctttgaagattataaagataaatggcttgctaatcataataaaaatcgtaaaggacaatcagatgaagtctgggaagaaataactaaaaattcctggaacgcagcaaaaactaaattcctcaaacgaatgatttacagtttttctggaattggtgctggtccaatgattgatattactattgcacgcgatggttctaaatatactccatcacaaaagcgtggcattagagaatattgcggttcaggatatactgacatcaataatcttcttttaggtcgttacaatccagaacgatacgatgtaatgagtgaaaaagaaattgaatctgctataaataacttagattcagcttttgaaaatggtgaccgtataccagaaggcattacagtttatcgtgctcaaagtatgactgctcctatatatgaagcgttagttaaaaataaagtgttctacttcagaaattttgtatctacttctttaactcctatcatttttggacgttttggaatcacacatgctggtattggtcttttagaaccagaagctcgcaatgaattaacagttgataaaaatgaagaaggaataactattaatccaaacgaaataagagcgtataaagaaaatcctgaatatgttaaagttcaaataggatgggcaattgatggagctcataaagttaatgttgtatatccaggaagtctcggaatagcaacagaagctgaagttattctaccacgcggattgatggtcaaggttaataaaatagctgatgcttctaataatgacggaacaacatctaataatacaaaactcattcaagctgaagttatgaccacagaagagctcaccgaatcggtaatctatgacggagaccatttaatggaaactggtgaattggttgcaatgacaggtgatattgaaatagaagacagagttgactttgcatcatttgtttcatcaaatattaaacagaaagtagaatcatctcttggaattattgcgtcttgcatagatattacaaacatgccttacaagttcgttcaaggataaatcatggaacttattacagaattatttgacggcgcttcggcgccggttgttaacttaaatcctaagcataaaatacctcaaatttttgctattcaagccggtgaagaaagcgtgcttcctggatttagattttgtacatacacctctggcggtgatacaaataaaaacgttaagccaggcgataaaatgatgcatatcgtaatgataggtgttaatgagaaactatcattagttaagcttaaaaacttgggtggaaatccaattggcgtcattaatgctgtttttgatactgctcttcaaacaatgaagcagtataaaattgatgcatgcctattccgcgtactaaaaagtaaaacaaatggcgcagctcgtcaaatgcaagttattgctgaccgtttagtacgtactaaaggagcaggtcgatatgttcttttaaaggaaatctgggattatgataaaaagtacgcatatattatggtttaccgtaaaaatgccaatttagaagacattccaggtgtacctcctatttcaaccgagttattcacaaaagttgaatcaaaggttggtgatgtttatgtagacgttaaaacaggtaatgctgttcctaaagctgttgctgttgctgcttctattgctttagaaaatgataagcgcacggatcaagctgttattcagaaaactaaaattagtcgtcgattagcagcacaagctcaatattctactgttgatgcttcacttcagggtgatagcttcgctgctaagaaatatcaagagtttgaatctaaagttccggtatataaagcagaaggaccaatgaactctggcgttattcagattggttcaaactttagcaaaggagctatcggtggtatgagaagtgcttctcgttttaaatctagtgattatgaactcgaaagcttccgaaatcatattgcattggctcatgcacgtttacgtgatccatctatcaagttacagagcgatataacatatcaaggttctcaagaatatttaaagaataaagaattctttgattataaaactgataaaattttaagcgatcttgctgatattaatatttctaatagctttgatgttattaagaaaattatcaatgatttagttaaaggttctaaagctacaccagatgaaaagacagctattattcaatttgtcatgaatagcatttataaattaattgatgaatccgctgctcaggcgtatgaatatgcaagcactgaagtaactccaaaaggattgactcaggctgaatctgatgtaattgaagattattgtgcagattcgtatgttgaaatgaactcattccttttaggtaaacctgattctacacgtgaagaatacatggagcgcgctattaagcacatcgagactatggattctgcgtttgctaaaggttcagttcttcctccaggaactacactttaccgtggacaagaagttacttttaaaactttgcgtcataacattgaaaataaaatgttctatttcaagaacttcgtatctacatcacttaaaccaaatatctttggtgagtatggtaaaaattatatggccttagatgattccggagcagtattttctggtgaaggagaaggttccgttgatgcagaagatttgatgcatatgggtagtcattctacatatgctaatgaagatgctgaaactagcgtgggtatggtaattaaaggagctgaacgagttaaagttatcgttccgggtcatttatcaggatttccatcagaagctgaagttattctaccacgtggaattttactgaagattaataaagtaagtacttactttatgaaagaaactgcttataacaagtatctaatcgaaggtacaatcgttcctccttctgaacaattagaagaatcagtatatgatggtgactatctaatggaaactggtgaagttcgtccaatggctggatttaatcaattccttgtagaagaatcaaaagaagaggaaaacgaagtttctcaaatcttagcttctttggttaacattaacggaatgtctaaaaagttcaaaatgtagtttacaagtccctcgtgttgtgttatagtagtcttactgacataacatgaggaacacaaaatgaaatcttctttgcgctttttaggtcaagaacttgtagttgaaggcgttattcctgctgataatgcttttaatgaagcagtttacgatgaatttatcaaaatttttggaacagataaaaagttcggaatttttccttctgaaaatttttcaaagccagaacagactgaaagtatttttcagggtgtagtaacaggtaaatttgagtcagaagctccggtaaaaattgaagtttatattgaagaaacctctgttgcttcagttgctgcttttatttcattccgtaaataaaaatatggggacctttcggtccccattgttatattgctcctaatattttactttgcgaattgacaattcctgtcatagtattaatatttgaaatgcttcctgcagttccccctaatctactaagtcgtgaaagcgaattagatagtccagtaacacctccactatttccgagaacgctttgaataccatttatagtagcagattcaagccattcaagggcagcttgtctatcaactgctccagcctgcatgactctataagcgaaagtaacatcaaatgtagttatttggttatctccatcatatgataactcaggagcactcacagatattggaatacagccagtgaacatcaccgcagtatgtggtaatccattgcgagaatgaagattgacctgaatatctgcctcgacatcttgcggtaaagcacgcaatccagttactgggtcttgaacagcgttaacccagtcttgcattgcacgatagttacaagcttctgaatccattctaaatgaaataaccaaagggtctaattctcttccagttatacgaatattaggagaattatagttccagtcagtttcataggataatctattctctggcatttttacagaatatatcatcaatccagatgagttatatgccatgttaaagaagtcaattaaatatgtaccaactgtaaatgagcctaataaactttgaactgtacgttgactcatggcaccaataagatatttactaacccctgattttcttatcagtttttgtgtgccagcggtaattagcgtggtaattccctgattaatatcgccttgagttaatcccaaccaatctgaatttagacccaagttattataagcaaagttactaattgagcttattaacgatgagcttttagttgatggagttgtagcaaaaacacagctgaacatattattacgttggaagtctgcgtttattgcttgattattaaattcctctaaagaatacattagaaagtccccgtatataaagaagcacggtttaacgtaataatctctctcatagtaatctcgagtgtaaaagaacttggaaggttaggagcaatagctaatccattaaagttaccatttggtgttttatcaaatctaatactctgtatttggcatggaccgaatacttcagtctttccatcaaatttagaagttgcaccaaagtttttcaccatccaaattgtcgggtttgaaactacaagaacgttcgttaaactcgatgtcattttttcaaataacgtcttgtttttaatcgcgtcttctggcgttaatggttcaataaatgtagaacgataccactcatctaaatatccttttatttcagcagcatattgagatttaccagtttcaccgtaagaaaaatagttaaaatactgatagatattaataatagccattaaatcttctgttgagcgcggagtcaaatcccaggtgaacacttttgttctattttcagcaccgccatacatacttctagctgttgtatagatctgttcattattatcagccattattccttgtgttatactttcaagcgcaccaaatactgcagttgaagccatattacttagcacaccagtagcagtacctccgccttttgtaataagactatcttgaacatcattaaatctatgtgatgacgtatcgacgtcagatttagatctaggtaaaagaatatttgcaacaggaactttacttattgttcctgaattattatctgatattagtccatttgatagttttgatactgtattactgatagtgtttctggctgtacgtaaaatactcgaagatgaagaagagtagttagatctcatcgttctaagacttccagaatccctagatgacatattgtatgcagtaaataataatccattcttatatagatctgttacctggaagtcccctgtagtgtcattaccactagcacgcccagttggaaactgggctgtgtatgttttagttcctacttctgatttagtactctgtccggctgaaattgtttcaccggactttttaattaaatcagcagttatttctttaacaattgccatattattccttaattaactcccgtcgcaccaaatacaccaggagcagttgtgcttgtgacaggtgtcatattatgaacgacagtatttttcttaataacattattagtattattgattgaaggagatgcttgttgaacaggagcttgttgtgctttgttcttttcaatcacctggacctgttttgcttctggtgatttagcagaagtttcaggttttgcttcaggttgatttttcttgagctcttgataagtggcatcaattttagcaaatctagaagcaagttcttttttaactgctggtgaattatttaaatccggatcattcattcgttttttaaggtcttcatgagcagcttcaattgatttaactgtcgagtctttgctcatatcagctgaatcagcgtatttttcaaaacgaatcatcgcagcacgagcttcattagctttcattaaagcatattttctttcttccggtgaaagttgttttaatttttcttcttctgctgcacgttcttcgtcagtagtcaatgcttctttattatctacaccacgaatccagttagatgcacgagttttccagttcgcaattttgtctaatccttttgctattgggccaagatcatcattcattcgtttatcttgataatttgctactttttcttggtcttctttattaagagatgctccagtagaattttggaaattttctaatgctcttccttctacttcatcagcagtatccttcatgccaggaatgactcgaagaattgctgcagataatttagccatacctaattgaataagctctcctaaattataaagaacacttccaagcccttcaacaatagctactgtcaatccgccccaatctccagcttcccaaagttgtttaattttatcaatagaatcaaagatgctctgtaataaaggaccccatgttccggtttcgctagagaatttagtaaaatcagtgctaaataaatcccatgcttttgaaaatttatctgaccagtatttaaaatgaaccatcagcaggtctattccaataacaacagccattatcaatgcaaccattttagcagcttcaatagcagcactgacggtatacttaaatagcatgctagatattttatcagtaattgaaatagatttcttaaatccgaaatcaactgtctttgttaatttatctaaagcttgagataattttaagttaaatgcgtctcttttttgcttctcttctggagactcttgttttggttcaattggctgaggagtagggaaaaaatcagcatcaggatcattattaactgcttcaggagccggtaataaaggacccacagattcagctgtatcatcctcgacgactttaacaggaatagcattttcgactactgctaaactcgttccagtttgctgaattccagctgtctgaattttttgttccagtaaacttgttaatttatctaatttgcttccgagcgattcaccgatttctttattaatattgtttccaatttcaacagtttcagcaattaactcagaaccggcagtagtatcacttactgcgctttcaacgttatcaattgctccaactatttcatttgatttttcttcaacagtctgagctattaattcagaagcagcttgagcatcatctaattttgtagaaatgtcattaagaccagataaagtattagaagcggatttagccgcgtcctgtgttggtttattatctgaaataacttttctacgcatcgtttgcatttcttgtggctttttcattcaaataatccaataatattgccaattccggttattggaccattagggccaggaattgctaaagttgtagaaatatcatttgcccattttaaaacaaatgctggcatctcaagaaaattaatttctttaacttcatcattgaccttaagcaagcatttggataacatatcgcttacagttaaaaattgttcaaattttccaggaggtctaaaataaaatgtatttccttggtattgaaattccaatctttgacatacataaacgtcattaatatcataagtataaccatctatttctttacgagatttaatctttccattaaattctagcaaatgaatagaaacgaaatcaacttctgccggtgataaatttggacaaatagaatcaataagaagctttaaattttcatcaggacctttaacatcttttaaaatgttataatgtttaagacccatcttaggaatagaaacttctttattacttatcggaagaactactttcttcagtggtagtatcagttttaaattcatttttaaccttaactgggtcaattgtttccagtttcgttgcattagtgaacatataaagatgagttacggaattattatttgataattcgtgaataacttcatcgacgtaaaattctgttttaaattggtttttactatcattaaaaataattttaacgccaggagtcaagttaaaattaccgacagtagaacatttagcgtaaccatcatactgcgccatagtctgaagacgaatagcttcttcatatccatttctataagtcatttcagaataagcacctgaccttgacactacaatagagttttcaccctttcctgtagtaatcattggtaatgaagaatctaaaaatgaatgagcatagatagtagcatttttcattggatcacgtttatgcgggtttgatttagtcaaccaaacaaaatcatatgctaatggatatttcaattcttggacgaattgacctattaaagttggctcacctacaatcattggatatggttcttgatttatcatcatatcatagtccatcatgttaactcccatgatgtcttgccatacaaatacgaacttatcgcttcctaccgctagagcaacttctcttacatatgacaaatagttttcaaatgtgctagtccatggaatatcaggaacataagcatttatagcatttattgctggagttaataatgtgcggtcttgataaatgacaccaagcatttcctttatagattccccggcatcagggaaaaatggtctaccaaatttaagattttctatagaatgaatagttcccaattcaatagcaatgatgttatcgccttttgaatctactgacacggaaaaatgcttgcatccataaattcgtgttttaacattattaatatcgtttgcattagctacagaaatctgaattatttcatttccatccatttttgtatgaatgtttttagaatcataaaattgtagcattccttcatttcgaccataaagagagtcacgcatagttaatgtggtaatagtagcagctaattcaacaaatctattattactccaagcgtcataactctcaaataatttaacgctgagatttggatatccggggcgttgcaacatactcattattgtttatccttctcaatcagttttaatacgaatccgcgctcggcaggaatcattttcattattgaatttaagctataattactttttacaagcgtgtgattaatttgataaaaagtaaatatctcatctggattaactaatagcttaaacacatctactatatcagtgtattttttaatgtacttatcacaacacgacatgtgcaatgttaaattaataggattcattgcatcgagaattttttctaatgtttctatctcgatggcgtcaacaagttctatttgacttgattcactaatttccttccaatcataccacatttcatctacttgaacagaatgaatattttcagtaatcatctttgctttattttcataaaactcagaaggaaaatttaatttaattttaacattagctacatcaaaaataggttcctttaattctttttgatatatttcaaatggaactgtcttttcttttttacattttggacatatgaatgtgactggtactttagttttacctattgaacctacaaatacctgcaaaaatataaatggttgccaagtctttggatagtctccaaaataatcagcaattaaatcagcaattacttctttttgttcttgtaatgaccgatgttctatatcgtttcgaactaacaaaaaatctcgataatcttctaccgtaaatggtttaaaacgatgaacaccatcaggtaatttacaacgaataatgtttgccatagatgctccttttattctatttataaatatgataaataaaggagctaaatatgtatgaatacaaatttgacgtgagagttggttctaaaataatcaactgtcgtgcatttactcttaaagaatatctagaacttattactgccaaaaataatggttccgtagaagtaattattaaaaagctaatcaaagactgcacaaatgcaaaagatttaaaccgccaagaatcagaactattactgattcatttatgggcgcattctcttggagaagttaatcacgaaaactcctggaagtgcacctgtggaactgaaataccaacccatataaatctattacatacacaaatagatgcaccagaagacctctggtatacactgggtgacattaaaattaaattccattaccctaaaatttttgatgataaaaatatagcccacatgatagtatcatgtatagaaacgattcatgctaacggtgaaagcattccagttgaagacttaaatgaaaaagaattggaagatttatattctatcatcacagagtcagatattgtagctataaaagatatgcttttaaagcctactgtttatttggctgttccaattaaatgtccagagtgtggaaaaacccacgctcatgtaataagaggactcaaagaattctttgagctattgtaatggcaaatattaataagctttattctgatattgacccagagatgaaaatggattggaacaaagacgtttcaagatcacttggattaaggtcaattaaaaatagtcttttaggaattattacaacaagaaaaggttcaagaccgtttgaccctgaatttggatgcgatttatctgaccaactttttgagaatatgactcctcttactgctgacacggttgagcgtaatatcgaaagcgcagtaagaaactatgagccacgtattgataaattagcagttaatgtaataccggtttatgatgattatactttgatagtagaaatacgcttttcagtcatcgataaccctgatgatattgagcagataaaactacaactggcttccagtaatagagtataatgcttcacgtataaacgtggtataatgaatctaagtccatccaataacaattgaatagagaacaatatgagattagaagatcttcaagaagaattgaagaaagatgtgtttatagattcgactaaattacagtatgaagcagctaataatgtgatgttatatagtaaatggcttaataagcattcaagtattaaaaaggaaatgcttagaattgaagcacagaaaaaagttgctcttaaagctaaattagactactactcgggacgaggagatggtgatgaatttagtatggatcgttacgagaaatcagaaatgaaaacagttctatcagctgataaggatgttttaaaggttgatacttcattacaatattggggaattttattggatttctgtagcggagctcttgatgctattaaatcacgtggatttgctattaagcatattcaagacatgcgggcatttgaggctggaaaataatgagatataacattgatgatgcttttaattatgaagaagaatttgaaactgagattcaattcttaatgaaaaagcataatcttaagcgtcaggatattcgtatcctggccgaccacccgtgtggtgaagatgtcctttatattaaaggaaaatttgccggatatcttgatgaatatttttattctaaagatataggcattgatatgcatatgagagatatggggtaaatatgataaattataaagaagtatttagacttttagcagaaatggaagcatgtttaaagcgtgtacaagaaatgcgctttaagatggggttataaatagatatataattcagaggagacaatcatgtcagataagatttgtgttgtctgtaaaactccaatcgattctgcattggttgttgaaacagacaaaggtcctgtacatcctgggccttgctataattacattaaagaactaccagtttcagaaagttcggaagaacaattaaatgaaacacaacttttgctatagtgtgacctttagtctatagttttggcccttccttttttggttgggccttttttaatttaaaagctttcttctacttcatcatcgtctgaatcttctaattcagctctttttcctgccaaagcatctctgactgagatgtcatcagtatcttttaattcagtttctttaactcttttcttataataagcttcaagttcttctaaaccttctatcgtttggcaagaggcaattttacccataaattcatcaatagaagcttcataaagaaattgtttaaattctagtaacatctttttctccaaagggccgaagcccttataaattaactgttttcattacgtaattaaatttttcatctgcgtagcgctgaatacgatcaatgccgtgttttaaaagatagttcaaatgaacatatttctttttcgtattagcagattttggcttaacaccacaatcatctatgaggtcccagactgttgctattgtcttagaaccatgcttacgtaatacacgaccgattgtttgtaatacaatgatttttgatttaacaccgtgcgctaaaacaacgtgatgcagatttttaactgaaataccagtagaaaatacaccataactagctactataattattcctttgccattttcagctaaggttttcattatattgcgggtttcggtatcaacttcccctgatacgtaataaactttatcatattcatttttaattaaatcgaaaatagctttaccatgtgatacatgtttaaacatgacaaaagcgttttcatctttttgtgcaagcttaatagctaatttagcgatccatttatttcttttactgagcccagtaataatttttatttcttcttggtaagtttttccctttaatttagtagtgaactcatcgggatagcgaagaaaaatactattaatttttaactcagttacttgtccatcttccattaatttagaggtcgttactggcttaaatatttcaccgaacattccaacatactgcatgatattggctttgccatcacgtaatgaaccagatagaccaaatttgaacatgcagttatttaaacctgatatgatagatgaaatactttttcctgtagcaagatggcattcatcattcatcatcattccaaactgtgagaaccattcttttggttgttttactacagtttgccatgtaccaacaacgactggtgcatcatttttatatttatcatcttttgatgctccgccaccaattttctttatcattgcatgactgaataaacgatagtcaacgaagtcatcagccatctgagttgttagagcagttgttggaacaatgataagaattttaccttcgtaattttccaaataatatcgcgcaagcaaagcttgaattaacgatttaccagcagatgttggaagattaagaattctacgacgattaactaatccttcgaacactgcatctttttgataccagtgtggttcaattcttttatttcctgaatagatttctaatttagaaagccattcatcaaaatcttttcttgataattcttctttttcgttaatttgtgggtcaatccaggctttatagccaaagttatcacagaactttttaatttgcccgactaagccgaatggaagaagtcgattataatctaaaagacggattcgtccatcccagttgccatatctgaagcgagggttaaatctatatccatcggcctcaaacgaaaagaaatctcttaattcgtggaacgtgctctcttcacaatcgatgcgtacatgactgaagtcgtgaaaatgtactttaatatccataattatgccttactaaatttgcctttagaatctcttttcatgagacgacctttaataaatccgtcgggaataataccgtctggttgtattaatttatttattgcgccattattgacccaaaaagttcctgtggtagtcggtttgactttacatccttttctggactttctattaggatgaaccatcccttttacaaatccttctggaacaagttctccaggtttaataaaaatatttttagttccattagtgtaacaagttttacctaataccgtgcctggtgagttttcaaatcttttagcggaagattctttcatctttgttataacctctgtagtcataacaattccaccaattccaccaggtttcatattataataatttttgctttttattagttcaggagttataatttcttcttcatacatgtacgcttcttcggaagttttaaactcttttagtattgttctagagaaattgttttcaccatatttctttatagcctgctgaattgccttaccggaaccaaggtaaccatcattcaagtcatcagtagagtgttttcctatatactttttaccatttattagatttgttgtttcatatacaaagtggtacatactattttccgagtaataaatatatctatatttatactgaggaaatattatgatagataaagattatattgcagagctgaaggctcttgatgataataaagaagctaaagctaaattagctgaatatgctgaacagtttggtataaaggtcaaaaagaataaatcttttgataatatcgttgttgatattgaagaagctctccagaagctcgctagtgaacctatgccagagactgatgggttatctattaaagacttaattgatgctgctgatgctgcagagggattaaaatatgacgatgaagaagtcaatccagaagcagcacttctgattgattctccagttaaatctgatattaaaattgaagtggtagaaacggataaaattcctgaaaatactgatgttttgattgaagatactccttttgttgaagaaaaattcgaacaagctgtagctgagattattgaatctgaaaaacctgtattcgctcttccagaaaactttagtccaaatcttcaattattaggaaaaaatccaggtttctgcactgttccatggtggatttatcaatggatcgctgaaactcctgattggaaatctcatccaactagttttgaacacgcgtcagcacaccaaactttgtttagcttaatttattacattaatcgcgacggatcagttttaattcgtgaaacacgcaattcttctttcgtaacattaaaataaggataacatatggcttttacagttgatataactcctaaaacacctgctggagttatagatcagacccagcagtttactgctacgcccagtggtcaaactggagatggacctattacctatgcttggactgttgacgatgcaccacaggaagaaacgtctgcaacttttgattatacgttaaaaggacctgccggtcaaaagactattaaggtagttgcaacaaaccaagttgcagatagtagtcctgaaacagctcaagcatcaactactataacggttaaaaataaaactcagacaactacattggcagtaactcccaatagtcccgctgctggagtaatcggaacagctgttaaatttactgctgccttagcttctcaacctgatggagcgtctgctacatataaatggtatgtagatgattctcaagttggtggagaaactaactctacatttagctatactccaactacaagtggagtaaaaagaattaaatgcgtagcccaagtaactgcagaaaattacaatgaaaaggaagttacttctaatgaagtatcattaacagttaataagaagacaatgaatccacaggttacattgactcctccttctattaacgttcagcaagatgcttcagctacatttaccgcgaatgtcactggtgctccagaagaagcgcaaattgaatattcatggaagaaagattcttctcctgcagaagggtcaactaatgtatataccgttgatacctcatctattggaagtcaaactattgaagttactgcaactgttactgcaactgattatgatagcaaaactattacagcagaaggtcaagttcaggtaactgataaagttgctccggaaccagaaggtgaattgccttatgttcatcctcttccacaccgtacttcagcttacatctggtgcggttggtgggttatggatgaaatccaaaaaatgacagaagaaggtaaagattggaaaactgatgatccagatagtaagtactacttgcatcgttatacgcttcagaagatgatgaaagattatccagaagttgatgttcaagaatcacgtaatggatacatcatccataaaactgctttagaaactggtatcatctatacttatccataatcgaaaggggcttcggccccttttttcattttgaaagcacacaaaacacaatcagaaaatgatgtatataatggcaccaactcgataacatgagattgattatgagaactgaagttgtggtgtttacgcttcatgagtctggaaagtcattcattgaaattgctcgtgaattaaacttacaggcgaaagaagtggctgtattatgggctcgagctatgactgcaaagaataaatttgagactcgagaaaaagttgtctatagaaaaagacacattaataaaaaggtgaaaaatggaacagtatgatctttatgaaaatgaatcttttgctaatcaattacgtgaaaaagcattaaaaagtaaacagtttaagctagagtgttttattaaagatttttcagaacttgctaataaagcagctgaacaaggtaaaacatattttagttattattgtactgctcgtgataaattgattactgaagaaattggtgattggctgagaaaagaaggatttaattttaaagtcaatagtgatcagcgtgatggtgattggttagaaattacattttgaggattaattatgtttaaaaagtatagcagtcttgaaaatcattacaactctaaatttattgaaaaactttatagcttgggattgactggcggcgaatgggtagctcgtgaaaagattcacggcacaaatttctcattgattattgagcgtgataaagtaacttgcgctaaacgtactggaccgattcttcctgctgaagatttctttgggtatgaaattattctaaagaattacgctgattccattaaagcagtacaagatattatggaaacttcagctgttgtatcttatcaagtctttggcgaattcgctggacctggcattcagaagaatgttgattatggtgataaagatttttatgtatttgacattattgtcactacagaaagtggtgatgtgacttatgtcgatgattatatgatggaatcattctgtaatacatttaaatttaaaatggctccacttttaggtcgcggtaaatttgaagagcttattaaattgccaaatgatttagattctgtcgtccaagattataattttacagtagaccatgctggattagttgatgcaaataaatgcgtttggaatgccgaagcaaaaggcgaagtatttactgctgaaggatatgtattgaaaccttgttatccttcttggctgcctaatcgcaatcgtgtggccatcaaatgcaagaactctaaatttagtgaaaagaaaaagtctgataagcctattaaagttaaagttgaattgtcagaagctgataataaattggttggaattttagcttgttatgttacattgaaccgagtaaataacgttatttccaaaattggcgaaattagtccaaaagattttggaaaggtgatgggattaactgttcaagatattttggaagaaacttctcgtgaaggtattactctaacccaagcggataatccttctttggttaaaaaggaattagttaaaatggtacaagatgtacttcgtccggcttggattgagttggtgagctaaataaaaagggacctttcggtccctttgttttattcatcaatgataatttttggtagcttaacacctaataaaacggacaaatctgaacgacccgccattttatccatgtctccaccatcaattactcttgcttctttttcatcttttgctacggtgtaaggatttgcagataaagcatatctaactaataaaccgatagacggttgtaagctttctggatcaaccacaactttaaatgcacctacatgttcagggtcatctaagtcgagaccttctgtatacggagcatagaaaattgatccaacaatttctttttcaccgatattttctactacgccaacgattacataatctaatgggctgttagtatcgcaataaagcggtaaaccattagctaagaacccataggcattttgtgaaagatacttgtcatcttctggtttatgttttaaccaaccagatgcagcaagaatcgcagcggcacgagcagaagcaacacagaacgttgctgtataagttgattctttttggatatgcgaaaccatttcgcataccattcggtataatgaacgaccagcttcaggtgcagatgcataactcaaatcaatgaatccagtatcagtaattcctgtaactttataacgttttgatactgtaatcaaagactgcagaatatctttattgatttcatctgccatttcagttgcaagcaaatcttctaagaaattaggagcatcgaatccatttgcttctaaatcttgtgctaattcaaccgtgataccagttttaagtttacgagatttaactgcggtttgccatttattaatctggaatctagcatccgcaatttcactatcagagctttcaaatttgcttgttgacgctgcgtcagaaaatagacgaacctttaaaagaacaattgcaatctgaagagctaattctaaatcactttcttcaatatcagcaaatggtgtatcttctaatactttataaacgatattattatatttgaataaatcgcctttattgagagttaatttagactcttctgttaattctgtgattttttctcggtctacatatccagcttcgccagcataagtagcaccagttttaaatgtaaattcgttgtctgggttaaggtatttgataccataaaaagcagcaacaggttgattagttctttgcgttgctacaatgtcagaatatattaatttagtggtagcgcgagtcaaagcaacgagatttgggcgaccgattgagttgctattcgttgtggttgattcgcgcagaagttcgttgattttagccattgcgctttcctttcagtttatatgatttatttataccataaaaacaactaaagggacccgaaggtcccttaaatcgtcaaagattagatacctttaacatatacacggcggaagtaagcgttcttaccaaggctgttcaggatagaaggcataccactctgaatgcgagaagccggagcctgagcagcggattctgcaaatgggttgataccgataccgtaacgagttttgaaccccattaccggttggaagttcttcggatcagaaccacgcagtggagtcagagctacatatggagcgtagtaaataccagcatccatttcgttcggacctttataacctacagtgaaataatcctgtttagcatactggtcgatatatacacggtatttaccacccagaacaccagcaaatactgacttggtagtatcagtgttaaagccggtagccagaccttgtgcagcataagaaatgccggtatcaactgaagccagaacgttaactacgttacgggaagcgataatgaagttaccttcaccacgaccggtttgacgagcaatttcaactgcttctttgtcaatctggaacaacagagctttaaaggattcacccgcccagcgagcaccacgaatatcgattgggtcctggaagtcaaatacaccagctttagaacccggagtcagggtcataccagatttaccaacctgagctgagtagttaatccaatcaacaacttcacggttgatttccaacatgatttcggtagccagaataccagacagttcagcatcagcatccataccatgaacagcgcggaggtcttgtgctaattcaatagagtaagcagctttcagctgacgagatttagcttcgataacttgcttatcgatacggaagcccatttcattccatgggttatcggtagaaccgttaaaaccttcctggagttcagcgatagaagtagccataccttcagcgatttctaccagtacacctgcttccatttgtttcttaatttcagcatctaatttagctgcgtcagatgcgcctgaatcaatttgtttagcttctgtagcttgcagatacacagtaccagtgtcctggaagaagtgggtgtaaatagttccaacttcaagagtatcacttgctttcaaagcagcgaatttcttagcagcaccctgaccagagaacattgcatctggaccatacattgggtggaatgcttctttagcgccggaagcgattgggtctttaccatataccgcacgaagtgcgaatacctgaccagtcgggctgttcatcggctgaacaccgcaaatatcgaaagcaatcaggttaggaatagcacgacgtaccatacccataacagctgggccaatctgagttactgcgccagaagtctgacctgcagcgatgttggtagcattgtaaccgtggtcaccaccgatttcagcttctgttaagaaagaaccgaatgcctgagcaattttttcgtctttatattccggagctgtctggaaatctttttcctggttttcaaagattttagcgataatcgcttgtttgctattagcaatttccggtaaaccttcaccttccagtaatggcttccatttgttcaaaagttcagctttagttttgatagtcatttgtgttaacctttaaaattagaaacgagatgcgactttcgcatatacacttacaatatcttctgcaccttgcgcagatttatcttctacagcttcagtgacgaaattcagtccggctgcttcagtatcaggagtatttatactctcagtaatagtgctttcatctttattagatttcttcaccatttctacgatggcactcaatttgcttgagaatgcatctgaataatcaataccttcgaccagagcagagactttttctttttgagactcagtcagatctttagtactttcgctcaatgccacttcacgctgcacataattgatatatgcgtcacgcttattgagttcttcgaacagacgagctgattcttctttgtgttcttgcagttcttcttccatttcagctacaacatcaactgattcttctggaacaacaacgttgtgttcaacgaagagctctttcaatccaccaagcatggattcaaacagttcggctttgatgcctttatctactgctaatttattttcagtgagccattcttttgcaagatggtcaaggaatttagaagcttgctcagcgattttcttctcagctttttcttctgcttcttctttatttttttctacttcttcttctgctttttcagcaattttagcgatatgagattcagctaatttaacggcgtgctgcttgacggtagcttcgaatacagtgccgaaagtttcttttgcttccggagaaatattaactgattcgaaaatactatcaagagcaacggaagcatcaattttctgcgcttcggcaatcagttgttctttaagcattttgtagtcctgttgtttagataataatatttataacgcttttttcatggcctctgcgagagccatataggcgtcatcggcacttgtatcggcttccgccgtctgtgattcggtaatttccttaggagttacccatgcatctggagcacttggaccccatactgcatcaacacctacagttaatttgaatccttcgtttacgatacgataacctttatttgtgtcagtcaatgaacctaatccacgagaagaaactcctggaatccatccggcacgaatattagctgctaatttatctccaggaccatggtcaccttcaataacacgagctcgtccgtatacgtcatttcctttccaccacatatcttctataatgatagcggcttgcatcgggtcaacattagcgcgtggaggatgatttaattctccgagagcttgtttagttaaaacttgctcattaatatagtcttttaccgctttttctaatatgcgttttggataaagacgtttatttctattgacgacttccgcttgcataaatattccttcgatgtataaaccaggttttaaacctaagtcttttccatcatgagattcaagcattggtacgccatcaataatttcgccaggttgaccccaagtttcaattagtaattggggttcattcattagcttaatcctaatgctttacggcgtttaagagcttttttacgcttacgctgagcacgagattgacctgctggattggcaatcttcgttttggtagctttacgagcaatttgtctacgttttgctttagacaacccggtagtttgaaatgcattacgttcgcgggttttgcgatctttagtgcgagtaatttcaccacgggcagaaacatgtttaacgatgaattcatttaacggcatattttcattaatagaagctaatgcaaccgctaaatcagtttcatcatcaagcatattctcgataattttatttatatcgtctttatttaaagcagaagacaactcgtcaaagcgaccctgtgcttcaggaataagtgcttcgacattctcgagaactaattcatgagtttcagggattagaatcattattcatcatcctcgtcttcgtcagagtctttgtcttttttgtcgtctttatcatcgctatcttcgtcatcatcttcatcttcatcttcaggttcttcaccctcgattaagaaattacgagcgatagtgattttttcttctttgattaaatcagtcgttcttgcagccatggcttcagcaaagaatttacgagcggctacgaggtcgtttgatttaatagcttcaattaaaccttccattaaaaatcctcttgttcttggtcggggtcttggaaacgagcctctttagactcttcttcaatttgcttggcttcttgttctatttcttcatcagtcatctgcaaaatatctttcatagcagttctgtgagaaatatatttaccaataaatggttctgccatggttagcatattaattcttcgttccaaaatttctgcttctttgagctcagcaaagtagctatcccgatgaaattctatcttaatattatttatttcatcattccactcatcttctgtgattatacctttaagcaaaagatttgttttaagcggatctaggaaaacttcttcaaacttatgctgcagctcacgaataaatttagcaaacgttaattcatcacgtgtaatgctagttccggaatcaaacatcacaccgccttgttggtcttgcggaatgcgtgaaagaggaacacgtaatgccatataaagagcttgtctaaaccaacgaacatcttccatattaccggtattatcagcaccaggaagagtatcaacttctgtcacagctttaccatcacggcgctgcaaccaatagtcttcggtcatagacatattatgttgttgattttttattttacctgttgatgcatcatatactactcggtttttcatcgtgttcataacatgttgcatatgctcagcagctttacgagcaggcatattacctgtgtctacataccacacacgacggtcaggagcacgagtaatgcgataaatgactacagcatcttctaataattttaattggttagcaggtttaacagcacgatgtaaatacccgatgatatttttaccacaacaatcgactaatccagaatgggcataaacaacagcagctttaggaatttttatttttgtgccagcttcatacattctaccatcacatgcatatgactcatgggcagtatcatatataaaatattctttgtaacctttaactatttttgtgccagcttcagtttctgttataatttcacgaacatactggacttgacgagggtctaatctacgtaattcctttatgccttcttttggacgttttggatcaatgattttatggaagaaaattcttgaatcgacataccaacgtctaaaatgatcagaaccttttcgttgaaacgatagatgatttaatacatcactaaattcatctaacatcatatttttaatttttgggctaaatttagatttatccaaatttaacgctacgacttcagtatcatcttcatagacgatagcatctgaaacgatttctgaaactgcattatctacttcatagttattcatgagattacgatatgtatcaataagctcacgagtagttttcattcctggttcatatgaaccaaaaattgtttggaatgcagcattataaggagaagcagcttcattcgagcttacttcaaattctcttgctccatcatcaagctttggggctgtaatggaaacaagatcttctttttcttggtctttaaaatttcgttcgtccattttagcccatggagcaaacaaacttaatacattaaatttcattgtattctccaaatgggaattatagttatatttataatggacttctctgctttaagcaggatggggatttctccccattcattttattcccaataatcgagagcaagagttacttcaaaggtttgaatttcattgtttgaatcccaatctaattgaagttcacccacgttagtaggccacagacctttaatttcaatttcttttgttactgttttagcgtcacgagcatattgacgaacgatagcgctctttttatactctgcaggttttccaccagtaatttcgtttccttgccccgcagcaatgctttgccaatcaacgaacttctggcgagcatcatgtgcctcatcgttcattactgtaacagtccagtcatcgaatgtacgatcacctgctacgttaattttacggttcataaatccgactggaattttttctacaataccagctggtaaagcagtggctttacattggaacgtaaaattttgtccaagataagaaatttctacttggaataagttaggtcgagcaaaatcaccagattcaaacgctcgtgttacatcatctacaaacatattagcctctgtagtatttatatccctatgtttaacctagggcatatagaattaaagaattaaggatatagtgtatttatatggcctgccgaaacaggcctttagaatgcaccgtattaaccagcaagaccagttaactcatcgaaatctgcaccagtagcagtcgcaacgaagttaagctggatataatttatactgcgcgccggttggatgtagaatgttgcaacaaactcatttctatcaattactgacggagtgttatttgttgtatcgcaaactacacgatattcataaattccgccaagagctttaattccttgcaagtactgagcagtttctgtacggaatgaagaacgagtaaacgcgttgtttaattcgaacaaacgatattttgaactacgtccgatattcgttttcaacatattaaacagacgacgaacgttaatacgatcaaatggagaaggaacagaagtagctgttttatcaccatacaatacgtaaccatcaccaccagtaccagttactgggttgatagcttcttgatataagcggtcacgttgtgcctggcgagtttcaatagcaagtttaataacgttaagaatctggccacgattataaccagctggtgacatccaagtctgagatacgttatcagttcttgcgcataaaccagcaatatcagctgctaatggaacccaacgattcacatcattatatttgtcatactgatatttgtagttaccatcaattgctgcgtaggttgaactaatattaaagttattatcagtatatgaacctgcggcagttctccaattgactaagttatcaacagcacgggttacaggaattccaactacagtttcacgcggtggtgagcacaatactaagcaatcctggcgagcatcaccaattgaaacgacgtgtttttggacagtagatgctgtttctaaagattcaccagcacaagaacccgcaataaacagttgaacgtcaacagattcacggtcagcaaagaagtcccaagcttccatcaaatctcctgctgttacttcagcatttgatgataatccaccagacagagttaaaattccagagaagccttctggccagttttgtgcagttgcaaaaatatattctgagccgccttttgcgaaaaagtcatcgatatagatgttactatcgtaaatatctttttcgccacgcttagttgaaagaacaacgctttgaacaatagcatcatttcgacgaactataatagcgtactgtgagtcagtctgcggcccatatccaaatactgccttggcagtagatgcacgagtaccaccacctggataaattggcagtaatgcagaagcgcctttcgcatagtcagctttagatacgatttcaatttcaattttatcgcctaattcgcctggatagagagctactactcctggaattccatatttttcaagatttgcctgaaaatcaactgctgtcatagcagtttcagcgctctcgatttcagctaataaaataccggaatcagtaataatttttccaagagttattactgcagctaaaccggaagaagatgaagaaatttctgcagtccagttagaacctaatgttgggtattcgccaacttctttagctttagcaataatttttgcagtaggaatattaattttcttaatttttccatctgcgtctacttcggtaattttaccttcagtttcaatatcatctgaaacatatttgaccgtgattttatctccaactgcatagttactacctggggtagaaattgtgtattcaatattaccagcaatcggagatgagtttttagcggtatctctatcaacagcacgaactactcgtaagtcatttccgtactgcaagaaattcatcgcagacataaaatagtcagcagtttcagcggttggttgaccaaaagtattaactaaatctacttcatttgtaacctgtttaatctgaaaagcaggaccccactggaatttaccggccaaagctgctgtaccagtagagttattaaccacggtgctttgaaccgtagtttctttgagctcaatgcccggagataataaagtcatttttaatcctctttaatatgctttaatatatttataccattgacataccatgagatactggaacatactcagcagaatgaaccgaatcaacaaatataactggagcgtattcatcacccatatcttggagttcttttgaaaacacttcagatgctaatcgcatgtcatctttgtcagcataatcaataaattttgattgtgttgataaccatccaaaaattactaaagacattactaaatcgtcgtgataaccttcttcagccgcccaagacacgcctttttcactaaacgttctaaactcttgaatagttgcacggtgatgaataataagcttatctttttcaataaggtcttttaatgtagagcatccgactgctttcgttcgtttagtttgcttcattcctaaatcagtatatgaatcgcaaataacgccttcgtattctaaatccatataaagcgatttagcaactgacacaccagtactatttaattcaatataaactgggcattcattatattctactaaataacgcataacgatgtcaggtagaattaaatgagaaatagtgtttgagtgtaaaacaccaacttgttcccacacatcatcggtaacatcaataatatgtaaagcgtggtaatcttgcccacgaccttctgagcagtctagagttgcaatatattttctatctggttcagggcttttaaatcgatgaaaaccgtgatcatctggagttacttcaatgaaatccataacagctaatttcattcctgaaattaatgtaccagaagtcccttcaaatgctgcggtgtgttcttgacgaaattgagctaaagtagaaccattaatggtttgtatgctccattgccatccatcgtcaaaaatatcttcatcgttataaagacgttctttaactgaattccaaatagcagtatatggttcaaatccagatttaccttcaacggctgcagtccaaatatcataaaaatgatttaatccattaggagtcgtagtaataataattttagaacgacgaccagatgaaattactggctgaatagcaagccaggaatcatggaagtttggaataaatgcacattcgtcaatgtaaatcattgcgaacgagttaccacgaactgcgtcaggagaggaagcataggctccgattgaggaaccattatcaagttcaatagaccctttattccattcaacaattcctggctgtaaaaagtcaggaagcagttcaattgcttgcttagtacggtctaaaacttccgcagacattgagcctttgtgcgcaagaatacctacagctttatccttgttaaaacatacaaagtgtgcaaggaaaatagctactacagttgttttaccgagctggcgcgatagattacaaacagtcatacgtttagatgacattattttgagcatatcacgctgatagtcacgtaattgaacctttatgacaccatagtcgatatgagtaatggcgcagtatgtttctgcaaaataaacaatatcgtctcggcattttttccattcctcaaccatttcacgagtccattgtgttttaatattagctcgttttaagttaggaagacccatataccgagatcttttattattcttatctttataagtttgaaataattcaggcttatcagaattatttgggatttttactattttgtgtaaacgaagataatcgctgaatttttcaggataccattttccatcccactgagattttatccaatgaattccgtcttcatcttttctttccgctaaacttgggtgttttattaaaatttttccagcttcatttaacggatggaaatcatttaatgcattaatcggttgttccatttatcaccttctcacgagcttcttgagcctcgtaagcatcaccaatttcgtccattaattctgttggtgaacccatgaatactgtcgcgttctgaatattcatttgacctgtaggaacagcgcctttggtgccaacctgctcagatgtaatatctttcatatctttatgaagcttcagtatttctctgttcgtcgtagtcatttgccccataagagttgcaaatacttccatgtgacgaggagaatcagcattctttgctgtctcaagaaaaatcttggccgcgtccattagcatttgttgttgaaaatgcatatttcgacgaactactccataatcatcttctaaatctggagtacggttttgtggattacttttaacttctactaattgcagaggttcatatactttaatttcctccccgtcaattccggggaggtcagaaatatctaaaagtttgtttatatcaagaccttccataataacctctatgttcttgggccaggaggttctggcggtgttggtctatttacattgttagtgaaagtttgttttactgttccatcccagtcttctgggtcaatatctcgaggaacaacttcattatcaacagattcaaaaacgccttcgccatcaggcaaatctctggtattagcatgaaaatctgtataagtagtacgaattaatccttctgcatcatctactggaggatacatccatccatttacttcaaatgttaatgaccattcaattctacgacgagataaattgtccccatctatagcttcgtctatagcagcagacatcagtacaattttaatatcccttttaaatggaatatcatttccaaactgctcgtacatagttgtattaaaatgaggttgaaaatatggaagaatctgttcaactatttgaaacatatcatcttcgtagcgagtaaagatactcaattcataaatcattttaataggagatggattatactgcgatactacagaagttgtacctttttgcagtaaattctgatttaaaatgtttgttttaaatggagcgttatagctaaaatcaactaaatgcaaatttatacgaggtagaatagtttcaactttagctacatcttcttgtgaatttattgatgtccatttattcaatttcatcataaagtgttcctttgatgcatacgtaataggaacacgtataaacttatcaccagattctaactgacgcttgatttggatatttgaaaacaaatcgcccatcaaggtagcatatcgtctaaaagacgaattataaaaataaccaaacatgatttctcctaatctgggctttaattagtttataatatttattaatccatgaaatcattatcaaatgggctagattcgaaagattttcctctgttattgacaacaacataaggttcaacatattctttagcttcagaattaatttgatctacttcagcatattgatcaatattaatatcatgaataccgttaagattgcgaacaggatttagttctaattcactaaattctggaatgttaattccttcgtttttctgtagaactggattaatttcttctccagaataaatgaatttacctgctgtaattttacgaatagcgttttggcctaattgataaaatgggtcatatggttcaacccagttgatttcaaataagctgttatccataggaaaatatatcaaatcaccttccttgggttcttttccatttacttgatgtttaaacaggtttggattaatggacaaagttacttcatcctgtacttgcataccaaagttaccaaagaacgatttagctccttcatatccttcaaatgaatttaaatatgcagcaaatttccatgctttagtaaatttattttttaggtcttcgccgaataccaaatcaggggaaacatactctcttggaacataatagcattctacacctcgcatttgaatgctttcagctactaatacatcagctagtatttggctgttttgatagtggttaaaatttacataaggatttagtatttctgtttcattggtctgagaataacctgtgcggttttccaatttagcaaaaagatttttatcataagtagccatattaacctacaaaaattccaaatggagggtcaagtaagtataattcttcgcgcagtgcttctttttctaaacgagcttcttctattaaacgttgaccatcaattgtaacgccgccgggaagcatcattccttggtgacgtgctaaaatttggccatttagttctttagctaaagctgttgcatagtctttcacccaacgattattataagcaccttgcttaacatctggatcttgaccaacaacacggcctactaaattgcggtctggattattatatcgttcagataatgaccagctatcttgtggaccgactgttccatatcctactgtatttccaaccattttgtttgtatcaatgtatgatttagtccagctttctacgataatcaaatcatatttttggaagtttcccatgactttgagttgttcatttgctgaattaaaccaaaagtctggaataggcgaaagcatatcttgcatcattcccatgtaactagtaagctgggtaaaatatcctaaatcagctccaaaggcatttggtccataaaatctattacaagaagttcccatgccaccattaataccagccattcctaaaagaaagtcagtaaaccatggatatgtagcgtttccgtccattgacgttattgatccaatattcgtgcgcaaaatgcgagttactgcaaatacgttagaacctcttaaatcaaatactccagtcttatacttttcttcatcgtctcctacataaaatacgtgaaaacctttgttaagtccatcaaaatggtattcaccgtataattctagggcacgctggatacaatcataaatttgatcgggtgttaactcaacattaataattggagcccctaaacgtcttagaatgacatctttgagttcctttggattctgagaattatatcctgacatttaaaatcctttagggccttgcggccccatgttatgctggtgataaaaaggtagaaagcaatacccattcgccgtctttacgaacgtaagcttgaccatcttttggagcttcaggaatataaccagcttcttgtaaagtctggacatccctctgcaatgaggacacattacctttaatactacgaatctcttgattaattgaagaaatgttagtctcattagcttttattgaattagtcaatccgcgttcttcgacagttgaaccattcggatttgttccatttactaatttatttagtgttacaacttgccctttaattccggaattgttattaccaatttcaacctgtaagttttgaacatcattgttaagtctattaacagaggtttcaacagttgaaactctatttaaaagagaccccggaggagaaggttgacctccgctagaatcagttccaactatctgatttagccatgaaacgttagctcttaaaccggacgaggtgttttcacctacaattccatttaaagattcaatagaagtggtattatctttaatttgacctttaatactagaaggaatatcatctgaaccaatcgatgtttcaataacagttaatcgctgtttaacgccgccacttttatttagttcatcatttaaagctgaaatttcttgactgttgacattgacatctgtgattattgaagtatttcctggatatccaatagaagttttgatagatttaatttcggaactaagattaatctgtgattgatctaaattatgcacacgtgaatatatgctttcactaaatgaagatggtctttgtccaagttcttgtctaagatttccgacttcaattgtcagtgaacctacatcagattcaataaatttttcttccaaatcacttagacgtattccttgggaagttatagcatctgtattattaatgatacggtgcttcatccccgtgctgctatttcctacaacaggaagaccattaatatcttgcccagcatattgtcctaattcccttttaatccataacaaatcatttctaatcgttctataaactgaatttgcttgcgaattaaatggaccaatatcagaaagaatattatcaaccgtagtatttgtaccatttaatatatcagtgtgttcattcgtaagcgtctttaaatttgatatgtcatttttattaacgctaatctgtgatagtgcttcgatatctccagatacatccaaaatgccttgaattgttttaatatcttcctgtgatgtttcaacgacagttttaagaattcctatattttcatcaagaacctcaacatttttaaatacggacacaattggtctattcattgatccatcgtttccatacttagtactagctcctagtatttcttcaccatttttaatccatgaaatacgttcctgaccttcgccaggaacgctatcaacaaaaggtaaatcttttaattcaatcatttgtaatccttagtaatgaattttaataatatagtttaatgatatattccatggtctagtttcataaccaattaatccttcacgattaagtgtaccaagggcatctctcggtccacctaattcaaatccatcattagtgaagtatgaagcattatcccagtcggcatattttctagttccaagatatccttgataaacggacgcgccaaatggagcttcatgtctttggtattctccccaaccgccagcatgtttatggtatgacatctgttgtgcttgaacaccgccaacatgcattccgtcacatcctacaccaagtctatccttcccgtaaccatcttgccctcgttgatttaaaatatgaccaccagtaccagctcctctgacgaaaagacctcgcatatcaggaatgccaggattattccaatcgccaccaaatctcgttccaactgtgtttctataatcaggatattgatctcctgacacagttccaccatggcacataatccaaccaggaggagctgaatctccagcaaacatcataatagttccaactggaaccctttcggacgcatatctttcagtggcaacaggttgatcgccatttttccacattccgccgcgtgaccaaattccatttgctgttaaatgatctaaatttaaagaaccattaatagtttgtccaccgcgagtattaattacatctgcgttccatgctaatgctccagagctatcaccttcaggggcaataccggcttgcgtcgttaattttactaccccacgcatagaaccggtagctcctcgaccatttaatgtttcacctgttacagcaacatctcccaaattactgttaatttccgattgcgttcctaaacgtataacacccttatattcttgtgttgcaacagaattcataaaggtatacggagaaattgcatatccttcgcggagagttccttgacgagtttgagcgacagttgataactgaactacgcctctaacagattctgatgctgtgtcttctgaaggagctatttgcgaaattaattttattgcaagtttttgtgtttttagcggagtcattgccgtagtatcatctacaccagctaaagctgcaggtgtagatgatattttaataaccccgtttgatgactcagacgaatatctattttggaacacatcgtcagtatggtatttcagtttttgcggcgttattgatgaattattatctgagccttctaatgtttcttcatttgttgaatagcgcgttaaaccatacttagtttcagtagcatttggatacaataatcttgttgctaatgtagctggtgtaacgattttagaattattagttccatctaaaacctcttgctctgacgcaatcattgctattcctttaacttccatagttgcatcaggaataccatttacgccgatgttacttatttttgataatgcagactgcactgtcgtgacagtgtcaggaaaattcgatcctacaggatcgaatttaacgtattttgattcatttgatacgtgctgatatgtattgttactcatgctattctctcaaaataataaaataccgtaggttgtgtgtgactatctagcgtgacagtttgcgcacctaataattgccaagttccgtatccaggtttagaaggagaaattatttcttgtttgaaagtaattcctttatcagagtattgttctaagatatgttcttggttatctaaatacttaacttgtattttatttcctatggtaggatggtcttcaaatgaatcaattgcaataaattttgcaaccatttcctgtagagctgttctaacggcactggtaaattcgattgaagtcattccattagtagctttaactggaataccaaacatgttaacaataactgtgtcacctggtttggctgaagtatcattgacagttccttcaaatgaccagtagtctgtttgttgcactccagtaggagatacgccacttatattaactaatacagctccaacctgttgctctgccatatttctaacatcattgatagcagattctacatttgggtcataaaaaccttttgctatttgtgaaattgttacagcacctactggctgattattcataaccgaaatatcatttttcttagttctaaaaccaagaaaatctgctaagcgggaaataactcccgctttattattaagtaaactcattatgcaatccttatccaacggtatacagtaatagacggttgaatattactaatattagcaggaggagtatgtgaagagtttgttgttgcgtagtcttcacgatattttgtatatataggaccagtttcgtctgggtcatattgacatcctccaataataactgatccattttcgtcttcaattaaaactctttcatcggttttagtcgcaggaagatttgcgttttcaagcgttactgatgttgtaccaactgttccaccggcggtatgtgaaggatttccgctagaatctaaatcattattgtttaatgcaaagtttggatctgtgacatcatcattccatcctactaaaacctgccctttaccaaataatttccatgaaccaaatcccatataagtcaccggattatttgggtttatagcattttcataaattgaaccaacaggataaatgctgtcgaaaatagatgatattgaattatacgttctagtataagaatcaactttttcaacatttggccaaccgattttatcaaaatctgttaaggctacatcaccagtaacttcagtagatgatcctttactaacataacggtcatcagttaattcaataatgtcatctttttctaaaagagttcctaaatcattattgaaccatgttataacgattatatcgccagattcaaattttctatcaaataaaagactgacaggttttccatcttcatattctatagaataatctgtattcgattctttccattcaccaccaagagatatacatcctgcttgcgtatctgaattagcaccttcacacaaaaacagaggatatccggctgttccagcttgttgttgtaaaattccattaaaacgaacttcaagagaattaggattaataggttctccaggaattaatccaaatgcagagaatggaattgatttcattgttgataaatcagtaacgtaaatacttccttctaaagaagtttttgacgttaattttgaatctaatacttttatttggcgtcttgtatatgaacttctccattgtgatacaccatccataaatgtttcaatttgaacagtgtcaccaatattacaaggctgtcttaacctaatattaaatccatcaagaggaatcaattctccttcattttccccaggagagccaaagtcactgttttcactaaatacatcgccgtaatataattcattaccgcggtgttttactcggatgttattaacattatagctagttccacggaaaacatctaaaaagtctgtttgtccttgaacttcgactaaaaattctttacgtgctacattactaatatctgaactaataattttgtcaatttgtttgtttttgacatattcccaacgtcctggagcacaataaactaactctaaatcgctaaattgaacattaatttcaactgatgacgatgatcctttaatagtatcaccagaagcggctactagagtaactggattaacattccatgttgcaaatacgtccctagctttaattactttattataatcattaactgttcctttaggaagttgtagagttactcttccagatgaagtgttaatggcatatgattttccccattctgctgctaatgtttgtcctgatgaagcattataagttttccaggcaccagctgaatatggaacatcaccgtcaccaagctcgtaataaagctcatcaaagttttcatttatttttataccacctttacgcaggtagtcaccggtaccatcatctacaacattaccgatattaatattttgtttcattattgagccaccccgattttctgcgtagcgataactttaactgctgctctcataccgacagttgaagaacttatagtcgccgttacataattttctttaatactaaatgcaatattagcgatttcgtcttcttcggtttcattcccaactcgcatgacagcatattcagaagaaatgacctctgaattaacaatatccacaagaatatttatttctgctgttttaatttttcttccatctgctgactggcatgtaactaacaatttagccatgttatattcagtgcgatgaaacaatggaatgtcaactgatccagatgtagaaatattccacgtaccttcaactggtgattccttttgaccaaacatgctttcaatagaataattccaaaccgacgtagaattatcagatgaaatacagcgtaaagtaactttactatatggagtagttactaccaaattacctgaaacacctttaattgaatcaatagcctgaatcgttaaaggattagtaactgatattgatccattagaattaatgaattcaacacaatcgccaagttctcctctttcaatgataactttaacacctacagtagaggtatcaatatcatgtctagtgccaacttttactggagttgcatactcagcaatagagtgtttttgataatatccagtagcatggataatttgaccatcagctccggtgccatttgctactgccattttacgctgatcgccaaacgcattataaattgcgttaaaatcactattaattttattaccaccgtcgaataagatatcaccagtagaagcgttaccaatttcgccggtatcaatcaatttctttggttcttgaatgaacatagcggtttccttatgagtttatagtatttataaagaaaaagggagcccatgggctcccttaatttaaaatgtaaacagaatattgatttcttctgtttgatccattgccataataataggtggcctattttccatataaatcatttcacccgaatgcctcattaaatcttctgggtcataataatccttttcagctttaacgtttgggtcatttggatgagctttagcttcaagaggattcgtaattattgatatttgtctaaatcctttatttcccggcaatgcagcttcaggaaaataaactgaatctaaatatgctttaaaacggatagtatttgccttaacacggtaaattaatccaaaatcatcttgttgccaagtgagattatcttcatacccccatctagtcgggtcttcttttaattcctcaggccaaggaaccacgatatattcattcgtgcatctatttatagatacatcaggcggaatctcaaaaagatattcccacacatacccgtctccgggttcaattgttccttcagcatctcctcggccttcaggcggagtcattgacctaacagaaggtgtccattttccacctaatttgaggcattcatccttatcagttaaagatgttattgaacacattccagtatcaggaacatctaaacaacgatatactaaccagcctgcgcctgattcagtagcattgtaaggagctgagttacacactacaatatcgttaattctaaatgtgtatggatccggatatctagtatctccccaatctcgacgaggaataacagcatcaagcattgatggaagaacctttactgttcccatcatatgcgtccacatgtcagttacgcctaatacagaatcagttggataaggtggggcaaagcccacctcattttcatttgatgaccacggttctgatcttccaaatgtgataaagatagtgtttttatccggaccacttccaattgaattataaaaattcaacattttttctgttctaaattttgaagtaactatcgcacgatagataacacttgaatcattcatctattttaacctgtgttggattttcagggtctcttggatttcctatattatcttttagacgtttattaactaaatctctaaattgcgcaaatgttgttccagatgcatcaaataaaggactcattaatttacgtcgttcagacggcaattgaccttgaaatattgaattattattttcagcattatagtcatcaggaagaggatattctacaccagccattgggccaggctcataaattgcttcgcctgttactgaatcatgctcaatttcaccggttggagttaatttagctactctatcagcatattcagtaggcaatccagaatcccatttatagtttttgtatttattaattatagtctctgtgtgtttaagagttaaaccaacattaataaacatcgttaaaagggtaattgctataaatccaaatcctactggatgaacaaaacgaataacgtcagatttccaacgggaagaaggtaaattggatttaattttcattacatagtatgatctacttctattaatatagtctatattgttttgaagcaaatcctttcctttaaccccacgaataatttcgccttcaaaaccagggagtctttctgctttaacttcttgaccagcaattaatcgtcctaaaagattatgaatagttacggtccattgcaatttaccattagaatagcttctttctatataagtaacattacatcttcctgttgccgtataaatcgtttgtcctactaaatcttcagtcaaagaatcagattgaacgattatgtcatattcagtaccggccccagattcaatttcaatttcaacttcttcattataaagaactttaaaaagaaacttgtatgatgcttcaattcctttagtagaataaaaatcatagctacgtgattcaaagaatctcgcaacagcatcacgtttatcagcatttaaataaatgtttcttttatatatctctgaccacaaatattcccacgcatgctcttctcgtggatattggttacgaattaaatttcgtaaattgttatactgagttccatatccgtcagaaagatattgaatatatgcttcacaaaatgcctcaaaattagaatcttgtaacaagtaagaatcaggcatcattgtgccaatcaatggtcttaaatcagggtcagctaatccacgttcttcttctggtgaccaaggagtttcgcgctcttggttttgtaaaaatgcttttaaaaatacttcagttggcttccaaataatttctactgtatctctcacacgatagttaaaatcataataagaaattatttcgccagaagatttgtagaaaagcattcctgatgcgtattttttaaatccggtgaatttaacatttggcattactatcgtgcaatcaccatcatcccagtattcatgaactaatctatctggtgatgtttctggaatattttctataactttagtgtattttaaatcagcataaactactacagctctatcagagttgtttatccaacatcgtgtgttagattttttagaccagttaaagaacggttctgcgtagtatttcattggttgaggagtaaaagtctcccaatcagatttttcatccgctataaatgccatcatatgataatgcttatcagctaaccattcacgaggaaattcatatttaacagcaccgattaactgatatttcaccacagtttcagggtcattaacaatgttatcacttaaaaatttaaaattactcgaagatagagaaactaatttaccgtcagttgacatattcgcatatccgggttgaatacgtcttctttcttcttcggtattaccgaaaactcttttccacgttttcgtatcatggtttaaaacatatattcctttatcagcagaatcaattatttttgatgttctaggattggcatttaatgtttcaacttcaccaataataagagcaaaaactttatcaccaacagaatccattttatagcatactgctttaggatttccggttatagtcatggtatcaggttcaaaaagtctttccgaatatgttggagataatggatcagaatctataggtgcattactcgtttttatgtatctaactttgtctctggcgacaacgtaaatgtaatcatcggtgcaagtaatagcttctgcaatgcggtatacattcgctggtaaagtcgcataagtggcaaaaatttctacatcaaatcctaaatgtaattggtcgccaagtttagcgaatgttatatcctgtgaactgaatctgacatcatcagctgaccatctaacatcagtagatttgcggccatagaaaatcttgtcgtatcctagaacgtatgtagtgttcgcagattggtaatatactgtcttagataaaggatatcctacacggtcattgaagagctttacagctttccaggtttgtcctttatcattagatactttaaccacgggttgataacgctcaaaaagatatagaatcccttctgattccatcaaataaactcggttaatatccttacatacttgctgaatggaaccttgtatttcatgatactcattttcaccaataataaaattactgattgatgaaacatcaacatatgatgggctgaattggaacgattcattcatcaatgcagccattatagtgtcattattaaaattgacataattagaattgttaagagtaaacttttcctgaatgaatttattggctaattgcatttcaatcatgttttgaaatgtgtaagcattcgtagcaaaagtttcaaactcttcagtataaatccaatcagactgctcaaaatcctgcgcagctgtagctactctaataatgtacgatgttaatggattagcattatcaaaaaagaagctgttatttgcagtatatcctaagttaatccatctgtattgattgctcgggagattttctcccgagtttgtttttgtctcagcgatttctacaaaatagtagaaattagcaccaacgtcatcccagcgtacttgcacctgatttgcggataacttggaaattctgagactagtgactgaaggtgcttttactgtcattgtgatataggctccaaatcgatagttaagtattgtggacgtaaatcattttcaaatacaatcagtgaaccatcgcgcgtgaaaataacatcgtctattggatcagaatatagctcaatagtctgaacctcaaatttatcggatgttaaattaattttagcgatattccaataaatcatgtcagctggataatttatttcaccgataacatagtatttgtcacgtccatcggaacttgctaatttattaaaatcattgcctgtatatggctgaatgttttcattttctgtaacatcgccagaagcaaatggaccaataataactttgccaattcctttagaatctcggtctgttgatactatatgaacgtcatataatacatcttcttctaaaccagtatcaggatttacaacctttcgtccagaattaaatgaaaacgtattagattccatagaacgatcttttatttgattattgtatttaatacccgcttcaggcgttttatagaagttttgtacttcacgaaccatttgaatagtcgctgatgaaccaatgacagaatgatctgcatcatctacatatgtcaacatcttagatttagcgaaagatgagttaaaaatttctacatcttctgtataatagcgatcaattttatcaattatttgaccttcaagccattgctcggattcttgcagtttatttaaagcatatgtgacttttaaattagtcttaataaaaagataattaggagaaataattgatggcgtaataggagctaaattatagtctttgagataatttttaatatcttcacgctgtacggtagttaaatacagacctgatttaggtttggcagcaatgaatgcatacccaggtttagtagaatcagtgaaagtttgaacagcttgaataatagaaccaaatctttctgagacaaatgtatcataatctgtcgcagttacacatcgctgctgagtttcacgcttaatagtacccaattcacgaatacgttcaatatcttctggatcaccaccaccatcagcaccaacaaaatctgggtcatcgtttggattttcattaatattgatgacagttatatttgttaatgtatctgcatatgaaaatccgactgcgccattcgcatctgcaccgttagtactgatgtactcaataacaatcgtagagttctgggtaggtttaagacctccgatataattagcggtcaaagctccttcagaagcattaacagaaatttcaccttcgccaaaatagaattcagtgtttccatcaatagtttcacgcatgtagtaaattgttgatgtagaaccagcatggaccattgactttcttgtccagttaatccattccgctccatcaacatataatttaacctggtttctatcaatatttttatcataaatgataataggtgttaatttatcataaatgatttcagttcttactatacgtccctgggccaatttcaatcgtggaaaatattggttatttttatcacgaatagcaataacatcttcggtagatacaaagttataaggattaacagaagtatcctttgcatatgctaaaaagcgagttccgcgaggaatagtaatgtaattcctattcaatgcgtcggtgcatgttaacataatttcggtctgcgcagcggattttgaagtaggtaaatatccgttatcttgtgcagcttgaacaactgaacttcgtaagttagcagtacgcataaagctttcatacacagcagcattaccaaactgctgaatgtacagcgtattataagccaaaaggtcgcacagaacgtttaatcttgagccttcaaaatcataatccaaaaattcattttggccattaagccattcaatgaggttttgttttatttcagcaaacgtacccccgacgaatatctcgggaatagcatttgctgttcttgttaattgataatttacaggggtatttgccattttaaatcctatttaatgaatactttagatgatgcctgagccacagtatcaccgcatgatattggatcagccatttgaacagctttctttccagtgacatataccttagaagttctgggttgtgtcactccgccatgtgtttcatatggctttttaatttctgtatgttctgtaattggatcgcctgctactagaacagcaattcctccagtgaatactttactttgtgtagcattcacaactgttggaggccacgcttcatgaccagcagtaacacatttatcataacttaatcctgacattattgcctctcatacacgtagcttctcagtttattagcccaacgactccaatttccaactatagttttagtataatttttaacgagagtttttcttattggagcaggaggatccgttggttcagtagtatcagaagatgacctagaattactgccagaacccccagattcactttgttcttggtagtcatatattaatgttacttcgtaagtgaatgtcttctggaggttttgaggagctttccatagatataactgagtattagaatcagttgggaggtcttcccatgaagcagcagttttaaattcatcgcctaaacgatatttcaacgcgtcatctccaaatccaaacacagattcatatgttccatataagcgattttcttctactaaaaccccaggagtttcttcgtaactagttatatttatagatactaacgtttcacctgtttctaattgagcggtaaaggtgacgtcgatagaagaaccttccatggattctcctaaatcagcgctcattggaagtatattagccaatgtcaatcctcgatccatcaattgtgtattgaccggatgaaatagaactcatagatgccattttttctgtccaatcaccaccaacgtcccaatcaactgtcccagcaactttccaagaaagatttccatttactgtattagtctgatttccttcaactaaagtggtagcatctcctttaactgtaatgtcagcattaccttcaactacaatagtaacattacctttaactaggatagttccattaccttcaaccgtcttagtttcatcgccacgtacaaatattgtattgcttccatctatttggtgaagacgattatccatgttgtaataaatttctgatccaccgacgttagtctttttatcgccggctaccaaaaaattaccatcagcgttggttatatcatacaaattatcaacagtttttcttgttcttctccctgacggtgatacttcttcataagttccagttggatgaactaatctatatcgttcttgcccaggggtatcatcaaattcctgaatatgtccactttcagtttccattgtatgcacatacggatattcgcctttatatgaagaaactggttctttgaataaaattcttgagtcatttggaataggagggtcagctggatcagaagacttcgccacagtagcagccattgccgacaaagacctagctggtgttttaacttcaataccatatgattccaaatttccggtaagaataatcatggtaacacgggatgcacggccttttgtttgttgataccacaacgaatcacgaccagctttatatgctttttcccaatctcctgctaacatagcagttaacattgtgttaaatttagctacaccgccaacacccatctgaaatgccatattttctaatgccatttgacgagaacggttgacagcttgccagactggtcctactttagaatgtgatttaatgtcacgttgcatatcagctaaatcacgttcaaataaagttaccgcttcttccattgtaatagaacctggatttccagtaatttcacgaccaacttgttttgataaaactttattaatttgagccatatcacgaactggctgtttcatgataagatgaccaataccaattgtcggatatccttcagtatcccaataaacttttagtcttaatccttcatcacggcgaagcatttcagccattgacatatttggattatcatcagttggaatctctgatagtggtctatcatcgggatttattgcggtgtctaagttactatcttggataacgttagaagacgaatcatatcctacttctccgccttggtttagtacgttagtatcatttcctaaacgtctaggatattgtccagttggatcagaaaatccttcaagtctattcggtttttcgcgaactattccaccatacgtgccaagaacaattccgttggttttccatttgtctaaaaaatgaccataaactctagttccttctaccggtccagtaacggagcctccaattccagacattgctgcagaagttataggttgaataactgacatccatggtaatttttcagttggaatacccattacatcaccttgtgctctttgaggtggatgcagaccaacaacacgaacacgaacacgacctaattttaatgggtccattctatcttcaacaacaccaacaaaccaattaaggttattacttatcatttccataagatttctccattatacgtataaggtcgttcataaatgaattaatgtctgattttgctattatttttatttgacgaagtttttcattttcaagaacagcagcttcatacgtattaacagcagcaagcgctccttcatattgaggatattttttagctttatcgcctttatcataccaaacatatggattatcatcgtatgatattaaattgtaaaatttttcaccgttctcattcacatgatatactatttggtctccacctacgtttttgtatttttgtatagatgcttgataagcagcttcttgcgaagtaatccatccataatatggatcataattatcattacacatcaataaaacccaatacaactgtggatttccatatatagtatttgctaattcttccgggcgtggtgaacctttgatataataagtacgtaagcggtatcccgcaagagcgcgtttaaaatagtctttatagtttctaaaaatatctgtcataggaatagtcggcgcgtttttattcaccgttttggccgcatattcaatcggatcaaaaaatgtaaagagcatgggccctcctgtttataaatatattatctatttataaggagaatccaatggcatattctggaaaatgggttcctaaaaatatatcaaaatatagaggtgaccctaaaaaaattacgtatagatcaaattgggaaaaattcttttttgaatggttagataaaaatccagaaattattgcatggggtagtgaaacagcagtaattccttatttttgtaatgcagaagggaaaaaacgtagatacttcatggatatttggatgaaagattcttctggacaagaattttttattgaaataaaacctaaaaaagaaacacaaccaccagttaaaccagcacatctaacaactgcagcgaagaaaagatttatgaatgaaatttatacctggtctgtgaacactgacaaatggaaagcagcacaatctttagctgaaaagcgtggaataaaatttagaattctaacagaagatggattacgagctcttggctttaagggggcataatggctatttttcaaataattaatgaaagcactccccaagttccaaaggttaagcaatcattaaacgaaaagaaatggattcagataggtcttgaatataaaaaggccaaagcaaaaggaatgacaggaaagcaatttgctgaagaaagaggaatcaaatactctacgtttacttcggcaatgtcaaaatatgcttcaggaattaaaacagctgaaaagattcaaaagcttgaatcaaaaccaatgaataagctcaataagcaagaaagacaactgcttatgataaattcattcagacaaacattgcgtgataaaattcgtaatgaaggcgcagcaattaataataaaaccagaaaatggtttgctgaaactattaagcaagtaaaaggacataaagttgttcgcccgcagccgggacgaatatatgcttttgcttatgatgctaaacacaaggaaactcttccttactgggataaatttcctttgataatttaccttggtttaggtaagcataatttaatgtacggattaaacttgcactatattccacctaaagctcgtcagcaatttctagaagagcttttaaagcaatatgcaaatacacctactattactaataaaacgaaattaaaaattgattggagtcaagtgaaaggatttaggggtgcagaccaaatgattaaggcatatatacccggtaatattatgggtagccttgttgaaatcgccccgaaagactgggcgaacgttgtgttgatgccacttcagcagttcgtttcaaaaggaaaacgtttctctgcaaacaaagtctggtcaaatatctaattctattatcttccattcttttctgttgtttgttctaaatggaattgaatggaagggacttagacccattataccaccaacagttataaagcattatgaggaatatatgtcgcaagctctgcaacaaatttttaaccaagcaaatacaactaactttgtagtatcaataccacatagtaatactacatctgcttttactttaaatgctcagtcagttcctattccaggaattagaatacctgttactgataccgtgactgggccgtttggactgggccgagcacaacgtccaggtgttacatttgagtatgatccactcattgtgagatttatagttgatgaagaacttaagtcatggataggaatgtatgaatggatgctaggaactagcaactatcttacaggtgaaaatactgcccaaaaaacaggtcctgagtacattacgctttacatcttagataatagcaaaactgaaatcgtgatgtcaataaatttttataagccttgggtttctgacctatctgaagtagaatttagctatacggaagattcagacccggctttagtatgtactgcaacgattccttacacctattttcaagtagaaaaagatggaaaaattatagcagaagtttaatgcttcagttttgtgtgttataatcttaattaaatttgaggagaaacacatgaaactaatctttttaagcggtgtaaagcgtagtggaaaagatactactgctgattttatcatgagcaattattctgcagttaaataccaacttgctggtcctattaaggatgcattggcttatgcatggggagtatttgcagcaaacactggctatccttgcttaactcgtaaagagtttgaaggaattgactatgatcgtgagactaatttaaatctgactaaattagaagtaattacgattatggaacaagcattttgctatcttaatggtaaaagcccaattaagggtgtgtttgtttttgatgacgaaggacaagaatcagttaattccgtagcatttaacaagattattgacgttataaataatattgaagatcaatggtcagtccgtcgtctgatgcaagccctaggtacggatttgattgttaataacttcgaccgcatgtactgggtaaaattatttgctttagattatcttgataaatttaactcaggttatgattattatatcgttcctgatactcgtcaagatcatgaaatggatgcggctagggcgatgggtgctacggtaattcatgtagttcgtcctggtcaaaaatccaatgatacacatattacagaagctggattgccaattcgtgatggcgatttagtaattacaaacgatggttctcttgaagaacttttttctaaaattaaaaatacactaaaggtactataatgtctgaacaaactattgaacaaaaactgtctgctgaaatcgtaactctgaaatctcgtattcttgatacgcaggaccaagcggctcgtctgatggaagaatccaaaattctgcaaggaactttggctgaaattgctcgtgcagtaggtatcactggagatactattaaggttgaagaaatcgttgaagctgtcaagaatcttactgctgaatctgcagatgaagcaaaagatgaagaataatggaatttaaagacttttcaacgggtctttatgtagcagctaagttttcagaattaacacttgatgcgctggaagaacttcagcgctctttacgtgttcctaatccagttcctagagaaaaaattcattcgactatatgttattcaagagtaaatgttccgtatgttccatcaagcggaagttttgaagtagcttcttctggacatttagaagtatggaaaacacaagatggatctactcttgtacttgtactagattctgaatatctgcgctgtcgacacatgtatgcgcgggcgctaggtgctacacatgattttgacgattacacaccgcatataacattgtcttataatgttgggcccttatcatttagcggtgatgtacaaattccggtcgtattagatcgtgaatacaaagaacctcttaagcttgattgggcagacgatttaaaataatttcacaaagttgtttacacgatgatggagaagtgatattatctccatcaacacatttattaatttaggaaaataaaatgaaaacttatcaagaattcacttcaacgactaacgcttctactttgactgaagcgacccttacttctgaagttattaaagcaaacaaaggacgtgaaggtaaaccgatgattagcctggttgatggcgaagaagtcaaaggtactgtttacctgggtgatggatggtccgctaagaaagatggtgctacaatcgttatctcccctgctgaagaaactgctttgtttaaagccaagcatatttcagtagctcagcttaaaattattgctaaaaatcttttgtaataaatttttcgcaaagttgtttacacagggttttagtagtgatactattaccctatcaactactgaggagaataaaatgaaacgttgtgaattaattcgaaatgttgctatcgcaatttctgcttccgcttttagtttttcaatgtttgttggatttatatgcggattattaactacagcagaaaatgtattttcacttgtagtagcatttttaattggtttaattgctatcgttatggataaaatttctaaaggtgagtaaaatgtataaagtatatgcagattatcaggctaatccatctgaaaagccagattgccaaattggatgggctcatgataccttgcttgaagcagtagatgctgctcaaaaattaggatatagttatgtagatattgttcaacctggtggaacagttattactcttgaagagttcaataacattaaaccaaactttttactttttgcaggtgataattattatcctcgcggtggatatgctgatttaatcgcgaaagccgctaccgaagatgaactccgtgacattatcaaagaaaatgagaacaaaccaatgtatggttctaatcgctttgattggtggcaaattgtaaatgcccacactcatactattgttgatgaagggtaaataatgattctttatgcaaaagtatcatccattgaaaatggatataaatatgatcaagaagcagctaaagccttgattgatgattatggtattttaacatgttttgaagttgaaaaagtttacattgaccgttcatcttctcaagttaaattagtgaaggaagaccataaatttaatacagtaaattttgatttctttattgaaacagaaaaaggtcctcttgaatatgatattttcaagaatcctttgggtcttgagtgcatcgtaaatatgtattatcataaatggtaaatatgctttaagaattatttgttattattaactcataccgcactgattaataccctctatcatcaagggctcttgcttgagagcctttgttaataattgggaattagccaagttggtaaggcactggattttgattccaggatgcaaaggttcgagtcctttattcccagcgcgagaatggtcaaattggtaaaggcacagcacttaaaatgctgcggaatgatttccttgtgggttcgagtcccacttctcgcaccaaatttgcggatatcgtataatggtattacctcagacttccaatctgatgatgtgagttcgattctcattatccgctccaatttaatttactccgtgtagctcagtttggtagagcgcctgatttgggatcaggaggtccaaggttcaaatccttgtatggagactggaggcgtggcagagtggtttaatgcaccggtcttgaaaaccggcagtcgctccggcgactcataggttcaaatcctatcgcctccgccagttttgctgatttagctcagtaggtagagcaactcacttgtaatgagaaggtcggcggttcgattccgtcaatcagcaccaaggccctgtagctggaaggttcaagcaagcgactcataatcgccagatggtggttcaattccacccagggccaccaaattatgaattaggggagttatcccgtagaggtagcggtgtggactgtaaatccattgtcattgcgactcgggtggttcgactccatcactccccaccaatttggatgtgtagctcaatggcagagcgatcgcctgttaagcgattggttataggttcgaatcctatcacgtccgccaaatttgtgacagttgtcacattaatatctacttgacgcgattatcggggttgaactgatcactcttctcttaatttcgtgtgaagacatcttgattgttggagtaggtattaatgtggccgtagttcagttggtagaactcgagattgtgattctcgtagtcatgggttcgactcccatcggtcaccccaattacggggcatagctcagaaggaagagcaaggaccttctaagtcctaggtcgtaggttcgatccctactgcctcgaccaattaagtgaggaaaatatgaaaggtaatgtttatttagtagttcatgatttaacattttacttcaatgaatatgaaaaacatatctcatttcgtgaaattaatttactttatcaacacgcggattatatttatattcaaaacgaatttggtcattggcaatttctcaaaaatcgttcatttggcttagatggttacgaatactttgaacgtaaagaccttttagataaaattccgttatctacacaatatcaaaatcacaagtctttatataaatgccgtccaattcgaaatgctgaatctgcgtatgaagcaattgatttatggcgtaaacgtcgtgaacagattgatgctttaaaagaatattaagaaaccgggtcgctaccggtaagtcgtcggactgatgttccctgagtaaggaattgtgttaataaatctttatgatttgttgatgccctcttacatcacagctgaaacggcgcaccgaattatcgattcgaggaaatatctttgccgtaagccgagtagcgtttttgacggaacgttcggatatggtcgagatatggccttttaaaaatattgagtagcgtcaactgcttgataaccgggttcgaatcccggcgtttcgtacaaacacttgccttagcaggtggaaccccgacaaggttgccgcaaggcttagccccagccgaaaggttggggctttttagtataaataagtatattaaatctacaaattaaaacaggaaataagatgaaatcatatactcaatttttaaatgaagcggtgttaaatgaagcatctagcaccgaaattcaagctgtcgcaaaagcagccattgcagcgggtaaatattcttacaaagatgcttctgatgaatcacgattccagttcgcacgcgacatgaaagcggaaggatttacgggaaatgcagttagtatggcctggaaaagtttagttgctactggcgctgcttttgcaaaggcttcgggtaaacctgctcctaaagcagatcctaaagtcgcacaagagaaaaatattgttaaaggaattatctccaaatatgaagctatccttaaagagcttttagtaatcaaaactgaaggccaaaaactagctcgtgcttatagtttcaaagataacccacatgctcactctcttgagtatgttgaagacatccaaaaaattattaaagaccgcatttggtctgctaaacaaatcaaataacattcttagccctagccgaaaggttggggctttttggtatctaggcctttccaggcctctctagacatcatttagtttctaccctttataatatattatcctatcctttgatttcccctccctgccctagaattccccaaaactttttaaaaattttttcacaaaactgtttacatccttgtccttccatggtactatacaactatcaactactgatacagaaaacaacttggagaatgaaatggataattacggtgaactgttcaacttctttatgaaatgtgtttcagaagatttcggtcgtacggtgaatgatattaaagttatcggtcctgatcatccgatgtttgaaacctatgcagtaatgggtaatgaagatggtcagtggtatactgtaaaggttgtgattaacatgttcactgctgaaggttatgttaaactgtcttctaaagtttaccatgataacgacgaaatcgcagaagaatatttcaataatatgaaataagtttacataggctcatgattgagatattatgagcctataatttgaggaaaactgttatgtctctttataccgaactgaatgaaacttccgaaaaatcttttgatggtatcttatcaataatgcaaaagcaggtattagaagagcttagtgaagccgcaagcaatgggtacaaagcttgtcgagttcaaagcccacatcgccgcatgtatgaacgccctcttattaattggctagaaagcgaaggtcttacggttaagatttgccctgctaaatttgatgttccacgtattcttaatatttactgggataaaccggttgagaaaagtgacacttgggttgttaatgtaaaaacatgcgataatggatgctcacttgaggtgtctgctgtaattaaaggcacccatggtgaaaaatcgtggggttggcctggtaaagataaagaaatcgtgttaagtgtaaaagctactagtattagtactcctatagctaaaagcattatcactgccgctgttgaagaagctgaacgaatttgtaaaattaaaaatagtttacatctcttaagtagcatgatataattcttctgcagattttaatttctaatcaacctaaggaaatactatgaacacactgaagaaaattgttgagtttattcgcactaaacttggttctgctatggctaaaaatctatctgttgaagagcagtatactgccgcagcagcaaaattgcttgataaaattaaagatctaaaaactgcttctgttaaatctattaatgaagaaaagcgtattcgtgaacttattgtcgaaaagaataagcaggccgaatcaaaagagcgtgaaattcgcaagctcctttctgaaggtcaagatgtaacaatgcatgctaaactcggtttactatatcgtcgaacagctgagcagttgactactaaagctgatggttatgctgaaatgcgaattgaaatcgccaagaaagtagttgagttagatgatgctcgccaagaacttgcagttaaattggaatatatccgtgaaactcgcgccgcgaatgcccttggaattagtactgctgatgatgtagttgaaattgcagcactgactaaggttgatattgaagatactcttgctcgagttgaaacctttaatggtaatatttctggggttgaaactacctctgccgatgttcaggaatatattaattctctgaaataatgataaggggcttcggcccctattacttggagtaagtaggaatgaaaatgcaaagtgatttcaattcaatgtttgaagagttccaaagacaggttgatgttccagaccaattattaaatgctcttaaacgtatggcagaaggacgtaattattattggggttcttcatatgaaactgttgaaaatctttctggaagattttctagaggtaaaaagtctttaatacgtcctggaatacttattaacagtattgaatcaattcatttattaacatgcgattttgacgttgaatttactgattttgtttctcctgaatggacggtctgttatttaaacgacgattatgattatcttggcgtttatagtttaagtgatgcatggtttaaacgtaatttacaaaagtcaaatttattctatattgatactactgtaaaatttcagggcaagaaatatttctttactcttatagttgactctgaaacaaaatatgaaaataaacgtattcttagtaaaaagaatatcttaactattgtcgatgatctttttgataaattcgtagaaaatcccaattttgaaagcgatttattattagaaaaatttgttaaggaatgtagagaatatgtcaaaaccatcactataccttccaagtaaaccgttgaagtatgaattaaagcgtcagataatttctactgatgtgttaataggtcctgtggtagttgtatcatttgtgattctggtgattgctggaatggttatagacgttatgactgatattgaatctggtgcaatatttggagcaacactaattcttccgttggtagttccgcttttattagtacctataaattggataggatattggtatcaaggaagacactatcgtaaacgtgtacgtgattggaaagctcagtgtaaaaagattaaaaaggaacatcagcttaaacttgctgcgcatgaatttgatgaaattatgaaatttgttaaggaatcacgatgcaaaagccaaaactaaataaagtcaaatattctcttcctgagtcatttttaatttttactgtagcgttggctgcatctcttgcgggtagtctttttggattgttaattgactgtcttattttaaacatccgtagtaacgtcagcggtataatagatataatagaagtttggagtgaacttcattatactatagcgatttcattattttcattctttggtacaatgttatattttcattatgataattttaaaataaattggcaaagaaaaaaagattacaaaatacaattaaaggaatataatagctacatgtcttatattgaaaaagaatcaatggaagagtttgtgagtgattgtaggaaaattaaatgattttaaaaactcgctggtatgatttagatgatggggatgatggcatttcagttgatagagttgactggaatggttgttccgaagatatgaaaaatttgttgattagggagtttagaatgggatatcaagcagctaagccatctattataacagatgataaattcgtgtgtattcataatggtcgcgctaagttaacgaacgctgattggttcacagataaaattatgattctgtggtatattattagtcttcctgtgacatcattcgtattttacttttttataaaaaatccaatggacagaataggagattggattcttttaaccatacttgttaatatttttactgcagcaatattatctgggatatggtgcgtgttcattgaaatgccatggcggctacgcagacagcaaaagatttttgatgaaaagaaatacactcaaaatttaaataactttatcactgaatgcaggaaattaaaatgaaaacattatcagctggtattatctttatgacagaagataaagatttatttatgggtcgggttactggttctcgtaagcctggaatgatggcacatcgctgggatattccaaaggggcgtgtagaaagttctgatttaagcgcattagatgcagcacgaagagaatgcttagaagagaccggttttagcaattataatccagaccttctagaagacctaggtgtatttaaatattctagtaataaagacctacagttattttattatacgattccagtagagcatgagatgtttagaaattgccattgcgagtcttattttgaaaataaagatggtgttatgattccagagatggatgcttttgctcttattcctcgtactcagtggcaatatgtgatgggtccttcactttaccgaataatgaacaacctcttttaattttataaataccttctataaatacttaggaggtattatgaatatatttgaaatgttacgtatagatgaaggtcttagacttaaaatctataaagacacagaagactattacactattggcattggtcatttgcttactaaaagtccatcattaagtgttgctaaatctgaattagataaagctattgggcgtaattgcaatggtgtaattacaaaagatgaggctgaaaaactctttaatcaggatgttgatgctgctgttcgtggaattttgagaaatgctaaattaaaaccagtttatgattctcttgatgctgttcgtcgctgtgcattgattaatatggttttccaaatgggagaaaccggtgtggcaggatttactaattctttacgtatgcttcaacaaaaacgctgggatgaagcagcagttaacttagctaaaagtagatggtataatcaaacacctaaccgcgcaaaacgagtcattgctacgtttagaactggcacttgggacgcgtataaaaatctataaagttgtttactttctcctagaattgtgatagtatattcacagttgcttgaagagacaaattactaattaaaatatttaaaggaaacccatgaaaacatatcaagaatttatcactgaagcagctattaattctcaaattattgctgaatcttttactgatcttttgaaatttaaaaaaggtcaaaaaattactgctgtattggatgatggtacagaagttgagatggatgtacagggatataattatgcagtagatggaaaattgtataataaatctcatgctaaatttgattcatttgacgactttgttaatgcagttgaagatgaaaaaactcgtcgatccattgcaactggtgatgctaaggttcttatggcacatggtcatgaacgcattcgcgctaaacagaataaaatgggtgaagataattttgcattagttggttatcaatctggtaaacaaacctatggttatcagcgtactgctaccatgtataataaaaatggtaaaattgcctttgtgaatagtaaaggttctattcagtacgttaaatcgttcaaataatatgggaacaacctggacctcatgattctatgagggattcccgccaacctgtaataaggtcgagtccaggtgcggtaatgggtaaatacagaaatggacaattcatgcgccatggaatggcccaaacttagagagaagaaatgagaacatttttaactggtccttatctatccctgatgaatgcttttacacaccattctgatgctagagtagaagaaatttgtaaaaacgaatatatcccgccatttgaagaattacttaaacagtactgcacacttcgactagacggtggacgtcaatctggtaaatcaattgctgtgactaactttgctgctaattggctgtatgatggcggaacagttattgttctttctaatacttcagcttacgctaaaatttctgcaaataacattaaaaaggaattttcgcgttattctaatgatgatatacgttttcgtttatttactgattctgtgcgcagttttattggtaataaaggaagcaagttcagaggtttatcgctttcgcgaattttgtatataattgatgagcctgtcaaatctcctgatatggataagatttatagtgtccatattgacactgtacactactgctgtaatagtaaatgttgcattggtggtattactcgtcctcaattttttgtgattggaatgcaatgatgacagacactcagcttttcgaatatctttatttttcgccaaaaactattaaaaataaattggtgaatcattttgaaattttggcaaaaaataacattttaagcgaattttatcctaagcaatacaaattacaaaaaggcgtattcaaaggatgcagagttttatgtactgctcctaatgcacggctaatgaataaaattccatattttaccatggaatttattgatgggccttttaaaggactaatcacacagagtttaatggcatatgattctgagccatttttaattaaagaacaatcttggataaatttattttctaattgaggtttatatgaaagcatatcaaattcttgaaggcacacataaaggtactatttattttgaagatggtattcaagcacgaattattgtctccaaaacctttaaagaggactcttttgtagacccagaaattttctacggtttgcatgcccgtgaaattgaaattgagcaacagcctacagttaaaattgaaggtggtcaacacctgaacgttaacgttctgcgtcgcgaaactctggaagatgcagttaagcatccggaaaaatatccgcagctgaccatccgtgtatccggttatgcagttcgctttaactctctgactccggaacagcagcgcgacgttatcgctcgtacctttaccgagagtttgtaatggcaaagataattattgaaggttctaaagatgtgctaaatgctttcgcagaatggtttagtaattcaggcgaacagcaatttaatgaagcgtggaacatgggtgatattaatggaatttatcctacgacagaaatttctgttcaaggatatggcattcatgaacctattcgtttagttgaatatgatttatgtactggtgaggaagtcaaatatgattgaagacattaaaggttataagccgcatactgaagagaaaatcggtaaagtgaatgctattaaagacgctgaagttcgtttaggacttatctttgacgctttatacgatgaattctgggaagcactagataattgtgaagatgatgaactcgcgaagaattatgctgaaagcctcgatcagttaactattgctaaaacgaaactcaaagaagccagtatgtgggcttgtcgtgcagtgttcaaaccagaggaaaaatactaatggcgcaattaagtgcagggtttggttatgagtattatactgcacctcgtcgtgtatctgccgcttctaagaaaattcaaagtcttgatgacttccaggaagtagtccgtaaagcttttcaggactatgcacgttatcttaaagaagattcacaggactgtctcgaagaagatgaaattgcttactatgagcagcgtcttgaacagcttaaaaacttacatgaggttcgtgcagaagtttcaaagtctatgaataaattgattagatttaaagaataactgtttacttttcctcttgactgtggtataatttttctatcagttaagaggagaataacatgactatcaatacagaagtttttatccgtcgaaataagcttcgtcgtcactttgagtcggagtttcgtcaaattaacaatgagattcgtgaggcatcaaaagcagcaggggtctcatcgtttcatctaaaatattctcagcatcttcttgatcgtgcaattcaacgggagattgatgagacatatgtttttgaattattccataaaataaaagatcatgttttagaagttaatgaattcctgagtatgcctccgcgtcctgacattgacgaggattttattgatggggttgaatatcgtcctggacgtttagaaatcacagatggaaatctttggcttggatttacagtttgtaagcctaatgagaagttcaaaaacccgtcacttcaatgcaggatggcaattatcaacagtcgtcgtttaccaggaaaggcttctaaagcagtaattaaaactcaatgaggtaaacatgagaaaagcactactcgctggtctattggccatttcaatgatggcacatagctccgagcatactttcagtaatgtccaactcgataacatgcgttacgcgtatcaattcggggaacaattttctaaggatggaaaatataaaacacacaaaaatatccacaagagcggattaggtcatataatggctgctattttatggcaagaaagctctggcggagttaatttaaaatctaaaccaaagcatcacgcctatggaatgttccaaaattatttgcctactatgcgagcaagagttaaggaacttggttataatatgaccgatgctgaaataaaaagaatgttgaataaacggtccaattcagcttcctgggcgtacattgaactttcttattggttaaatatacataagggcgatataagaaaagcaatatcctcttataattcgggatggaatgttaaagcaggttctaaatatgcttctgaagtcctagaaaaggctaattaccttaaaaataataaacttttggaaatagtaaatgactaaaattttggttttatgtataggattaatttcattttctgcgttagcagatacatcatatactgaaattagagagtatgtaaaccgtactgcagcggattattgcgggaaaaataaagcatgccaagctgaatttgcgcagaaattaatatatgcatataaagacggagaaagagataaatcaagcagatacaaaaatgatacattgttaaaacgatatgctaaaaagtggaataccttagaatgttcagttgcggaggagaaagataaagccgcttgtcattcaatggttgaccgtttggtagattcttataatcgaggattgagtactagatgattgtaaaatatatcaagggcgatattgtcgccctatttcttcaaggtaatattattgcgcacgggtgcaattgttttcatactatgggttcaggtgtagcgggtcaattagcaagagcctatcccaaaattttagaaatagataaaaccactactgagtacggttctcgtgataaattaggcgatatgtctattatttttaaacataaacctaatggttttggcatatgttataacttgtatacgcaatacgaaccaggtcctaatcttgattatggtgctttagtaaactgcatgatagaattaaatctacaggcagaaacccttttgtttaaaccagtaatttacattccacgcataggttgtggtattgctggcggcgattgggataaggtttctaaattaatcgacatgtttactcctgatattgatttaatagtggtggattatgaaagtacattacccacatccgtttgatcctaaaaacaaagtggaaattattcgtcaatgggaacgcatttgccgtactaaatgtccaattaatagtccacatgatgtagataaagactacattggaacattcgttgaatatacctttattgataagaaaggtcgtaaacaacatgtagaagaatattgcttaaaggttacatggttatgagccaaactagtattcttaaaaatgcccactgtgaaaagtgtaagtggcctgttgtttttgctttatgtaatgatgaaatggcttgtgatttcgattattggtgctattgttctaataaaggatgcatcaatcataaaggtgaaggattttattcgggattttatccttatcctgatttcgttaaagaaggtaaaccaaaatgaatgatgatttaaaatatcaattattacgtgaacttgatgttttgattgaactttctgcacaaaaaggatttataattggatcgggtcaaaaagaccccaacggtcattcaatcgtagcggttatgaatcagaaacgagtcattttaaaacttttggggattgacatactgtgagcctaagcaaagaacaaaaagataaattgtttgagcttatccatgaacttctagatgagcatgcagaagcaaacaccttttatgatgaatacggcccgctatctcccgaacagcaggaagaatttgctgatcggtttgataagaaagaaaatgaattaatagcttatgtgaatacgctttaagaaggtgatatggcgagtttaatttttacttatgcagcaatgaatgctggaaaatctgcttctcttttgactgctgcacataattataaagaacgtggaatgagtgtattagttcttaagcctgctattgatactcgcgattctgtctgtgaagttgtttctcgcattggaattaaacaggaagcgaatattattacagatgatatggatattttcgagttctataaatgggctgaagcacaaaaagatattcattgcgtatttgtagatgaagctcagtttttaaaaactgaacaggtgcaccaattaagccgaattgttgatacatataatgttcctgttatggcttatggattaaggactgatttcgctggaaaattgtttgaaggttctaaagaacttttagcgattgcagataaacttattgaactaaaggcagtttgtcattgcggcaaaaaagcgattatgacagctcgattaatggaagatggaacaccagttaaagaaggtaatcaaatctgtattggtgatgaaatttatgtttctttgtgcagaaaacattggaatgaattaactaaaaagcttggttagtgcaaaagttataaataggtttatctaactaaaggggtatatatgctacaattaactgaaaagcaacttcgcaatcttactgtgcttcaattagatgaaattcgtagggaagttggaaatatcatttcagctttgcgtcgagaagtatcactcaaccaatctccggcagactatactagattgcgaaattttgaaaaataccttgataaagttaaggccgtgcatcggcataaagtaaatacaggacaaaaatgataggaggcctttatggccttaaaagcaacggcactatttgccatgctaggattgtcatttgttttatctccatcgattgaagcgaatgtcgatcctcattttgataaatttatggaatctggtattaggcacgtttatatgctttttgaaaataaaagcgtagaatcgtctgaacaattctatagttttatgagaacgacctataaaaatgacccgtgctcttctgattttgaatgtatagaacgaggcgcggagatggcacaatcatacgctagaattatgaacattaaattggagactgaatgaaattcagcgacttttcacaaagtggaaaaccttcaaaggcagatgaatacttaggtttattaatggctgcacaagcttattttcattctgcacattttgaaactaaaagttatgctagacacaaagcatacgattttattttctccgagttgccagatttgattgataaatttggtgagcaatatttggggtattctggtagaaaatacacaccttcaattccagatgccagtaaacttcctaccgacacaattaaaatgattgatcacatactagaccaatctaacagcatttataaagaaatgcctccagccattcaaagcacgatagatgatattactggaatgttttaccagagcaaatatcttctttccctcgaataacattagtctccttcgggagactttttcattttaccagtttactttcaatttgagctgtgatactatacaaccatcggataaagaggagaacatcatgaaaattgaagcactcaatcaagaaggaaatatctacgtcatcattaatggtgattttttcgtcgacatggatgaagttactagtgaagaacttgtagaacttcttaagaaacgttatgatatgtgtgatgaagctgcaactcatatggcgtgtgcaatattctctctttcatatgtggtggaataatgactagaattgaacaagcagataaaattaaagaattggtagctttaattcgcaaagcagatgaggaacttagtgactttgcttggttttcggcaggcattgcaaataaaggtattgaaaaatttgaagctaaagttgataatgctgtagaagctttagatatgtttcttgatgaaattatcgatcataatacgagagtttaagtatgctaacacgtgaacagtttgaaaaaattattaaattagcacgtgatgttgaaatagattcatatcaattagcagttgagcactgtgaaggatactcatacgatggtatagaagcagctaaaaaggatttggataaatccaaagctaagttagttcaatatcttgaaatgattaggtggaataatgaaaactgaaaagcagatgtttttaatgaagctaattgaagaatatgctaatgcagtttctgactatgagtattctgctagggaaagaggtacagctttcgcaaaggaagaaatgaaaatcatggttgatgctcacacaaagcttcagaattttattgaaaacgtcatttaatggtttacaagttggcaaggttatgatatagtaatcttgtcaactgccaaggagaagagaatgaaagttttgtttgttgtgtatgtgatgattcaatataattatccaatgtttacttataatctggtgaacaacattattgatattattcaaaggagtatgtaattgtgggtgagtcgaagagaatcaatatgaaacgattagtattagaagttagtccgctttttggtgaattagctatagaaaaagtaaataatatgtatcgtttgacacaagaagacgatatgctatattttacgcctagtgaaatcattcatttaacccaaattgaatatccttatactgataaaatagtaagcatcaatgatgagcacaaaattcatttttattcttcatgcccaggatttaatattaaaagtgagtcaatgtgtttatcagttatccattgggatagttttatagataagattaaatatttttattattctaatgaaagaaaacatagtttaaaatggctcaaaaattgcaatgctattattactaacgcttgcaatcagaatgatgaaactgttttaaacgtatcaaaatgctatgaagatggtgatgtcttaactattcgtcaaattgatgattttcgatcgtatattgtcacatttaccaaagacgaagctattgcgctaaagacttatctcgattctgtcattccaactatgatttcaaagtgaggaaatatgtttatttcaagtggaagtggtttaattcgtgttgaatttaaaaatgacatcttccttattcaaggagatgatattattaaaatgagttatgatgaaattaagaaaatttgtaatgctcttgaaagtcatggaaaagtgaacgctgttattgatattggtgatttatgggtgacactttatgaagtatccgaaggatttaatattgaagatgaaaacaacatcttagctattgataaaagaagcgatttgtttgatgtgttaaaagtttatgaacagtcaaacggtggaagaaaagctgtattgatttatcaaaaaccacattcatgtggaactgcttcaatcatttcagatattgaagatgaaactgatacttatatgtgtgttttaaaagctggtggtgaccgtcatccggattttatttctattcgtcaaaacaatggagaaatttcattatcaaaatcagaagctgaagctatgattaagtatttaacaactgttacaccttcaatgaaaggataattatgattattaatgaaaactcttggcactataaattattcaaactgtttaacgacgaatggaaacgacctaagacactgtgtgcatacttttggtctattgttatccctacatttttcgtttcttttttcggatgtactatactcgcaggtctaactattatctgtgcagaaatcatacagaaatggcttatttttggtagtttatggactcttattccatcagcatttatacttgccattttgcttattttacttattattggttcatttgttattcctgcacaactgcatgaaaaatataaagattataaatggaaaaaggattatgctttacatgtagaaaatattgatagggcgtataaaggtttacctcctattcaacccaagaaatctattatcgttgaatttttaaaagctcgtaaagctaaagtatgtcctgttattgaatataaggctgaatgatgaaaacagtaatgaaaagctattttggtagtcatctttatggaacttctaccccagaatctgatgtagattttaaagaaatctttgttcctcctgctcgcgatattcttatcggaaatgtcaaagagcacatgagtaaaaacactaacaacacatcatctaaaaacactaaagatgatattgaccatgaactatacagtcttaaatatttctttaaattagcagcagatggtgaaactgtagcgttggatatgcttcacactccacctgaactagtggttaaatctgatttgcctgatgtgtggaagtttattcaagacaaccgttctcgtttttatacgactaacatgaaatcatatttaggatatgtccgcaagcaagcttctaaatacggtgtcaagggttctcgtttggctgcattacgtgatgtattgaaagtagttaatcaaatccctgagcaatgggttgattaccaagaagatggttctattaagcagcgtcgtactaaagttgaagatattaagcatcgtcttccagaaaacgaattctgtgaatgggtgttccataatcatgagaaaacaggcccacaaacgttctacactgtgttgggtcgtaaatatcagacaactctttctcttattgagcttaagcagtcactgaacaaattagatgctgaatacggtgaacgtgctcgtaaggccgaagccaacgaaggcattgactggaaagctctgagccatgcttgtcgtggtggacttcaactattggaaatttacaaaactggtgacttggtttatccactccaagatgctccatttattctcgacgtgaagttgggtaagcatccatttaaaacagttcaagagtttttggaagatgtggtcgatcaagtagaagcagcatctattgaagcttctaagaacggtatgcagcaaaaagtagacatgggtttctgggatgacttccttgagaaggtctatcttgaaaaccatcgaagttattataaatgatagggagccttcgggctcccttttttatttcaaaaatttttcacaaaacggtttacaagcataaagctttatggtactatacaactatcaactgatacggatttggagaataaaatgaaaactgtaactatcaataagggtatctacttcggtaaagaaatctctggaacttttgagctcttaggcgaatggttcccggataatgctccggtagatgcacaaggagatggtaaagtttttgttgaaattgacggtaaacgtcgcggtgtttgggtttacaaatcagacatttcatatgatggtgtaaaagttgaagaagttaaagaatcgtatgaagatatgaaaacccgcattaataaaagatttaatgttatgggaatgatgacgaatggtattattaacggaaacattcgttcattaattatttctggtgcggcgggtattggtaaaacgtattctttagataaagctttaaataaagcaaatgatattggatacattgaatataaaagcattaatggtaaaatctctggtattggtctttatgaacagctttggaataatcgtgaagagaattctgtccttttgattgatgatgtggatgttttctctgatatggacattcttaatcttctgaaagctgctctggacactggagagacccgtaaagtctgctggagcactgcatcttcttacttagaagaaaaaggaattgagcgtgagtttgaatttaaaggaacgattgtttttatcacaaacgttgacattgaccgcgaattagaccgtggtactaaacttgctccacatttacaagcattagtgtcccgctcagtttatttggatttgggtgttcacactaatgaagaaattatggtcagggttgaagatgttattctttcaactgacatgatgcaaaagcgcggtctttctgatgaagaaacttataaagcattatcatggatgaaagtcaatgttaatcgtttacgcaatgtttcactgcgtactgctctttatcttgctgactttattatgaccgacaaaaacggttgggaagaaatcgctgaaacggtattattaaaatgaaaaaatattttactatctataaaactgaaatattaaatttaaagacaggtattaagaaatactattatggtcgacatataactaataacatcaatgattcgtatgttggtagtggaaattatatagaacgtgtaaaagtgtcaaatgaccacatattatcaaaaacggttttagaagtttttgataattatgataaaatggtacaagctgaaatattatatatcatggcaggtaaattgaaatatggaaaaactgtgttaacctgtcatacgggggtgaaggaggaaaggttgcagatgaaactaggataaaatgcaaaaagcattctataaatttatggaaaaatcctaatcatatacagcatatgaagaaaaagatgaaagaggtcacatccacgccggaagctaaattaaacacttcaataaaaacttcgcaacgatataaggacccagtttttgttagaaaagttagtaatattattaaaaagaaactaaacaatcctgcagtaaaagaaaagcatattaatggaattaaagctgcaaagcgttctcatttaactatatggcagtatgaagatgaattattcgagctttggaaagaaactaaatatggatggagaaaatttacaaaggaagctataaaacacggatatccaaatgaaccgtatcgtagtatggtatcaacttttcagaaaagacatttagaagaatcctaagacaaaaaggcagttcagaaatagattatatggactgccatcaaaaagatgactagaattaaactggtaaatggaggtaatgatgttatactcaaaggctcgtgaaatttacgaaactaaaattaaagaagcagtatttcagttcgcaacaacaatgcgatggacaaatgattgggagtattcaaaaaatcataagaagcctttggtgacaagaaaggctcatatgttagtgttaatagaccgtgagcagattaaagcccgagaagccctccagaatcataaaaaggctgcctttgaatggtttatggataacactgctcctgagactaagaaagcagtgagcgcttggttcagtggaaaaaattgtgaaagaagtttcttttagttgtttacaagaccgttcctctgtggtactatacaactatcaactacggaggaacagaaaatgaacgctaaagatattttcaacctggtaaattataacgatggtaaatttaaatctgaagctcaaagcaagttctttaatgatgtctcaatcggtggtgaaatcactgttgatggaggacaaatttacaaatcccgttggaattggatcgttattatcgatgagattggtattgtagaaatttacaagaatacgaataaaaatcgtacattacactggtctcgtgatactaacgaacagtacaaaaaggataaagcatctaaattatctcgtgtaactcaagaagatattgagttcatcaagaaagatattttgatgtatgataacttaattgctgaagagcaagctgttattgataaatttgacgagattaaagcttctcgtgaaattcctgattttatgaaagaatcagtaaatgaacgatacactctcatttcagagcgtattgaaacttacaaaaagcaaagagctgaacgccagaatactcttcggaagtttgaagaacggttaaagacggtactcgcataaccgctttataccaaggatggtataatggttctaagcccttttaattgagattattatgaaacagttgataattaaaagattgaatttattgatatgttgtttatgtatagtaattgcatatggttattatgctattaatgattatatgcattataaagattatgatgttactgtagttaataccattacaggaactcaaggaaaggggtctaatttatcgtttattgccgtatatgaactcaaagacggttatagatttagtgaatatatttccccagagatgtattcttcaatagaaaaaggtgataatattactgtaagtttacgtcctttcgacgtaaaacagacattgtttgataatatcgtttggttctttggaatggtattagttcaatctgtatgcggtgcttatatagtcttttccatcatattctgcgtatttagtaaaattgaaaattgagtgaggaaaatatgtcagtagtaattaataatgtcaatgcagtaattaaatctttagttaataaaaaattgaatgaatggactgtacttcgtcgtggagagccagataaattttttcatagatttaacccaactttggatttgaatgttattgacagagatgttcatgctgaaattttagataaatttaaagttgatattggatttggattagaaaaacatttacagcgaacaaacgggtctggaatgggtttatctaatcgcattatgaaagcccttaataaaattggagcattgtctcgtattaatgcaagtgaaattcttcgcaattataataaaggatatgacctttatggtcgactaatgccaaaattatcattcgatcaaatgattgaggatttgtgggaaaatcaacgacgattattagcattaggcgctcgattagctaaaggtctagataaacaaatgatttttaagaccaataatacagaagaccttaaatgctttaaatttagcactcgtggagatgattattacgtcagagctcgctctactgattatgtcaatatgggtcatcatctctgtttagcttttgaagttttaaaagaatctggaacattagaatattcatctggtgcaaaatgtccaattggttcaagttgtattttaatttatcgcccggatgaatccagttcaactaaattgcctacaaaacctgtaccagttcgtagtaacgaaaaacattctgagcaaattgattattttaataagcaaattgaagagctgaatatttctattcaacaatatgacgatgaaatctttagactatctggattgagtagtaaagctaaatctgagcgtgaaaagttaattaaaattgttgatttacttaaatcttaaggaacactatgaaaactcgttctcaaattgaagatatggttcgtaatgccagctatactcgtgatgttatgacatttttgtgccaaaataatttagaccctgatagagttaatagttccattcactttaagtatatgaatagcagtgaatggttacgtcattttagtaaagcgggatatattacacaaatgactgctcgtgagcagctcaccgatttctgtaaaactattgattataaaaatcctctatttgttcaaggtgttggtcagagtaaggttgatttatcatctggatttttcaatcccaatcattatcgtattgaatggagatttattgctctattccgtaaacagttaaagcaaattttgtcgacagctagtcgattaaaaggttctgacattaacttaaagaacctgaaatttgatggttatactcttcagatggaagtaagaccattaaaagaaaataatagaactgcacgaattagctttaaacctaatacaaaaaattctctttcaatttgtgaatgccttaaatcacagttgacagaagcatttaagtatatggatgttgttgccgctgttcagtctaagattttacctcgttttgaacgatttaagttagatacaacatcatatgaacttgatatgatcgtttcatttaaatacgattttttgagaaaggacgaagtcccgcaagagaaaaagcaggaagtacaagataacttaaatttatccaattacttatcaaacgatcctaaattttggatgtatagttcaggtaataatgatgcatggaaattcaataaagtgaattttcttcctgttgaaaatccgagtcttaaacctgttgaaaaatggcacgcggatgcgattgagaagtctatcaaggcagtagatgatgaactcgttaaagcgaccaatgaagtgctagaagctgaaaaggcgttagaacaagctcagtcaagggttcaaaatctgacaaagcaacgttctaaactgaacaatgcattaaatgcactgaactagtttactttgccacaaggatgtggtataatgttcttactttctactgaggagattaatatgactcgtaacgaatatatcaaatcattcaatagggttattgatgataaagttaaacccatgtctagtcagaatgctgttctttctattatcaatcaatggctcagcagtattgatgcaagtattgtttcttccagtaaatttattcatgaagttcgtgaaatttctagccgtgtagataaagatgatattaagataatctttaaagggtctcgtcttctttcatatttagttaatcgggatattcttggtaattttgggaaagaaattaaacgaactaaaggtgtaataggatacaattggttcggtgatgttaattcttcttatcatcttaatatcaaagaagaccctaagaatatttttactcgtcgttggattagtaatttcagactttttaaaggacaaattctaaaatcagcttctaaattatgttatggcgattatcgtcaaattcatcctttggcttctgatatgattatcgtaaaagaatatgagcttgataaaaataaagtagctatttttgtgaattatgaattttttacaccagaagctaaccaaaagaatattaataaatttttctcaattgctagcactataactcatcaattagagacggctttggattgtatggaaactgtaaaaagtattcatacatatcctttcaaaaatattggtggttgggaaggatataaaattgtaattagtcttcgtgaagtgaaatgtgcttattcactaactgataaagaaatttaccaacaaaaatgcgaagaaattgtgaatactcctaaagaagaaactacccttgaggaactaatggataatcttgacgattcacttgaaccggtagaaattcgtccagaagttgttgcactagaaaaagcttataaagaagttctagaaatttctaataaagcgcagaaagaatatgagaaggctaaaaagatttgggaagaatctgttaatcgcttggatcgtcttgaacaagctttacagttaattaagtaatttaaagccaaggatggctcggagtataaatcattaacaagtgagaagaacatgaaaactcgtaaacattatattgattattttgacagtcttattactaaacatcgtaattatcagatcggacacagagcagtaatcaataatattcttcgtgatttccttcaatatgttggccaggaaaatcatatctgtaaagatactcaaaacgcttattctcattcgttaggcaatttgcttgaatggtttaaacgttctcgtatgttatcttctactatagctcgtgataacattaaaaactttatgaagccgagcttcattaaatctgagacatctataaccgatttggttcaatttactattgttaacgatgttaaaaagacccatttggctgattggttatctaccattcctgaaactaaatttgctgataaatttgcttgtcaattcaatacccaagtgaatatgctttttaagcatgctcgcaaactgtttaccgcgggcgatgatcgtacaaatacagttcatgttaaggactgggttattgctgatgaagtaacacacaagccaggtggttcatcagtattaattaatatccaggttccttattattactcacgtaatctcggcaccatgacagctaaagaaattaataagcacaacaaaactattcgttcgttgtcttataaactccgcatgatgttaaaaattatggacgtagtggaaatgtatgacgaaaccgaagataatggttcaatgttatacagttcacgcattctaattaaactgaagaaccctaatacatataagacagttgtaaaagagcctaaagtagaaaaggttgataatctgggtgaagaacgtgaatatctcaatgcccgtttgactgaagttgaaaaacaaattgaagagcatactaaagttcttaaggctttaaccgccaaagcaaatggtttacgtaatgctattgaggtattgaaatgaaaaagcgtttattagaagacattgcagcttcaagtaattctagtctaattaaaattattatggctggtgaggaagatgatatggaaatgcgtggaaagattcacggctgcgatgatttagattttaaacctccagcatgggatgctattatggctatggttgaacgtcgcgaaagggcttctaaaaacgttcctaattgccctgaatgcggtactgaacaagttcaattgattaactggcgtaaaccagagcttgaatataaatgccgtcaatgtaaacataaattcagtaagcatgctccggaaatggttaaattgcctgactctactgagttctttaaagaacttgtgagtgttcaaccaatgcctaataatattttggattaaaaatgaccaagcgtaaagaatatatggaggctgctgaaaaggcagtccgtgaattagcaatagcttattataatgaacatggtaaatttcctgatagatacagcgtgcttaaatctgctgtaactcgttcatacaaaaatatgctatcagaagtaagtgatattatatacaaacataaagaacaaacgggccaaagtcttgattacgacgagacttttaaacaagtactaggaattaaggaataatatgtttaaagtatatggttatgatagcaacattcataaatgtgtgtattgcgataatgcaaaacgtcttttgaccgtgaagaaacagccgtttgaatttattaacattatgccggaaaaaggtgtttttgatgatgagaaaatcgctgagcttctgactaaactaggtcgtgatactcaaattggattgacaatgccgcaggtattcgctccagatggaagtcatattggtggatttgaccaattgcgggaatactttaaatgatgctcgaaggaaccgattatatccatgactaccgcggaagcgcggtatatgtaggtgatgaggttgcagtttattatgggtatggaactttgatgacagccaaggttattcaaattaaaaataatcgtgctaaactcgaagtttattattctaatggtgaaaagtctatttctaagtggaaatacggcgattgcatggtcaaactggggtaaatatgatttacgatattaatgtatcacgagctccgtcaatggttactattccagccgaagaactagatcgtcttcagaaaattgaagaacttctttgggaaattgaatgtgacctgccatcaggactagaatcctggattgatgatgaagaacttaataaattgcgaaattaattggtgaaaaatgactattgaaaataaattagatgttgatgcagttctgagcgaaatcattgaagaccatgatgcattttcggaaaactatgatttcgatttttccgattatcttaaacctgtcgaaatagaagactgggtgcaagatggtaaatgtcaatatcgtcagtgtgtttactttagtccaaaacataatgtacatgtagcggtaaatgaatctcggtcaggttcttatcattctgactggtattatgcagttccgactgttgaactagtcgagccccgtgaacgtgtagtaactcagatagtacgagaatggattacgctataaaaccttggtgggcggctagatgggaaactgtagagccagagccggaagaaccggtttacactgatgaagaaacagtatataatgaaccaacgataaatgacttaattgatatggagatgggacatgattacagtagataagtggtttagaattaatcgtgctgatgcagggctgtgtaattactggccggaacttagtgcaggtactgtctttaaagttcgtgaacttgcaaaagaatgtgaagatgatatagagcctgatactggaattattgaaattgaactttctgatggaaagattattaacatctacgataagccaattacgtattggtgcttgtggaatactgaatcagtcgaaaatggcgaaattgaagaagttgtagagcgaactaatcaagttgttcagaagcctaaagctgattttcaaggtgaacgtatttcatacgcattagctaaattagctgcacaagaaaataacgatggctatgaaggaaatttgatgcaagctgccgcagaatatattgaatggcttgaaacccaaatttctttttctgaccaaaagattcggcaatataagcgattgaatcaaatgttttacaatacttgaaaataataaatacccttatctatttaaggtaagggttttattatgttattgactggcaaattatataaagaagaaaaacagaaattttatgatgcacaaaatggtaaatgtttaatttgccaacgagaactaaatcctgatgttcaagctaatcatcttgatcatgaccatgaattaaatggaccgaaagcaggaaaggtgcgtggattgctttgtaatctctgcaatgccgcagaaggtcaaatgaaacataaatttaatcgttctggcttaaaggggcaaggtgttgattatcttgaatggttagaaaatttacttacttatttaaaatccgattacacccaaaataatattcaccctaactttgttggagataaatcaaaggaattttctcgtttaggaaaagaggaaatgatggctgagatgcttcaaagaggatttgaatataatgaatctgacactaaaacacaattaatagcttcatttaagaagcagcttagaaagagtttaaaatgacaattgaaaaagaaattgaaggattgattcataaaactaataaagaccttttaaacgagaatgctaataaagattctcgtgtttttccaactcaacgggaccttatggctggtattgtgtctaaacacattgccaaaaatatggtcccgtcttttattatgaaagcgcatgaaagcggaattatccatttccacgatattgattattcccctgctcttccatttactaattgttgtttagtagatttaaaaggaatgcttgagaacggatttaagcttggtaatgcacagattgaaactcctaaatcaattggagttgctactgcaattatggcacaaattactgcacaggttgcttcccatcaatacggcggaacgacttttgctaatgtagataaagtactttctccttatgttaaacgcacctatgcaaaacatattgaggatgcagaaaaatggcaaatcgctgatgcgttgaattatgctcaatctaaaacagaaaaagatgtatacgatgcattccaagcttatgaatatgaagtaaatactctctttagttcaaacggacaaacgccttttgtaacaattacatttggtacaggaactgactggactgaacgaatgattcagaaagcaattctgaaaaatcgtattaaaggtcttggtcgtgatgggataactcctattttccctaaacttgttatgttcgttgaagaaggtgttaatctttataaagacgatccgaactatgatattaaacagcttgctctagagtgcgcaagcaaaaggatgtatcctgatattatttcagctaagaacaataaagctatcactggttcatctattcctgtttctccaatgggttgccgcagtttcttgagtgtatggaaagattcgactggcaatgaaattcttgatggacgtaataatctcggtgttgtaacactgaatcttcctcgtatcgcattagattcttatattggaacacagttcaatgaacagaaatttgttgaattattcaatgaacgaatggatttatgttttgaagcattgatgtgtagaattagttccttaaaaggagttaaagcgactgttgctcctattctttaccaagaaggtgcattcggggttcgtcttaaacctgatgacgatataattgagttatttaaaaacggcagaagttcagtgtctttaggatacattggtattcatgaattgaatattcttgtcggtcgtgatattggacaagaaattttaactaaaatgaatgctcgtcttaagcagtggactgaaagaaccggatttgcttttagtttatattcgactcctgctgaaaacctttgttatcgcttctgtaaactcgataccgaaaaatatggaagtgtaaaggatgttaccgataaaggctggtacactaacagtttccatgtttcagtagaagaaaatattactccgtttgaaaagatttctcgcgaagcgccgtatcatttcattgcgacaggcggtcacatttcttatgttgaacttccagatatgaaaaacaatttaaagggtcttgaggctgtctgggattatgctgcacaacatttagattattttggtgttaatatgccggtagataaatgttttacatgcggaagtacccatgaaatgactcctactgaaaacggatttgtttgttctatctgtggagaaactgatcctaaaaagatgaacacaataagaagaacatgtggttatttgggaaatccgaacgaacgtggatttaatctcggtaaaaataaagaaatcatgcatagggttaaacaccaatgaattatgatagattttatccttgcgattttgtgaatggccctggttgcagggtcgttcttttcgttacaggttgtttgcataaatgtgaagggtgttataataaatcaacatggaatgctagaaatggtattccattcactggtgaaacactagaacaattaattgaatgtttgaataatgattatatagaaggattaactataactgggggtgaccctctttatcctgataacagagacgtgattcactctgtggttcaaactattaaaaatctttatcccaataaaagcatttggttgtggacaggatataagtttgaagatattaaacaactagaaatgcttaaatatgttgatgttattattgatgggaagtatgagaaaaatcttccgactaaaaagctgtggcgaggatcagataatcagcgactttggtcaaataccgatggggtgtggacacatgattaaattgaattacattatggatactataaatgatatgatttttcattttggtccagaattttattcgcaatatagtttagtgcttatcaatgcttggttaatcaattaagggtaaaatatgtataaatttcgtaaaggtttagctgattttcttacaactgtaacgttctttctgtttatggcagttggagctattttccttattccttttattgttgtatttttcgtgattagtttaatttctccagaaaagggcttatcttctagtgagttcaatgagcgtctggataaaattactaacaagctgaatgctgttcttgataaaaaggcttaattatgattagttttgagcggtatgtagtagagagttggaaggggtttgatatgtttggtaatgactattatttttacgaatgtagtctaaatcctagtttttgggctggacgtgaacgagatctcgaagaaattaatgctcgtgccgaattgttaggtgaactgcctacggtttattttacctttgatgcatctggatttgttatccaggtttattttcctgaagaaaactctggtgaggattctgttaatcctccttattgggcttaccaaggaattatttctcgtggaacaaaactcgaacttaaagaataagattgaagtctatggaattccagatgaagtaggtcgttgtcctggatgtcaatcagttacaaaacttctaaaggagctcaatgctccttttactttctataaagttcttactaataatggtaagattgagtacgatcgcccattgattgtatctcttgctaaacgcgctggattcacatctcttaatattcggtatcctgtcattttcattaatgattctagacaaaagaacattaaacacttcaaagaaactctcatttcacttggatatgatagagatatcatagaagactaagacgggccctctgggcctttctttcttacattctgtatattaccattctaagctatcgttcccttcttatcattccctaaaataattttcacaaagttgtttacaacaagttcaaaccgtggtattattaacatatgaattacctttgaggaattgatatgattgataaagagattaaaattgaccagtactatcgtgttaacggacatatcgttattgttacagcagtcgatgatttagatgttcattatactttgcttaaatggaacaaacgtttgatttgtgatcgtgctgtatttagttcagttgctaaggaaattaaactccatgggtaaaacgtatcgtcgtaaagacttaaaagtgcgtgattatgactatttcggaaagcgtaaagctcctgatggtgtaagtcataaagatatggttgaaaacatttttcgctcagataaatggcgtagaatgaaaggtattgattcagaagttaaagatgagctaaatcgccaattacgtggtgaagtaagaaagttgaaaaaatcagtttacattgacgatgattttgattataatacttctcaacgagttgctaaacgtaaatcaaacgagtgttatcgttatagctgaggaaaatatgaatatcaaacgaatgctttttaagcaggggttatacactttaaatgttactccaaaaggcgatacaactaggtggtcagtaaatgactggattaaatttattgatgaaaatggtaattgggaaatttaaatgaatcctgaatctaaattatcgcagcgaattgccgaagctcgtcgtcagttctttgaaaatatgatagcacaaggtattgatgacgaagtttttctaaactggttttggaatcacaaatattcaagttgctcaggagcaatattaatatcagccgcaatgatgtacgaaggctggaagggtgccaaaaagtttagctaagggcttcggccctttttggataataaaattttaatgcaattgaggataatgtatgactattcaaattaaaaacgccatcaattcttacgcatatgataaagtagtttctttgctagaaaaaggcgatattgtaactcctcaaattttggataaatgggaaaaagagcttcatcaaacgatgaaacagaatgatcagaagattggacgcaatactgtccgtgaattattggttcaatatatcttgtcagaatttgatgttaaagcttttggtgtagaatctaaagcttatcaaaagcatgaaatttccgataaaactattcgtcgcatgaaaaatcaacgcaagaaaaaatttgcagacctgaaaattactaaggtataattatgaacgaagctcttattaacgatttacgtcttgctggatatgaagtaaatacaaatggcattggtttaactcaaattgaaggaaacggattcatccttgagtatgaatttagccaatggtggttatatgccaattatggcgaattgattgaatatgttgaccaatttgattcactagatgcagctcttggagcggctaagctgatgaatgtatgaaatttattaatatttctattactattgaaaattatggcattttctatgttgatcaatacatgaaaatttcatttttcccaaataagactggtgttggatattgggaaagccatgtttctgaattaaatgaaagcgaatatgctagtacacatgaaaagtttttagactttttatatcatgctgatctaactagtcattatatagatattcatgaatttaaaaagatgatggagaaagtgttccaagcatactgtttacttagataactgatatcctctatgctttaagatagatcttcaaatattatgatataatagatctatgaattgagctaagaggtgaaaatgtcagaaactaagcctaaatataattacgtaaacaataaagagcttttacaagctattattgattggaaaacagaattagcaaataataaagacccaaataaagtagttcgtcagaatgatactatcggattagccattatgcttattgcagaaggcttatctaaacgtttcaacttttcaggatacacccagtcttggaaacaagaaatgattgcagatggtatagaagcttctattaaggggcttcacaattttgatgaaacgaaatataaaaacccacatgcatatataactcaagcttgttttaatgcattcgtccagcgtattaaaaaagaacgtaaggaagttgcaaagaaatatagttacttcgttcacaatgtctatgacagccgtgacgacgatatggttgcgttagtagatgaaacttttattcaagacatctacgataaaatgacgcattacgaagaatcaacctatagaacaccgggggctgaaaagaaaagtgttgtagacgattctcctagtttggattttttatatgaggctaacgattaacctctccggattcttggaagaaatacctgaagttgaagctattccctatttacttaaaatgtatctcagggaagttttagctcttgacattgatattgatccagaaaatccgtatgataccgcttttaaatctaatggtgtagaattaaactatcggtatcatttaacagatgatgatttttattttatattagagaaataatatgactgataaacccgaaattaatgatgaagtggaaaagcttatttcttctattgaagaaaagaaccgtcttgaagcagaaagaaaagcaaataagttattgtctaaaaacaaacgcgaactgaatcgtctttataagcatgctcagatcgcagctgaaaacaataattttgctcaatacgaatatgctatcaagaaaagtcgggatattctaaaacagccatataacgatgaactcatcagtattctttggaagactactagatcgcagattgaggatatgattgatgcttacacacgtaaaattcaagcgtcttaaaattaatgcaggatttactgaatctttgaatggtcatctttgcgtgaaaatttctgaaaaagaataccacgatagttcaattaaagaagttaatcctcctattgtaagagcagaccctaatatgaaagtgtgggttgattcctatcaagtcaaaaaatggtggcagctgtgaaagatgaacgcccagacttgtgaaatagattataataaaattcgttcctctaaagtagaaatgatgagacgctttaaagaatctcatgataaagctaaagcagaaggaactataacatataagcgtataaaatttagaagctctaatgagcctctgtatggtgtattatgcggataggagcttcggctcctatattgctttataaatttttggtaaaataaaccaaaacaatgaggatattaaatgaaagtatgcatttttatggctcgaggtcttgaaggttgtggcgtaactaaattttctcttgagcaacgtgattggtttattaaaaatggtcatgaagtaactttggtttatgctaaagataaatcatttactcgtaattgcgcgcatgattataagtcattttcaattccggttttattggcaaaagaatacgataaaacacttaagctggtaaatgattgtgatattctaattatcaattcagttcctgctacttcagtcgaagaagacactattaataactataaaaaaattattgataacattaaaccttctgttcgtgttgtagtttatcaacatgaccattcttctctttctttgcgccgaaatttgggattagaagaaactgttcgtcgagctgatgttatttttagccattctgataatggtgattttaataaagttttgatgaaagaatggtatccagaaaccgtttctctgtttgatgatattgaagaagcaccgaccgtatacaactttcagcctcctatggatattgcgaaggttcgatcaacctactggaaagatgtttctgaaattaacatgaatatcaaccgttggattggtcgcacgactacatggaaaggtttttatcagatgtttgattttcatgaaaaacatcttaaacctgcaggactaagtactattatggaaggtctggaacgttctccagcgttcattcctattaaagaaaaaggaattccatacgagtattatcgtcttcatcaagtagaccaaattaaaattgctcctaatttgccaacgcaaattcttgaccgttatgtaaatagcgaaatgcttgaacgtatgagtaaatccggttttggttatcagttgagtaagttggataaaaaatatctacaacgttctttagaatatactcatctcgagcttggtgcatgtggaacaattccggtattttggaaatctactggcgaaaatttaaaattccgtgttgataatactccgttaatttctcatgatagcggtatcgtttggtttgatgaaaatgacatggaatcaacatttgaacgtattaaagaactgtcatctgaccgagctctttatgaccgcgaacgcgaaaaagcttatgaatttttgtatcagcatcaagattcaaacttctgctttaaagaacagtttgacattattacaaaataaagggcttcggccctttagctttatacggagtttgatataatgatatttcttggatatgtgatactttttcttgcattttatctattcactagagcatgttgtgttgggttctttagcacaccagatgggtttatttcaataattttattttgcatttcaatgacggttcttgatatatgaaaaaatatataacttatttaacaatatatagtggagataaacttcctcctttttatattggatctacgtctttagataggcatttagctggatatcatggttcagttttatctaaaaagtataagaaaatttataataaagagctttcagataatcctcacttatttgattcatgcattttagctgaatttgatacgcgtgaagaagctattaaatgcgagttatattatcaacgattgcatgatgcagttaaaaatccattattctttaatatgtcatatgctgctccaaaaggttcatttggaagagataataaaggacaaaatcatccattatatggttctcataattgtaaaggaaatatacattcacatgatccattaacaggaaaatctgcctttttgccttatattccagaaggttatgtaaaaggaaggtcaaataaatttaaagcttcatcacataataaaggtaaaaaatggtataataacggagttaagcaaatgttatgtcatgaacctcctggtcctgaatggacaaaaggaaaaattaaagaactggtttcaaaagcagctaaattaggatggaaaagacaccatgaaaatactaaacttgggtgattggcatttaggcgttaaagctgatgatgagtggattcgcggtattcaaattgatggaattaagcaagcgatagaatattctaagaaaaatggaattactgtatggattcaatacggtgatatttttgatgtgcgaaaagcgatcacgcataaaactatggagtttgcccgtgaaatagttcaaatgcttgatggtgctggaatcacattacatactattgtaggaaatcacgatcttcactacaaaaatgtaatgcatccaaatgcctctactgagcttttggctaaatatcctaatgttaaagtatatgataagcctactacagtagattttgacggatgtttgattgatttaattccttggatgtgtgaagaaaatactggtgaaattcttgaacacatcaaaacttcatctgcttctttttgtgttggtcactgggagctgaatggattttatttttataaaggaatgaaatctcatggtcttgaacctgatttccttaagacttataaagaggtgtggtctggtcacttccatactatctctgaggctgctaacgtcagatatattgggacaccatggacactaactgcaggtgacgagaatgaccctcgtgggttctggatgtttgatacagaaacagaacgaatggaatttattccaaacaacactacctggcatcgtagaattcattatccatttaaaggaaaaattgactataaagattttacaaatctatcagtacgtgttatagtaactgaggtagacaaaaatctgacgaagttcgaatctgaactagaaaaagttgtgcattcattacgagttgtgtcaaaaattgataactctgtcgagtcagatgacagtgaagaagttgaagttcaatctcttcagacgttaatggaagaatacattaatgcaattccagacatcactgattctgaccgtgaagcacttattcaatatgcgaatcagctatatgtagaggcaacacaatgacttttgatgaatttaaaaatgttatgatgagtcagcattttgaatgcgaagtaaaagatgatattgggcataaagaaattattgaatattggtttgaaccgttagaggttgaagataattgtattaaaaaggttacggtatgcactgattgggccgtatcttttaacttcaacattttagataatgatacacctaaatcattacaagatatggctgtatcttgtattaaggatgcatactgtgaagttttcgacatttgacattaatgatgaattcatagcaaatattgattataccgaagaagattctagatatgttggaataatttatatcacatcaaaagtagcacaaggtgttgtttgcatggctgaatttgatgaatactttttagattatgatgatatgatagaatggtctaaaagatacattaaaaggaatcttttgtgaagaattttaaactaaaccgagttaggtatcaaaatataatgtcagtaggtggaaatcctattgacattcaattagatagggttcaaaaaactcttattactggccgaaatggcggtggtaagtctactatgttagaagccatcacatttgggctttttggcaagccatttcgtgatgtaaagaaaggtcaattaataaacagcacaaataagaaagaacttttagttgaactgtggatggaatatgatgagaaaaagtattatatcaaaagaggacaaaaaccaaacgttttcgaaatcaccgttaacggtacacgtcttaatgaatctgccagcagtaaagatttccaagcagaatttgaacagcttatcggaatgtcatatgccagtttcaagcagattgttgtccttggtacagcagggtatacccctttcatgggtttgtcgacccctgcgcgaagaaagcttgtggaagacctgcttgaggtaggaacattagctgaaatggataagcttaataaagcactaatacgtgaattaaattcacaaaaccaagtgcttgatgttaaaaaagatagtattatccaacaaattaaaatatataatgataacgttgaacgccagaaaaaattaacgggtgacaaccttactcgtcttcaaaatatgtatgatgatttggcaaaagaagctagaacgctaaaatcggaaatagaagaagctaatgaaagattagttaatattgttttagatgaagacccgactgatgcatttaataaaatcggtcaagaagcagttttaattaaatcaaaaattgactcgtataataaagtcattaatatgtatcacgaaggtggattatgcccaacctgcttgtcacaattaagttcaggtgataaagttgtttctaaaattaaagataaagtttctgaatgtacgcattcgtttgaacagctttcaacacatcgtgataatttaaaagttcttgttgatgaataccgagataatattaaaacccagcagtcgttggcaaatgatattcgcaataaaaagcaatctctgatcgcagcagtagataaagctaaaaaggttaaagcggctatagaaaaagcatcttctgagtttattgaccatgctgatgaaatagcactgcttcaagaagaacttgataaaattgttaagacaaaaactaatttagtaatggaaaaataccaccgaggaattttgactgatatgctcaaagattctggtattaaaggtgctattattaaaaagtacattccattatttaataagcagattaaccattatcttaaaataatggaagcggattatgtgtttacattagatgaagaatttaatgagacaattaaatcacgtggtcgtgaagattttagttatgcttcattcagtcaaggtgaaaaagcgcgaattgatattgctcttttatttacttggcgtgatattgctgaaaaagtttcaggcgttaaaataaacacactaattcttgatgaagtttttgattcagcgaccgacgttgaaggtgtaaaagctatttcaactattttagatagtttaaaaaatactaatgtttttgttatttcgcatagagaccatgacccgcaagcatacggtcagcatcttcaaatgaagaaagttggtcgatttactgtaatggtttaatttataagagattatgctttaatttattagagtataatctctatggaggaaaaacatggaatattcaactggacagcatctattaactattcctgaaataaaacgatatattctgagaaataatttttctaatgaagagcatatagttactgaatctatgcttaggaatgcatttaaagcagaatatacaaaaataatgtccaatagaaatgaagcttggactgttactgattattatgactaaaggtgtattatgactaaaattactgtgaattatactgttgatgtaaaagatattcagccaaaacacgtgcgttctgactcaaatccgcaaaaccaaaataaaattcgtcgcgcatgggttttgtctctttctgataacgcaatggaagttattcagaacaaaattaaatctgcacctgctcgtcatgcatattatgaagctatcgatcgtgaagtaagtaataaatggattgaactaatgcgcaaacatacgacagaatctctaaatgctggtgctaaatttattatgacttcatgcggtgaacgccttgaagatgattattgcggtaatgcagatgaacgtctaattgttgctgctcaaattgttgcggaaacaattgctgctgattttaatcgttaattgctttattaaattagttataaaattaaatctcatttgaattgaaggaaattacatgaaactgtctaaagatactactgctctgcttaaaaatttcgctactattaactctggtattatgcttaaatccggtcaatttattatgactcgtgcagttaatggtacaacttatgcggaagcaaatatttctgacactattgattttgatgtagcaatttacgatttgaatggttttctcggtattctgtctttagttaatgatgatgcagaaatttcccagtcagaagatggaaatattaaaattgctgatgcccgttcaacaattttttggccagcagccgatccgagtacagtagttgctcctaataaaccaattccattcccagtagcatctgttgttactgaaattaaagctgaagaccttcaacagctgttgcgtgtatctcgtggtctgcagattgatacaattgctatcacggtaaaagaaggtaaaatcgtaattaacggttttaataaagtagaagattctgctctgacccgtgttaaatattctttgactcttggtgattatgatggtgaaaatacatttaatttcattatcaatatggcaaatatgaaaatgcaaccaggaaattataaacttctgctctgggcaaaaggtaaacaaggtgctgctaaatttgaaggtgaacacgcgaattatgtagtagctcttgaagctgattctacccacgatttttaatagagggcttcggccctttataatttacactaaaacttgaatgaggaaattatgattaccgtaaatgaaaaagaacacattcttgaacagaaatatcgtccatctactatcgatgaatgtattcttcccgcctttgataaagaaacctttaaatctattacaagtaaaggtaagattccacatattattcttcattctccttctccaggaacaggtaaaacaactgtagcaaaagcattgtgtcatgatgtaaatgctgatatgatgtttgtgaatggatcagattgtaaaattgatttcgttcgcggtcctttgactaattttgccagcgccgcttcatttgacggtcgtcaaaaagtaatcgttattgatgaatttgaccgttcaggattagcagagtctcagcgacatcttcgttcctttatggaagcttatagttcgaactgtagtattattattactgctaacaatattgatggtattattaaaccgcttcagtcacgctgccgagttatcacattcggtcaaccgaccgatgaagataaaattgaaatgatgaagcagatgattcgtcgattgactgaaatctgtaagcatgaaggaattgctatagctgatatgaaagttgtagcagctttggttaaaaagaattttcctgattttcgtaaaactattggcgagctcgatagttattcatctaaaggtgttttggatgctggtattttatcactggttactaacgatcgtggtgctattgatgatgttcttgagtctctcaaaaataaagatgttaaacaactcagagctttagcaccaaaatatgcagccgattattcgtggttcgtgggtaaacttgccgaagaaatctattcacgtgtaactccacagagtattattcgtatgtacgaaattgtcggcgaaaataatcagtatcatggtattgcagctaatactgaattgcatctagtttatcttttcattcagttagcatgtgaaatgcagtggaagtgatatgagcttatttgaagatgatattcaattaaacgagcatcaagttgcttggtattcaaaagattggacagctgtccaatccgctgctgattcttttaaagaaaaagctgaaaatgaattttttgaaataattggagctattaataataaaactaaatgctctattgctcaaaaagattattcaaaattcatggttgaaaatgcattatcacaatttccagagtgcatgccagctgtatatgctatgaatttaattggatcaggcttaagcgatgaagctcattttaattatctaatggctgcagttcctcgtggtaaaagatatggtaaatgggcaaaactagttgaagattccaccgaagtattgattattaagttacttgctaagcggtatcaagttaacacaaatgatgcaattaactataaatcaattcttactaaaaatggaaaacttcctttagtattaaaagaactaaaaggtttagtcacggatgattttttgaaagaagtgactaagaacgtaaaagaacagaaacaactcaaaaaactagcattggaatggtaaaatgattgaaattactcttaaaaaacctgaagattttctgaaagtaaaagaaactttgactcgtatgggaattgctaataataaagataaagttctgtatcagtcctgtcatattcttcagaaaaaaggactatactatatcgttcattttaaagaaatgcttcgtatggatggtcgccaagttgaaatgacagaagaagatgaagttcgtcgtgattcgattgcatggctgttagaagattggggactaattgaaatcgttcctggacaaagaacttttatgaaagatttaactaataacttccgagttatttcttttaaacaaaagcacgaatggaaactcgttcctaaatatacgattggtaattaagcaaggggcttcggccccttatttggagtataatatatcaagagcctaataactcgggctataaactaaggaatatctatgaaagaattttatatctctatcgaaacagtcggaaataacattgttgaacgttatattgatgaaaacggaaaggaacgtactcgtgaagtagaatatcttccaactatgtttaggcattgtaaggaagagtcaaagtacaaagacatctatggtaaaaactgcgctcctcaaaaatttccatcaatgaaagatgctcgagattggatgaaacgaatggaagacatcggtctcgaagctctcggtatgaacgattttaaactcgcttatatcagtgatacatacggttcagaaattgtttatgaccgaaaatttgttcgtgtagctaactgtgacattgaggttactggtgataaatttcctgacccaatgaaagcagaatatgaaattgatgctatcactcattacgattcaattgatgatcgtttttatgttttcgaccttttgaattcaatgtatggttcagtatcaaaatgggatgcaaagttagctgctaagcttgactgtgaaggtggtgatgaagttcctcaagaaattcttgaccgagtaatttatatgccattcgataatgagcgtgatatgctcatggaatatatcaatctctgggaacagaaacgacctgccatttttactggttggaatattgagggatttgacgttccgtatatcatgaatcgcgttaaaatggttctcggtgaacgtagtatgaaacgcttctctccaatcggtcgagtaaaatctaaacttatccaaaatatgtacggtagcaaagaaatttattctattgatggcgtatctattcttgattatttagatttgtataagaaattcgcatttactaatttgccgtcattctctttggaatcagttgctcagcatgaaaccaaaaaaggtaaattaccatacgacggtcctattaataaacttcgtgagactaatcatcaacgatacattagttataacatcattgacgtagaatcagttcaagcaattgataaaattcgcgggtttatcgatctagttttaagtatgtcttattacgctaaaatgcctttttctggtgtaatgagtcctattaaaacttgggatgctattatttttaactcattgaaaggtgaacataaggttattcctcaacaaggttcgcacgttaaacaaagttttccgggtgcatttgtatttgaacctaaaccaattgctcgtcgatatattatgagttttgacttgacgtctctatatcctagcattattcgtcaggttaacattagtcctgagactattcgtggacaatttaaagttcatccaattcatgaatatatcgcaggaacagctcctaaaccgagtgatgaatattcttgttctccgaatggatggatgtatgataagcatcaagaaggtatcattccaaaggaaatcgctaaagtatttttccagcgtaaagactggaaaaagaaaatgttcgctgaagaaatgaatgccgaagctattaaaaagattattatgaaaggcgcagggtcttgttcaactaaaccggaagttgaacgatatgttaagttcagtgatgatttcttaaatgaactatcgaattatactgaatctgttctcaatagtttgattgaagaatgtgaaaaagcggctacacttgctaatacaaatcagctgaaccgtaaaattcttattaacagcctttacggtgctcttggtaatattcatttccgttactatgatttgcgaaatgctactgctatcacaatttttggtcaagttggtattcagtggattgctcgtaaaattaatgagtatctgaataaagtgtgcggaactaatggtgaagatttcatcgcagcaggtgatactgattcggtatatgtttgtgtagataaagttattgaaaaagttggtcttgaccgattcaaagagcagaacgatttagttgaattcatgaatcagttcggtaagaaaaagatggaacctatgattgatgttgcatatcgtgagttatgtgattatatgaataaccgcgagcatctgatgcatatggaccgtgaagctatttcttgccctccacttggttcaaagggcgttggtggattttggaaagcgaaaaagcgttatgctctgaacgtttatgatatggaagataagcgatttgctgaaccgcatctaaaaatcatgggtatggaaactcagcaaagttcaacgcctaaagcggttcaagaagctcttgaagaaagtattcgtcgtattcttcaagaaggcgaagagtctgttcaagaatattataagaactttgagaaagaatatcgtcaacttgactataaagttattgctgaagtaaaaactgcgaacgatatagcgaaatatgatgataaaggttggccaggatttaaatgtccattccatattcgtggtgtgttaacttatcgtcgagctgttagtggtctgggtgtagctccaattttggatggaaataaagtaatggttcttccattgcgtgaaggaaacccatttggtgacaaatgcattgcttggccgtcgggcacagaacttccaaaagaaattcgttctgacgtactgtcttggattgactactcaactttgttccaaaaatcgtttgttaaaccgcttgcgggtatgtgtgaatcggctggtatggactatgaggaaaaagcttcgttagacttcctgtttggctgatagaataaatctagggacctccgggtccctttttcatacaagtaatataaatctatacttatgaaaaacagatgattctggacatttagaattccctaaaaaattttcacaaaactgtttacaagactgttcctctgcggtactatacaactatcaactactgatacagatttggagaataaaatgaaaattgttatgggatgcttagcagtttgtttagttgcattagcagcagttccatttgttagtgttgaaaatgatactcaacctatgattgaatctagcaccgttattcacactaatggtaaaatatcagttaaaattgatgataatcttcatgtgaatacaaatggatctttaggtgtacaaataggtaatacctgcataagcactactggagtaattactacttgcatttgaggaaattattatgaaaatcgctattttggttattgcattaggtcttactggttgtgtagctcaaggaccggtagtaaatcagtctgatgtaggaaaaattgtaaattgttcaagtaaattttataatcctaatgtcaagtgttataaagaagctccaaagcaaacagtagaacaaatgcaggcgaattttgacgaagctattcgtccagatgaatctgctcaagcatatcgtaattcggatgtaattacacgcgaagaaaaaattgaaaactactgcgccgagctttgggctaattgggctaataattaccaatggcgtaccggtaaaaatgctccgatggagtatgtagtgaattcttataattcatgcgtaaaaaatttaactaagtgaggaaaacatggaaactttagtagcaggttcaatttttatggttttagtttcaggtgtgttggctattattatatacatgcttccatggttcatcgccttgatgcgtgggtcaaaatcgacagtaggaatctttttcacgtctttactgtttaactggtcaattattggttggtttattacatttatttggtcaattgcgggtgaaactaaaaagtctgcacagccaaatcaggtaattatcatcagagagaaggaatgaaaagcaaaattatagcagtgttgcttttaatcttgatgattataataagtatatactatagtgtaacggttcctcttatgattccaactattattttaggttggagcttattactgttacaagttaaatatgaatgtatcaattgaggtttaaatgattagtgactctatgacagttgaagaaatccgtcttcatttggggcttgcattaaaagaaaaagatttcgtagttgataaaactggtgttaaaactattgaaattattggcgcatcatttgtagcagatgaaccgtttatttttggcgctcttaatgatgaatacattcagcgtgaacttgaatggtataaatctaagagcttgttcgttaaagatattccaggtgaaacaccgaagatttggcaacaagtagcatcttctaaaggcgaaattaactcgaattatggttgggctatttggtcagaagataactatgcccagtatgatatgtgtttagctgaacttggtcaaaatcctgattctcgacgtggtatcatgatttatactcgtccatccatgcaatttgactataataaagatggtatgtcagatttcatgtgtactaatacagtacagtacctgattcgtgataagaaagtaaatgcagtagtaagcatgcgctcaaatgacgtggtcttcggattccgtaatgattatgcatggcaaaaatacgtattagataaattagtatctgatttgaatgccggcgattcaactcgtcagtataaagcaggttctattatatggaatgtcggaagtcttcatgtatattctcgtcatttttacttggttgatcattggtggaatactggcgaaactcatattgtgaaaaaagactataaaggtgtgtggaaatgattcaattcgtaattccaagttatcagcgtgtcggggcagtttctgcccttgatatgtttccgactgattatgaaccgcatattgtagtgcgtgaacatgaagaaaaagcttattatgatgcctatgggtctagagctaaaattataactattcctgatgatgtaaacggaattgccggtactcgtaaagcaattactgatatgtatgcaggtcaacgaatctggatgattgacgatgatactactattcgtatgagttcaatgcgaaaaagagatgaccgtcgttgtgtagataaagtcaatcaattaacccgtgaacagttctatgaattgattcaatacgtcgaagatgccatggattgcgggtattatcacggtcatgctcgtctacctatttttaaaattacttcatcttggggtaattatcgtgaaaattcatatggattcacgaatacatggtatgaccttggaaaacttacgacagaacaaattgggtatggaaaaattgatttgtgcgaagatatgtatgcatttctcaatttaattaatcaaggttatccgcatttggccttgttcaaatatctggttgtatctggaaaagcacaagctcctggcgggtgtagttcaattcgtagtaattctaaacataatagagcgcttgagcaaatcaatagagagtttccagagcaagctcgttggaaaacttctaatattgaaaaacgaaaatcgttgggtgaagaagacgagccattaaaggttcttcgcatgtgtgtttcgcgtaaagaaaaatcagaagcatttcataagtttaatgctattcatccaatagcagttgattaatgcctaaatttattgtgttataattactctatctttaaccagtgaggaaaatataatgatgcctatggaaaaaatgaatgtctattgcagatttaaaatcccgtttgattaaagcttccacttctaaaatgactgctgaactgactacatctaaattctttaatgaaaaggatgtaattcgtacaaaaatcccaatgcttaatattgctatttctggtgcgattgatggcggtatgcagtctggtttaactatttttgcaggtccttctaaacactttaaatcaaatatgtctttgactatggttgcggcatatttgaacaaatatcctgacgcggtttgtctattctatgatagtgaatttggtattactccagcttatttgcgatccatgggagttgacccggaacgagtaattcatacgccgattcagtcagttgaacagctgaaaattgatatggtgaaccagcttgaagctattgagcgcggtgaaaaggttattgtattcatcgactcaatcggtaatatggcttctaagaaagaaacggaagatgccttaaatgaaaaatctgtggcagatatgactcgtgctaaatcactgaagtcattattccgtatcgttactccttactttagcattaaaaatattccatgtgttgcggttaaccatacaattgaaacaattgaaatgtttagtaaaaccgtgatgacaggtggtacaggcgtaatgtattcggctgatacagtattcattatcggtaagcgtcagattaaagatggttctgatcttcaggggtatcaatttgttctaaatgtagaaaaatctcgtaccgttaaagaaaaaagtaaattctttattgatgttaaatttgacggtggtattgatccttattctggattgctagatatggctcttgaattaggatttgtagtaaaacctaagaatggttggtatgctcgtgaatttcttgatgaagaaaccggtgagatgattcgcgaagaaaaatcttggcgtgcaaaagataccaactgcactacattctggggtcctttatttaagcatcaaccattccgagatgctattaagcgtgcttatcagttaggtgctattgatagtaatgaaattgttgaagctgaagttgatgaattgattaactcaaaagttgaaaaatttaaatctccagaaagtaaaagtaaatcagctgctgatttagaaactgacctcgagcagttaagtgatatggaagaatttaatgaataaagatgatttagatttagatctagaaattatcgatgaatccccctcttcggagggggaagaagaaagaaaagaacgtctttttaatgagtctcttaagataattaaatctgctatggaaaatgttatccaggagattgtcattaaactagaagatggttctacacacattgtgtatgtgacaaaattagattgggttgatggaaaagtcgtaatggactttgctgttcttgaccaagaaagaaaagctgagttagctcctcatgtagaaaaatgtattacaatgcaactacaagatgcatttaataaaaggtcaaagaaaaaatttaaattcttttaaggagtaagtgtggtagaaattattctttctcatctcatatttgatcaagcttatttttcaaaagtttggccatatatggattcagaatattttgaaagtggtccagctaaaaatacattcaaattaattaaatctcatgtcaatgagtatcatagcgttccatctattaatgcgttaaatgttgcattagaaaatagttcatttactgaaacagaatattctggtgtaaaaacacttatttcaaaactagctgattctccagaagaccacagttggttaataaaagaaacagaaaaatatgttcagcaaagggcgatgtttaatgctacgtctaaaataatcgaaattcaaactaatgctgagcttcctccggaaaaacgaaataagaaaatgccagatgtaggtgctattcctgacatcatgcgccgagcattatcaatttcatttgatagctacgttggtcatgattggatggatgactacgaagcacgttggctatcttatatgaataaagctcgtaaggttccatttaaactcagaattctgaacaaaattactaaaggcggagctgagactggaacactgaacgttttaatggctggcgttaacgttggtaagtcattaggattgtgttcattggcagcagattatttgcagctcggacataatgttctttacatctccatggaaatggcagaagaagtctgtgctaagcgtattgatgctaatatgcttgatgtttctcttgatgacattgatgatgggcatatttcttacgctgagtataaaggaaaaatggaaaaatggcgtgagaaatctacccttggtcgtttaatcgttaagcaatatcctactggtggagcagacgctaacacattcaggtctctcttgaatgaattgaagctcaaaaagaattttgttccaacaatcattattgtcgactatctgggtatttgtaaatcttgtcgcatccgagtttattcagaaaatagttacacaactgttaaagctatcgcagaggaattgcgtgctctggctgttgaaaccgaaactgttctttggactgcagcacaggtcggtaaacaagcttgggactcttccgatgttaacatgagtgatatcgcagaatctgctggtcttccagcaacagctgattttatgcttgcggtcattgaaaccgaggagctagcagctgctgaacaacaactcattaagcaaatcaaatcacgatatggcgataagaataagtggaataagtttttgatgggtgttcaaaagggaaatcagaaatgggtagaaattgaacaagattctactccaactgaagtgagtgaagtagcaggttcacaacagattcaggctgaacagaatcgctatcaaagaaatgaatccactcgagctcagttagatgctttggcaaatgaattaaaattttagtttacaaactgacaagactatggtatagtagtcttgttggttaaatgaggagattgttatggaattggtaaaggtagtttttatggggtggtttaagaatgaaagcatgtttactaaagaaatcacaatgatgaaagatgacgttcaatgggctactactcaatatgctgaagttaataaagcattagttaaagctttcattgatgacaagaaagtgtgcgaagtggattgccgaggataatatgcatattgttttatttaaacctactccgtataacgtcaggaaaaatactcaattcaaagcacttattgcggatacgtgggaattggtgttagatattccggcagaagaaagccctccatttggtcgagtggaatttattaagtttgctgtacgtcctacgaaacggcagattcgtcaatgcaaaagatactttcgtaagatcgttaagctagagaaacagtttgtaacatgtgattatgcagaagttttaaaatagctgtttacttttgttggaaattgagatactatataaacataaactactgaggagattatcatgaaaaaatttatttttgctacaatttttactttggcttcttgtgctgctcagcctgctatggcaggatatgataaagacttgtgtgaatggtctatgactgcagatcagactgaagttgaaactcaaattgaagcggatattatgaatattgttgagcgcgatcgtcctaaaatgaaagctgaagtgcaaaaacaacttaagtctggtggtgtaatgcagtataattatgttctgtattgcgataaaaactttaataacaaaaatatcgtaaccgaaatcacaggtgaataatatgatttattatatgcacaaaaatccaccgtttaaatacggtaaatttcctgatgcacaatgttataatataacgcctgatgaaaataaccgtggatatcttattgacacaatttttgtaattgttaaagataacgaaatcgttgcctgggcagattttaaaggtacaacacatgatgttaatccggtgcctttcacatattacaatataatggatttggcatatgattataattggtttaatcatgacgcgttggctcatattgaaggtgtaggatttgacatctcttattctagctactcattgtgtcctatgagtcgggctcataaagaacctgaatatttttcatttaggaaacgtgtaaactttaaacgcaacacagagtatgtcggtggaatatttgttaaagataatcgaattactcgcattatttatcctttgggctgtggtcaaaaagatattgatgttgatttagactttactgaaaacatcattaatcatattgcttctgtgtattttgatattgatcacaaaattgtaatatgtggatatgaattaccatcagaaaaacagtctaaagtaaatgtttctattgatgaccaaatttttaatgcttttatgaatagaggttaatatgaaaacatataaagaatttattaaagaagatatggtagctggagattcaggtggtaatcctgaaaatatctctactggaacaacgtcaggcgctgtagtaaataaaggtcctgaacagattcctaaaaagaagaaagaggaatctaaagaaaaagaagagtaaaaatgtcatcaataccttggattgataatgagtttgcgtatcgtgcattagctcatttacctaaattcacacaagtaaataatagttcaacttttaaattgcggtttagatgccctgtttgtggagattcaaaaaccgatcagaataaagctcgtggatggtattatggtgataataatgaaggaaatattcattgttataactgtaactatcatgcaccaatcggaatatatttaaaggagtttgaacccgatttatatcgtgagtatatctttgaaataagaaaagaaaaaggtaaaagtcgtccagtagaaaaacctaaagaacttcctaaacaacctgagaagaaaataattaaatctcttccgtcgtgtgttagattagataaactggcggaagaccatccaattataaaatatgtaaaagctcgttgtattccaaaggataaatggaaatatctttggtttacaaccgaatggcctaaattagttaatagcatagcaccgggaacatacaaaaaggaaatttctgagcctcgtcttgttattccaatttataatgctaatggaaaagctgagtcttttcaaggacgtgcattaaagaaagatgctcctcaaaaatatatcaccattaaagcttatcctgaggcaacaaaaatatatggagtcgaacgagttaaagatggcgatgtatatgttctagaaggacctatagattcactttttattgaaaatggtatagctattacgggtggtcaattagacctagaagttgttccatttaaagataggcgtgtgtgggtcttagataatgaacctcgtcatcctgacactattaagcgaatgactaaattagttgatgcaggagaaagggttatgttttgggataaatctccctggaaatcaaaagatgttaatgatatgattagaaaggaaggtgcaacccctgaacaaattatggaatatatgaaaaataatattgcccaggggttgatggctaaaatgcggctatctaaatatgctaagatttaaattaacccaactaaagcaaatgctaaatctacaaatgtatcaagagtaactactggaatattaataccatgcgcaattgcaacaggtgatagaataaagttccagagtaaaattcctaccatagcagaaatagtaaaagctatacgttttttattaccttttatggcattaacaagtgccattaatttttgtaccatatgtcctccttattgctttatatatttattgtataattaatctactaatccatgaattgaaaggaaaaataacatgccacatttcaacgaatgtagtcaactgatcgaaggcgctgataaagctcaaaatgaatattgggatattctcggtgatgaaaaagacccgctgcaggtaatgcttgacatgcagaaatctctgcaaattcgtttagctaaagataaacctgaatataatcgtcatcctgatgacttggctactgccggtgaagttgttgattggttgcgtaatcagaaagattatattgacgatgaattccgtgaacttctgacttctcttggtggtatgagtaatggtgaaaaagacgcatctgctgtatggaaaccttggaagtcgcagcatggtgaacgtcgtgaaactctgattactgatttgtctccacaagaccaactggaaattaaatttgaaatgattgatattctccatttcgttctgaatatgttccaaggtcttggtcttacagcagaagaaatctttaaactttattatctgaaaaatgccgagaactttgctcgtcaggaccgaggttattaatgtctcgtttaaataaacgtcaactcaaaaagttgaataagaaaagggcgaaggcccttactgctttattacgtaatgaaataactaaagaaatagaccaagagattattgaacaactctggaaagccgctaaattgtaagttataaatacacctgtaattaaacaataaaggagtttattatgggtggtttcgttaacatcaagacctttactcatccagcaggtgaaggcaaagaagttaaaggtatggaagtttctgttccgtttgaaatctattctaacgagcatcgtattgcagattctcattatcagacctttccttctgaaaaggcggcatattctactgtagtttctgatgcagctgactggaaaactaagaatgctgcgatgtttacgcccacagaaataggtggttaataattcaaggactccttcgggagtccttttttcatttaaatagtttactttccaaaatgagtatggtataatagaattatcttatagaggagagcactatgttaaatcgttggattaaaccaaatgaagatttagatattatcatttcacggcatgtaatgaagaaatatgaactgtagccatggtccacagaagttgttgtgcattcatttatgatgtacgcagatggttctgtcgaatttaatgcagagattcgatatgattatgacgataagcaagtcgaattcaaaagaggctttttgtaatgtttatctttaattggtttaaaaatttctttacggattttttctctacgactcctggagaaggtgtagttcctatttcaaatgactaccttcctttaactgtagttgagtatgtttatatgggagatggaacagtagaagcagttactatgacttatgaagaagcccaagaatattataaaaatccttggcgctggtcagtacctactacatcatctaacgtacagaatacacagtctagttctgattcatatgatactaatgttcctgttcatgtatggacgggcgattcatgtggaagttcttgtgattctagttgttcatctacatcttgtgattgaggaaaattatggaagcgattttgtttgaaatgtatattagcagtaatagcatgtcgtttgctaaagatgttccaattactgtagcagtaatgattgataagggttattgtgacccaatgtatctcgtaaaaaaattcgtttcaatgccagttccagaagatgctgaaataaaacttaaaaagattggtattattgaaactgtacaaaatattccatttagagcaattgaagcatttactaaatcagaatacattaatgttaacacagaaaaatataatgatgaacctttatctttttattcatatgattcagcatatagttgggaaatagataaaggaaataagtttataattgtggacgaagatgctttatcatattttattacttctttatggaataatttaaatccaaatttgctaaaaattcatgaatttgacggtgtccatactattgttttaggtaaaataaatgaaagctctcaagaagattcttgaatggttcaatagaccaaactcaatgtatattgatgatggttgggttgaacaagcaaataaagaaatgcagaacgagtcagaagaatggatgaaatcaatgattagtgctgagaaagaaaagaaattagaacgctcaagcgcttaaattgatgagagacatctatggtgataaatc

SEQ ID NO: 3 - DNA, 155887 bp, unidentified
  source 1..155887: mol_type = genomic DNA; note = Bacteriophage
aagcgcaagcccgtgctgaagaagttcgccgtcgtatgctgggcacaaccgttgctgaagcaatcgccgacgttgaaaatccgaaggatgatcaggctttagcgaacgcgaaggcggttctggccaagaaatcttctgaaatgcgtcctgcgtcttctactgaagaacgcaagcgtcgtcataacaaacgtgaagaaatggttgaagccgctcgtgcttctaactatggtccaatcctggcggcggttgaagcaggggttactcctgaagtatatttgagttatgtgaaccctgatatgcgctggttccagttccctgtcactgaattggcagacgaagccaatccgcatgcccgcactaacacctatgttgatctggcacctatcgtttccggcggctggggtttcagtctgtatgtgaatggtaagtcattcaccaagcgtcagaaaatcaaagaaactgacgctgaatctctggtaaaggctatcaacgaatggctacctcaggcgttagccgaagcgaaagctgctgcgtaatttaactcagaagcattcaatagatgaaggggctgctataatagcagctccttttgtttattggagcacaccatgtcgtattcgctcaaagggttgttgaagcgccccgtacatctgtttgtaaaaccacctgctgtcgaaggggaatatccggcacgaggagagttgtattacgttaaaggctccaacggtagcggcaagtccactgtcccttcctatctggcggagaatgatcctcaggcgtatgtcgtgacctacaatggcaagatcatgctcacagtttgcccttcctataacattatctgtattggtaaatatgacaagtctaagtctaaaggtgttgattccctgaaagataccgagcagatgttgttcgccttgtccattgctgatcagccggaatatctgaaatatgacgtgttgtttgaaggaatcatcccttcaactttgctcagctcatggattccccgcctgacgcgcccaccgcgcgaattagtcgtgctcttcatggacaccccgcttgaaacctgtgtttcccgtgtgaaatcacgcaacggcggcgcagacttcaatgaaagcctggttgtcgaaaagtgggagagagttcatgaccaccgccaacggcataaaggcttgttccctaccgttcccgcaggtatgatgaagtccaatggcctgactatagagcaggctgtttttgcgtttctcaatcgtgattttgggagtattgattgatggaaatttttgctttaaacaataatgatatgctgaagaaggctgtgttggccatccgtgaacacggcattgagtcagatcctggcaacgcagagattaacactgacggcactcgtttcattgatggtgtaacaattacagtatcagatccacgtgaccgttggctttccgttgaaggccgcaactcgtcggcaattgctgccatcggtgagacattctgggtgttgtccggacgtaatgatatccgtttcctttcgaaagtcttaccccgcgccgccaacttctctgatgacggtgtaacctggcgaggcgcgtatggcccacgtctgtacaataatggccagttggatagtgtcatcagccgtctgcgtaataatcccaacacccgtcaggcgtatctcaccatctatgatccggcgctggactcagatgaaggtttggccacccatagtgaaactggagaagccaagaccaaggacatgatctgtaatcttgctctattgttcagtataacagaagggcgattgaatatcactgtcattaaccgcagccaagatgtcttgtggggcatgagttcaatcaatttcattgaattctctatcctgcaagaagttctggcgaaagtgctgaatgtcgaagttgggtattacaaactgttttctaacaatcttcattattacaacaatgaagttagtcggaaacaactcgggggtatcactaagaagaccaaaacttcttctgggatgtacaattctttgctggaattctcttcagaaaaagtgaagtcccaaaaagatatttgtaatctattcctcggcgtgttacaacactgtgacatcggcagtgattttggtaaaatcagagaacatctgaaaggatatcaagcagacacgggattgatgatggatatgtcatattgcctgtggtgtcgcctcaatgataccaaaattgacaaacgccgtatcgctgatgacggtcttcgtactgcattactgcattcccctgttgatcagaaatttatgtgggggtatggcgaatgatgtaccagaaccttgaattcttcacggggcgcaagcgctccggcaaggacttctgtttggagtccctgatcaggtttcatcatattcagggtgataaggatattcgtcgtctgtctttctcagatgaactgcgacgtgtcgccaatttcatctacccatggctaccagcggaggtggaggatgctgtgaaggacgttccattcgtacaccctgataaccccaagggcttaaccccacggcagatctggcttcatctgggcagcgatacgggcttgcgttatgtacaaccagacctgttcctgtcattctttaaacgatatcaacttcctttggtggagcagaacccacacgttcattatatcgtgagtgatttgcgtacccctcaggaatatgaatgggcgctgagcactaaatgtcccatcacccgcatttcaaaggcggatcgcggtggtatcatagaggatgacattgaggctttcattgataagatggaagttgattatgaatttgtcaatccgtttgaaggttggcaaccgttcgtcaaattttatagggatcggaaatgatcacagcagagcatattaaaagtctgctcgaacttcaaaaggccaccaacgtggcctattttggggaagagtggaagaatgtatggagtcagaacgccgtcgtcaactccatatatcgtgagtgggcagagtttcttgacgaaacgaccgccgactggaaggtctatggtaacgatatcggcttcgaccgcacgaaggcggtctatgaactggtggacgtggttcacttcatgctgtgtttcatcctcgtcgatcgcaccaaaggagagatcgaggaagaattagaggaaatgtctaaggatagttatgccatgtctatcgttaacggcatagttgcccacggcggggtgacctaccgctttggacagttcatgggcgatccttgcgttcgttccctgatgtatttcctgagcgcgtcttgctcttacgtggggctggacgccgaaacttacatgctggcacacaaacgcaaaaatgatcgcaatcggctgcgtgctgctggcggtgcgaactacgataagtccgcagaaacccctctgaccctggagttctaatgtttacagttgtcgtatcatattatgcggaacatgccccgcgcatggcagagaacttcgctgagtttattaaaccaggcggtcatgtcgttattgcgaccaatttcaagaaagagccagatggtgcggctactgtaacacaacatattacaaccaaagagccggaaacactttttaaacgcattgaagattttgcttttggcggggaagaaattccagagtcattacagcatatctattttgaagatgcgatatattatgttcatgctataccaaaagatgttacaaccgaacaactaatgtatgttttgaatattgcgttaaatttgtcatgtgtcaatagggcacagcataatcgtctgcgcaatatgtatttaacagaacgcgggggttccggaatcagtgatgaaaaactcccgtcggtaatggcaattcaagaagcaattaacatattgtccaagaaagtcttgggcaccgatttattaatcacgatcaacaagcaagaggaaacgaaatggccgattcattgatggctcgcatgctcaagacagcaaagaaattggatccgaatgccgaagtgctgtctaaaaccgatgcgctgaagcctgatattatttgtagcacgggtattcctatcctgaatttggcctggtctggtcgtattgatggtgggctgatatctggtattaagcagttggtgggtgattctcgtacattcaaaaccatgtttggtctggtagatgtcaaggcttacatggacaagtttcccgacgcaatttgtatcttcgcggattcggaaggcggcgcgaatgaaaactattggacgtccatgggtatagacatggatcgtgttttatatctgccgatcgaaaacgttgaaaagacgaagatcaagctgactcaacttctgaatgatgcccaaaaaggcgataaaatcatcgtctttattgactcaatcagccagttgccgtctaccaaagaagtggatgacgccatcgcgggtaaagatacacaagatatgacccgcgctcgtgctctcaatagtttttggcgtgtcatcactccgttggtcactgaaaagaaattcgttttggtgtggatcaactcttattatgatgaaatcgggaaccagtatgccgaaccaaatatcaaaggcggtaagcaaggtttcctatcatctaaccaattgtggttcatcacccgttcccaagttaaggaagacaaagatcttctgggttggcaattcactgttaacatcatgaaaggccgctttgttcgtgaaaaagccaagttccctgtgacggtattgtatgaaggcggtattgaccgttggtctggcatgttggaaattgcacgcatgctgggatatgttgatctggtgagcggttcctggtatcagcgtacagccaaaggcgggtttgatccagagaaagagaagaaatatcgcaagtcagagctgggggatgacttctggtatccactgctggaaaacccagattttgttgacgatgtaaacaatatgttcggtatctctcagagttcggttatgcctgcagatatgctggagcgtctcgatcatgtcatcaaaacaaccgagtaatattgcagggggaggagactcccccgttaattacaaaatcatcgaccctggttctgatcagttagctattatcgaaataacggaaggcaagttccgcggtgttcaatttcgtataggaaaagtgggtgtccatgtagacaatggagaacctcggttatcctttacgacggatatattgaagaagccatggcgtttgttatttgttaatttgaaagaaaatgacttgttcactgtggtgtctggggatatcttagttgatctgatacagcaaaatgctcaggattacaacaaaattttggtggggtgagtctggagtcctatgttaacacagcagtctcttaaagaaatactacattataatgaagacactggttggtttacatggttggtggaccgtggtggtactgctaaagccggaactcgagcgggttctttgaaacctgacggatacagggcaataagaatcggagataagaaatatttagaacatagattagcatttctctacaaaactggttcttggccgaagaaattagttgatcacaaaaaccggataagggacgacaatcgttggttgaacttgagagaatccaataaaagtcagaatgcggtcaacacagatttgtacagccataataccacaggtgcgagaggcgtgtttcgatacaagggtaggtggtgcgtgcaaatcagagttgacgggaaatatagatcgttcgggtattacaatgaattcgaagaagcaaaggaaatggcagacaaggtctacaaagatttgttcggagaattttatcaaggctagtgattgtcctctgtcaaaccaaataatttaggaacacaatatgttactcgaatctgtcgtgctttcccaattaatctacaatgaagaatatcaaagaaagatccagccgtatttgaaagccgattatttcgataacgaaggcgagaagattatatttggtcttatagaccattacacttgcgaatataatgctcgtccttcggttgaagcgctgtctattatgctggaaaagacttcgctcaacgaacacgtatttgatcaggccatttctgctcttgaaaatattaatgacaatacttttcatcaggactggttagtaaaagaaacagaaagttgggcgcgtcagaaagctgttcataacgcaattagacaagccgttaacatatacggcgatgagaaacgcaaagatgagatgaacaacatccctactttgctgcaagaagccctggcgattcagtttgattcatatctcggtcacatctattgggaaatggctgagcaacaatacgaccacatgaactctaatgaagcgaagatccctttcgccgtagagatattcaacaaagcaactcgtggtggtgttggcaagaagacattgaacatcgttacaggcgcaatcaacgcaggtaagacaacaactctgattgatttggctgctggttatgctgagcaggggttgaatgtgttcgtattcaccctcgaagtggccgagaacgtctggcgacaccgccttgatgcccgtatgatgcgcagggacttcgagtccttagagaagctctcacggcacgagtatattgccaccatacaaaaactgagaacccgtcaagacggttccatgaagggtgatattgttatcaaggaatatccttcgggcgcggggcatacaggtttgtatcgtcgcgatattcttgattatgcgactgcaacaggaattacgcctgatgttatcatcattgactacctgggggaatctgcgtcttctcgccttcccgctcacttgatgcaaaacaccaacgtgtattatacatccgtggcgcgggaattccgtgctcttgggtttgagtttgattgccctgtttggacaggcatgcagtttaaccgtgaaaagcaatcagcaactgatggtgatatcagtgacttggcagacgctatcggtatcccgaaggtcgctgactttatcatggcattttatgccccagacgaactggcggctgttaagaaagccagagcatcaatcttgaaaaatcgttatgccaacaagcagaaactcaaatctttcttgttcggtatggaccaagataaacagatattgttcgacttggactggaatgaagtcaaacgagacctgactgatgaagaagcccgttatgttgaaaacgtgcatatcaagcatgacttgaataaaacgggggactcaaatgatgttaaaaaagccgagaccgtcaatagttggaatttcggttaacttcaataatggtggtcatagggtataatctctatgatctgaatattaacccaggagcacatcatgtcggatgttgcatttatagctgacctgattcaggcggcacaacaggtgtgccagtctggacaaaccagtctcagtctctctacagagcagacattggaattttacaagaatcgacggcattggggtaaggctgttaagatacattgcgaaccaggagaattggttcagctcgatcttccatctttcccgttgccagtgcagaccaatactcaggaatattataaagttgaaggtgtcgacattattcctaagtttggtttccacagtatcgtttatttcttcctgcgccgtttggggaccagtattggcccactcggttctcgtcattaatgaggatatgttatgtcagttgaacaaatcgggttttaccaacttccttctgatcccaagctgcgtcaaaagatgatgcagacattggaaaattgccgcgccgcccagatccgtatcaagtctgaacagacatttgttaccgaagctctggccgaattggcaaaagaaacaggcatcaaagccgttgatctgcgcaaagtcgttactgaccgcgctggcggaacatacaccaagaccatcgagaccagcgccaaataccaagatctctatgagtctctgtatcctaatgcagcgcctgatcgcgcagccgatcctcagccataaatcccccagaaagcccaacaaattgttgggctttttatttgaaacctttacttcaataaaacatacggctatcatttaacctactttgaaaagtttcactatatcatgagaggcaaatatgaaagccaagaccttcagtattcctatcgacaacattggtcgtgtcaaggaacgtctggccaaacttgaaagaacggccaagcgcctgaatctcgagttccctcttgtcgaatacagcgaaccatacaaaacccagcatcgcgattctatcactggtgaaaaattctatcggtggtggcaggattgtacactcactggtgaaggcatcgaccgccctgtttcctacggcgggtggagcattattggacaattcaatcaccaatatccaaaagtcatcttgaataaattagcagatgacatccaccctaatttcattcaaagattcgaagccgaaaacgtgtcttggtgtgagcattgtaataaatctgtgcgtcgtcataacacatatgttgttcgtaacgaacaatccggcgctcaaatgttggtcggcagtagctgtatgcaccattacgttcctcatcagaaatcgcttgatgccgtgatgtcttattatatgtcgatacatgaaatgttctcacctgatgaagatgaccctgaaggcatctatcgagtgaacgaacctgattacgttgacactgaaggttatttgcggaattgctttcaggttctgctatctggtatgagtatgaagagcgatgattttggtcgagttcttggtcatatttctagcgggactcgtccggagaaaggttctgacattgagatattctataacaaggcagttaaagcccgtgaagatgctcaatcagagatgtaccatatgatgcttttcatcgccgcgttgtctgagaacaatgatttcaacgttcgtctgaaacgtatgtgtgaacctggatatcacctcgttaaggactccacgacagttcgttggggagcagcaaagtattatgattatatccacaccccccgccaaatgcgttctgtatcaaattgggtggggggagtcggtgaaatgttagaagtgcaagtcaaattcgaagcaaggattttcctgtattcatctgattatggtgacacgtatctgtatactttcaaaaccaaagaaggcaataccattacatggaagacttcttatatggaaaccgaattcctggaaggtgatatgatcattcgtggtcgcgtcaaagaattgaccgaattcaaagatgttaaacaaactcaggtaacaagggcaaaactgaggaaattatgattcttttacaatttgttttgaaagcatttgcattcggtcttatcgcatgcggagtgatcacagctatgctgttaatcatagccctgatcatcccctttaagaggaagaaatgaatgaagcgcgaaattaaggatgtgtatgatcctggatcttgttgcccaggacacgacaaatatccttcagaaaaatataaaaatcgtcgttctgtagctaaacgttccgaggggataaagaaagaacatcgtcacgctcgccgtgttaagcgccttgcgttgaaaatcgatatattctattcacctcaggaggatttttaatggaatgttcatgtggagggcaaggcacaagtgcatacttgccatttaacaccgtcaaggaagccgaagacgctggatatgccgtggacaaggctccctgtgtaattgccattaaggattgtccgtgctgtaagaggcactctcagaacgtttggtatgccccagaagcaaagggcaaaattaatttattgaatatgatgaggaagtaatatgaccacactcgtagaaacattattcccaggatgcacccttgggcgcggcggtagcgaagccaacgggctggcaattgacaccgaaccacagaactgcgttgttatcgacaaattccatgacaatcactatcctctgagccatcgtcgtgatcattatctggtttctgtgggcgaaacattggtctctcttcgtatttgtgaagcaatgcctggttatggttatgacgcatttgcatatatccatagcgtttccaatggggaaatggagaaagaaaccctgactgaacaattcaaaggatggaaagaatctttcacttctgtcaaggaaagaagttagaataattctattgaagtctctggggttataataacaagacgaataaccctggagacacatgatgcttcctctcttagaaataatccaaaatcttcgtgataccaaaggcactaatgctaagaaagccgtgttgaccgaagcgttcaggaccaatccggaactggttgatttccttcaatacgtttatgatccaatgcgttcttattatcgcacccaattcaatctgaatgcattccctcgcatgctttcgcgcggcgttgtcggtagctgggatcaggtttatgacgttcttgatatgatgtcagaacgtcgtattggcgggatgaaggccgaccaagaactggcaaaagccgcaacgaatattcaccctgattatcacaccctcatccagatcattctcgaccgagacattaaagcggggattgctgagaaaggtatcaacgcagcattcaacgcagctggaggcacaggtcgcctgattaacatcctcccatatcatcgttatgacaacatgacgattgatttgttgaagacgatggacttcaagcgcggcgtattcagtcagctgaagtctgacgggatgttcgccaacatcatttgtcgctatggaaaagatccagagattcgttctcgctctggttctttgattgcgggtggttccgttgataacctatcattggttttcaaagatatcatctatgacgctgggattggggaaagcgtttttcatggtgaactccttgttatcgatctgaagacgaatacagtccttcccagagctatcggtaacggtaaacttaacagcgtcatccagactggtgaacctctggaagatcgttatcgagtgatttatcgtgtatgggatgttgttccatacgacaaatggttcaacgcagagcgggttgacaccccgtatgaacgacgcttcgatattatccagcagctgtttgatgaagacgacgggttggttcaggttcaggagacccgtgttgttcactcgtttgaagaggcggttgatcatttcaaggacgcgctggcccgtggtgaagaaggaacgatctgcaaatcggctgatatgccatgggaagacggcacgtcctctgaaggtctgaaattgaagatggaagtggaatgcgaccttgaaattgtgggatttaacgaaggggataagaaaggcaaacatgccaagacttttggttctctgttatgtaagacttccgacggcctattagtcgttggcgtctctgggatctcagatgagctgagactccggatgtgggaaaaccagggcgactacattggcatgatagctgcggtgctctctaacggtgtccaggataaaacagatgacgccatgaagtccttgttcctcccgcgacttgtcgaagtgcgcacagacaagaaagttgccaacacgctggctgaggtctatgctatccagaaagcgtacattgaaaacattctggtgttgctggaggccgcgtgagcaaagaaaccagcacggaactgaaatggcgtgaccttgctcttcagttcgacggtcatcgtatgcagtcaatatgcttcttgaaacaaatcttgagcacattgcccgaaaccgagtttgtggaagcgcgtgaattcctgaaagcaggtccattgcctggagaggaagtattgcgccagcgcctggttgatatgatgaacaaggagcctgacaatgggtgagtacatgagtttctggtgggcgaagttccttgttgaacttcaaatatccggcgcggtgtttttgttactgttcttgatagccatgatacccgttacacggaaagcgatcaagcaatggcgttgcaagcaccccacctatcgtgagaactacgcatgccaagccatctgcacacattgcgggaaggatatgggcttcgtcggtattattcgcaaaatgaagaatcacaaagaggtgtggtgatgaaccagatgtgtgagagggtgaaagaattcatccaccggaaccccaattgctcctacgcgcagatcgctgaaggtgccaatatccccatgagtatggtcagtggttgccttcgcattttgatacgcgatggtcatgttgtgaagtataaccataaagatgctcagggtaaaaccctatctaattcgtatgatgttgtgggttagaattgaattaagggggcgtaacccccctttcaataaatctatggcagtataattccaacaaatcatcacaacggtattgttatgaaacttgttgacaaacaaccaagaaagaataatgactcattacatctcttaaccattatgcgggagttgggagtttctgaattgcaaatgatgaagttgatgaattgctctgacgttgtctataattggacaaacggttatcgtcttatcccaaaatcattccgtcgttttattttggcattggtgtttattcatcgccaaggaaaaacagaagagtttcaacgtttcgttaaatcagaggagagatcacgtggttggtaaaaatccgccgtcatatgtaaaaatcgggatcgctttccggaacgcaatgcttttcattaaagagaaaggcttgatgtcggagttcaatcagttctgttctgaacgtcacagattacagaaacagaaaagcagggagaaacgccatgataactgagtgggagaaaatgcaatatgaacgtgcatttaacgtttattgcatatatatggcgatcaagttgcatttcaccaccaaagattttgattatggtctgtatgggccgatgaacaattacaagtttgaaacattctattctaaacagggcgtggcaaagcaatttgctaaacttgctcgtcggtttgaatcttcccagggtgaagtggtggaaaattatatcatcgccaactttgttaaatcacccaaaacatgggtgacaacattactcacccgtcaggctcaagaaaattataatgaatatcgccgcctgtacgataacttttcatataattttcttgaccattttgaacgttatatgattccggaaataaaagaaagaggtgtgaactttatccagtacatcaaaggaaatgggaatggtcacccaccgttactgacagacattatcatcaaacgttatccgatttggtttttggttggattgaataaagtggtgggatttattcatttatatgatacaatactcaaggaagatatttattggaattctgagtcgttcttattgaaaaagactaattctgtggttccggatgaaaatacagaatacacgaaaggaaaactccgtgagctaatccttgcccacggaatttgattaccaatcccaaagaatacgacctgcgtcagtacggttggacggctccagttgtgaactgttgaagttgtttgtcgtactgactttcttcgagttatcaacattttgttgtacaggcattaccacactggctggcgggttggcgtaggaagatttaacttcctcaatgttgttagccgctctcccctgaaccggatatgccccacccagagtacctcccccgatcccagcatatgcatctttcaattgagaagtgcgatctgttacgttcacagcatccgaagaaaccccagaaggcattggagtcagcgtgctgatatctgcattaggttgtgctggcgattgactgttcggtggtacagaggcattctcaggagccttatcgtcttggcttatcagcatagactgtcttgcttccgccatcttattcctggccgcttcaccacccaacgcatcggggataatactgacaaacttatccacgatatcaaacatcgcattggtgatagcattcaacatgttgaagaaaggtttcttcacattttcagcaaatcctttcttcatgtcttcaacgactttgtttgcagccgccatgccgtcatctaacaggccagttattccacccgttacccaatcaaccaattttatcaattggccttgcaagttatctgcaagtttaccaggtatttcggtcagtgatgtgctcttcccaatacccgcaaacaaatcgttggtgacccaatcaacaatcgagttcaaccaacgcacgggcgcttcggtcattttcagcacttgttcacggaacgcctttccaaacccagcattgttccatccgaatatttcagcaacccaatcagcgagatccccgaaactaccgaccagctctgttatccctgcctgaacccgatcaacgatcgatacttgcgctttaccaaggatttctttagcattaaagaaccctttaccgaattcaaatacagatgttattaacgccagaggtccaactcgaagcatcttggttgctgctttcaggggcgcaagcaacccgccgatcccagcaccaaaccccatgaataatttaatgaagccaccaacaagttttaatggggacatcaggagttttaacgcgcccagtccaaggagtgcccccagcgccgcgcctaacccagaagattgaccatcttgtttaggctcagacggagggatagaaccagcctctggattgttcttgttttctttaccttcttcaccacgacggaatttctcttcttctctatatttttgatcactttcatagatacgtgtcagagtatcattaagatcttcgttcatgtatcccaatttgtcatcaatgctgtcgaggcgggtaacagtttcgttactggcatctttattgaaatctagcgtgtccttatgcgtttgaagcattatgtcagtcaggtttacgatctcccctccgaccaacgccagcttatcctttaatccatcaaggcgttcaagactatcttcgttggccgcgcccaattgatctgatatcatgtcaagatattcaactgacatgtcgtcaaccggaccattcgctgcttcaccaaatttttcgatcttggtggcgatacgcatcagttcttcattgccatcctttgttagactgacggtgtctttcatccaagtgctgtaatcccttgtgaaaatgggactgacgcggacgaacccaccaacaatatcattcaccgatgggaatttaattggcggtactttctgctcctgattagacgcctgtcgtgtcttcagagattgcagttgtttgttgacatcggcgagctgtttattcgtctcggattgggcgcgcaattgcttgcgctccatcatttcttttttgattttatccaaaaccttgacgatgtcttgctgccctggtgtcggttgattgttatctgccatttgtcacctctcagaccttctgggcttgtcctttctctattggcgtcggagttggtgtagaagccacagtttgaccagtctgaggtggtaagaccttctggatcatactatctttggcaatctcggcgacgcattgataatcatttgtcaggcgtgaaatcttgtggcgtatctttgtgacgatataatggcctgttgataacttacttaactcggcaatgttttcattagatttcgggcggttcgacaaatcttcaatataataaacctgcccaacattcaggcggttatcccccacgaccagcaaacgcataaccgttgacgcaaggctgaattctataacgcggcgagcataatctatttgttcagcaccatcgggttgcgcttcaatgtacctaacattctcacgatcaaactgatcagaaaataatgggaatggatcaaggtgagcagtgctgtcaaaccattctgaataaatgcgttgggtcgctgtcgtggattttgtactaaagtcatacacgcgctcgttcactgccaagatatcccgataattcgccatgtactgatcacgcgccagtttcttttcagccttgatgatcgtccgcataaaacgttcggagttgaagttgttgtcttgcatcaacggggcatcttgaggatcacggaagaatttcttttccgcgcttgcctcttgtttctcttcagcagtcatggactgattgccctggttgaacaatgtcgtcatgctcttgaagtgatagcctgtgaagtcttcatagaaaacaaatggcataaacaattcgtcgtatgcgcggctggccatataatcgatggaacgaagtacaggccaaagaggagttgcgaatcgttcttgcacaccatacgacggatctatgtcttcaaatttggttcgagaatttaactgctcaaaaatcttggccgccatttctgaataagaaccgctcagaccgatagacttacgcagcatgctgtcacggtacgcatctgttgtcaccaagtgcagccaaaatgcttttttagatgatgaattggattcatccgcaactctgcccacccgactcacgcgcaatgagagagtagtgtaatcagaagccgctggtgatttgaatgacaccactacttcttcaccgccaagtatcggcattgtgtccaatatatcccaaccttcttttatcaagatattggctgtcagtgaaggagaagcattaccttccaaaccaagatcttggtagacgttgaattcttgaaataaagaagacaggtcatatggtcttggcgtaccaccttctggagtatgtggtaggatcgccatatacttcaaatcaaatgtggtggacggtgttaaaataccgtcttgagattctttattttcgatcatttggtcagctcctgttccagctgattaacaaaggaagaaacataatctggatcgagcacttggatgttacgtttggcttcgtttttgtttattgcatcatcgtgatatgtaatgcccgtcagcccgtaattcgcgatgattgtcgcgtcgtccatagcgccaaggccatatgccaaacggatagcgcggggatctgtttcattcccaaattcgtcaacgtagtattttacgtcccacatcccatcgagtccataacgagcagttagctcttcaatgattctgcgttctggttttggccagtcttctgtgatatccatgataccattgataagacatggtatccagaacaactcgaaggaaccatagacccgttccgcaaaagaccttggggtttccccatcgaaaacagtataaggcaggagaagcccttctatgtcccgaattttcttaacaaccataacccgtcgtgtcaagttttgcaacaggacttggtcattttctttgacaccaattagttgatgccatacgagtggaaatttctcaaaatatttcatggcatcttccccttagaagctgtcgccttcggtatcaaagcggcctttgtgcaacggttccagttcgataaatgtcatatcaatctgggttgacacgatgctgtcatctttgtggacagcatatgaagaatccggcgtctcgttgacaaacatgtttgacaaaacacaagtcgatattcgatgtaaccatttattgcgttccccctgcaccatgaacgtgatatcaaacgtggatggatgaagatagaatgcgctggaattcttgttgtatttgtattctggatacatatgcatcttgaacagacgaatgatttccctcaccattttcgcctctttttgcgatcttggagtgaatttgaatgtgaacgggatttctcggttgcgcaccccttggaaaatcatttccacatacgggttggtcatcgtacctgtaaacaattctaatgtatcatgagcattaattgtcggcaagaaaggaattgcttccgaagcagattggatggcctttgtcgccgcaaatctccccatttctttcccaacattcaaagcatccccaagtttgaattgactcatatcttgggcggcgcgtgataagaattgggcacccatacctgctaatcccaactcggagccgttccagccaacgccatagttggttgtaatagattcaggcatacacaacactatggactcgttagagcggacatgacgtgcccaagcatatttgctgatagaacctgatttagaaccataaaccacaggagtttttcccagtgggttttgaatagggttctcaacggtttgagtcgttgtgtcgccgtatgacgatcctgatatgcggttaatattgaataaaacataatgaccgagggttttaccccccgttatatccaacggatagaccaattgcttctgagacttggccaagccttttgtgttcagaactttgatcttatcgatggtcgacttgaaattcgccatggtctgaagcccttaattagaatagagtaatgacactatttaggactagaatatggctatgtatttgcaggggaagtatgttcccgtaaatcccaagaaatataaaggcgatgtgaacaaaatagtttttcgttcatcactagaacttgtcgccttcaaattctgtgatatgaatcctgctattgtttattggtcatccgaagaaaccgtaatcccatatatctctccagttgatggtcgcgcccaccgctattttatggatttgaaagtatggactcgtcgacaagactctgaagagttacagataacccttattgaaataaaacccaaagaccaaattaaagaaccccgaaaaacaaaaacgatgaaggaatcaacgttcaataattcgatgcgtacatggctggtgaaccaagcgaagtggaccgccaccaaagagcattgtgctaaagttggttggaaattcattatttggacggaagagcatttggtaccaggagaagatcctgaagttaaaaaacaatttgcgctacgttctaagaaaaagcgcgaggtcgaaatggaagatcgacgccgcgctcaaagaattaatgcgttgaaagagcagatgaaaaaggaaacttccaacaaacaaacgacaaaacctgaagatgacggtttgttgttaccctgatttccttggatgtccaggggcaaaacaagtttgtttgtccctgcatatcacccatccttccgaacgggcttgtctagcacaatcagcccaagtttcaccagcataagacttcataccgcatcttgggtggtgctcttgccaagcccaatcacaagatttgcattccacgcattcacaatacaaatctaatgtgtatcctgcggtaatcatttaatccaccaggcgttcgtaaattttttgacggaactcattcccttttgcacaacctgtggccagcaaacgagtaccgcctgtcccgatgacactgatcgttgaaaatcttaaaatacggcctgtgatggactgatccactttgatggtctcaacttttcccaggcgaagttcatcagcatcacggcggataaaccctcgttttacgataacccgtttgttggtgactgcgaattctgtggtcatcacattcaagatagttgggatcaggaataacaaactgaaaccaaatgttggaataatcgttaaagcgacgagaacataaatccagaatccactccaccaagtcggacgagtgaatgcaaccacatattcattttcgctcagcatacggtcgacgtaacgcattttaattccttactttgtcaggattagataagatttgttcgtatccgacttctccttgatagtcgaaaacatgctgaagaatcgccatatcgtctaccatccgataaacttcaattccagcgttcatcaattttttgattccatccataatacgatatggctcacgataatatactttatcgattttaccgctatctattatcttttgagtgcaatcagggcaaggactatgtgtcacaaacataaccaaccctgtgaaatcatctgcattttcggggatacgcattagagcattttcctccgcatgaacgacacacgggtttgtaacaatttgcccgtgttgttccatctcacaaacattcggcatgccaggaggcgttccgttccatcctatagccacaggctgatcagtttcagggtttacaataacacaaccgacttgcagacgccgagcataactggttacaccatatgctgcagctgatcgcatatgagcaaacatcatacgtggtttgatagccattattcacctctcagaagacgagcgaagcgttcaacaatgcgattgcgcggcttctcgggaacgatgtcttctaccgtaatggtcagtgtgtagtttgtagaaaaatcgcaatcggactttgtgatgattttccgttcaccaggcagcggatacatcgttaaagccctatcgacggaatctagagaagaaccgaccaagtccaccaccctttggtgcccgaacgataatggacctacgacatccttttcaaaaatttcaatcacgtcggtgactaacacacccaatgcaagtcttagtccaaggttctgatttttatcccctagtgtgtacacaaactgccgacgccacaactctttccggtcttcagaacggacgagagaataattcaaatggtattgtttcatttcaccaacaccttttcaagtaaaaagatcatgtcatcctggtcggcgcggccaaagatctgaccgagggataccatggaatgaacctctttgacaatcaggtctttgcctgttacaccgctgttgtccaattcgagaattgtagacatgcgctcaaccatatgaggtggatgattgatataacgttgaagatacttttcacgcaacgcaattttttcgataagatctgacatttctgcattctcttctgaatacagacgttctttagatttcagttcttcgaggcgttcgtgcatttcttttaatgaaatcatgtttatctctcccaataaattaaggggaagtataccttcccctttcttcaataacctaaaatattattcagtgcacgctttaccgaggtctgtaagtctatagaatgttacttcagacttcggccaatgtttatattcttcagagttgtcatgaaccattgtaaacgcaggttcgatcagacctttagataataatgattgccctgaacgtgtatcccacggatcaccttttacagaacgaaacccaccagcaatcggaggggtatggaatgtccatactgtcttgcccttttcattcgtccaagacttcggattgaaataccaatgacaaaggcgtctggcttcacccttttcaaaatttaacattcctaaaaggacgcagagttgatgtcctgtgatgcgtggctttttcataagatccccaagtcagtaaggtaacgataaaacaagattatagacagaaatccaattaacatccaagacgacaggataggcttgcaatttagcgtagtctttccattgggtatgttgggacttccagtaataacaaccgaatccgttttagctctcagcgtcatgacagaacccatgagaatgattgggcgcacataatgtccttcattattgcgctttacgatacatggtgaagaaaatcgcaggaacggatcaccgcttttataaccgaataactcgaccttttgtatttcttcgggtactttctgaccaggaaagaatattttcataatacaattcctaaatttgtgtataacaactcagccagggttacaggacgcttgaattgttcccaactgcagttcaggtattcgcgctcttcgcggatattttcgtgaagatgaccatggatattatacgcccaacggtctaattgataaggaacatgtgttgtcaacacacggccaacaggcgtgtcacgttcgtacatagcgccgaaggagctaatccagccgtccatatacaaagacaacagcatcttaaagctgtcatggttgccttgtgccaccctgatgataaagttcggacgccagtcatcgggaacggggcgtcgcttaaactcatcaatgtttcgcttggcaccctcccgcatcaggcgaatgaacccttcagccccgatgaagcatatatcaccagccagttcaagaacgtcccgagacttcaacccctgaaagatactgtcaatgaccgcagcgtcatgggcttcctgtgtatcaaatccacgcggtttaaagaccttcttatgcccaagatgagtgtcaccaaggtgctttacaattcccgccattatggtttcctcagccattctaaaattggtacgttcacaaaccctttgtgccagcggtgaggtttatggcctctgttgtgatccagtaagcacgtgtcaggaggaacactgaagtgaatgccttcaatgtcataataattcccatcaatcagggtgtatacatgaccttcaatctggctataatggatctctgcttggggatatacgtgcttcagaaccagagcaacctgatagcactttccgttcttcccaacatactcgacatcgacctgttgtgggaacacgtctttgataagatttatcaatctcgtgatatcatttacaacaaccgaacatttcatctctatgataactcatggcgatgtcgaacacaggcagggcaattacagtcttttctcaaaattggcatttcacaccacgtcatcaaaccatgtattggagacatgtggtgataaaaccatgattttttgagactaatcaaaggatataaaatttcaattggcctttccccgtctcttgtcatcatattgtatccttcgatgaccttattgaaagctgggatccagtggatcgcgtcgtccccgttgacatatgccaatatgtatgtgggaggcaaactccggcctttaaattgtgtgtttaataaccatatcggggattgtgctagacacgcacgaaacccgccgtccggcacgtgaatgctactaatcggttcttggtgatgagtccaacgatgcataagaagcggagcattcttgttgatccaattataaattttatttcgcgcccaattttcgcgtttagatttaaatttattattttcgaggtggcaatacacagtatgaactgaatatcctttcgataacaaccaaatcaacatgtatgtgctatccaccccaccagaccagaacaacgtcacttcttgattattctccggcaatggagcatccccttgaagaacacggtatacaaaatcacaatcagatattgagacaacaatttccattaaaatattcctcttaaagaatcatttatcctgcgcaattcgttttccacttttatcttcatatttttatcgtcgttcacgaaaatcattttatctaaatcttcgccattaatctttgtataaaaataaatgtcatgctcaaagaaaatagtttttccaacagaacgcatggcaaagaacgttgcgatagttcttggtaaaggtttgaatttaatggcattgaaacgcaaagcctgatagagaggttttaatgttcctaagcgaatacaatgctggataatttcttgctctttcatttgttgtgcgccataaagatatgatggtgcaatgataatacattatacaaatagaaaagcccctttcggggcttatttgtcttcagattcatacaaacgccaattgccgatgctaaaccattcccctacattatttgaatatctggttgttcggactcctccgacttcatacgcccatttgaagaatttccatttggacgataaagaaggacattcgcttccggatttcagagaacacgctgtgagccaacgcgtaatactgagagcctgaaaaatcggatggataaatgaagaatacaatatccaaagcgttatgacaacaaaaataatggtcgtgatcaccccgacaatttgaaagaacatttaagaacctccaaaatattgaacacagactatgaagaaaaccatgattgtaaacaaccatgaaaacaaggaaagattcatccttccaccttcagtcccatcaggtcttccatggttttctgaatgcgtagccaatcaagcgccgcatcataacgaggatcatgatagaccataccttccagttcccacgccttgataccagcatatcgatcctgctgcccaacacccttcagccagcttacgatgtcaaatgtgtggtgataatcccaaggttcgttggaatcttgcttacacgtcacctcaatgatgtgctgagccttgcggaggtcaaacagattcctatcacaccagtcaaccgagcgcgggtcgatcccaaggcgatggcatcccttgcgaatttcatcggccaattcgaaaatagaaatatccttgtcggtcggatatagactgatagctcgggcggcgtcgcacgggttgcgtttacctgtacccagccaccattccacagtaccctggtctttcacacggccaagttcaatctgttctttgacattcagtttgataaatgtcgtgcgctcttcaacaagctgctgaagagtataacgctttgtcaggtcggcgatagtctgtgcccaagacaacatgacagcatcatcccaacgaccgagagtttcgctatcagcgacggccaagaaattagatttatacatcagtttcccctgctttctctaaaaggagtttcaaaggatatgtcccacaagcggtggcatactcgactttgaaatatgaattatcgttcatggcgaaatttcgatgtgggaaatattccccgtcatccaacccgcctaatcccccgccgacgcgatattgtttcaactcaacttcgccgccataacggagttgggcatatagtccgataggagcgcgaacgtaatgctccagccatacccaatttccatcgtgtaaacgtgtagggataaatgcaaaacgcaccttgggatgaagaacggctttggcgagcatctcttctgttatttcacgcttcatccaataccttcctcttcgatgatatcttcttcactagagacgacagagattccgtagtctaccatctgggaaagttcttgtaaatcttgttcaaacccgtcaccctttcggatataaaagacgggtacgacacgacgcctggtatcttcgaagatagtctccgcgattttattaatctggctcactacatgaaacagtttcaatccaccgatgccacagccgatggcgggtaaaccgacatagactgtttcgtcggtttttgcctcaatgatatcgtgaacttgttcaaacataatctgaactgaactgataatggaaggcatgcgtgcgttaggaccagggtagaactgagtatacaaattgaatccccagacaccagtgtcatgatcaaacgcataggacatgttccccaaacgttgttccggaccacgttcatctaattggtcggcgcggtaaatctctgggaaattcaatcgaacgtgattggcgatacccgcgcccattacagaccaacaatttgcgccgtgagcaaaggcattgataagattgaaactcgtggcggcttttaataaatcgccatttgtgataatataagtggtcatgcgtaacgtctccattgtataatttcgttgtcgacacagctaacctgaatgatacacatattctggcgatggtcggtgagtgtaataataaaactgtctggttgtgacatgacacgattaacagatatcccgcgcgacatcaaacgaaagcaaatttcttgatacaaacgttgcatatatcctgaaacagaagccgattcatatgcgcgttcaaattcgtataattcgttcatttattcagaccattaatagcattttcagagatgtattcgacgaattgattgtcagccgccatttttaaaagttcaaacaattcagttccaactgtactcccaaacattccttccaatacgccttggacatcttcatctatttcttcgatgatgagtccataagatttcaggcgtcgcgaaatagcacggaactgggcaatatctccaatttcatttgccatggtattatctcaatatggggttaggcggcaattataccgccattttcttcaatagaggtcttcggaaaagagttcacggaaaggagtgttcttcaaacgaacttgattggtttgtccagcttcacgggctacaatagccgcctcttccctagacatccattgcccatagttatcgataaacccttgttcacgcgtgcgggtgttttcgataacacctgcttccttcaatcgtttcagttgcgcattcattaatttgtcgtggtgacgggcgcttacaacgatgacatcaccatatctgttggcagcggccactacgcggcggtcaaatttagttgggtctggtatatcccgagcatatagggcgatatgggtgtcgtcaacatcccctttgacttcagaatataactggcccccattaggagatccagtgggtaaagaacgaaaataaccgagtaatttcataatctagtctatccatatttcaggttcggggatatcaaccatctccattgacatattacgcctccacttcttcatcaggaaaataagggcgtaaatccccaattaatttagggaagagggcaaacccgaacagattaaaagaacgcatggtcccaaccataccaacatcatccaattcggtaacgtcagaaagggcacacaacggtatccacacgtcttcttgtccttcgtaatttacgaaggcaacttcctctttgatgaatcgaggacacagaatatcaaacagccaatagacgtatgccagtaatgagagactgcggaaatatacaaacatggtgaacccctcacaggtttggagggaattcaccctccagatttataggcttaatgccttttcaagatcatctaaatcatcattgtagagatcaatgtcttgtttgttatttaattcttctctttgttcttccagattctttatctgattcaacaatcgttcacgtgcgtctaacgtgattgatgataaagacatcttgaataattcatcaagatcaccatcaaaatccggataatccgtttgaaacaacgctttcagttctgtaatgctgcgacgcatatccgtaaccacagcccaactgatgaataatgcacggttgttcaggcgatgaattttatcagtcatctcctttaacaggtattgacgacggtcttcataacggatcaaacgatagttgaagaaggaaagcagcatctccttccacccagaataacgtcggatgacaccatcttcatcccatgccgtccagacgggtttgttcgcagagtacagtttgaacatctgctcgatttgtgcatcatccagcttagacaattcaccacgtttgaacacgacagtaatgtcccagccgtcttcggtcgtgtcattggcgtattcagtgagcaaacccgccttatacaacggcaacagaactttggtctcatacgatttagcgaaccaaccaattgggacctcggtgatgtgaagggttgtagcattgatgcgtttgaagatgccgcgactgtatgctcttccttcctcagtatagcctgtctctcctttatatccattccagtacggttttaagtccgtagggtcttcgccacggagaagtgctctcagagcgccgaggactgatttaacgcaatggcatggggtgtccgttgagtacccagatccgataccattaattccgttcaccaagaacatgggaataacaggcaaaaagaactttggttccaattgttcttcacccaagtagttgtaatccaaaatcccctcatcttccttacggaatattttacggataacatcggatacagaaacagagatgtaacgagcagacgatgcatcacgtcccatgatagaaccaaattgaccatcacggtcaaagtatggaatattgttcgtaccaggaaacgcctgagccatgttaacaatcgtattgcccatgttctcaccaccggacttgtagttggtacgagcggcggcgaacatacccaggcgatcaacaatctcttccttgccatattccaatgcagtgaaaagaattttgcgctgacttggtttcaaactgtcaatcaactgaggtatttgacggatgctgttgaacacagaaaatgccttgtgatcattattgacataatccgtgacattgatactttgagcgaccatcttattctccctctgtttcaaacaaacaaacatcactaaaccaggttttgcggtaatcggcggcctcatcaccgaagccgttcttcagtgattctttgtagttgtcatccaacgtgaccgtcgtagtatacgcatccagattgtttagaatacgtttgaagtcttcagtactgttaccgccaagacctttcagatatttcttcttcgtaatcttcttggcgtcaggggtttttaaaaattcttcatattccgtattgctcataaactcatacatcgttttaccacaccaaatacgcatgtatggtgtgcgcaggagtttcagcctaccctgacggacgtactccggccaatatgtgcaatacagagtaatcaacaaaccacggatgtgaatgccatcatcgtccgcatcggtggctacaacagtctgtggatagcgggtgatatcaatcggctgacctgggactgcgcctccatgaatcgtgcaaagattcttgaactcctcattattcattacctttgatcgcggattgtttaaacaattgataaatttaccacgcaaagggaataatccgatctttttagtatcacgggcgttcaggataggattggacgcgctgtcaccttcagtcaataacagagagcacccgctgcgatcaccacgggcggtcgccggataatacttctctatctcacgataatcccgtttgctaatatccttcttggccttttcaaattcagcatcatcttggtcgttacgcaaggcggctaattctttactcaggccagctgtgacaaactccagagccttccggattagtttatcgctgggcttgtaggtcgtaccaaactggctaacaggagtcgtcatgcgctccttggtctggctatcaaaacgagggttgttgatatcagccgagatgaaaagcatcatatggtttttaatcatagaaggcttcagatcagtcttaaactttttcaccaattgtgggcgaatcgcattcacgatctggtcagcaacgtaatccacatgaggaccaccgatatgggtggcaatgctgttcacatatgatgcatgaataaacgtcccctgcgaaggagcaacagcgacagaccactcctctgtttcatcaacagaagaaccttggcaaaagtaatccacaaaatggccaaatcggtcgatacggatttgtttcccgttcagaatgactttcaggcgcgggttacaagcagccacttcaaatgctcggcgatagatcatcaacaagttattttgatcaagacctttgacacccagacgcgcataatcaggtatccatgcgatagacgtcccaggggcattcaaattactgatcaccggattagaacgtttgctcaaattgtcttcaaatgtttgagtgtaagacttcttcccgtcatttgtcgtgacacggaaccattttgaaaatacgttgaccagtgaagccccttcaccgttctgaccacctgattttttgttattgtattcttcatcgtcgtcgttgaagttgctacccgcatagagggagccaaataacatttcagggagccattcctttgtctccccgtgtttaacaaccggaatacccccattgtcagccacgatgatctgaccattcataggagaaacggtaatggtgatttctgtgaggcttttaccttctggcgttttgctatgatctacagagttggtgataatttcatcaaactgtttaatcaatgcaggagaatattcaaaattatccttgaatataacttgatcttttattggatcatatatccaagttgttccaacggctgaacggatactgcccagatgacgctctgggcgaagaaggatatgctctatgtgagtgagctttttgtattttcgttcaatatcaatcatattagacatcggcctatcccgtcaatggtaaacacaagattatgtatggggtcggcttatagaccaaatttaccgcgccatttctgtactttgtgttcaatggcatgctcaactttgactatggtgctttttagattatggttgtgatcacaagccagtttggcagtgtgtcggatgacaccatttaccaaatcggccagattatcatctgttctttgtggttggttgagtaatctggtaatgcgacctttgtagtggttgatcatcgctagttgctcgtccaaactatcaaccccaccacagaaccaataatcttctaaggtgtgttgtgtgaggtcacttgtacagttggcccgagacagagagaacacgatcgaatcaaatatctcgtctttgttcatagagccgtattgttgaatatgctgatattcacaaacatacaacaagtccagtgctgaaataataacgtctgccacttcaccattcagaggttctgaagcagaaatttcatataacacgtcagacaattcgcaagtttcttcaacaagtttacagaacacaaatacagatgggcgattcggttctttctggctgagttcataagatgtttctaagatgcgacgaataaatttcatttcaaatatccttctaagcgaaagcggttaatatgatggattataaaatccatagaagattgaaaactatatttcccctgatgctcatatgcatgaataaggggaagtcggctgtgagccaattggaatcgcagtcttggtggcatagacaacaacttggctgatgtcgccgcaataatacgagaacgcaatatgggggaatctgaatcaacgagagggatttgtattttacccaaatccaatccatacttcttggcttcataaccactcagatgccgaagagcatcatcgcgatattttatccagtaccaataaccctcgatagaatcgaagtgcccgtccagggtttcaatcggatgatgggtaaaatgcgataggaattttcccagatcagtccggctttggctgtatatgttgtaatgattgacaccctcagttataggcgtcatcgctttctttgaaacattcagtaatataagcatgatatagagtctcaaatcaaaattgctgtgtgtaatcaggtcaaatctttttatatgctttcagggtacagataatagagacgccgccaaatagaacgagcaatatgaatcgttccatcctaaaatcaggaagggaaagcagtcccacaaggctgtctaaagcaatcatgaacgttactccagtcagcatgcccaacttaaaggagttggaacctcttttgcaacgtagcataaatcacctccgattcaaatggggagtttcctccccatacaaattatctagcccagaaacactactaaagtcaaatttgtaaagcagctgcgattatcttttaacatttgatgggcagcggaaaggcgcttagcatcatgcagagtatctgaaaacacgatgatattggccttgtcgattttggttcccataaaattggccatggtcgacgttttctcttttcgatcaaatttttcatactgctcccctgtggttacatttttcgattgagtgaaaaacgtatcacgttcacgagcagtgtgaaatacaccgaatacgtcgatttcattttcaatttggaatttttcagaaaggagtttctttaacgccgcagtgtgaccagtctggcgaagcattcgatagtagagcatactcccactgtctgcgggatcaaggtcgtgacgaatccggcgatctttttgaatttcatacgcatctagaaccaattgttgtagcatttcaatatgcgcattaagggatacttctacttttcgtttaactaaagatttcattgttggagcctcgaatttacgttccgctcatggggaacataaaaattatacccttagacacaacaatgaaaaagaggccgaagcctctttaatcgtcatatttgcctgtcctgaaattccatttcctggccttggataggtcaagatctttgaatatattaccgaagatctttttaaaccctgactcttcttcaagatctttcggtctcttgacaggttcttgaaccataccgccttcgggtttccccacatcagcggtagtggtggccggagccgaagagtcttctaggtaatcctggaagctcttcatagattacaccacagccagttcacaacgggcggaagttacagaatggccgtccgcgtcttctacaacacaccagtactcaccctcagaagcagcggtgacagcgtggttgaccagagacgcagtcgctgctgttgggttaacaccagcatcaatcaccacaccttcgccgccaggggtgtcagagaaataccacacataagagtacggcgcggtgccgccagcagcaacgatagtgaatgtcgcatcagcgccgtcggccacgtcctgagaagcagccagatcggttgtcaacgacaacagtcctgcgatggtaatgacggtagaatcggtgtgatttccttctacagtaacaccataaactgtaacagaaccagactgaagcagtttgaatttaccaccgccgatatggatcgccttcgtcaggtcgccagaataccagttcactccttgatatgttgcgttcacaggagcaacagtggccagcaattcaacgatttggcctatttccccgctggccgtagccggagtaatggtcagacctgtggtaagaacagcagtcggatcatggatttcataagaaacctggaaacctactggcttggtcggatcaactttggtgacaccgtccgcttgcagattacttttggtcagcaacgcatcaagacctttacattcggtcaggatctcatcaggatcttgatttaggctggttttttgccaaccctttgcaacacccgtcacgacatgattgcgttcggtttccggtttgttgcggacaacttccggcgcgactaatactgtgattgttggcatggtttcctccactcatccaaatcgttgttcagagccatcatcagctcgttatctggttcgtatgaaccggaacataaatcatctctccaaactcctaacgtcaacagcgctgcctttagacgaggatgttggttcttatccgttttcaacaccagcattcgacacacagcttcagtttcgaaagtattgaaaagaataatcaattgattgataagcacacgaggacttttccctggcacaaatctcttcaatgattgattaattagcgaaattcgctgaatgtctaccatcaaatcatcccgtgagacaccagcagtgatatagtgtctcagggcatattccatcaaattgctttcgtcaacgatagccatattgaattataatcagcagttatacactgggattatttaatctggagtttttcagtcaattgagaaataatttcattccggtcggtagaaacaatccccaaaggttcggccaatgttttaagatcattgatgtcgagcaaaagaaggacttcaaccgtaatatctgtcgataccacagtgcctggtttcataggcttaccgaacttgtctagcccatatcgacgtaaagtacccgccagccccgtaagttccgcgagaagctctttcgtgccgtctttgtattcacagacccaaccgtagtcaccagcataaacaatgccgccgtcgccaccctgagggatgttatccaccacacgctgaagataacttggaagtttgttttcttcctgggcggtgatttcctgggacgtttgggtattttcaattttctttggtgtcttcgccatgatcagttctcctatagactgtaatgtatttaccacagcaattgcagcaataaacgtgcatggaatatccgtcgtcattgaaatcacacccaccacaacgacagaacaatttcacaaactggtcaccactgacttctcttggaggaaaattcttaaacgagtattctctgagttcaccctcaaattggaatgaatcaacaaggaactttgtttttcggcgcagtgatccaaatgcctgttgctcatacacgaagtaaacatctctggtttccatattaacctcaaagaaaaggaggccgaagccccctcttttatgtcagttcgcggttacttgtacagaccgtcaacgtcaatagatttatgttggcggttgtattccgtggtgtagtcatccaacacgagatattcacagcagcggattttactagaaccgtaatcatcagtgatagacacgatgtctttcgggttcagcttacaacgcacaacccgctggcccaggctaccgaaacattgtttcagatacgccagagaacaaacgtgaagaccataggagcacaggtctttgttgttatcgttcacgaatgagcgagccatccggacgatagtgccaggtgcgttactgactttacctgtgcgcttatccatataatttccgcgaacggatttgtatagaataatgtcgccgtcttcatgaatctcaatgtctgagtatgccatgaattcatagatccgaccagactgaaccagcgcggcgctcgggttttggaacatcttgtcaaggaaacggcccaagcgttccaagttcggataatcgccttttaatgccaaagccaaaatgcgtttggcaacggaagttccggtgatatcatagccaccccagcgaacacggttgtctgaaagatcaaccatgcctgtagtgaattgtttgatggcttcacgcggtttcatcagagtgtaagctgttttgacgtcacgatttttaatcgcttcaacaatacggtcatacgcctggtggctcgttgtgatcgtttccggtttaccatcgaccacaatcacgatactttccggcaaaatcatgatctggtgttcgttcaggaacacagacagttctttttcattaaatgaattctgagtttgcttgtcgcctgtttcttcaacaactgcaaccgtcttgaccacgaataatacggatgacaatgccagacgaagtgtacggcctaaaattgcatcaaagtaacccacgcgccgacgggcatcaagggaaccgcgcatcggatatgtttcaccgtcatgctgtacgctgattttcacaccatcattgactttaaccacgccatcaaccaatgcagtagccagttctgctaacggcgaaatattttcttcattgaagcggcttgaccgagtttctaacggtttcagttcttcgcttctgaaaagtttggcctgaatagcagcagagctatcataacgcatacagataaacactgtttggttttccaggaaacggccaacataccattcttcgccttcagaatcgacagacaccgaaccttccagaacttcataggtgtcggggattttgccttcttcagtcaattctttaattggcgaaatcacgaccacgttattagattctttgatgatacgaaccatcagggcacgggcaattgttttggcactgacaccataaactgatgccagagaagaattgctttcacctttgctgctcttggtttgtatttcttgaatctgttcagtggtcagagacttaatcgaagccggatccactcccagttctttatccgacacgcgttgtatcattgagcgcggtacgcccaattcactggctattgaactttgacttctgttctcattcaacagcttgagaatacgtttttctatagcattcatcttgtagttctcactatggggtaatggggctattatgccccgagaatacgttttgaaatagccttcacaaatctagtggcatcttcaatcgcattatgcgccgttggctgatattttgacatcatgaatttcaccaaacgatgacattcaactcggtatcggtttttggccttttgcttttgagatatagtgctagtgacctttatctcatcaaatccttcagcagttaatttttcattcaataaatcaacaacttgagttaactggttacaggcgcggaaaaatctcgtagttgagaaaggcatttctatacgataatgtaattcctcaacagccttgcggaaataaccgtaccccgatccagacgggtttcgtttggccagcatacgaacaaccttccattcttcttcagtgatttgttgtttcgcataaccgatgattgcttttgccctgtgttgatgatgagcatagccatccgtaacttctccatgggaatcaatcaacagcttggcaccataatcaatgcgatgagacatgatgtttatgcccttgcgaagtttcaggagtttcttagattcaagcgcctgaaattcatctttataatgctcaagcaacatatcaaacactgcgtcaatggggatgaatacttctgggaatttcttcatcatcggaacagatttacgacgcgcaagaacgattgttggatgcgctcccagcaaatcaaacatgttttcgtttccattctcgccgaacttcgttctttccatcgcttcacgcaaatgtccagccgtggattgtcgtaaggtattaccatgaattttatggataccttctcggccagatacttcgatatatacgatctttttgcctgaatcaataatatctgaaacggtttttccatccaattcataaacgctgtcgaagttaaatgaattgtaatggaaactaaacatcccaggatctttttcaacggtcgggcgaacatattccatttcatgcatgaaataaacttcatccggattgaataaatcgccaaaaacctcgatcagtttatcggcaaagaccaggtagtccggtgttgtttcttgtacaccgatccatgattcaaaataagacttcaattcctcatggttggtgaatgattctttatgtgccgccagctgattccgattactgaaataattttcattcggcttgacgacgataacgacattgaagttattgcgtgctgtttgtatcttcaggtttcgagcacgattcgagttatcaataataacagcaatttttgatttagatttcaaccaatttccgacagagttcacataaatgctgtcgtgtactacaccgtcgtcgtgtacttcaatttcgtaaggactaccaatagaccggaaaggtgcatccacagagtaatcgcgcacgacgtccggcgctccgatataacggaacgtgcctttgtagaaacggttatctgcaggaaattctcgctccactaatttacggaaaccactaccgaacatatcggtcaattgagccttcttcatataaagcataagaggacccatagattcggcctggcgaagttcatacatcttcgcctgcataatattgtcagccacatatttgaacactcggttcaggctttcacgggtgaactcgtcatatgacaagtcttcacgtgatggaggaacattcaattctccaagttcaaagaatgtataggaactcgggaatttctccagcaccgaacgtatttcagaatccaattgctcaagatctatcggataagaaaccccgcccataacggcatagtgggtattatcagaatcagacttcggctgaatatacgtgttgcccacacggttttcaaaattgatgtcctgccagcggaaagaataactggcattttcaaccttcgggcgcattgatgggcgcataactcgacccagacggaccagttcttgttcaaatgcggtaaatcgctgattctttacaggcactttaacagtcagaccattgaactcatcagtgtcttctggtttcggatcacgggtatccaaatccttggtgatcaggtccacagtaggaatcctgtccgcattcagatacataagaaagcgatacagtttaccttcataacgactttctacagtaaatgagtccgacactgcaaatggggatttagaacccagcccgaacgcgccgatctgcccgtcgtcgtcctctttggtgctgtggaagagtgtggtgtacaaaccaggtccacggatgatctcgccatcctcagcgcggaataccaacaccccattgtaataaccaggcgtcccaataacagcagtgctgtcagggatttcatcttccttcacaacaatgttgccttcgacaagcacttcatcttcccgcgctgcgataggttccccgataatcatttctaacggaaggccaatcccataatccttgatttccaaccatggttccataacatcaggtaaatggaccacaacgggcgtccctttgggagcaagatgcttgctgtatctttggggcataggggtaagaggggcataatgggacggcaaccaacgctgttggcgatcacgcatattatgcgagtctataccgttacataacgtctcacgcaaagatgcggcctctttatatttgtagagacttgaaaataatgtttcaaataatttatccgtcatcttgacattaaatgtctgtccgacgatagtggaagaagagcgcacgaaatgctcagacttgcgcattttcatagtatagaattcctattcagtttcaaagggaacacattattggggtatgggtatgatactagcgggggactcaatagaaaaagcctccaaaaggaggcttaatcaggtttaactctggtctacatttacaacggcagcgttaaagttgaatgtcggatgggtagcgcctggctctaacgtttcttccagataacctgcgatattccgcaccacggttttaggctcagaacgtgaaggcttcgttgctccaagttcaatgatactggaagtaacttgttcgacttgggtgtgagccacaacaaaatggtaacgactaccaaacgccgccacttcaacttctgtaatttccaaaccactcaattcagagctgctcaactgcttcacattcagttgtgaacgaaggtgttccacaatgttgctgtaaacatattgtggcgtgatcagctgttcaatagaactcatgacatatcctcatttctttgggggttattgttacttaaccggacgacctctgctcgaaggaaaaggatttcatcggccattatctttccctcttcaaacatcacatggcaacgtgataatctgggttcgccttcagataatgcgtgaagacgatattcagggatgacatcatttacggcgtttctttttgttctttccatgcgtcttggcctttttatgacgactaacggaaggacgggcatgcccaggctccccaggggtacaaccttgggcataacgttggtcatcccagaggcgtcctttgtaagacatatcttcagccatcgcggacaaggcgagggttgctacagccgcagcagataatggtttagacatctgtttctccttttgctgcatatgccagtgcttttacaatcatcacggccaatccgattgggtcgcacgtaccttcgaattcttgttctaaaatctcacgtatttcatcagaagattgtacttcggagcagttcttgctctgtcgacgttgttcttgctcaacgaggtctttgaacagaaggtctttgaaagaatactcatcgaagttttccagatactgctggatacttggttgtttcgcatccatcgttctacgcagattgctgatagttgaactcgcctgttcgcggtgtttgttcataacggaaaagttcacgggtttcccccaagcgttcgtcactgtgttggaacacacttgtccgaagagatcttggaaggcgtcatccagatgaatcagcgcggcggccagttcatcataagttggtttttgagcagtcattttcgtttattcctatttttagatttcttggcttgacgtttatgctgagcggctccatacaagcgcggaggcttttgataacgctgtgaggcaattatatccctgtagtcggcttcggcttcacagcaagaaacatccgttcttcgtaattgcgcgaccgcttgtgacaccagcattttacgttcgctttctaccaggtcatcacaaattatcatgcgctcgcctggttcacaacgacgtttgggaatgtcgtcaacactgatgcattccacctcaacgcctttcaatttttcttgaaggatgatggccatagtacgagcgcccacccctcctccaataattgcaatctttcttggcttgtcagacatattacattccataaaaagaggatagagcaataataccctatccccctttattgaagaacgattagtctttggattctactttatcaccgcgcagatcctggacgggtttgccatccggcgacatgatgtagatggtgttaattgtctcaaaccaagacaattcatcaactccttcagtgtatggagttaccggaactgaagttttgatatgatcaccaacgttatttggggcaccatcagacttcagcactggctccaggaaggcgaaacggccatttggataaatcaccccgcctttgaacatgcgaatcttcttacgaggattattcgcctcaccaaggcacaccaccagatgatggatcatctcaggacgggtaagcaacgtgtcgtcacggatgatcccctgtttgatagcggagatcaccacttcatccaacaaagcctgttgctgttccagagtcaaagattcagtaagtttcacgctcatggtctaacctttgaaattaattgggttctgaaccccagagtcaaacgcggcctgggtacgggtatcgctcaccacctgacaggtcttgtcaagatcgatgcgagggtatccaaagatgccagcaatgacagatccagggaactggccgagtttggtttcatagatagcacacgcgtccaatttctttgtctggctgatacggaattcatcacgtcctgaactgatgaccacctgaatttctttgtacaggcttgaatcaaactggatgttctgttcctggatccattgcataaccgcctttgaaccatcagcaccataacgaccctggaaagtatctttgatcacttctttcagaccgtctttgtacaggccaggtatctgagccgtctcttgtactttgtttgtatagttgctcagatggttttcagaatctttattgaactttttgacttgctgttcaatacgattgaaatcattgaaataactgacgaaactaccaacaccgattacaacaaaagccagaatcgccaaaccgatgatccaaccaattgaaatacctttacgctgcgacatctttacttcctcttgttaaaacaaacgtttgtaagtgcgaccattgtactgcatatggcctaatatgaaaattgcggtaaacggaacacaacctaataaaacgacgataatcacttcccatgtcttgaggtcacgccatttcagatactccatttccttcatttcaacgcggttatagcctttgctgatattgactgcaacttcttggaatactgatatccccatgggtttgccagtcaatgaaagaccgtttcgggaatggagttccatgttattcatcccatcagcaaatgacgtggatttaccccaattgattttcatatccttcgatatgtcagtaaccacaatgacatcgttctttttaccaccggaccatgcatatagcagcccctgaaaatattccacaggctggccagaagtcactacccacacgatattgacctgacgtgatgcccccaatgttttcaaagtgttgttcagataatcattccaataatcaacgggaatatccatccctgacatattcagaacacgagtcacccgataataatcatacacccttggatatgacgggatatggtctttgaacttctcagcgaattgttggtcagatttggagaataaagaatccttgttccccagaacatagttcatatatgcatgttcacgagctgcaggttctccgattttaacttgcgcccaacgcggaggctctgtagtaccctgacgatcaatacggtcaatgctcagatcaccgacggttgttaatacgtcccaatcaacgtcataagagtgctcatagcacgtgctgcaatgacgcgtttgtgtacaagatcggttatttcctgaaccagagcaagaagttgtgtaatagcacatacattcataagaatgactacagcttaccttgtttcgttgcttatcggtaacgtatccattcaaaatttcaacatctcccgaactacccagggaaaatgcagcgtatgctcccgcctggataagacaggataatacaaccactatcgcagtagcaattagcgtttcttttggttcatgggttttgcgatggtaaaccacgaacaatatggctaaaataatagggaccacaaacagaaggtaaatcataacacaccatccacgagttgttccttcgtcataccaagaagttgatttttatgacctgattcagctccatttttcaacaacagatcccaacgttcttcatgatactgtggagtcattgcatcgtcaaaagaaacatggtggcgatgcataattgttgcaacggtatgaagaacgattgctgtcgctctttttaaagcacgtgtatcagtgatatggtcttcatattcaatacccatcaaatcgagttctttccgaatatatgcaatagacaaatcaccttcagaatgcaatttaacaatagtcgctttcttaggatgtaacacgccgtctgtttctttcatgaacagatcgtacaaatattgtttgtattcggggcgcatcatctcgtaatccctccggcacagataatattcagttcaccgcgacggaaaccgccttgcccctcgccatgtagtagaccaaacgcactcttgccggaatttttgttgccgttcagtatattccaaagagcatcagcatcgcctaactcagtcatctggtattttgttggatttttatgatcttcttccagaatcaccgataccatagattcaatatcgagggagtccatatcttcaattcgttgcattgccatgatgtatttccttgtaaaagaaaggggaacgggctaataataacccattcccctttattgaagaacgcaatcgcagtcagattactttttcttcagctcttccagcttggcttcaatttggtccagaatctctttacctttctggacgatggcttcaccgtcttgcgcgtgtttacgaaaaaccagagcgccagcaatgaaaccaacaaccacgcctgggagcgcatacaccaacagatcttgtaacatgatttatatcctcacttttataagcaaggatatttatatcatcgtttcaacaaagacatgaggtttacactggtcgacttcggattgtcctctatcttccggacgggcattttcagattagtgtaaacctcagatggttcccaacgcgtaccctgctgcaggtgccatatatgtagaggaacacctgtctcaaagaacacaggcatcttccacttctggacgccaggaacgtgccctgcaggagctttttcgcataacagggtatctggtagccagagggaacgggcttcccataatgccttacaggagtactcaggccagatgcgttccatgtagtcgatctcatcccaagtacgctcggcatcccacccgatgtaacgagtgtttttcagacggaagaatttcttaagccaacaagcaccggaagtctcaaagttcaaacgattgatgaacgtgatgtgaccaaactcggcgacacaatctttgaatgcctgttcaagatcagcctcaaggatgtcgcattcttcatcggtgataggacagccgttgatctttttctttccgtgcttggtcacccagtcgtcacggtttgacaagaatgccgcgccgttgcggttggactcactaccatcacggtcacgcaacatgaacccagggacgtcgatctgatacatgttgccgaataccatgttcagggcttccaggaagttccaggcgctcaggcgaccgaagtatttccaccccatagccgtgtcccatagcttaacgaattgatcactttgcagtttagtctccaacaacggacggaaagcatccatctgagtacgaccagccagccaatcaatgtaggactgaacacacgggatcatcttcgatttgcgatagcggcaatcagtatcgaatcgcatacgctcgaagttcaggttataccaatcacagaatcgcttgaactcttgtggtgactgaggcggtacaggaaactcactataaatcgtccatgggccaatcccattataacaacagccccaaaggaacgccatccagatcttgcgctcgatcttctcaagctgcgtaatgccttcagtgacttcaatcgcataatccatcagacgaacttgttggttgtgttcttccgtatacgacagcgcctccacccatgctttgagcagatacatccgattctctggcttacgataatcgattgacaaatcaattgggtacttccattcagccggaggcgtataacctgtcggcattttagatagatgcgtcatccttcacctcggcttcgagttggatatcttcagggtttaaaacgggctggcccaaagaccaaattggaaggaaccgacggtcattggatactacccagatttgcttggcggttttcgttttttcagaaatataatgctgggcattaacctgagtccgtgccagcccaatttgccgccatgcaccgaaatctttagaagatggattggtgttcagctggaattggggaatgattttattctgttccggataatatcggacagcgttaatcatattggcatcgccacgatagtcaacagcaatagcaatggcattgcgtggaataagaattttcattttaattccttttgaaactttcaattatgttgtaaacgaacggcgttaataaagcaacaccgcccgaataatataacgcgtaaacattcaatatttcggttgagtctaattcaacgatgcccaaatatttcataacaccgacaagcatagctgaaaagcacatcaccagaccgacgaacacaaaatatttagacatctttttcattatctacaccttacgcattagccccagaagggacacggatgaggtctttggttcttccttggcgacggccttgtttttagttttggatgccttctgcggcttctcaggcactctgtgagccaaaggatcctgaccgccgacttcacgcgtcatcggattggtttcgcaatagccgtagaagtctttcgacatcagattgttagcgcggcaaactgtgccaagctgatgatagatagcattacccaccacccacaacggggcgtcgaacggcaacatagccaattcgctatcattcccagtcttgaattcttgggtgttcgtaaccgtttggtggtacaaaacgtcttgcccactgtaatcatccacggcaacattccagcccagcgccagcatttcagatttcgacttctcgaactccttggagtaagattcgttgcgcgaccagccggaatcccccgcttctttaccagagcgtccacggatgcgggcatcgtactgatagcgatcaggatacgcaaagtacagcagcgccaaagaatcaatcggatagttcttgaacatccattcaggacgccacttgtccgacagcatcaggggttcgccctcacacaccagggtatatccctgagccagccaacccttgatgaggtcgcgagcaatatccccagagcctgtggcagcatgaatggcgtccatagaggtccaagacgccaggccggatttattggacactgtgtattggccgacgaagattaacttcagctcttcaaatttcaggccgaaggggcgcgtcttatccccaacagtgtacgacaattcagtcggcttcagcttagtgcggagccactcgatgaactgtacgacacgagtgcctttacccgtgccgctggttcctttgattacaatgatctttgccattatgcagcatcctcaatgcgccaactcactccacccaaccaaggtgcccaaaacacggcaacttgcggatgaccattctcaacaaacttcactttatactggtcatcaaagttatagaaaatttcgtcgtcatcaggtgccataccgttttcaatcagaactcgttttaattccaacgctgcgccaacggaagttggtggattctgattacggatgaagttgtttacatggataatgataccatccatatcactgtcaccggaacgcttgtcagggctttctacaactttattttgttcttgcacgatgtgctcctatcggacatattgataatgagaaaccaggcgattcaccacttcatcgaggttacgatttaagaagtcaaagtggttttggccttctacatgcttaatataaagcatatcctcgcaattcgaaagatccagtttttcataattgccctgagggacgaagatgacgaagatgttattcagggcacacggctcaacatacttcggattgtcgtcgatcaggacatcgccagcaagcagatgcttttcatccgtgctcacgaacccattaatcaacgattcaaagtgatggtatatgaactgacgcttgctgcgttcgtgttccggttcgcattttgtaactgccaccatttccacttgctcaaacttctgaaggagagcggcctttaaattcgtaaggaattcaacagcccctggcagtggtgacattctggaatacagatcaggcatacgccaccaatccatcggatcacgtcctgttggcgaatccatccactgaccagcaacgaatacacggcgcgttaaccaggccggatgcgcgcgttcacgcatgaggatcgccagatcaccagcgtgtgacatataacactctttggtgattggttgaaattcagatgggtagtcgtgacatcccatattttccgcagcggctttagaattagaaatattgaaccattcaacccacggcgatagggaatcaaccagagtcaaatccacatccaccagaacgcgatataacccgccaattttagaacatgcgtcatacagaccaacgtgattattcatcagatttctccacattgtcataaccagttgcggtgcattgctcttcggtgcaatcggcatacagaaccacagaagattccttcacgtaagtggtgcagtgataggtttcatcttcctggatggaatcatcataattgcaaacagtttgcaacggaccaggagtcgtgttgtccaaagctgcatgtgccttgatggtgtcttccatcttattcaccgcttgctgattggtgaacgtcggcgcatcgaggtcaacagtagacgcaacgtccagaagggtagagcatgcagtggtagagaaagccagagcacacaccagaagaatcttattgagtttcataacaatacctgttgtcaaagggataacagggggaattatatccccctttgttttattgaatcaacctttcttgcccatcatggacaacagattaactcgacgtttgtgagaccaaccaaccacgtctagaatagattgaatcggctctatgaaggtcttttcaaacgtggtgtgataatctacccacttatccagccccaactccggagggaggaagtcagggaaggctatgcggtcgttccccacagggttcccaggtttcaggttaatgattttaaccttatcgccagattctatcggcgggagaccgaggtcttcatgcttgtctatcagcttgttgtacataatacaagccttggcagcaaagtgtgtgccactgatataatttccgttgccgtctaaccacttctcaatatcactaacgccagaggcttgagcaatatcatccacggttaattccatatattccttcttataacccgcgatcaattcctgaacttcagcctccgtccccagaagaactttctcatagcatttaaccagacgctcacgacaccattccggcgttgtagatttacgcgcttccagacctttgaatttgatctttggcttctcatacttgatgccttcgctatcgtataccgccatgcaatacatcttcttggctcgccatacagcagacgatgcgatgacctcacgttcccagaccatgcgctgctcaaacccgttcatggtattacataacaactgcgcccattcgctggtttttggctggtaattctctttgatccattggtcaatgttatcaacgagtttgtgatggtccttttcttcaggccacaattgcttgaccaggcgttcaatgcaaatgtagttggagtcagtatcacccgcgataacaaaatcctgaccagtagtgccgcaaagtttgttcagataatcatcggtgtggcgtttgttccatttgttgatcagctggccggaagtggtgattgcttcagcgatgttgatgttgaagtattctttaaaccagacgttggagattgcgccataacctgcgttcatcaagatcttcagaccttgttgtaacgtgtcttgagcaacccccagatcttcccatttatgcatgacttcagtcaggtgatcaagatcgatatgcttgtgttcttcataccattcaggatcgtagaaacgagacttcatggcagactcaaggtggaaatcacctttagacatttccttacaccaaccagcccattgctcatacttcaggccagttgctttctctcctttacggtcagcgtatatgcctcgcataatttcggaaaggaaggacatcttctcattactgaagaactgaacgttcggggtaaacgaaacgttatatcggcgtagtgtttcaaaatggaattcgcccagcgcgaccaattcatcaacaacttgtatgcgttcatcaatagcacgctgcaacttgtcgtgaagatttttgagatgacgacgtttattcatcggcgttgtcatatcattcatcgctttggtcaattcctcacacatggcctcaataatatcacggcgtgtgtgcttatcagaaacgatagtctctggaccaaggttgtattgctgtatgatgtggggatacagggagtttaagtcctcagagaagacccagaaatatatccctggtgcaacctccatgacatatgcgccttcaaaatctgttggcccatcatagacgcgctggattttcgggacaatccccttctcataaagacgataatagcacatcgccagccacggtgctacagtgccgagaccgtcttcatagttggatttagtgcgataagccaatacaaacatcagctgtatcaaacgcagtttttgttctaagcgccacaccagcttgacgtctttgataccatatcgcgtatgcttgcaataatcattgaaataaaggtcatacaatgatttgctttcactataatccaacttcttctcaccgagttcacaataagcgatccagtccagtgagtatttttcgcgggtggtgtaagtgtgtttcttgtacacttgcatgtagtccatcattggacaacccacaaactgataagacgtgacgtcgcctttacggtctttgatgaaacgtttcttcagtttaccccaaggactgagacgctgggcttcggattcaccaagaacctgcgtaattcgttcaaccaaataggggctatcgaacgtttcaatgttccaccccgtccaaccgtcaaattgacgctgcgaccagtagttaaggaatgaacgcagcagatcctgctcggtgacgaactccatatactcaacctgaagcccaccgatttcaggatcgttctcatcatacctgaacttcgcgcgatccttggaacacggcatgccccagacatagaatttattggtattcatatcttggagctgtatcagcgtaatagggaacgccgcattcatgttctgtgtgattttaccgttactgtcaattatagggaattggtcagtcacgttgttggaaataaaggaaccagggaaatgctcccgaacgaagtcatggttggccagtacttgcttatggaaacgacgaacccgcgcctcgctgcctttaaacgtgtgagattcgatcgttgcgtgtgggaatggacctttagtcatttctccgtcacgccaaccagccgagaacacttcgatatccacgttggcgatgtggatatttgaataatcaggtgtgatcatcccaggaaaactatgggcgatgaattgatatgcataatccgtttgtccatacaccgcagcgccttcgacctccttgtactcctcaagatagttgtcagcgtcgcgcattgatgcaaactttttagacaccaacggttcgttgaggaggccaatcttttcaactttggaataatcagctgtgggtaaatacaaggtgggttcgaatttcttacgcaacatgcggcgattgccgttgtcgtctgcgatacgaatcagaaggtcgttaccctgtcgggcaacgttcgtatagaaaacagtcattgtttgtctccggttaaccgtgccgtcagcacgtccccgtaatttaaccgaggaacgttcaatgaccaaggagtttaaaatggcaatcccctttgtgagcatgattccaggggcgctaaagcaactctggaatcttgggactgatttggtgcaatataagcgggaaattgttcaggctaaacacgatgttaaactggaggcaataaagtcttcttcagaatgggaactttctaagattaccgaagtcggcggatcttggaaagatgaattctggacaatcgttttggctgttcccgctatccttgttatgatcgctccggttgtggaacttctaatgttccccaccgagtatcataaaggggatttcatcaaagcagtaattgatggtatgcaagcccttgaaacagcgcctgattggtatactgcttcgctattgacagcaattagtgcatcattcggtatcaaggggtacaaccattacaaaagcaatagccggaaggcgcaagctgtggatgctctcaaacagtttggggtcaaagtggttaacaaagatcccactgttgttactcagtcgggttctcccgatcttgagtctgctcccccagctgggtctactgcgacctctggtgcgtggcctgatctgaagaaataagttcaaggggaggaaactcccctttcttttggtgttagggaatgaaagtggtaaacaagagattgctcgtcacccaaggcggggcgacgatagtacgtggacggatatatgaacaaaccaagctgggtgatcataagactcgtctgacggttaaagcccccgacggcggttggccaacaaaggtgattgtcccagacggcctcatagaacgagggacggcgacgtataagggagcagagttcaaaatttattaaaatatttattgaataaagtttacttcttcaataactggcgtatacttctaattgtagtacgaacaaacaacaatctgaagagaaggaaatacatcatggcaatcgaaattatcgctcaatcaaaatcaggcaagactgtaatgtacggcactgaagaaggccgctgcatcatcaactacggcactatcactggcgagtctccagagaaaggtggccgctatctggttgagtacggttcacgcctgctaatggtaagcaaagtcgatggtcgtgaaatcatgattcctcgtaaagaatactttgactcacgctctgccgcgatgactttcttcctcttccagaaacgccacgaactcttcgttgaagcgaaagacgcaggtctggacgacgcgacggctgagctgattgccgaaggtaaaatgactctcgaagctgctctgggagacatggattgcgaagccgaagcacattatattgaatattccgcatatcagaacgaatgtgaagatgatccggaagacgacggattcgaccctgacctccacggcggtgaagacgataacaccccttccttagaagttctgatggctggcggccaggccgcgtgggaatggcagaacgagaagaacgcctggctggattcccgtatctaattgactgaagccccttcgggggcttactttgaactgaaggactatatcatgattttaatcttatggtttgtattcacatttctggccatggccgacatcggcaagtatcacgttctcccaagcgacctgaccgtcctgctatacggcatagtttcgtttgtgctgggttccttgcgcgcccatatgaggactatatcatgagcaccccaaaaactcctgtccgtatcgctgttgagcctctgctggttgatgctgagaatgctgctgagcagtacgcgaacaaaatcatcaagaacataatggacgatctagcatccaacggctgggacgttgagaagtgcgctccgtggccatcttcggttggcgtcaagtttggtgatccagattaccaacgaatgaaatcaaaacacgcactctaccgttctttgacccaggcagtgaatccttcttacacacctgggaagccggatattgtcaaagctgatcctgagcgccatgctctgtttgtcaaggaagcacgtcgaaacgctgcgatgcaatatgagtctttcatttgtaaactggaaaacaaaattggtggtcatagcgcggctgtactgaacggttcccatgtatggggttattcaatcctgaccgtgactacaccagaggggatcgagcgctggaagactcagacgattatcaatatgtccaagttgggcaaactttttaaccaatatccaacgaggaaggtgaaatgatgaaaaacttcgcacaagaattacagactacgattgatcaggctcagatttgggaagccatggggatagttgaaaaagccgtcgatatctttgtttgcaaaatgaagaagcaactcgtttcccaagcaaaacgcggagttgaaattgatgacatggtgtacgctgttggtggccataggtttgacgaaatattaacagcattccaacaggcacataagtctcaaaaatacccaacagacaggcaagtcgaaaagttcaaaacattgttggtggcgagtcttctgaaggaaggtttgaagttcgaattacacaaggatgaaaggacagatgtcgtaacaatgagattcacagtatacacaaacgatatctaaaataaaggggcttaatgcccctttgtttatttcccatcccaaaagtcgtcttcatcgtctacttcatcaaacgcagaatccaattcttcgtcagagtactctggaaggcgaccattctgtttcagatattcaatctcgtcatcttccaattgaagatccgttgctataaacagtgctccggtctcttcatcatgttcaatgctataagacgtatattcagaaccctgaactgtaatgatgatagatgtcggatcttctggatccacgtgagcttcaaacgggataccgtcaattggggcgtcttctaagaacgttgcaagggctacggcagcgcggacaacatccgtgccctgtaccgatgcataaatttcttctgcacccgtttccccgcgctcaaagaattcaatcgtagttccgtcttgaacaccagtccagataggcatatctgctggggaatcagattcaaataatttcgaaattggcatggcaatgtcctcttgaataaaacgctttggactatttaggcttctctggtcgccaaccctggcttaaacaccaccatttcctcaacgttattggcactcatagaggctatagaatcgaaccctttgttgacaagttctttcacattatcatgtatcttaccaggctttatccccaactctcgcattttattttcccacaccgaaacattgttggaatctattacaagagtcttcttggccttgacacgaaatgccatgacaataggttcaccagattgacgatagcgcacggcattttctgcgtagatctgtgcctttccggatcccctgtcaggtaaaacccagcacccatcgtaccagagtcagtctggccaaatttttcaaaatcaaatatatcgaattcaacattagacccatgatacaaaacgatattcgtaaaatcaggtagcgttgattcgcgataaaattctagaaaggttttcatagataccccacactggtcgtatggggtatttaggatggattagctggggacaaactcattcacagggacatgttcggggattgataattcaggacaagcagctttcatcgcttccagatatggcttgctccccacgatccaaaacaatgagttctcattaagcaattctggtttgtgttcctgtatccatgtcatgaccttaccttcataacgaggatgcaactcaatatccccccactggtaatccatgaggtcgttgtaacgaacccaattagtggtatgaagatcccaatggtgtacctcaaagcgaggaagcacgatatccggctcgttctttggcctgataccacttaggaggccagccaacgaaacgctcttagaggcatgcggaagatccttcttaccatacacatagtctgggttgtcgtgatagcgacgagtgaaatcagttatgtgagggaggtttgcttgcttaccgataatgcgcaaacgactttcaatgaaatctaatcggttaggtccaatcccgatcagatagacattcttcagatttggtttcggatgcatcgccaaacctgtcaggatactggtgcacgaattacaagaaccagcaggaatgatcagatcagtgatatggtctgggatattggcaacctgctcaccacccagcatatggaatccagcaatgcgttctggtgaatgaacggtatgatccaatgtaatgccatattccagataatacgcctttggatttaattgttcaatgagtttcttacagcgcggctgaatggtgctattgtaaccagatccaacaaagttaaattcactaccaaaccatgctgacattgaaaccatatcatggttcatgcatgtggttggtttagtggcacccagcacagtggttgtcttgccgccgaaatgccgtgagactgccgtcgccataggggactgcggactaccaacgacagtaccatggataagatctggggatcctcccgctttaagatgctccatcatgagccagatggcctgacggagtttgctgccattgatcccctgcttgccgttctcatagtgggacaggggcgcaaagtaatcttcacgcttgaaccaaaccttctgatcggtttctggattggcgacgagttcacacggggtgtgcttgtagaggtagtcttcccagtgcaccagattgcggtcgagggagagagtggtgaatatagacttgctgtggtgctgggagattacaccatttggtaatggctgcacagaacagagttcttcacaaattttctttgtgtcgatcgcctgaggtatgccagcggctgacatctcttctttcaaggcggcggcagtccattcttctaaagtatcgcgatcgataccgtcgggaaagataaaggggctggcggtttcttcgactgcctgtttggtgtgatctataagtttctgaagtttgtcgttcattctatttctcctgtaggcattggcttatcatagcgtatctacaggagtatagaaaaggagaatctcggcttacttcttgaacagacgaaggaacttcatcatgttatccatgaaagtcggcttcttgaccttttgttttaccggagcgccctgatcattcagaacacgactggccggaggagtaggcggtgtcgggattgaactgtcatgtttaacaaccggagcaccactggccagaggagttggcttcaaagatttaattgcctgatccaaataggaattgtccacgacactcacgtcttcaccgaccgtaacgcatggtttgattagaacggatttcttagattgctcatattgatgagcaatatccatatatttcttgcgctctgcgtagaaatcaacattctttactttacggcggcgagcagtagaactctcttcaccatagaaatcataaccatttttgaataaccaattcacggcttgctcaaatgtattgaacaccatatacagaccgcgtcgaccatttaccatgtcgtcttcgctggaataaccaatgaataccgcttgccctttcttgttgatatcaaaaggagtacacacaccagtttcttcaagtgatcgcggagaaagatgcccgtcccgatagatatatgccggatacaagaactcattcagagtacgtttgttccctttgtatgtgtacaggcgagcaaatggaggcaggtcacaaacgatatcgttgtaatcccaaatcgtaacattacgcatgttgtttcctcaaatcaatgaaggggtttataaacggttaaattttaaacccctcttcctcattgaacaaatcaaatactagtcatatagagtgggacaacctgctcagcgataaacacgacctcggtaatggtaaaatcaccaccataagagtacagtttcatttgcgccccgttggtcgccaggttcccatccttgtcaacactgaagaacgtaatgaaagacaaagtgtccggcggcggtacgctagcatcacgacttcttgataactggttccccacagaacccacaaaatctaatatcatggttctattagaagtagacccagaccatgatccgacaatattaaccttcacaggaagaatagaatcatagggaaacgcatgaaatttgtgatctgataatttaaagaacggagccaaaccagtaggagaagaagggtttggtgtcttcccatccaactcagttaacaaatctattccagttgccccagaagttagcagttgtgacaatccagaaaatctgacttctgttttaatgcggtgggtctgaagcatattccccaaatagggagatgtcaaaatagccatatctcccccttaagaaacgataaccgtcgcatcagaattaattgccttaacccatcccaccgtcggcggggttaaaacgaccatatttccttgatatatgatatgcgcgttgttacccgttggagcgctggggctatcacacagatacacagaaccgtgagtcacttgaagagttttcaaagatgtgccgtctgagacttgcgtccatgtacttgcgtttggtgtgatcttagcagttgccataatagacctcctttacaagtcagatctatttattacagacaaaaagaagcccgatcgtcctgatcgggctatgttaaaccatcgtggggatggaaagactactcacagcaatttctgataatgggcgttgggagagtgcgtcgggaatcgaacccgaaatcgtcaggacgctgaaagctatttgcatttcacttgcatatcctgcgactttcttaacttccttaaccccagattgttatgaagtcagcatgcacaccttcttcatcttcaggaagtcacactcattattctgattgcttcaactggattcgaaccagtgacctcaggcaccttgttctctcgaaactttcctgcgctctaccactcgagctatgaagcaatttggcggaaggtgaaggattcgaaccttcgggggctaaaagccccactcgattagcagtcgagtcccttaaacctctcgggcaaccttccaatttggagcgggagaacaggattcgaacctgtccagtgatcgtcgccgatgatcagtcggctactggccaacccctatggctcccgcgaaatttggacgggcttcacacccgattgagcatttgatctgccgtagcccaactccccgcgcttcacagcgccgattggttaactggcgaccagtgccttctggttcgatctcctggaactttacaggcttcgcgcccgaccatttctggaagttggggcatcgtttccgatgtggagtcaccccattttatacacagctggctgcaacgcttactgtgtttggtctaccgctacgtgctgggccacgctcgaggacatcggccatccttcctcagctaatccacgcagcggcagatttgtatcagagtgtccggtgtagctttccgtgaggtgcgcactactgtagcctctacactctgaaattgtttgaagtaggtctcactcccctgggtatctgtatcctgcagccttacattggcagctatcgcgcgattcgcacttccgaaggcgcttcgagatactctacttcaatttggcgacgatggcaggacttgaacctgcgacatacgatttaacagaccgccgttctaccgactgaactacatcgtcattatccgtgcttgtttgcttcaattgcccttaattgggcatcagcgtcagctttggtgtcatgtgtcccaagaaccttcgtcttggtgtggtcaaggacaacccacttgtcgcctcgcttttcaacaaactcaacaatctctttgttgtcagtagagaggccatcccactcagcaagaaactcaccgaacgttttcatcatactcaccaatcgtttattgaacaactggtttatttaggtgctcccgaggcggtattttaatcccgcgatcccatctcgcctgtcgtagaccggagcatgctgacagtccaacatatttggtgaccctggctggacttgaaccagcgaccaatcgattatgagtcgactgctctaaccactgagctacggggccaaaattggcggggtctgaggcttttcacctcacctgctggattggcctccagctgttctctctggggactcgctgtccggacagctatcccttaaactatcgccccttaattcatattgctgtgggtaataccaaagcggaggtttggaatctctcgccctatagcgacctttaaaccgtttgatcagtccgatccaaaggactacccacagcaatatgaatttgttgtgtccaatcaaggactgtcaaccttttatcccgtttgtgcatgatcagttcatgcaggcctctttgtgcgcctcacagcaacgcgcttctgtaggtcattgacttgcaccgtagtctcaccacaggcgtttatttttccagggctttccacttacaagtcttaaccagatcgggatacgggcttgtaagatcagattggacacaactttgatggctctaggactaaagatcccgcgtcacccatagacttggtttataaaatctagagccatcaaagttgcaaccaatgtcgcaactcgcacttcagcgtcttcgtctcgcttcagcacttgtcctctacgctcggaccaaacgaggctggccgacagaaacatgacaccactcatcactagagcgaggatgtggttgatagccacgatgttacatctctgtcgcagagtgatcacacttgtcaggatgatcactgttttgcgaacttgaaaatactgcgcctgtctcgggtttccccttagttcggcctctcattgttcgtctagtcagggagctacccttgatgaccagcttattttgagatcttcgccagcacccactagtgtctagaatatgtaggtgtcgaggtctgcaaggcattgccctatttcatacaacgctctctcaagtacgcaaatcgaagttctctgggcttaacaggttcgacccaacatgttgtatatcgcctaacttgctcatgggacggacacatctgtgtacagaccttgagggttaaaagatatacccaaagaacttcgatttgcgttgtgggggcaaacccccacacggctacttctttgacagggattgctagccctggggattccactgtttaaggctttatcccataacgcctgaggcgcgttttaacttattgagcttgcataactgcaaactgatgcgctgtgagacacatctttgagggcactcagtgtacaacaggctccgatatttcatagggcattactgcctcggatttaagttgcctttctcctgaaactccatgcagtctcgcgtctgacgcggaatgccctcaaagatgtggagccgttttagggagtcgaacccaaatgcttggccagcgctgaccttacatcccatctgacttaggcaacccacctctgcttactcaggagcttatcccctttggacagtcagatataacggctttgggattggaggaaggactcgaaccttctcatgtacgctcctgatttctcagaagtagcctgctcccatcgacctattaagtacacgctccgctcattgttcggcgggagcttattaatcacaccctcccgcctattgttagtgcgccgaattttgttatgaggaatcggaagacctcactggtgtatggctattaagctactgccaggaacgtgttatcattagcgattatttttgtggtcagtttctaaaaacccgcaaagtcacgcacaacgaaaacgaaaacctgtttgagtcctaaacctacctgtcttggtctcacatcgctatgagtggccaggactcaaacagcttgccgttcagtgagttaactatactttgtaacccgttattgataaaaccttttatttttgttgacgagggtgtggaatcgaaccacacttctgagcgctgccccgtgtcaccaatgcaccacctcaccatcgatttgccggaacttacgcatccggcctcgcctgttggtgatccttcgaatccgggaagattagtaggaactatgaatcaagttaattcgaccaacaggaatcctcgtgttgccctttgacgcaacattatgaaccctctatgaagactcataatgttgtcggcgtgatggagattagcgcccccgacctccccttatatgttggtgtcgacttgccaacggggtgctctcattcttctgagctaatcacgctacaacagggagtttcgttctccgacggtcaagtgcgtccactgacaccagcgaggtggatgtgacgcctctattcacacaagccccagagactaggggttcactgttttcggtttcaagcctactcacactgtgctgtttccctcatgtttgatactttactgcaagccagccaatttgaaaaatcctctcccctttgaattacactgcccacagcagtgcggacgaaccttcattcaaaaccggatgcgccgaaatgggcgactgtgtaccggactgatcaggtcgacccaggagagagcaggtaatcccgatcaggaggatgaggagtaacatcatgtaaaacatagtgttccctcccttccatgcaactaactttaccgcatttatttattgaataaagcgttattttatcaattctaacttcacaacgcggcgcagaagataaacatattcaccagacgcttcatgcgacatataatgtgtagaaggggtggaatggttgttctgggtttcttttaacccaacgcgttcagcccaaccattaatggagttcccgtaaaacccgataatcgcgccgaaataaatgcgctggctttctggttctaaaatatagttgacagtgaagcagatgtcagaacgaatagattctaattttataccaacccgaccttcagagaattcgttgcgaacatacaaatgacgaaaacgatcgcgccgcgcttgtgatgactcttccaattcttggcgaagtgaggatgttgcatcgatattaaatgaaaatgtcgggttttgtagatcggtacgggctaactctaatgctgattcaacaacacggcggtaattaatatttttgccagtcagacgtttcacttcttctgtaattgttccgaaaggaatttttgtagcacgttccccttcgtattttgctaacaaaggaatgatgtgtttcattattgctgtattaatgtgcatttcctgagcctcgtcaaattaattaaatgtccccactattatattgggaatatgtttattgaatataaccccaagaaaggccaataaaataatgacagcgattattaataggtaaaaactacttttctacaacatcaagaaactgtatcttaacgcggtgcccttcgagttgtgagtgttaacgaacaacgagaagcagcctccatggctgcggaatgggactgcctgtcaaggcggtgttacgagcgagggcgtcagccccctcgcgagtatattcgaataaattaacgcgcgagagcgttttgattccctttggctaaatatcttcaacaccatattcaaactcaggtgatggagattatcatggccacgcaaacagttccttcactcgacgtcagagcctttgagtacattatcaaacagcgaatgaaagctgatcccacctttaaagattatgactttgaaggttctggtcttagtgctattattcgtttgttggcatctgatgcgaacgccatagcctttatgcagaacatgcttaatggtgaaggccatttaaaaacagcaaatcaacgttctaatgttggtttgtctgctgcttttctttcttatacccccgacaactatcgggcggcatacatgtatgccaacatcaaagtaacgccgtatgacgccagtacagctcctaatgagatcattatggaccgccgtgtgatgttcgttggggcgaaggatgggagttcctacaactttactgttgaaaaacccgtgtccgccacgttgacaacagacggttattacatgttcaacaacgtgaaactgattcagggtaactggttgtacaagacatatgatgttgaaggaagcgcgatttcgacttatacgataccttcggaaaatgttgatatcaatcatctggtcgtacaggtacaggaatcagagtcctctgatgtttcaacgacataccagagatacaacagtccgttcgacctgaatcaatatgcatatctctattttgtggaattagggatagatggcctgtatgtgtttgagttcggagatggttatctgtctcgccgagtagaagatggcaacgttattttcctccagtatttagaaacttctggtgctgaaggtaatgacatcactagtttatcttccgcatcttcgataggcgggtttaaccaggttgatatagaattggtttccgaacgcagtgcgggtgggggagatccggaatctattgaagatacgaagcgtctggcacctttggcttatcaggctgatggcgctgccgttacggaaaccgattacggtgttttaactgaacgcttgttttctaacgttgcccgtgctaaatcatatgggggtgatacgctttcccctccggattctggttacgtttatatcgctgtgataccatctgttggggagactctttcagacgcagagaaggctgatatcgttgccgcgttggacaaatacaacgttgggtccatcacccccaaagtggtagattctgagatcacatatatccaagtcaccacgactatattttgggatccgacttcgacagtttatgttgaagaacagatgaaagtcgtagttggcaacagtattgtcaaatggggcgaaaacaatctcggtggatttgatcagttgttcgacaaagagatattgcaggaagcaattacgaaaatggaacgttccattaactcaaatatcacgtctgtcggttataaacgtcatttcaaacccgattatggtgtgttggatagctttactttcagttatgggagaagcattaaacctggttctgtgaaaataacagggtttaaaccgcttccggccgaagttgatttcacatattatatgcgcgatgacaatggcgatttgaacatgtataaagtgaacaacaacgacaccactaaagaatttttggttcaaaagactggtgttgtggattacgccaacggcgtcgtagatcttcagcagattacggtttcaaactataatactgaaggagtaacgatcgttgtgctaccagatggcctgaatcagaatatacaggcttcacaaaaccaggtattcaaaatcggtgatgtggttgttacgccagaggtgcgctatgtccaaagatcttaataatggtcacaatggggttaagtatgaaaccccgttgttttaccaaaacgattttcctctgttcatcgagttcatggacacgttctttaattggctgtatcggcagcagggcttcactcaagaagaaattttggcatatctggcagacacttcgagttggatgaatcctgagagtgaagattcacctgttaaacaactcatagatttaaaggccgacaaaacgccaggttctgaagccaaagattatttgtcagataaatttttggtcagaacatttgagaacatgatggctcttgatgccgagatgctgctagacgctgatggaaggccgttgctttctattgaagacaagaacaagcagatagacgactggtataatgattttgggtttcaacgaactgtcgataaatcatttcaagaatttggacattttattccagtcggttcggattctttattgacggccacgggcgatacgttctctgtttatattgaaggaactaaacgaagaactctggatcatccgcgttggctgaaattgttgaaacacatctataaaatacgtggaacaaagaaagcgattgaattgttcttttggatatatttcggttgtccagtcagcgtctattttacgaaagaagacataggcggactggacggaaattttgaatgtgacggcacgacagggatgcgtgatgattactattatgatgaatacacttatgttataggagtcccaggcgacgtatctgattttgaaggtgtgtttgagcgcgtcttccgtcagcatttccatcctgctggatttactgtattcctagaaagcacaaggagctaaaaatggccgatttcctgagtcaatatacaggtcagcagatagatcagatattgggttctgttgacgacaaagtaagtaaaaacgacgtcattgatgattttgaagtcagtgacccattgtttccgccttctgctcgcctgacttatgagttgaaaaggtctgtggatacgattaatgataccctgaccaacaaagttataagaacggatagtggcgaacagacaatcaacggtaagaagacatttaatgcgctgttggttactaacgggggaatccaagtccccactgggaaatctgtttcaatcgcggacgaaccaaccaactcaacagacggtgtcaacctgagtatgttgaggaaacatggtgtcagtgaagacaccgcgccagatgggtctggtttggctggggaattattgacttccggatattacaaattcaaactggattttaacagattggctgcgtatacggaattaactctcccaacatcagttcccgtgcaaacagattctggagttcgcacatggtcaatgactgcattttttaatgcgttatttgccgacaaccgttttaacggttatgtaacgacgaccacgtatgatgccgacatgttgagtctggagtctcagcttaacaatatcaacacgacgttgaacggcaaagtcaacacgtcaacttacaacacaaaaatggcttcgttggatttgagtatatccaacattaacacatctttgagcaacaaggttgaggtcagcacatataatacgaagatgacttccttggacggcagtatcagttctcttacgactggtaaggtcgatgtatcaacgtataatgccaaaatggccacaattgataacagtttggggaacgctgtttatcgcaccaaaacaaacttgtcatcactgacggtatcggcttctggaacaacagctctgccagactttgatttggtgaaactttgccagatattggtaacagcacaatggggtacaggagatgtcgtagacacatatcgagtcaccataactcgagatgggacgattaagtctgagctgctgatccagaaaagtacgagcggaactgtgacgtttacgggttctgtagttggtgggaaactgaggatttctgttgtcaatgccaatacgacaacaccttgctcagtcgattattctattacagcatctttctaaaactaaatacccctagatgacatgaatacatttaggggtattacaaatggccaacaaaccaacacagcctcttttccctttgggtttagaaacttcggagtcttcgaacataaaaggcttcaacaactccggcaccatagagcattcccctggtgcaatattgacgttccctgaagatactgatgttgcgggtctcccatcttctgtgcgttacaatcctgatagtgatgaattcgaaggttattacgaaaatggcgggtggttgtctttgggtggtggcggaatacgctgggaaacgctcccatacgctccctccagcaatctgttagaagggcgcggctatctcattaataactccactgggacttctacggtggttcttccttcccctacgcgcgttggtgactcggttacaatttgtgatgcctatggtaaattcgcgacgtatccattgaccgtatctccagcgggaaataacctgtacggctccaccgaagatatgacgatatccaccgataacgtgtcggcgactttcacttggtctggtgctgagcagggttgggttgttacatctggtgttggcctcggtcaaggtcgtgtgtacagtcgagagattttcactcaaattttagcatctgatacaaatgaagtgactctcaacatccctccgacaattgtggacgtgtacactgatgggaaacgccttacagaatccaaatattctttaaatggaaacgtaatcactttcgacccttctttacctgctggcactgaattgcaggtaatagaatatactccgattcagttaggtactggcggcgggggttcgggcgcttctgcaatcacctggatttacaatggcggatccgctatcggcggcgaaaccgaaataacgctagacatcgttgtagatgatgttccggctattgatataaacgggtctcgtcagtacaaaaatcttggattcacgttcgacccattgacgagtaaaatcactctgacgcaagaactggaaccagaagatgaagttgttgtggttataaatgggacaccaaacatttataaccagatcgatcgttctttacaggaagttgctcgcgcctacaattttacaaatgctcaagttattttgagcaacgattatacttctgcactggataataaagtcgttatttatgatacgacatcccaacgttcttgggggattccatctggaattccagcaggttctacagtggtttccgtcaatggggatcaactgaaatacaaaaatggatcaactgaaaccactgttacattggtgagcgtaccaggtagtgcagagaagttgaagaacgatctggcgtcgaactctggcgcgggcatgttgaaaacttctagaggtgataccgtagaagaagaattgacttttattaatcagaatatggtcgttcaaatttcagatttcgcgagccttagaactttagtgccagccacagcgggcatccgagtcaaattgaaaggttattattctggttctgaaatcggtggcggagaattcatttccgtcgctggtgatgctgttgatgatgctgggatgattgctcgagtgtcaagcagcttttattggaagagaattatcgacaaacatattactcctggtatgtatggcatcaaagggaatcttccagaagatccggcagtgaaactgaacgctatgtttacggctgcggctgcggctaacgcttatgtcttactgaacgatgttgatactctggtctgtaaaagccctgtcgtgatcccagcacattcattagtgtacggcaacaaacggaccatattgaaacgtgatgcatcgttgtcatatgctaatgatttcttgacaattatgaatgattcatatgtcaaggggatctatgttgatggtagccgtttagattctcagactcctctagaagcagttttggtgcgcatcgctgggaatgtgactatccataagagcgagatccgaaacaatgctggctatggcgttgttggtaacgcagtccctggcattcgtatcaccaagaacattttttccaacttcgccaaaggttgtgtggctatgtttggcgacggaacggcgacgagtgatgatctgcaaattaatgataacagattctttaacctgggcgcgggaaccatttatgttggcggttactcaaacttccttgtcgacaaaaacgtcggaagaggcacttacgtgggttctccaggaaaccgtctgtatgttgtgaccaataccaacgggactatcacccaaagcgctgggccagattttggggcgctgaaaacgggtatgttctttgtattgcctggtggttctgaacatatgataactgctgtcaacagccttacaagtgtgacagtaaccccaccaccgccgtcgaacgccacagttcgtgcaatcgcagggacaggggatcttctgggcatccaatcatgctcctacggcaccatcagccgtaacaacctgaccgattgtgtgacttacggaactggtggaggcacgatggaggggagcactctgggttgcaattatgtcacttgggaagacaacttcatccgcaacacaggtaagaacggaatcaacgtcgggcaatcaggtgctggggtcgccacgtgttctatcctgaggaacactctgatacaggttggtaacggcggtcagggtgttggcccagcgtatcttctgccttggttcgatacggtaggtatcgctctgtatcagggaaacccaggttatatggcgaatatggacgtcagtgataacaacgtctctacatggtctggcgatctgaatgacggcgctgcttggtttggtatgagcggttgtacggaagccactgtgacttgtacaggcaacaaatcttctggttattccgacggctatgttcgtggagatatattagaagtcacgttgacaggttatggtacaggggcggctgcgacttcattcgttagtgatggtgagtctatacagataaccgttcaaacaggtacttcaccaacagcttctccttatttttcagtgcggaaggcgatccgcacccgccatcaaccgatgataacggcgcagatcgtcaccacaactgggaacatgtctcattgttggggtatgcaatctagtacagcaggaaactggatggtgggtcagaatacaactccttcaggggtcacgacgtattacgcaaaagggtaatattgaatgcctaaataccccatattggacatcctttatggggtattatcatgatttctcaattcaatcagccacacggctccacttcaatatttgttaacaaacaatctatcgctcgaaatttcggtgtcaaagaagacgaagtcgtttatttcactgctggtattgatctcagtggatttaaagtaatttatgatgagtctacccaacgggcttattctcttccttcgggtattgtttcaggaacgactgcgataagtctgaacgaacaagccatcctcactcattccgctggctctgttgaccttggggaattagcagtaagccgcgaagaatatgtgactttacctggttcttttaatttcggccataccattaatgtgaaaaatgaacttcttgttcacgatgataaaaaatatcgatgggatggttcacttcctaaaactgtcgatgctggctcaacacctgaaacatctggtggcgttggtattggtacatggctgagtgttggtgacgcagcatttagacaggaagccaacaaaaaattcaaatattcagtaaagttatctgattattctacattacaggatgcagcgacagcagccgtagatggattgcttattgatgttgactacacttttactgcagatgagagtgtagattttagcggtaaagttttaatcattgaatgcaagggaaaattcattggcgatggtatgttggtttggaatggtttgggtgcgggttcagtaattaagaaaccgcacatgcatactaaaactacgccgtatactgtttaccgattcgatgcaaacggtaattgggttacagacccaacacaagttctggcatctgttcagcagcgtttggatgttggatataagccaaatattaacgatttagatatttgggacgaccttcctgataatgtaaaaaatcaggttgctggtgctactttgcgaataatgagcggcgataatatcatcgtagagaacccagaagctacatttggtggttatctatttactttatgtaataggattcttgttaagaatccacgcaattttattgctttggaatcgggcattacatttgagaaccatcatacaactgcatggggtactggcaactgggttgttgggggtgagattaaatatggctctggttccgctgtactgttcattcgcaatgatggtggtacagaccatgatggtggagtaagggatttaatatcctatcgcgttggagaatctggtgttaaaacctatcaaaatgaaataggtggtcgttcagccaggaactaccgtttagtgttcgacaatataactacaatccagtgttactatgatggtattgatgttaatgctgacacagggtcgccaactgaacgtgtggatgactactcactcgcagagtatccatggttccacctacctactcaacatatcatccgtaatattattactcgtgattgtatggggattggtgcttggtgggatggtcagaaaaatattattgataatgttgttacttatgaggcccataaggaaggcgtcttcgataggggtactaacaatgatattactaacattactgtagtaggtgcgaataaggatttaactaacctaaatcagattacctgtgagggaggtagtagacttcgtggtattaacatccatgcatatactacacaaggttacgctatatacgctccgtcttcagaagtaagtaatgtttcctgtgctggttccggtactaagaaattactatgtacctatataagcgatattcagggaggtaatatcaatgttcagcatagtgccaaccaaatgacacttgcaatgcaacctgctatgggtggtactacaaacccatctttgcttatgacggcagattgccaggttgctacaccagggggtgaggcaagtattgtcaagctttcggcaattcaggagggtgtacgtgtaggtgagtttcagcttaaccgcttaggctttaagcatatgagtatacctgctgcccctttacaattaccagagagcgctctggaacataattcatctataggattcttcttcggaagtgacggagcattgaggttgcttgctaaaaaaccagatggaagttatgtaacatacacactttaatttatcttgagcaaaaaccccgctttggcggggttcttattatacctttgttcataaattttattagttcagactaaataccccatattggcaaatccttatggggtattttcaaatgaccagaaatgtagaagaattattcggcggcgtaatcacagctccccaccagattcctttcacgtataaatcaaatgtcggtggagaaactttcatttctctacctttctatcctgtcgctggtgttgtcacaattaacggcggggtgcaagttccgttagacaacttcgaaatagacgggaatacgttaaacctcgggcgcgcattgtctaaagatgacgtggtttattgcctgttcgacaaaatcctttcaccagaagatacttctaaaggtattcgaatatacaaatttcaagccgtgggtggagaaactgaattcacgcctgactttacttcttatggtgtccagtctctttatatcggtggtgagtacaaaactcctgatatagaatattcatacaacagtacgactgggaaagtttccttacaaactgcgctgactgcaggggtgtgggttgttgcggagatgtctgttaaacaaccgaatatcagtccggcgttcgatcgcagtatccaagaaattgcccgttcggctaacgtaaaagactctgaagtcattgtcagcactgataccatatctttactcgacgacaagaaagttgtttatgattcagcggcgcagattatttatggtttaccaactatccctgatggttcagtgatttcttctgttagtaatgggcaacttacttataatccaggtaatgttgtagtggatttgttgcctttagaagattcatttattaatgtgataaacactctggggcgcaatgatggtgccaagtatatcggagaatgccattctgttgctgatctcaggaatactgaacccactatggatggacaacgcattattcttaagcaacacactgcgggtactcttcttggtggaggggtattccgtgcgttaattgatggtacaggaaagactgatgataacggcactgtgatcaaaactgttggaggcgcagcctggttacgtgttaatgctgatagagttaacccattcatgtttggtgctttgggcggttctaatgatgatactattccagtacaatcttgtgtggacagtggtaaggccacacaattaactggtgtacattacgttagcaatatccggttaaaatataatacgtcgtctatttatgggtctggattacattactccaggttgcatcagttgccttctgctactgggaattgtattaccataaaagatacatgctcccttattgtattagactcctttggggtatatggcacaggtgcacaacaaggcacatcatttactgcgggtacaacaggtatctatatagaaactccttcaggtctctcagctgattatccgttccacactaccgcagacccaagacgcgacttgtgtatttctaaggtccatatagcaggttttgatgaatatgggttaaatattgatagtggtaactttagtgttactacagattctcttttagtcaaccacatcaatcaggtgggcgtccgttgtgctactactgattggacttggacaaatatccaggttaatacctgcggtaaacaatgtctggttcttgatggttgtggtaatggtcgtattattggtggtaaattcatttgggctaactggcaaccttatggtacagtaggacagttcccaggcattactattaataatagccagaatatggttattaatggtattgaggtacaagattgtggcgggaatggcattgagattagcgattcatattcaatttccatgaacggattgaacaccaatcgtaacggcatcaacgctaacaacactttctacaacatcgtatttaacaaaagtgatgcagttatcaacggattcgtaggactcaattatgccgcgaatagtggttcaggtgctaactctagtgcgggcaattttcagttcctgtctaatgattgcagcgtcaccattaatggtgtggttgagactggttatatgggcattaactttattggtgataacaatattatcaaccccacaaattccgacctgagcattaacggattggttaattattccaagactggtttgcaaaccatgaacgagacccctacatttgatggtgttagcactacacctgtttatgtaagtgtcccatcttctgtagggcaagtaaatggtctgagactatcacaagccaacaaagataaattactgtactcaagaacagcaggtccagaaggtattaccatggctgctgttgtagtacctaccatatctggagctgaagtatttaacttcatggccattggttcagggtttagtgatacatccaacagtcttcatcttcaattagttatagacgcttccggaaaacaaacaattgctttgctattggggggcgatggtacaacccaaattttatctggggatttacctaacgaccttaaactacaaagtggtgtgccatatcatatagctattggtgctaaacctggatatttctggtggagtattcttaatattcagacgggtaagagaatcagacggtcattccgaggcgcttatttagctgtaccatttaattctatattcggattaacttcttcattaacattcttctcggatagcaatgctggtggggatgcctgttctggtgtaggtgctaaagtgtatgttggtatgttctcttctgagaatgattatgtagcttcacggtactacaacctgattaatcctgtagaccctaataagttaattagttaccgtatattggattcttctatttaaaaatatacccctcttcggagggtatttactagagaaaaacaggagattcttattatgaatccgcaatttgctcaaccgaaaggttctacttcaaaagaaacgaacaaagatagcattgcccgtaaatttgggtgtaaaaagtcggaagtcgtttatgccaaagcaggacagtccttgtctggttataaagtgatctatgataaggtgtctcaacgtgcctattctcttccttctaacattgatgctgtaacggtcaccagtttagttgacggcatccttacgcactctggaggcaccgtagatttaaatgaattggcaatgacacgccgcgagtttattactttgcaaggagactttgtttccggtgttaccgtcaatgctaagcatgaagtgatcgcatacggaaatagccgattccgttgggacggggatctgccgaaagttgttccccctaattcagaacctgtcataccagattgggttaacgttgatgccaacccagcactgtctgaattagcgaaatctgttggctcgtctctggtgggatataaaccaacggggacaaccactacgactggtacggtcaaaggaaaattggatagttttattgattttgcaaatgactattgcggtggcccaattacaggacaacctgatctcacttctcaattacaacaagcaattattgatgcttttaattctggtattgccgacattaagattgttggggatttttatatatctggcatgatattatggtatccaggtgttcgattgattggtggacgttatgactctacattgatacgcgtttccaatactttcccagtgggtgggacaatgtttcgttcttaccgtcctccactgtgggcggcgggttgtcatggtctatctcttgaaaatgtgtatttggtaggccgcgccgccaaggacgtttatggcatagatatcaatgatgcatcttattttaatcttgaaggtgtgcgtctggatctgtttgataaagcaatttcttttaatagatggattgatgaaactcgtgtagacacgagcggtacaattacctatcccaacgcgcatagtgccgaaatgggtggccaatcatattttggtacaataactcgttgctacgcaggcaattgcgtcacgtgcgttgattttaatggtgtggtaaaccgttgtacatttattagcaacacatggacaactagtgatctggcatataatttcagcaacccgcgtggagtttatgaaactaacacattcatcacttgtaatattgaaggtgtgaagagtgcatttgaatggttcttttctataaattcaccatatcacaatgtgtggattaatacgagcatcgataacgggaacccagatatcacgagtttagcgaaagatggtggtcgacagacatttattggtttagcgattttcccttatggaaacccatcgttggttaattggtatggaattaaccccaatggtcatcggagtactgtcttgggtactgatcttggggaaaatttaccagaagatcagttaaaaactcaggttcgggaagaattacacactttaacaggtattgccaacaagcaatgggctggacaacaagtaactgagacaattcccgccaatggttacaaagcattgaatattagcgtgccaggattaaaaggtaactcagcagttattgcttcattatctgccatatatcatggtattacagttgatgccgccagcaacaacactggtacagtttcagtaataataagcaattacacaggttctccaattgatgtaaatgcttacctgagtgtcacaggcatagccaaaagtttcttgtgatcaaacaaccccgcttcggcggggtttttatttctatctgtctgttggctgtatactaaatatctctataaatggcactcgatggagcatcaaaatgcaatctacaaatctaaaccgtcgcccgtattgggacgactggaatcctggaaaacggttttcacggattctttttcgcccgatgcctattaaggttcagacacgtgaactgaaccagatgcagactattctacaagaccagattgaaaagctgggaaaccatctgttcaaagatggatctatggttattcctggcggtctgacgattactaacgccgctgtgtctatgaaattcaccctggctggtggcactgagttcactgatctggaagggattactgaactttatgttctgggaaaagacaataacgccaaagcgcgtgtgctatctcttgaacgtgatctgtctgctccggatacgatgttggccatccttgaaatgacagaagtgggtaatgcagatggcttccacgtcaacgacaatctgtatttccaaacttatgacgtcaatgacaacttcattcgtattggttatggtatcgcagctgcggtgactggttctatcgtcgctcgtatgacgaagggcgtttacttcattcgtgggatgttccttgatgttgatgacgcgactctcatcgtcgataaaacttcaaacacttcttcccatcgcgtcgggtttaaagtcaccgaaaccatcgtgactgaatctgaagacgaatccctttattcaaacgctcagggaacgattaactccaaagccccaggcgctcatcgtcttcgaatagatctggtcttatctcgatttgattatgatgctgtggttgaagactttgttgaactggccaaagttcgtgatggtaaaatccagtctatggtgactcagtccacctataatattctggaagacacgttggctcaacggacgtatgaaaccaacggcgattacaacgtttcaacgcatcagatcgacatccgcgaacacctgaaagtcaataacaatggtggtgtattcaatgctgctgatggcggcgacgaaagtaaattcgtgtctgtaatgaaaccaggtatttcctatgttcgcggtcgtcgtatcgaaaacgtcggggaagagttggtgatcgttgacaaggcacgggataccgatgtcctgaataacacgcctgtcgcagttgctacagggaattatctggtcaccaagaactccaaaggtgttccagtaatatctcgcacagtgcgatacaagttattgaacgcatctggcgctgctcaagccacggcgttgtgtatatccgctgaacgcaattcaactgaattccgtctgtacatgcgtgatttggttgtgactggggacgcgtccactatcaccaaagtgtcttatgaagaaagtgggatcaccatgttctctgccgaactggaatccaaccagtttaaccagagttctatggtagacctgatcttctctcttccggtcttcggtgttaaaacattagcgccaacaggttccgtggatatcaactacacagtcctcagaacttataaggtgacgctagacagcagcggttctggttctatttcagccccgctgggatatagtttctctcctgaattttcgttgtattcttctgcgaaatccgatggttcagaagcgcaatttgatatctctggctctttgtcactgactggttctccggtgggttcggctttgcagatttccctgggtagcggtaatgctaaccagtcaatcaatttgctggcactgatgatccgcactacggccacgatcaaaacgaagacgataactgaaaccacagagtctgtgacttttacttcccaaaccagtagacctctggcaaatcacgacggctggaaattggtatctgtcaaaaatgatactggcgcggacgtgacttcgagcttcaccctcgacggtggtcaacgcgatgcgggatattacaaggctaatctgttatccaccgccggagccatttcagggacgtatacggtggtgtatcaatacttcgcgcatagttcgggggatttcttctcagcggattcgtatacttcaatggattacaaagatatcccgaattatacatcctccacatcaggggcggtgtatggtttggccgatagccttgatttccgtccgaaaattaccaacgggacatccgacactgatatggttcgcccgaatacggctgtcatcctggacacagagtattatttgcctcgtatcgacgcgatttatctggcagataatggcgtgtttggtgtagcccgtggcatcagttccaacaacctggcgtccccagcaatacctgcaaactccatgcgtttgtatgaactgttgattcccccatacacgccgaatatcgatgatatacaaattcgcaccatcgataaccgtcgttatacgatgcgtgatatcggtaaactggaaacgcgtatttccaacgttgaatactacacctctctgtcgcagctggaatcttcggcgatgacacaacaggtatttgatccaatcacgggtaatccgcgattcaaaaacggtatcgccgccgatccattcaaagacttccgtcttattgatgatctgtctgaagattggatgggatctatcgatacggaaaatggacgtttgcgtccgtttgttcaacagaacgccgttgacatgactccagtaggctggaacaaagtgatggatggtatggtggtttgtaactacacgcctgaaatctccgtaaaccaagaatacgcgacgacgacgatcaacgttaacccgtatgctgtattcaactgggaaggtttcttgaagattaacccgaccacggattactggtttgaaaactactatgtcgccccgcgtgttatcaatgaaacgatcaacactcgtggtactgtacaagaaggttcggtgtacgggacgtggcgtactgtttctgtttctgatcgtgtttgggagccgcatggcgctggtggtgtgtggtggggataccgctatcgcacgactgtttctgcccgtgatgtcaccacatacacatacacggacaaaacaaccacgaccatgactggcgaacagattgtggaaacgcaggttatcccatacatgcgcgaaattgatatttctttcgacgcttctgggttgcgacctttcactcgcatgtatgcattcttctcaggtcgtgatgtgaatctgtattgcaagccgaatggcggtaattttggtgacccaatcaccactgatgccaatggcgcagtcaaaggcgtgttccgcgttcctcagaacgatacgatcaagttcaacacaggtgacaacgtgttccgcttaacggatagccctgttgacagtaaatctgcagatgatacgctgaccaatgctgaaattgttcacaagtctttcggtaagaaacaagggattcaaaagacctttgtcaacactcgagttctgggttataccgcaagcaccagcaccgaaaccagcacttctgaagttgtggtcgatcaatggcgtgatccaattgcccaatcgtttatggtggcaactaagatcggtggtgaatatatcgaaggcgtggaagtattcttctccaccaagtcacgtgatgttccgatcaccctggaaattcgcgagatggagaatggattaccgtcccataccgtcataactcgtaaaactttgaacccgtctgaagtgacgatctctacagactcttccggcggaacgaagttcaccttcgattatccggtgtacttgcaagcatcgactgagtttgctattgttttgttggcgaatactcaggattacaacgcgtatatcgcggaaatgggcaagaaaaaccttttgtccaacgaatatatcgccaagcagccgtatacaggggtgttcttcacttcttcaaacggttcaacatggtctcccaaccagatggcagatatgaagttccgtgtttatcgttgtaactttgctgctggacagaacgttgtaacatttgatccgaaacttggaccgaaacaacgtccgctgggattgaacactctcaactgtgtgagcgggtcttctgtcgtgaccgtgtttgcgcctggtcatggtttggttgctgggaacaatgttactctgtctgaattgacaggtggctgtggatttactcctgaacaacttaacaaaacatttaccgtgacagatgcgagctatacttcattcaagatcgatgtcggttcagcggcagacagcaacgggcaaatcggcggggataatgcttcattcttgggcaattatctggttgacatgttctatgccagtgtcaccaattcagctctggaaggttcaattctgaaattggaatatcgctatcgcgatgctacttccaattctatgtctgattgggctgaatttgaaactgacactgacgtggcgttgccgactgaagggatctatcgtcaggttggggatttccagatccgagccaccatgacgcgcagtgaaaacaacgtgtacaccgctccaatgattgatggcgatgacctgagcgtaatcttcaactcatacggcgtggatccatttgaagatgtcttcaaatatgtgacaaaagacatcggatttgataacccatgttcgactgtgaagttgttctttggtgcaatgctcccatctcagtcttctatgaaggtgcaggtgaaacttcttagagcaggtcaggagatggatagtgtggcttgggaagatgttactccaacgtcgcctctggtcaacgacggttctacattctttgaatatgaatacgacaagactgtggcgagcaataacccgtttgttggcctgaaagtccgagcgcttgtacgggggaaccgcgttgctcctccatcattcaaagacttccgtcttattgctctggcataaataatgttgaaaggtgggcgtaaagcccaccaaaccaatagaggataaagatatgcgtggaatgaaagtacaggggcacgcatccatgttgagaagcagttcttgtccaggcgcgatcatttgtactgatcatgctgcaggtatggctgcattgcaagcccgtcaacgcaacgaaacccgtgaagccaccattgttgaacaatcaaaccagatatcgaatctggaggcgacgatcaaaatgatggccgaaaagttggggatagaaatccccgaaggaggtcagaatggcgacgcaagctgaaagatctgggcttgatatccgtggtatcttgactgctgtcgtggcttctgcattggtctcggcggcgtcgttcctttggtttatgggtgggatggaaacacgcgtgaatgtattggagcgagattctaataaaatggatcaggtccttcaaaaggtgaatgacatgagtgagcgcatggcgatcatgaatacagacctcgcatatgtcaaacaaaatatggctgagttaaaactcaatagcagcagcctatcagacgatgtccgcacattgcggattactgtatctgatttgcagcaacgagggaataacaatggccgtcaataaaattcgcgacaagaaatcttttatgaactatgtcttgcgtaaattgggcgctccggtcatccaaattaaccttgacagttcacaagtagaagatgctgttgatgatgctctacagaagttttgggaatatcatcgcgacggtagccaagatgcgttcttcctgtaccaagttaagcaagaagatatcgacaagggttatattgaattcccagaagatattgacgatgtgattgaagttatccctggacctcctattgagtcaattggaaactgggcaactcctcaatggcaaatggcccaagccatgcttgtaccaaaagcggcgcttgtgtctattcgtctcattgattacgtttccatgcaacagcgcctgtctgatatcacaagcgtcttgaatgttcgtcgtaattttgtgtacaagaagttccagcgacgcctttacccgcaatttgctgccaccattgatgaaactctggctttccgttgttatcagaatatcgaccccgaatcggaagaaaacgctgaagcatggaatgacatgtggttgaaagcgtatgcgactgcattggttaagcgccgttgggctgaggtgcttaagaaggcaagaggcatccgtctcccaggtggtatcgaactggatggtgatagtatgttcagtgaggccgagaccgagatagagcggttggaggaagaattgcgtactggtcagcaataccctatcgattttatgatgggataagagaaggcgggataacccgcctttctttttataacttgatgtcgcgttcggaaggatttgaatttgctagaatataaccatcaaccacgttcttttcatcccaccattcaataaaatcattcccatctttgcctttaataagacccacaatgttataagaattatttgcgactttagcgtccacacagataattctcacacgccttccgtcacgagtttctttaatatcttcaaccttcatttatcaattcctcaaaagaattaatgattgactaaataccttcaaaattgagggtaaagtcatggctacttcaaaatatttcaactatacagcacaccaaggtactcagaaattaattgacgacttggtggtcgagatgatacaactacgtgggattgacgtcaagtatatcccacgttccatcgttgaaaaatacccaattttaaacgaggctgaacataaattcgaccaggcgttcgatatcgaagtatacatgcaagattaccaaggctttaacacccaaatgtgggagaagtttggcggtattcaattacaagatgaagtgacgttcaccattgctcgtcgccgtttttcggaagtgattggtaatgggccaggacttgagcagatgcctcaagaaggtgatttgatatacctcccaatggcgaacaagatattcaaagtgaataacccgaataacgacgaagaatttatgcaattcggtaaatggtacacatattccttaccgtgcacgttgttccaatatggtaatgaagattttgatactggtgtatctgaaattgacgacatcgataaacgtttacaagatctagacggcgatggtgtctataaagatgccagtctacagacagataaccaatttgcggacgaaatagagaaagatctttcgcccaataaaatgaagatagacttcggggaataatcatggccagaccatttgaaaaatatttctaccatgaatcgttgttgaaatacattcatgtcttcaacgctatcatgtcagatttaaaagtcaaaaccgaacgtggcttgatggaaatcccgctgcatatggctattggccgtcgcaatgacctcaacaggaacgtgccagccaatatgctgccatttgcgactatgtccttcggtcaattcgaaattaataaacaagtgaccaagtcgtatcataatcagatatcgactgcaactgcgcggtccaaacaacgaattccgatcattatagattttgaatataacatcagaacgaagaaactggtcgaaatgctacaggtgttggaacaaatttattctgtgttcactccttctattgattgtcagataaaagacaacgatacgttatctcaagatcaaaacgtgaagattatgttgacatcccataccatttccgataactgggaaggggatgccgctgaatcaccccatatagattgcacctttaattttcaattacatgggcacatttatggcgaagattattgggtcgatgacggctctggcggtggtgatccgaacgttatcaaagagataattattgaaatgtctaatgatttgaacacgccatggtcggaactgcctgagtggttccgagttgacaaagatggcatacatcatccggaggattgatcatgagcaatatgtcagaaaggttacttgcgacgctagacgcggtgactgccagagatgaagttggtaaggaggcgatggaagccgtctctccacgtccaggtattgacttcgatgaaaaaacgggcgagtggtttggtgaacgcccagaaggttatcagccgattccagaacctccatcattggaagatatcgcgtccaaagaatccaaggttccggagtttgcggatactgatgccacaacagattacaagcggatacgcgatacaacatacgccatgcaggaagccacgatgttcatgatggggcaagctgctaaattagctgcatctaccgaagccccaagagcgttttccgtgtttcgtgaattgggcgaactcatgcgcggcctgaacaaagacctgatggaaaaccaaaaaaccatcaaagctgtgacaggggataaagaaccgccgactgatgacacaaccgtggatgttaccacttcacccgacgggaccacaacagtatctgttgggaagaaagcgagatcttcgcgcgatttattgaaaacgatcgaagatgcacgccgccgcgctgaagaacgcgcccaggcaaaagccacccaacaaaccgaagatgaaattatcgacggtgaaacggttgacgtgaaggaagaagacgatggcgtatcagaagcgtgatatagaatatgctccgataaaaacggggtttaaaatcgacgacgtgaaattgcgtatggaccagacgttcatgcgcaagccttctgtgcgcgctccacgagtcgagttgatgctaacagatgaacaagaagacgaatttgttgaatgttcaatggacgcgcattacttcgctgccaattactacaagataacgacaatcgataaaggcttcatccttttcgatatgcatgattatcagaaacaattatttcacgattttcaagattatcgtttcaatgccgttgtccaggcgcgtcagtccggtaaatgcgtccgtggtgatactcttgtttatgtctatgatacagtcagtcaacaagagttgcatctcaccatagaagagcttcacaagcgcttcgaaggaccgaaccatgctgtgccactgaataccattggcaaccacaataagtttgtagacagccgtttcgggaaacgatattttgttgagtccgacactggctgggttccggttattgccgctcataaaacgaaagaatacgctgaatacgtggtcaggactgaaaccggacgcacaattcatgtcgcagatgagcacatgttcttcaatgaatatgggcgcgaagtatttgctaaagacatggaggccggagatgccataatgacccaggagggaatagaattcatctctgagatctgggagactggtgagtaccaccacatgtatgacttgcaagtcaagtccagcgatcaacgttattacacgaatgggttcctcagccataataccacggtcgtggctgcgtttcttctttggtatgcgatgttccactcagacaaagaaatcgcggtactggcaaacaaagagaaacaagcgattgaaattcttgaccgtatcaggaaggcgtatcaggacctcccattcttccttcagcagggttgtgagaagtttggttctacactgatagagtttgagaacggctctaagatatatgcttatgcaacgtcttcggactctattcgtggtcgttctgtatctctcttgtacgtggacgaagtcgcattcatcgaaaatgactttgaattttgggaatcaacattcccagcgatcgcatctgctgatacttcacgttgtattctgaccagtactccgaaaggccagcgagggttgttctatgatatcgtcaccaaagcagacccgcgccatccgcaatacaacgacttccatcttaccgaagtcccttggtacaaggttccggcgtacaccaaagaccctgattgggaaaccaaacaacgcgctcgtctgggggatgcccgttttgaccaggaatttggaatcaagttccgtggttctgtaggttcattgataccagccaaatgcttggataaaatgacatccaagttatatcgagagcctaatgagttcaccaaaatttacaaggaatatgacccgcaacgcatctacttcgggatcgcagatacggggaaaggtgttgaaggagattattcggttctgacaattctggatataacggaatacccgcatgtcatagcggccaagtacaggaacaatacaatacctccgatgatgtatgcatacacgatagctgatatgtgtactgaatacggggaatgccctgtccttgtcgagacgaataacgacgttggtggacaggttatcacgatcttgtatcaagaaattgagtatccagagatcatattcacgtcgacggataacaaagggacagggaaacggattggtggtcgtaaaccagagccaggcattaacaccaacaggaaagtgcgatctattggttgtgctaacctgaaagcgcttattgagaaagaaatgttggtgattgaagatcaagacacgatagatgaactcagcacattcgtgttccgtggtgcgcgttatgaagcagatgatggatgtcacgacgactgcgttatgccgttggtattatattcttgggcagttaaacaagaatggttcagtgatctgacttctaccagtatttctcaagacatgagaaatagaatgtcttcgactgagtcacaacaagtattcccatttggtggtttagtcgtgggagacactccatctggaactgagcatctccctgggttcggtggtgttcaagtcttcgacgaacgttcagggatgacaatggacgagtggtttaagaattgatcactaaatatctttcaaattgagcgaaactctaccgagaaggaataacattatggcaactcaaagcttcagcgttgcgccgtccgtacagtggactgagcgtgatgccacgcttcagacttctccgtccgtggttgttcagggcgcgacagtcggcaaatttcaatggggtgaagtcgaactcccagtgctggtgactggtggtgagacgggtttggtgaagaaattcttcaaacctaatgacagcactgcgacagatttccttgtgattgcagactttttgtcttacagctctatggcatgggtcactcgtgttgttggtcctctggccaagaactctgtcaccaaaggccagacagccatcacgatcaaaaacaagctggattttgaaacagcaagtccgtcagcgtctatcacttgggctggtcgttatccaggatctctggggaatgacatcgccatcaatgtttgtgactccgctggattcccgacatgggaattccgtaacaactttgcgtacgcacctcagtctggtgaattccatgttgtggttgtagacaaagttgggcgcattactgactctgctggtgcggttggtcaggttgaccgtatctctgtttctggcacggcaaccgcagctggcaccatcagtgtggcgggtgaagatatcgcatatctggatactgatactccggccactttggcgaccaaaattggcgcggcgctgacggcgctaacagatgtttattcttctgtcgttgttaagtctaacactgtcatcgtgacccataaagccattggtccccagacagttactgctattgttccagatgataaggggctgactgcaaccgccgtgattaccactgttggcgcatctggttctattatcgaaaaatacgaactcatgcagaacacacagggttccaaaaagtccgatggcgcgaatgcgtacttcaaagatgtcatcaatgatacatcaaattgggtgtacaccttcgccaccgctctggccgcaggtgttgttgaattagaaggcggcgtagacgactacaatatcaaccgtgtcgcggctatccaagttctgaacaacgccgaagcatatgatgcgaagccagtatttgcatactgtgaagagttgattgaacaacaggcattaatcgacttgtctaccgagcgaaaagacaccgtttccttcgtgtcgcctttgcgtgatactgtggtcggcaaccgtggccgtgagatggacgatgtggtcgcttggcgtgaaagtctggtgcgcgactcctcctatttcttcatggacgacaactgggcatacgtgtacgataaatacaacgataagatgcgctggatccctgcttgcggcggtacagcgggtgtttgggcacgtagtattgaaatcgcgggtatctacaaatctcctgcgttccacaaccgtggcaaatacaacaactacaatcgaatggcgtggtctgcgtcttccgatgaacgtgccgtgttgtaccgcaaccagattaacagcatcgtgaccttctccaatgaaggtatcgtgttgtatggtgacaaaactggtctgactcgtccgtctgctttcgatcgtatcaatgttcgcggtctgtttattatggcagaacagaacatcgctgcaatcgccaaatattaccttggtgagaacaatgatgcgttcactcgcagtctgttcagcaacgctgttcgtccttatattcgccagctggcaaatatgggtgcgatctacgatggtaaagtcaagtgtgacgaagataacaacactgctgacgttattgcggcaaaccagatggtcgctggtatctggctgaaaccggaatacagcatcaattgggtgtatcttgactttgctgcagttcgtccggatatggagttctcggaaattgaatccggcggcggcatcgttgctgcttcttaaaagatcaaccccgcttcggcggggttttcctttatcccacatatttgagttattgaatgatatacgcatacaaaattgaaatggtttgtaatggaaaggtctacatcggtgtgaccaatgacccacaacgaagatatgatcagcattttgcgaatgcctttcatcatggcgtccaaagtgaattgtatgacgccatgcgtaagtatggaacatctggatttactttcgttgtaattgctcagaccgatgaagtccataaatgggaattagagaagcaactcatcgctcaatataattcttatgagatgggttacaacatgaataaaggcggtaatgatggaagtcatatgctgggtaaaactgcagcaaaattgacatccacaggtgaacacattggtcttgtttctctcatagatccccgttgggaattgtgtgaaatcgaacctgtttcaagtaatgttcatcatagtttcgatctttcttctatgaagattaaaagattgacgactaattagaattgtgtactcttatttggagatataaaaatggcgacagtcaatgagtttcgcgcagccatgtcacgagggggcggcgtacaacgccaacaccgctggcgtgtgactgtaaactttccttcttttgttgctggttccgacacaattcgcgacgtgtccttgctggctgtaactaccaacaccccgacaggtcagctgggtgaaattctggtgccctggggtggtcgtgaacttccattcccaggcgaccgtcgtttcgaagcactgcctgtaacattcattaacgttgtgaacaacgcgccgtacaacgcatttgaagtgtggcagcaattcatcaatggtagtgaaaacaaccgtgccagcgcgaatccagatgattatttccgtgatatcatcatggagcttctggatgcgaatgacaatgtcaccaagacatggactttgcagggtggctggcctcaaaacctcggccaattggaactcgatatgtctgcgatggactcttatacacagttcaccgtcgatctgcgttatttccaagccgtgtcagacaaatctttataatgtcctcggtgctgggggaatcatattccccctcactaattacaagaaaatgttgaggacttagatcatggctggatacggcagagggttcttcggtttgtttggcggcggcggtttggtgaacgctaaagtcgacaccgataagttagcccaaaaacaagatgaacggttgctaaccaaagcgacagttgttgctctggacgacgctcaagacggttctataatccttcagggtggtgcgaacacctacaactatgttggcgttgaaagtgaacttctcagcgttaaaacagttgtggaagaatatcagtccatggctcaacagcctgaaattcgcaaagcggtggacattattgtcaatgatgttgtcacctgtgaggaagatgaaactccagtgacagtaaaccttgacaaagttgaagggatatctgatactgttaaagaatctatcactgaatgcttcaaagaagttatgcacttgatggactttgacaatacggcatatcagaagatccggaaatggtatgttgacggtcgacaagcatatcatgtcatcgttgatcccacgaataaaaaaggcgggatcaagaaattagttatgcttgattctcgttgcattcgtcctgtctatatcgttgagaaggcgatgcgtgaaggcggtattgaagcaatagaatccgtaacattgaagtattattacaacccgaattataatcgaaaccaattcactggccaatctggtacttcccagaacttccagccttcacagcaagaactcgtattcgatgacgaaagcattgtttacatcgatagtggtgaagagccattggccaatggtattgtcccaggccttttaaatcctgctatccgtccgttgaacaacctggttacgactgaagatgcgactgtaatttatgccatcactcgcgcccctgagaaacgcgcattctatcttgacgtcggtactctcggtaagaaatctgctgaagaatacatgaccatgatgatgggtaaattcaaaaaccgcaacgcatatgaccgcaccactggtaagataacaggtaatgcccatcttatgggtattgcagaggattattggttgccgcgccgcgaaggtcagaatgctacagagatcgcgactgttgggggtgggaatcaattgggtgaaatggatcacgtgaactatttccgtgaaaaactctatgatgctctaatgatccctaagagccgcctccaagaggaaggatctattaacattgggggttctaaccttgcggagattacacaggaagagctgcgtttcagcaagttctgtgctgggttacgtcgtcgttactcccatttctttatggagtttttacgtcgccaattaattttgaaaggcgtgactgacgaaaaagattggaatgaaaaaatcaagccgtttattaagtttgaattcacttctgatagctatattcgtgagcaacaagaaaacgccatattaaatgatcgcctggcttctctgaacactgttgagccttttgttggttctatattctccatcgattatgtcatgcggaatgttctgcgtatgtcagacgaagaagttaaagaacaacaggctaagatcgcggaagagaagaagaaaggtctctatccgaaggttcaagcagatgaaactggcaattatagtggttcagatgttagtccgttgaagtttaaacctgagactctcccattctccggttcaacagacgatagtatttaaagttcaactaaatatagaaaattaattcggagatcaaaatcatgagcgcaattgatatcgttcgtgcagtaattgacggtgacactgaaactgctgttgcagaatgcaacatggaacttgatgcacgtagtcaagaactattgaaccaaggcacagcatatgttctggattctatcgcagccgatatgaattcaaacaacaacggccagtaaggagatgcatgatggaaatcacagagatcgccacgttcgctgatttcctggcctctcgtatggacgagcagcgagtcatcgataaagtgaacgctcgtggtaaacgccgccgccgcttgaaatgtgcccctgggttcaaattgtcggctgatggttcacgttgtgaagttatggacgccagcgaacgtcgtgttcgcaagatcggcaaccgcaaagccctccgctctaaaaagcgtatgggaatgggctatcaacgtaaaatcgagcgccgcaagaaaaaggctatgaagttccgcaaaatgatgggactgagtaagaacaagtaaggagtttatgatgaaactgttgcgtgagatcacagcgatagggaaggatcttcaaatcggtgaggccacaacctcaactggtgggaaggccatgttcatcgaaggtccgtttgtgatgtgtaaccaagtcaaccgtaacgggcgtaactatgatctacagaaagtgggtattcctgctgtcgaagcatacgacaaagagtatatccaagatcgtcgcgcaatcggtgaagtcacacatcctgactatcctttccctaatttagtggaagctgcgctcaaaaccgaatcccttcgatgggaaggcactaatgccatcggtcgggcgagaattttaaacacaccaaaaggccaaatcattcgtgcattggccgaagcagacttcaatctggccgtatctactcgtggcctgggtgagactaagtcggtaagcggttatgatgatgttcagcctggctttatgcttaccgccgttgacgcagttgaccgtccttctggacaagtttgttatgttaaggctgtgagtgaatctgttgaatggcagctggacgaggcttcgggtatttggatgcctcgtgatgttaaagggaaagttgtagaccagttggtgaaagccaatatccaggttgaagacgatttcctgcgccgccttgatgcagcattgaatcatctgggctgaatttcagctcactaaatactgaaaaatcgttcagaaggaaactatcatgaaacctgaattgcaaaatctgtttgaaggcgttaacggcatcagcccagatttcctggataaagtatccaatttgctggaatcgaaagttgaagcggctcgtctgaaagctatccaagaaaccgaagcagctggcaacgtcgagcgcctgaatctggtagaatctcaccagaaagaaatcgccgacctgaaagaaaatttcattctgcaattggctgggaaagttgactcattcctcaacgcggtggttgaagaatgggctaacaaaaatgccccagctattgacgctcagatcaaaactgaagctgctgaacgcttcctcactggtttctccaacgtgctgaaagaagcaggtgtgagtttcgctactgacccagacggtcagattgccgccctcaccagtcgccttgctgaagcagaacaacgcgccaacatggctaatacagagttgagccaactcaaagaaagcgagactaaacgccagcgcaccgacgtaattgatcgcatttgtgaaggtatggttgacaccaagaaagacactgttgtcaacctgctggaaggtattgaattccagaccgagtctgaattcgaatcgcgtgttcgcaccttccgtaatctggtagaaggtaaagatgacttctctgacaaagtcggtaaagacaatgaaaagggtaagccagatggcgacaaggctgaaaaagacatcaaagaaggtaagaaacagaaaaaagaaggtgatgatgttgacgacgataaagacgacgacgacaaagacgaagttggcaaagaagtcaacgaagccgtccgtcgccagatcagcgccattctgaacggctaattttagcagccacagccctcgaaagggggcttgattttgaactactaagtaatttcaacttaactgaatatcaatgtaaggaacgagcatcatgactaagaaacttgtaaccgaagaaatgcgcaaacagtggctgccagttctccaaaaagaatctgaagccatccaacctctgtctgccgaaaacgtaaccatccgtctgatgcaaaaccaggccgagtggaacgctaaaaacctgggcgaatccgacgctcctggttctgtaaacagcactgtcggtaaatggcagccagtcctgatcgacatggcaaaacgtctagcgccgatcaacatcgcaatggacttcttcggcgttcagccgctgtctggtcctgatggtcagatcttcgcactgcgtgctcgccaaggtgttggtgacggttctactaccgctcaggcgcgtaaagaactgttcatgaacgaagccgattccggctactctggtgatggcactgtacaggctggtgacccgtcaggcttcagtcaggctgagatcgaaggttccggctctgtcgtgaccactatcggtaaaggtatgccgtcaaccgacgcggaactgctgggtactaccaccaatccgtgggcgcgtgttggtattaccgttcagaaagcgaccgttactgccaagtctcgcggcctgtatgctgattacagccatgaactgcgtcaggatatgatggcaattcacggcgaagacgtggacaatatcctgtctgacgtgatggtaactgaaattcaggcggaaatgaaccgtgaattcatccgcaccatgaacttcagtgctgttcgtttcaaaaaattcggcaccaacggtgttgttgatatcgcacaggacatctccggtcgttgggcgctggaaaaatggaaattcctgactttcatgctggaagtcgaagcgaatggtatcggcgttgacacccgtcgtggtaaaggcaaccgtgttctgtgttctccgaacgtggcgtctgctctggcgatgtccggcatgctggactatgctccggttctgcaggaaaacaccaaactggctgttgacccgactggtcagaccttcgcgggtgttctgtccaacggcatgcgcgtctatgttgacccatatgctgtagcagaatatatcaccctggcgtataaaggcgcgaccgcgctggatgctggtatcttcttcgcaccgtatgtgccgctggaaatgtaccgtactcagggtgaaaccaccttcgctccgcgtatggcgttcaaaacccgttacggcatctgtgctaacccgttcgttcagattccggctaaccaagacccgcaggtttacgtgactgctgacggtattgctcaagacagcaacccatacttccgtaaaggtctcatcaaatctctgttctaatcaacaggttttgataaacaaaccccgccatgtgcggggtttttctttatagggattcactataagaccgctaatagttttcaataagtgggcaatgaacgatcattacctaactttcacaactggaacgctactcatggctaaagaaactctgactaaagaaaactctaacttcgcagaatggcatgcagaattagagcgtgtcgctcgttctcaacatggctctgccgccgatgctatggcgtggcatgaagattatgagtctggcaaaaccccgcgtcaggcttgggaagatgaatgggggtggaatgatgcagactctaattcgtaagatatatctggataagaactatttggaatccaaaatcgaagttttctcggctcaaaaaataattgaagacggtgaaacgagatattatctgaaacctgacctttatcttccaggaagtagtttgaatcgcgatataattcagtcggacggtattactgttcgttgggaaatggcgtcacaggaagacaactgtcatagcataacactttgaagagagatctatattatgtcaaaagaaaaatacatattcatcagccccgctacgaaggagttcttcatctccgacgtgaaagatcgtgttgagaagttccgctacatgcgcagctatggtctggcatccggtgatattgattatcagtcactggacaatgatcgccaagatctgatcgacgacggttggcgtttagttccgtcccctcagtacattcatatcacgttgtactatatgaaggacagcgggaagttctattccgaaggtgaactcgtactcaatcgaggagaagcagaagccgaacctgccaaaacctggatgttggcgatggatcagatccgtgaattgctggactccggtaatctgcctggcctgatcaggggttcaaaattcgacgtgttcgttactggccagggtcatcctggtggatacccacatctgttccgtattaactgaggtcaattatgagcaatttaccactgcttcaagtcggtgacgtattcattctcacggcagaaatgaccgttgaaactcaggtgccaaagcatttcttgtattctaattgcaagggtgattggcaaatggactctggttatatcacccctgagggattcttcggttacatgcaaggacattacgtcgttaccaaaacagccctacaaggcggtagcactggtcatgatccataccctgacggccatcatgtttgggcacagcatactgaaaatgaacgcatcagaatctgtttctatcagacaggtagtttcaggaatatgcaccggaatgttccagtagtcggaaaggctaaggcgcaaacatggacttgggagaaattatgaaaatcaaaaagataggcgcaagcccattgacgggaacaatcttccagggtactttgaacaccaagaccagtatgtgggtcggtgagaagactgatgtgactaacgactgtgtagcggctacggctgaacacatgcgtcacatcaagaaagactactgcttccctactaaagacggcaagttcctggtgctgagcgctgaggttcacgacacccttcctgatcgcttcaaataaatttaaaatttattcaataaacggggtttacttcttcaataaaccccgtatacttcattccataagcagcacaacactgactctgaaatgaaggaactacaccatgaccacagcaaaagttaccatctacgctttcgacgctttctctaaatttgacgcattcgtctcctccgcatttggtatcaaagaagttgaagcagagaaaactttctgtggtggactcggcaagtttgccaacctgagtttatccctgacagagttcatgaaatctcttgaaggtcactttgacgccgaaaccattcgcacttccttactcaagaaagacggcacccctcgcaaactgtctggcaagactagaatttacgatgctgacggctttactgtgtctgtgcattttactgaagaacctgtgtacgaacttttcagcgggtctgaggacctcactcactatgtggtcaggaaaggcgattcccgccctgtgttcgaatctggcgacatcctcgccgctatcaaagaacttgcgcgtctgaatagcctctaacagacgtttgttctaaacctttgaacggggtatggttaatccataccctatcccatacaaatagaggactttatcatgactacaagaatcccaccgcagtcgattgaaatctcgaaggtcgtcaatatcgaacctcgtcgaggaacccgtaaaccccgcgcggggcgtcatctgaccaccaagctggatcttctgaaacagaagttcggcgaccacagtgtgtttacccgtatccgcaacgcattaaaggaagggcgaaccgagttggagctatatcgtccgaacggttcgactcgagcgtatcagaccaccgacggtttgctggaactgatccgcctatcagggatgaacatagaaccccgttcatcaggaaccccgctatgcagcctgtacgtcatcagcaatcttggggcgctgtgagttctatgaagaagtatatcgttcttataacgggttcccgttctataactgagcgggacaaaatatttgccaaattagatgaactcttaatccctcacgagatagagacgtttattgaaggtgaagcagtgggtgtggatctgatatcccgtgattggtgtgaaatcaattatgtgcatatcacaccaatgccgattcctcaaaattaccatacgttatatgggaaaggtgcgggcaatcaacgtaacaaagatatgctggacaaggctctaacattggcgaagcaaaaagatcttgaggtctttggaattgcattatgggatggttcgtctactgggacgcaagacatgatcaagcgcatgaagaaagcgggaataaatgtcaatatcactctcatgggcaaacccaaaaccaaacgattgttataaataaatttgccacccatgaggagttataccatgttacaaatactgttggtgctttgtgtcgcatttaccatgtacttcatggcacgcatcggttttattactttagatgacatgaagggtatcccacgtgaacccaaattcatctttctgaagaagtttgccaaaaagttctcagaaaactttatttatcatctaccatgattaaatacccccaaaattgggggtatttttatgaacgcacaaatctatgatgtcctttcctctggcaatgcatttagcaatccgcttccatccctctccggtccttctcaggatttaataaccagtggcatcaccaatataccattgataactgccaacgctactccggagatgcaggcggctatggcagctggtggactaacaccagaaaaactcacagcagccagcaccatgtatagcaccgccaacacagggataacaactctgaacacatatggcgaccaaagtatcaatgacgcgtattctcgcatcggtacttccgtgtcatacaaatcaggtctgaagggtatatcacgagagccaaacaattgtgatttgatcaacaaggcttttggtgttgttcaggatttaggacgccaatggctgaatgctatggaaggcgctctacagaccgtaacaaacaagatatcggaattgtatgagatgatcatggaaggtgcctcagaaggaatggcaaagctgcaggaattggctgcagaagtaacgggacacatcaatactgcgataaccgcagtcaacggggttataagtgatattacagatggtatagccgcagaacttgcacacattgaaagcatgatcaaagagtgcttgaatttctcttttgctaacgtgctcggggaatgggctaaagatctgtgcgccggaggtgttattgacaaaatagggactccagcattaaaagaatccctgaaataaatcaatgacgagcagggaggactcccactggttcgatgccttcgatcatcagggagtccactccaacatcgctcaggcgaatccaaaccttaccccctggccttatctcatagatgttcatctcgtttccaacagcagacccattcacaaaaacgccttgagtagcaatccgaataggcgttttggtgttgttgattatctgaaaagatatcgtgatatcccttgtcaattccacatttcttgcttttgtatatctcctcaatttttcaaagaaagatggattgaatgtggctatgatgctgttgttttgtgagtagtaaatactggcagacagataagggatttttccctcatcacctttcctcaatagagggatcgtggttttccagtacggtctcaacgcctggttgttatcatcaaaacaaccgacggcttgtatggtttggaaattctggaagtctgccacaacttgatccagtaactttttattacatttgtcgacatcagcagaaatattgactggtatagttctgatttcaggatcccaaagatcggtgacagaaatcacggacgtgacaaagatacggcagcttgtaagtacaagcaccattatcatcgtcatgatccattttctgaatccgcgtatcaggaacactaaaaaattctccacagcaaaggtgctaacatgacaggtgtacaattcaaaaacgaaaatcctaatttcgcggcttccgataaatggcgcttaaccatcggagacctggtgctgctaacccgttcgatacacgacttcagtatccctggcgtgtattccgaaggtatcgaaggcccatcccctggcgatgtcctaaactctataccttcagaacgtctcacgtatgaccccatcgtatttacgttcgttatcgatgagagttggtacaactgggaacagatttacaattggatcgcttcgaacgcgggttcagatttagccgtcagtagagatattacgattgaacttttagacaacatgaaccgtcctgttgggcttcagttgcttctagaagaagggcgaccaacagcgctagacaacgtgttggttgacgtggatgcatcggttcctcagttggtcacaacggtgacattcaaatatctgaaactgactccgacccgcgtaacccgcgaaattgataaacaataagttataatacagagagttgaaacagcaggaaataaatcatgagtgaaacaaaatttgaaacaatgaagaccgaagatataatcgcggaattagatcctcttatttcggtgaatcctgaagacatgaatttagaccaaatatcattgaaaattggtcgttcatggatggctgtccagcgccattacattcgtgaagggcgttatctagaatttttgacaggaaaattccgtcaaatcgatctttatttacgtcggtattatgcaggggaattaccacccaatatttatgtcgaacgtcccctgaaagttcgccccctgaaatctgatatagacgtttgggtaaaggctgatgacgattacattgagttatcgagcatgttgcaagaacaaaaagccaaagtaaaattaatcgaaagtagtttagaacgtttgaataaactcggctatgaagtgaaaaatgcgattgattggcgaaaatacttggacggcatgtgatggtttccctgcaggctttgcgtaaactcaatgattttgagaaccgccattacacatttcagcagtatgccttgccctttaaggaactgacttgtgtaacaacggaaaacggacgccattatgtgtctccaacaggcgtcaaactcacatctgtaacaacgatgttggggcgtaccggagatcatacatggttagaagcctggcgggacaagttaggacacgaagccgccgatatcgaaacagagcgttgcgcagaccgtggggaaaaacttcatttggcctgtgaattatatctcaaaaacaggccgatgaaggaagtgttagaagccgcaggggaatacgtgtttatgtttaaacaggtcttcccctacctgaacaaaatgagcaagatatacgcgcaggagattccgttatatagcgaagtccttggtcttgctggtcgtgttgatctcatcagcgtttacgagggaatccctagcattatcgatttcaaaacatccaacaccctgaaaactcggagcatgatagaagattacagcattcaactatgcctgtactcagtcatgtttcaacagatgtttaatgttaagatcgaacgtttggtcaatattatatctaacgaaaattctcttgtcccgactataatcgaatttaatcgaaaagacgttatttctaaaatgtttgatcgtgttcgcctgtatcataaaatggataaagaacagaacgggatctgggcggatcgctaaatactccataatattgggggaattagtgatgtcagaaattcaaataatcaaagttaacgaagtgcgcatgcgaatcttggcggaagactatatccgcgaagaactcaatgattacttcaaatttgaagatcctaatttccaaccgaatcccttcagtaaatgggatggcgtggtacgcttgttcacaaagagttctggccttatagacatcggtctgttgtttgaggtgtttaagttctgtaagatgaatggttacaccatcaaattagatcccgccttaaaatatatccaagatataccagacgaagagatacatacatttatcaattccctgaacgctgtttaccgtgatgaaaacaagcaataccaaatcgcaacagtgcgcgactatcaattcgactcagtggccacagcaatacgccaatccagatgcgtgctagaagcggctacgagcgcgggtaaatcgtttattttgtacatcatggcgcgttactacaggcaacgcagagaggcactggaaagcaatctcaagacgttgatcgttgtaccttccattcacttggtgactcagttgtatgacaactttgaggaatacagccatggcacaaattggaataccatgatgaatgtccagatgatatgtgaaggtgcaacaaaggatatattcaagcccatagtcatttccacttggcagggtattcagaatcagcccaaagaatggttccatcaatttggggatattgttgtcgacgaagtacacacatctaaatctgaaaagctatcttatatcctgaacaactgcattaatgctgaccaaagattgggagtaaccggaaccctcgcgaatacaaaggttgcagggttgcaggtcgttgcccattttggagcataccataaaattattacagcacgtgatttaataaatcttggttatgccgcagatatccatgtcaacatgattgaagtcatacatcccatggcggattgtatagaaatgcatggtgctgagtaccaaaaggaaatagaattcctcatagcgaatgaacgtcgaaacgagttaatagccagaatggcattgtctttgaaaggtaacgtggccgttatgttcgaacgcattgacgcccatatgatgattgtttatgaaatgctttcagccgttaagaaaaacgtatttgtcattaacggcgaagttaagacagaagttcgtaaacaaattcaggcagcgatggaagagggcgaagatataacactgctggcatcttatgggaccatgcaacagggagtgtcgatcaaaaaacttcatcatttgatattggcacacccgagtaaatcctatattcgtgtcatacagactttagggcgtctcatgcgccagcactcttcaaaggatgttgccaagatctgggatttagtagacaacttgcgttacaaaggattttcgtacaaccatgccctcagacattcgcatgagagatataaattttatcttatggagcgacaccctgtacaaatgaagaaagtgtcgctccagtaattcatttatcttgattcacataggcgggggttgcctcgccgtctttgttagaaccgaacaaacaacccatcgtgttgatgcattgttgtagaacttccggctcaagaatctgactcatcagttcttcattagattccatcagcgggatgactttcacttcccgcgcctgtccttgttggataatgccgatatacagacctttggtgtctggcagtaacgggatcaccatcattggtgtcacaacaaacttagaagtgcgatagaggctgtgtaagtaaacacctttcaatgtcggttgttcttctaccaacattgaagacattgaaaggttggattcttgaacttcccccactgcatataaacccacgaattgtttcatatatcactctcttgttagtaaacataactacataataatctcatgctgtgtttagaccaaataaattattttgctagttgaataaaaatcgctttcttgttgaacaatccgtgcgtaatatacgagttgtgagctttgcgagcaacgagtattatcacgagcgaagcgagtagactgataaagaaaggttttattaaaacagaatattataaatgccgtcaggcatttatttatcgcgcgtagggcattttaacacttgttctataactgcaaacacgcgatactaatttgcgtcaaacatgacttgaaccccactatattttgaggatataaacaatgcaagagatcactctctctgaacgcactttacaactgctggccaactttgaaaccatttgcccttctatcgttctggcacctggtaaaaagctgcgcactgttaacgactccagcaccgtcatcgctatcgctgacatcgacgaagacttcccgtttgaattccctattttggacctgaccaaacttctggccattcagcgtctgcctagtttcaaaggcggtaaaatcgaaatgtcggaagatcatattttactgaaaggtgaaaactcgcagctgcaattctggcgctcagcaaaagaattgactgtcgttcctgccgacagcattgatctcccgtccgttgaattcgaagcgacggtcactcctgaaaaaatgaaagagctgacccgcgcctgttctactctgggacacaaaactgttcgtctggttgccagcggcggtaaaacccgtctggtcggtacgactacgactatcgataattccaacgattacaccgttgaattgggtgaaactgctctggctgatttcgctatgcctgtcgacgttgttaacctgaaaatgatcgagggcaactatgtgatccgcgcctgtgctgaaatgcagcttgttaacttccagtcggcagatggtaccatcaactactacgttggcatgcagctggattaatcaatatcgcttcggcgctttattatcggtggcggggttattatcccgccctttgaaatgttttatctaaagaggctgcaatgtccaacattactatcgttacagaccaatacgcgtgggaaaacaaatatcgtcctgataatttagatgagatcgttctcccctcagacgtccgtgccaagatccatggttatgttacctctggcaacggaaacatcccctctatgctgttctactcccctcagcctggtacgggtaaaactacttctgctctggccgtgtgcaatgaaatcggttgtgtgaaacctctgatgatcaacgcatcgcttgacaacagcattgatgtgattcgcgatcgtgtcattcaatatgctactggcgtcagcgttttcggtggtcggaaagtcgtcatcctcgacgaagttgagcgtctttctatggcggctcaggaatcattgaaaggtatcgtggaaaaggtttccaagaactgctcgtttatcctgaccaccaacgccaaacagcgtgtcaatgagcctctgcgttctcgttgccgtgaaattgatttcatctggtccgaaaaagaagccgacgaagttaaacttcaatttatgcgtcgttgtgccactatcctgacagaagaaggcattccatacgaagctggtgttattgcgtcgattgtcaagtcgtatttccccgacaaccgtaagattatgggcacccttcaggataatgccaccacgtttggtaaaatcgatgaacgtgctttggctcaggtgaaagcgggtgaattccaggcgttggtcgatttcttaaaggccaatgattgggctggtatgaagcaatgggtgactgacaaccaaaactacatcacagaggacttctactctcgtttcttccatttctgtgttccgcagaacaaagagaagaatcctttgatccaaaacgaatctatcccagatttggtctgtgtttgtggtcagtcacagattgaacatcgttcagttggagacgtctggttacacggcgtttacttcctgaccaacgtcatgctcaacatcaagtggaagtgatattatggccgctccgtcactgttcgattacctgggtgctctgaacagcaccaaagagaatctgttgttgaccgaagaccccgaaatcagaaaggcttttgatcctttcatgactcggcgcggccttgctcagagtaaagacacgttggtggttgcagaacgaatgaaccgcttccatgccattactccttggatgcaatggaaccttgcgtttcatagtattccagcaaaacgccgttatgacaaatggtctaagaaaggagccatggaccctgatgtaaaattgatttctgagtattactacatcagtcctgaaaaggcttctgagtatgttcgattcctgccgaaagaagtcctggctgaaatcaaggcgaaggtcgaacgttccaacagcaacgaaaaagctaaaccgcgcaaggctaagtaattcaaattattgttaaaggggtacgagtcatggcgcgtaatacgctggacatcttgaaactttcagctgttaacgatgaggccagcatcgttgattgtatggttgaagttcaattgaaccaagataaaccaggaatttttctgggtatcaaggaaacgctgagccgcatcggggtgaacactcgtcaagagccgaataccctgtaccaaacatgtcatattctgcataaatttggcagatatcacatcgtgcacttcaagcatcttttcatgctggatgggcattacaacggctttacgcgtgaagatgtcctgcgtatgaatcggattattcggcttttggagcagtggaatatggttaaaatactgcatccggaacagattaccgaagttgctgatatgtctcgaatcaaagttgtaaaacatgaccaagtttctgattggaaactggttccaaaatataccatccgcccgactcgggcgaaaagcgaataactggagtcgctatattatgacaactacatttaataaaaacgtgagactgaatctggcttttggtaatgctgcaggggacgtgaccgaccccgactttagcaaaattcgcaatcaggcaaaactcgtgctggaagagacccgcgaacttctggaagcggcttattacgatcatcaagtggttctaacattagaattgaatcctcgagaaagcgagacgccgaccactaccgaagatctgatgaaagccatcatggacgcgcagggtgatatcactactgttaacgacggtgtggcccatatcgcgggtttcaatggtgatgagtgcttacagcgcgtgtttgcttccaacatgagcaagtttatccgctctgaagatgaagtcggtccggctctggactattactattctcgcggtttccctgatggacaattgcgtgtggaaggcgaattccctcaggcatgtattaaagttaatgaaactgtcgtttggaatggtaaagaatatcctaaagggaaattccttaaaaacatggctgttttccaagagcctgatttttctgacatgctgaccaaaactccttcccgtcagatcaacgatatcgttgccagcatggcgattgaagaaggtcaggtgtttatcgatgatctcaatggcgtggcgttggtatccccgaacacattgggtgtaatcctttccatggctgttgaaaaaacttctcaacctgtagagatcgacgggcaaatcgaaagttatgtgccagcgttcatcgtggatgacaccaatatgcctaagctgtgtggggtgtggggttctaatgttcgtgtttatgcgagcactccagtgcagaacacggtaacacacctctcccgtatcgattaatataaaccgttattcattaaacaacagaggctataatggcctctgtttttgtattctagagaggataccatgaaagataaaccgtgtgacattccttattgcggtgttcgggaaaccaaaattaaatctgcacaccccacgttaagtccattccataaacaactcgcatacgaatggatgagcgagcgctataagatccacgtgagaaaggacgtccaacgtcttcctgctccttggacggataatcccatactacggcaggtcaagttctgtaacgtccggagagagcacgacaggcagtctcttaacctgatcaacaatatcgtcaagaacgatgctctgggcattgccgacaagatgttcaactgtgttctgttccgcatgttcaacctctgggacccgatcaaggctatcggtgacgctcgtactatcaaagagttcgcaaacatcgatcttgatgcaacccgcaaaagtcttcaggaatttgaggccaacggtggtaagatcttcaccaatgccttcaacacaggcggtctaaagcagtgtctggcgttccccgaactcgttgtcaaccacaaggagcagcgctttggtggtatgatggttaaggtacaccgtgttggccagggtatcgttgatgaattggattacaaggtcgccaagaagctggttgaagaaaatcctggggagtacacgatcgagggctgggagccgtacatgccgatgcgtgttatccgttccctgaaggcatttgtcaacaagtatcctgattatttcaatgatcttttacgtcttgactctccgctgaaagtttaccagcgcatgtatgacgacattgaagggcttgggccattcctggcatatcagatatgggtggacttcacatacatccctgagtatccattcagtgagaatcacttcaccatcgctggcccaggttgccgtgcgggtattgacctgatgttcctggataaggacggtatgactcatgaagagtgcatcttctgggttcgagatcaccagtatcaattgtttgcccagtatggttatgtccaagagcagttctggtcagccgaagcgccggaagatcagtgcatgaatgtcatgcagctggagaacatgttctgtgaattgagcaagtacacccgctgtgttgaggccattgagagaggggaaaagccacgcggcaaggtcggttacgatggaggcggagaatacaaaaagcggtgcgatgaagttgtcctcaaacgcggttccgtcaatttgttagatcgtttgaagaaacctgaataaagaatcgctttacttcattcttcaataaccccctttcaacgtatattgacctcactttaacgacgcgcccatttagggcgctcaatttgaactgaaacaggaagactataccatggcagaattcaaacctttcgcaacggctgttaatgataacgtgctggcgatgtccgcaactggtctgtttatgaccaacgttgataaagatgctctgtgggatttgtatttggcatccttcccagctggtaccaacccgatgtaccgcgaacgcactgaacacgactgcacctgctgtaaacaattcatccgtaatatcggcggcgtcgtgactatcgatgctgacctgaacgttatctcgatttgggacaacattgagctgggtaacgaatacgacgtggtggctgcggcgctgtcgaccttcgttaaacaacacgcaatcgtcgacgtgtatttcaacgattctgctaaagtcggcctgtctcataaccacgaatctggtgaagacggcaaaatccgcacgtataaccatttccacaccgaactgctcggcagctatgtcctgcgttctgatgctattgcatctaaaaaaggtgaaatccgacaggctatcgaattatttgaacgcggtctgcgtgaaattactctggaatcagcagaaatcgttctggaattgattgatcagaattctttgtatcgcggcgctgaacataaagctgcggtgatgggcttcgtagtagccaagacggcatatgaagaaatcccagaatctaaccgttctctgtgggcatggcgcactggttatcgttcaaacaatcatgttccacatggcattcgcaacaccgttatcggtactctgttgactgatatcagtgaaggcgtggaactggaaaaggccgttaagtcgtttgaaaccaaagttgccccagcgaactacaaacgcccgacggctctggtctccaagtccatgatcgaaaacgctcagaaagaagtgattgctctcggtctggaagattcgctggcgcgtcgttatgcagtttatgacgacctgacgatcaataacgttctgtttgcagacctggctgctcaaaaagcgatggatccattcgctcaactggccgccgaagtgaaaacgccgaccaagtcgctggataaagttgaagagattggcattgacgacttcctgagcaatatcctgccgaaagcgcattcaatggaagtgctggttgaaaactcgcatactggcaaccttatgtctctggtcgctccggctattgcaggtgctcctaaccttttcaaatggggcaacggtttctcatggtcatacaacggcgaagtggccgactccatcaaagagcgtgtgaaagcggctggcggtaaagtcgacgggttcctccgtgtatccctggcttggcacaacaacgatgacctcgatctgcatatgttctttaacaatatggaacatgtgtacttccgcgatcgtcgttcaatgactggtgctcatctcgatatcgacatgaacggtatggacggcatcgacccgaaccgctctccggtagaaaacatcatcttcactgatgaaagaaagctgcgtgacggtgaatatcgttttgagattcacaactacaaccaacgtgaaaagatcgacgaagggtttgaaattgaagttgaatacaaaggcgctacgcaacgttttagccacaatgggttaaaagaccaggggcgcatcactgccgtcgttctgactgttaaagacaaacaagtcgttagcatcaagtctgagctggcgaatgttgataaatccaaagaaatttggggcattaagacagcgaccttccagaaggtacagctggtcctgaactcaccaaaccattgggatggtgaacaaactggcaacaagcacgtcttcttcatcttggaaggttgtgcgaatcctgaaggtactcgtggtttctacaatgaatatctgaagccggaactggataaacatcgtaaggtttttgaaatgttgggtagtaaaatgaaagtacaacccaacgaaaaccaactgtctggtttggggttctcgacaacgcaacgcaaccatgtgttcgtcaaagtgtccggcgcgtttaaccgtactgtaaaagtcattttctaattgatttgacaacaacttcgtaataaggaacaaagaaatgtttgatcaagcaacccgtttaaaactgcgttttaactctaacaaaggtctgctgtctgttgagcaggtttgggatctgaatctgaacgcactgaacgagctggcgaaagatctgagccgtcaggtgaaagaagcagccagcgatgaagaagacttcatcggtgtgaaaagcgcggtcgattctcagttacaactgcggttcgatatcgtgaaagcgatcatcggtgttaaactgaaagaacgcgatgaaagcgcgaccgcagcagagcgtaaagcgaacaaccaggcgatcatggaactgatccagcgtaagaaacaacaggaactggaaggtctgtctgttgaagaactggaaaaactcctgaaataatctgacggctccggcctgagaaaccccgccgtgtgcggggtttcttttggacaaaaatcatgaaatctattacgtgtgattactgcggtcaacctgcccaatacgtcgggtgtgatgttgtatacccccaccgccccgacctccgcaatctgaagttctgggtatgctcgccatgcgacgctcgtgtgggttgccatacccatggggatggtagacaacctctgggaaggatggcgaatgccgctctccgtgctgctaagcaggaagctcatagatcgtttgaccctatctggaaaggtgggcagaggagcaggagtgatgcttacagctggctggctcataatctggggatcaagaagcgcgattgccatatcggcttgttcgatatcgatatgtgccgtaaagtagtcgctgtgtgtaatcaactaaaggcacaagataatgtcaaaggaaatacaagaagcatttgaaaaattatctcaggtaatgaccgactgcaaccgccggaattattgtgatgaagcgtataccaaattgaaaactgctgttgaaaccgagctggcaggtctttccgttttcaaagatattcagaccgaagtgctatatctgtcggatgaagatttaaggcgttgcccccgcaagatttaccaattactggatcgtttggtgaaagtgggggtgaataatgggtaatcgtgaagggattaaactcacaccagaacttctgaagtcacatattgctgaagtgatttatgaagaccgtgaagttggtggtcaccgcgtcattacatgccatttcaagatggacaatgggttcgttgtgtggggtaagaatagttccacatcggtagaccctgccaacttcgatgaagaaataggcaagcagctggcctatgaaaagaccttttctcaactgtgggaactggaagcatatcgcgcgatggtagaaaagcagatcatcgatgtgtctgccgaagatgctaagatcaacattgatgaatatgtgtcagaaagggaagcgatacatgagatgggtggttctgaatttcttaagggtaaaggacaatccatcgtgtttcgtcatcccaaagatccaagtcggggatttcgcgttgaatttgttcgcaccgataatgaacgttataacgaacgacgcaatggtgaggcacgatctttggtggaagcgtatcttcgctcgttcatgacttctgaagaactaaattctgaacttgtcttgcgtttggcgaaaacgtgtaatgctgcttcaggggattatccgtggtgtgacctgtcagaagacaaacgccaagcagcctgtgatcgtgttaagaaattcttgttagatccgaaagcccaacctggtgattccgtaggggcactgtatagagccattgtggtgtcctacttgtaattcttcaaaagtctttcaaaatcaataacgtccggcaaaatgctgggcgtttcaattaataagccaaggtataatggcccaactgaaaacgcgggggaatttctttgtccaagccattcattcgttccggagttgattctctagactcccaatttaagccgagcgaagttttaatccaacaaattgaaaccaatcgtgaattcatagaacacatgttggcaaagccggaaggcgaagccgctcaaaccaccaaagaaggtaataacgatgattgaatcattaacatttaaaaatgcggtagcaaccgtcgcgccgtctaaccatgtaatcgttgtggatatctccggttccatgtaccgttcacttcctgaagttcgcaagcatctgaaggaaaatctcccgtctctggttaaaccagaagataccgtcagcattctttacttttcatcccgtggtgatttcggtacggtattcgctggccgtcagatcaatagcgcgaccgatctcagtgaaatcaataaactgattgaccgcttcctgcaaccgtccggttgtacgggatttgtagaacctctgaagttggctgctgaaaccgcgttgagcctgaacaaacctggttatgtcaataacctggcgttcatgaccgacggctatgacaactgctggcgctctaatgaaattctggcagcagctgaagtactgcccaaggcgttcgataatattacattcatcgaatatggctggtattgtaaccgcgaacttctggctctgatgtccgagcgttctggcgcgactcacgtgtttgccgaaggtcagactgaatatcagaccgaactggaaaacgtactgaaatcatctacgccgaaagtggttgttgatgttcccctggtttacactcatgccatctacgttgaaaacggcgtggcaactgtgctagccgtacaacccgatgaagaacacccgattggccacgtcagcatccctgaaagcgtttctcagctgtgggttgtaaaccctaacatgatcgaccagctggataatctggcagacacccaggcggcatacgtgctggcattctatggcgtgtacaccatggacgccgatctggtctgggccgcgctgaagaagacgggtgacgtccgcttcatcaaacaatacagcaactgtttcaccaagcaggactattccaacatcaaagtcgatctgacccaggctatcgtggatgaaactctacgcggcgtagatggcatcgattacaatatggttccagctgaagatgcaacgaccattgttgatgtcttgacctacctggccgaagccgatgtctctgttgtcactaagcatccattgttctcttacaacagcatcggtcgcggcactgtgcagaaagttgatgacactgaagacaagctggctgaacaaatcgccgaagccaaaaccaaagcagagcgtaaagcgctggctctcaaactggctgagcacgaagattggacaccggaatttactccggttgatgacaaagggattgtcccaattagcaatctggtctacaattcagagcgccctaacatcagcattcagaccgttcagcatgggacggttgttgtgccggagttcgtacagaagaaatatgaactccctgaagaactgggaacctggcgttatcgtaattacaccatcgtcaaagatggaatcatcaacctgaagacgatgccgattactgccgaatctccgattgtccgagctaaggttgtccaagatttgatcgccatgggcgttaatgtatttggtggaccggaaatcttcatcgttaatttagaaagcgttccgatggtcaaccgcgccatgaccaagaacatcagtgctgccgaattcttcgctgacaacgttcgtcgcgaagcgctgaaggcaaaacagaaagttctgaaattctatcgtgatgaactggttggcaaaggcaatgcaactggtctggcttctaaatatggtaaagaagccgccgatttcttgtcggcaaatggcattcgcgactacgggttctcgccgaaaactacttctgtggaatcaaccgatgtctacatgagccgtgagctgaatgtgaaaatcaaaggtgcttcgtcgctgccgagcattgcgtcagttatcaaaaagcaagcagatggtaaaaaactgaatgtcgctgatcagttgattgccaatgctttggccgaatacgacgcttttgttaaatcgccaatgatcaccagcgtacctgaagagactcagaaaaagctgattgaaagctggatcggagacgcagcaaaagcggccattaacgaagtccgcgccctgaacaaaactctgtctaaagtagtttacggcatcgtcgctggtcatggttggttcactgatttggatctggaagagtctacgatggatgtagaagttgatggggtgaaatacaccgtcactgccgaactggccgaaaaagaaatcaaaatctaaatctaattggcggggaaacccgcctttactttcaggaaatgaatatgagcaacaaaccgacttatgttgatgtcatgcgcgatttggcccaatacgcatgtgacaacctgaacatggaaacttggcgcgacggcgatcataaagaaaagttcgataatatcgacgacaacatcgatgcgttggctcctcttcgggccaagagcgatctcggcggtgatgatcttgacatgattgaactcatcatggaagttgaagaacaatatgatgttgaaatcagtgatgactgggtaggcaaacatggtgatgatcccacgctcggcgaattagctgaattggtagttgccctcagcaagtaaaccaaaagaatccccaatatttattggggatttttacttttaggggtttacttcttctaaagaatatcctattattcaataactggctaacgtggggttggctagaaaaggagatacaaaatgaaactaccaggtcatattgaagtttatatgcggttatacaaagaaggtaacgccgctgctaaagaactacacgaactgattttgtcatacggcgttaagtctgttacagaagtaaagaacaacaaaatagacggcgaatatgtgtcaatcaatgggcgtggtctgaaaaccaacccaacatattttgtgggtgtccatttagacacagggaaagaagtcgtggagatcgtggcataatgtctaataagaaagttgaaacgacggtagaaattgatcgtaattggggattactccgcgctaaaggttttcgtcgtgtttatatgaccaaggaagtttatgttcagcgtcgcgacgttattatcagcaagaaaggtaaagagtgggttgctgaagaagttgacattgaaacaggtgtcgtgggcaaagtaaaagcgaaggcggctattttgttttctcttctgaagcatatcaattaattctataaactccggcggggtatgattaccccgcctatttgaaattgcaggaacttatatcatgatctctgtcgagggtataatatgacaaaggaacaagaactcgctttgttctctatggttcgcagtaatgttaaattgagttttagacaagtggactccattattgatcttatgaaaattaagaattccatagataacgatcatttgcgcaccgctcttttgggattgttggaattcgttttgcgcggatctgatcttggaccagtgaatgatattgacgacctgaccaaattgttaaacactggcggtgatggtttcgataccgctcgtattctgattaactgtggtcctaacaaagaagaggtatgatgtggacaaagatttggtttgggaaatcgtgaaaggtatcctggtaataattgctgtggtctttatcctaggaggaaacttcttctattggttattgcggtatgaagagaagcgtcgagagcggaaagaacagaagagaaaagatcgatggatgaactaactaagggataaccctatatcccgaggtgtgtcatgttccttacatcctttattgactggatgaatatcataaatggcctgaggatccctgttgggaacctcgggctttctcgttcttctatgccgcaaatcgatgccgacaaacaagaagattttcacaaatatcttgaagacaacggcgtgtctatagatgctgcgcaagtgcccatcaaaatgcttcgtcttacccagaacgaaatcaacaagatgaaggtctggaagattatgaagcaaatccgaaataagaaacctatgggtcgagtttgggtgtcttctgacaattacgttgttgacggatctcaccgatttgtcgctgcgttgaacatggacggcaaacagcgcatgaaggtctacaaagttgatttgccagcgatggaatttgttaaactcgctaaacagtttcaaggggtgcgatacagaacggtcagcgatacccgtttctgaatacgatagtttattgaaaccctttactcaacatggtgacagccctatcattttatccaatgaaataaggagaatcaccatgaaacgtttaatcctcggggttgtgctggcaggtctggcattacccgccgccgccaagctgaactccacaggtgacaaactgtggtctgatctttcctactgtgctgggttctcccaggccgtggctatagacaagtccggttccatagagaatttcgctgaactttggaacacgggtaacgtctccactgctgttgttaatgctgggattgagttcaaccgttacaaacagggagcctacaacttgaaaggctacctgaatgacgatgaatttaaccgaggaggcatggaggctggcgatctcatcatgactggccgtatgggcacacaaggccgtatgactgtaaggcaatgccgttctctcccctctccggcattacagaagggtgtgcaaatcgggcgtcgacatttggatctgatcaatgatggcaaccaatgcatcaaggtgtttgaatatagtgccgaccaagaatcagatcgacgtttgaaaaaggaatggcggacacgtgcccttggattgagagcatggctcgtagaaaacgattattactacgaggatcgtgttcagaacggattgaaagatctttccactaatttgtctgtcaatctagacgacccaaggctttcgcgtgaactcaagcaaacacggattgactgcgacaatatgatgaaggagaacgaaagatgagaaacataatattcttgttattcttttctatgttggcatcattccaagttatggcgactccggccagcaaattgaatatttgcgcggcctggttgtctacttatcaacaatttcaccctgatgataaagatacatctatccttttatcggattatgaaggtgagttgaaacgccttgatctctacagcgtggatcaaatagagaatgctttagatttgccaatgatggagtccgctgccgacaatagcaaacagacgatcaaagatctgactatgtgtcgggaaatagccactaatttcgttggaaatcaaagataattttcaggttaaaatttaacttccattgaactgaaaggaaatacttcgtgatgattattactaaaaagatcgccgaatcgatcgttgaaaagaataagatgttcgcgcccaacaaagcaaaggtcattctgattcacgaagacgggcgcgtcatcttcgctgagaagtttgagcagggttgtgctttctggttcgcagatactatgcgcgaaagcaagctgcagggcatcatctataaattgaccctggttgccaagcgcaatgatcagggtgattgggttacggtctgcgatttcagctctaaggttgaaacgaaagccaaaacccgccaagaaagaattttagaggctcaggaaaaacttcgggtcgctcgcacatcatccgcggagaagccggaaataaaaccagccgttatgcctgtcaccaataatccgctcccacctgtgggtcaaaaggctttcgaacaatatgttgaaaagagaaaagaagaatctatgaagactccggtagaaaataatgatgagtctgaattcatttcaatacgacagttgtgcccgcaagaacaacgttattcttctccaaatgatttatttgtgtcattcagatgcaacgggaagatcatcctgtgtaaaactctgcgtgaacaactaccatggcctaacatcaatatgatggtgagtcgcaattttaaacgatttgctattagtgaagggaacgattatcccgtaaacaagagcggaacgtatgccaacaaacatatgtgtggtaagttgtctttccctaacgattccggaaccatccgcgttcgtttagaatgggatgaaaccctgaacatgtatgttggtgatataaaataattcaataacattagggcggttattattaaaccgccttttatttgagagatctatattatgaataagaagagtcgtatcatcatcggcgcggatcagggaaatctgtattttgatacaactggtgctattgcccacgccatccttccaacaggtatttctgtttattgtgtcgaacacccagatccagcgatcaaacgactttgttacatccatcgtgaaaagggattgattgtattaccggaaggcgcaattatgcgcgctcgttcttggccgatcgattttaattccatgaccgccagagagattgacgcccgtttcttcgcatacaatatgaatcgaactttaaggaacgaaactcaaagagttgcccatttcgataatgaaacatataccgtgacattagaatttcgcgttcttggtgaagctcttcaggtaaagaaaacgagttgggcaaccgaagggacgtttaccaatgattatgctcttatcgatagattgtgtaatcggttccgaattagcctgactgcgaatgaagatttgggagaatatgtatcatggacagctcgttctccacatatgaatgggatcatgatggagagcgggactcgtaatcgtgctgttcttcagctgctgttgttagatgcgtattctcgcaatcctctggaatatttgtctcgagagcaaatcgacaacgctctttcccattgcaatcatctggaggtgtgatatgtccggcgtatggtgtgatgtattattagtgagattttcaaacgggatggataaccagattatttcattcttttatcctgttcccttttcttctatgggtctgactgtcaagcgcgttcttgacatagaagaaaaactcaacgctatgccgtatatggcgcgaccacacgtgctgggttgtgaactgttaaacgaaatgatgatcgatgagaatcttgttgaaacaattatcacgcgtgaacagatgaaaactatactggctcagggagaaccccatgtcgaataaaccgtttgaacttaaatttcgcgtttggcatattccccaggttcctgggatcgcttttcatgttgaggttccaacatttgaagaagcgcagcgcctgaagaatgctctgggtgattatgatttgttccagttcaacaacaatatcaagccggattattgcaatgcttcgggcattcagatttatcaacatgatctgaccgatgatgatcttaatgatatgggtctggatgatcgttgggttgatatggaagatgttgacgaactcaacgattatttcgagcatctgcgcagtacagggtgggaaatatgatttcaaaatatattgtaaagcctggggatactctgtccagcatcgctctaaaattatatggtgatgcccaacagtatatcaaattagccagattcaacaatatccaaaacccaggccatatcacggttggtcaggttatttgtttacccacacctgtcgaagacaaagaacacattatgattccggttcaaccgactcagaagtgtttggatgcgatagctggtttatttcggccaggtttttgtgatggaactattgaacgtcacatttatcaagcgattgtggaaaacgcaggtgtaaaatgatcggattaattcgttacctgttcatattcgtaatttcaacattcattttatttttctttttcaaatacttgaaagagactaaaatctaccgaagtaaacgttggcgcaaacgggtgagggtaactttcctgctgctgatttcttcggttacaataaccgcactcacattgggtgcatatatctacatttttgaaggggtacattgatgttcaagaaattggtgttcggtgcaattatggttctggctgcaagtctactttctggttgtggtggcgttatcgatgaaggtaatgtcggcgttcgcacccaatggggtgaagtagacatgaaccctgtaacagcggggatctacaccagctttgtttcaagcgtggatgtttacacgaccaaagaagccgttgtgagtctgactaaaatgacgccaaaggccaaagacaacctgactctggaagacttggatgtggatgtttattacacacccaatgtcgcaaaggttccgtggttccataccaaatttgctggccagagcgctgaactcgatgatggcacgatagcggtgggtttcaatctagttaaaactgctgcagcatcttcttcgatggacgccgtttcttctcttgattctatgacaattcacacccaacgtgctgaattagagaagatgattaaagaccgcactcagcaacaattagagactgctgcgcctggcatgttcacaatcacacgcgtgttggtcaaaaaggcgctgacggatccttctattgagcagtctatccgtgataatgttatggcggacaaacgattggatacggcaagaaagaacgttgaaattcgcgagcaagaagcccaagcgaaccagaaactgaccacttcgctgacgcctgaatatcttcaacatgaatataacatggtattacaatcatgtgccaatagtggaaaatgtactctgatcgttgacggatccggctcaggtaaaatgctgaacgtcggcaaataaacgctttattcaataatatttgttaggtaagctgtagtcctcttgtagtgaaacattgtaaacaactaactggagtaatccgaatgaaccgtactgattttatcgctcacatcgcccagactcagggtatgaccaaaggcgaagcagagaagatcgtggcatcctttatcaatggcgtaaaaactgcagtcgcagctggccaatctgtccaattcgtcggcttcggcgcgtttgaagttaaacatcaggaagcgcgcactggtcgcaacccgctgaacgggcaagcgattcagatcgaagcgaaaaatgttgttaaattcaaagtcggcgaaggtctgaaagctgctgccaacggccagtaatcacatccagagcggtcgtgcctagcacagcgtgttaggctaaaagagaaagccgaggttaatgcctcggctttttcgttgttggaattcgggtaaactataacagaacccaaaggaggaacaacgtgacagcatttaatcaataccgaaaggcgattgacgccataggccgtaaggttcctatagaagaaatacaaagccttgccaaaaacgaatttccatatgacaaggcgaaccaaggccagatggaatgtatcgtcgaagccgttgatgccttggtcaacaaaaaggtcaaacacgtgatcattgaagccccaactggcgtggggaaatctttgattgggacaacaattcataaagtgattcgccatttggttttacaggccgatccttatgggcaattccgcacgtctatcagtacgcccaccaaaggtctacaagatcagtatgctgctgaaaaggcggtggcgatagacatcctgaaaggcaagaagaactatcgttgccacgtacacccagatgtgtattacaacgccgtgcaatgccgtatagcatgccgcgacgggcattgtagcaaacgccgttgtccttatgtacaggcgcgcaatttatggactgatatttcgtcattgcgttgcactaatgcagctatgatgattgagatgtgcaccaccatctgtatgaagcctgaaaaccgttccgatatgctgatcttagacgagtgccataagatgccctctacgctcctagagcacacgataatggaatacaatacaaaggctgtggatgggctgcgaactatccccgaagggaaggagatagtctctatcattgccgatatcgtagaacgcaccaaggactatgtcttgggcaagttgtattccttgtccggtgaaatgcattcgatgtttgaagacctccatcttaaagtggagtctttgttggaagttcttgaagaattggttgaagatgaccgcctgagtgaaagccaggtgatgaaactcgccgacattatcgatgtactacacaatctgagtgattattgtggtattatgtctcagacaaaggccagcacctttatcgtacaggaaaagggagatgactttattcggtttaaacccgtcatgccttctgatgtaagtgaatttggattgttcaggaaagccgattaccatgttcatatgtcggcaacaatctgcggaatcgattcatatgcccgttccttgggtatccgccagggggattatcattcaatacagattggaaatccaatcccaattgaaaatcggaaagtcaactatatgcccatcgttaagatgaccaacaacatgggcgattatgaaatgaaaaggctgacggaatacattgatgaaataatcgcattccatccagggcaatctggcatcatacatacggtgagctatgatcgcgcattagcaatacaaaaattcagcaaataccagaactttatccatgtgccacgcactcgaaaggcgttgatggatattatggaaaatgccttcaggacaaaaacaccttgtgttattgccagtccggccatggaagaaggatatgacttcaaaggtgattacagccgattccaaatactgatcaaagttccttacgattatttgggtgatccattgatcgcacatattaattcagtggatccgtctgcgtacttccgcaacgcagtattgcgcattgttcagatgtgcggacgttcggtcaggggtgtggatgactgggctgcaacatacataatcgattcttcctttgaatctcttatgatgcgcaacccagagttcttcccaacctggttcaccgatgcagtatttgaagtttaataacctgcaataaacgataaatggcacttgatatttggttcaagtgtcgttatcgtgatgatcatattatccacatcttgactaaaagatatatttgctattggtttagtctgaacgtggttgtacaggctgtatatcaaatcttcaccacggtatccgactttaccatcagtatcctgccaatggcctaacgtggattcatgagcatgtactcggaattctatcatatggaacaatgcttttgggattgttttcagtatgctggttccgacaacagggatatcaatagcagagtcgttggaattgccctcatatactgaagcccaaccaaatgttggatctcctcccgccacgggagttctcttccagtattggaattctggattacccgaagcgttcttttgcagaaggattacttcaccgccttcaaaattagaagtgtctatatcatcacgtgctaataaatcagctggagaaccaataaatttccaggtttcgcgatctaaaatagacttataataatcggtgaatacgttgctgtcgttagctgcattgattgcgtctttaagaccttcacctgtgaccatctcattgagttttagccacatgtttttgacagctgacgcagctgcgacttgaccttcagtcccacccgccgtgtagtcataaacgatgtccagcttttgtatggaatcatttatagacaagagcaaacgctcaatttctggggcggtcagactgaaacgatatgcgcgttctgacatgattcgttcctcatcgataaaaccagtttgggatacttatacatgattgaaatatacactgacggggcatcttccccgcagaagacacaagccgcaggttgggcatttgctatcggtccaatcactggagagcaatggaaagtgttttatgggtatttacccccaccgtcaacgaataatatcgctgagttgcttggggttataaacgccttgaagattctatggaaattctctaattgtggggaacgctgtattccctcagccagtattatatccgacagccaatatgttatcaatagtgttttggagtggcgtagaaaatgggaatatgaaggaatgccgcctaagaacacagaacttctgctcgagttgttcgattattatgacaaagtttgttcaatttgtgaacttgaattaaaatgggtgaaaggtcatgcggggaatatcggcaacgaaatcgcagatcagtggagcgtcagagccaaagaaaatagctcgatggttcttgaaaataatcgtttcctggtcagaaaggttgttggttctttcaatgaattcattggtatttgaggattatcacgatgggtatgaattttgtagacaggggtgataacgtcaccaaatatttcactgacgaagataacgatcgagttgtggggatattgcgtgattggatccctgctcgtaaaaaggcattagccgaaggaacacccctaccaagaatccccaattatgtggccatgaatgttcagatgataatcaagaatatgagtatgcgttacaactaccgtgattatccatatcgtgaagacatggtgagtgaagcggttgtgaacatccttcgttaccttcacacctttgatgttagccatatcggcaagaaaggaaagatcaacttcttctcttgggtgactatgtgtgccgatcgctcattcgccaagaaactcacgagtgaagaagaacacaactacattaaattgcgttcctttgaagaagcaggtgggttcgcagctttatcggatgacccagacttccaacaacaaaccttcgttgatagcactgggataacgatggactttcgtgaacgcatcggtaatttcgaaaataagaaagaagcccagcgtgaaaaggaacgccaaaagcaaacggctatcaaagaagaggaaaagaataagaaaatccctcgcggtatacttcgatgtcttacgaagagtgtaaataccattaccgctgacgccgaagacaattcagataatgacttcggctctactcaatttagtcttgaagatataatggacagccccacattgttagccgaatcagttggagaaaagaataatggcgattgctaaaattggcgatttacacatcgggagccgcaatggttcccgttacgttcgtgaattcataaagaattatctcatcaactattttattcccgaattggtagatgcagacattaaagaagtttggcaattcggcgacacatttgatgttcgtaaatttatgtacgggcgcgacaaagattggttgaaagatgaactgactccagccctgagaaaggcgggtatcaaatggaatggcatcgttggaaaccatgatatcaccttagaagaatctaaccgtatcaactggccatcctatctgaatgaattagcgccagacgtctatcattattacagtgaacccactgaagtttcgattgaaggcgaaaaggtattgcttctcccatggatcaataaagagaattatgatgcatctgttaaagcgatacaagatacagacgccaagtactgttttgcgcatctggagttggcagggttcaaaatgtaccagtcctctgtatgtgataaaggtcagatcgacgtggcgctcctttccaagttcgaacgcgtggataccggacatttccatacccgttcgatggaaggtaacattcagtatattgggactccttatcaccttacctgggaagaccataaagacggaacgaaccgaggattctatgtagacgacatgaaaggcggggaattgtttattcccaacaacgagcaacaaacgttgttccgattcatcgaatacgactacacccaaatgtcttccgacaccgaaggaaactggattgacccagattggctaaacaatggtttgggtattgaagggcaaatcgttaaagtgattgtacagaaccgcgataatgccaagcattatgaaaagttctgcgacgcgctgaagcgctgtaaatgcatcgattacaactttatagatcggactatcacagtctcgtctgagaaggtagaagtcactgaagagatggtggcgactgatgctgtggaagtcctaaagaaagatatccgcgccgccaataatatccagcgccctgaaaatgtttgcaaattagcagaacaattctataatgctgcccaacaacgcctgaacacattggatgcataataatgagcaacgtagatctattatcaaaatttggttcgctgttgaaagaagcggacgctcctgttattgttgaagcaccatttgaaatccgtggggaaattactcacaaactgactttccatcgtggtcgcgctaagaactttcgttctatcggtaatgaattcatggaaattgattaccaacgcaattcggcaaccttggttacatccgatgacaatggcgctggtaaatccaccatgcttgtctggctgctgttcttcgttctgtacaacgacacgtacagcaagaaagagaagaaagccggattggttaactctcaaaacaaaaaggaatgcgtgggtgaagttgagttctcaactcgcggtagtgaatggaaagtccgtcggggtatcaaaccagattttgtagaagtttatcagatggtggaaggagagtggaagcaagtcgtgaacgatgcggccaaagctgatatgaacaagtacatcgtgaatctcatcggcgtggaccagaagatgttcgagaactctttggttctgggcaaggagaagtttattcctttcacagagatgtataccgctgaccgccgcgccatggttgaaacaatttgggacttgggtttcttcagcctgatgaatgaggatgttaaagcatctatcaaggcggtaacgaacgaactcacgacggtcaccaatgattacgcctttcatgatgtgaatctaaaaggccagcaagcgcagttggaacagatcaataattccaatgcgatggttcagcaacaatctgcggatatccttgtccaagagaaggaacgtctgaatagcctgaattcagatatcggcgcagtgcaggaacaactgaccgagtcccgtgctcaagactctaaattggaatcagaattatcagaagtccgtaatcgcctgaacgctgaaatgaaggtggatatcgatgccatcaatgacgagtattttgccaaaattcaagcggtacaggatgaggctgataccaaggctgaagactatgaacgtatcgaggtgtctgatggcgaacgttcattacaagaaattcgtgagagaatggcggtagtcgcggaacgtaaaaatgatctggtcagccaacggaatgctaacctggatgaactgaacaaagcattagcgcggcgtcagcagggtgagaacttccgtatcaaattcgtaacagaaatggaaggacatgaatctgccatcaaacgttttcacgatatgggcacttgccctacgtgtacgcaattagtgtcggacgataccaaatcacgcattgaaagtcaatattatccacaaatcattgaactgactgacaagattcttcaggttgacacagccctagaagaagttaattccctgattgagaattacaacgtcaaagattcagaattatcttctcagatttcggttgttgacaaagaactggatgctttgcgcaatgaaatccgagaagcggaatcagcgatcgctgcattaaaacgcgacatccaaggattctatgatgtggcagctgttgagaaaacatctttacagcgggaacaacagtccaaaattaacgatatcctcaaggccgtagacacccgttacgaagatatcacgacgtcaatccgtcaagcgcgcgagcagctctctaagagtatcaacgacacggctgataaactcaccagcctgaaatcacgccgtgcccctctggaggcatccattgccgatttagagcgtaaattggcggttaagccgacaccaactgacaccctggaagaagagatcgctcgcatcacggcgttaatggaagatctgaatgcgcggcgcgtagagttggatgagaaattgcaagacctgaaccatcttctgttcttcctaaaagatgatcagaccaaggctcgaattatcagcctgtatttgcctttcctcaatagtaaaataaacgagtatcttgaagcactcaatatgttcttggatattgctgttgatgatacatttgaaatcaccatgagcgctgcaggtcgtaaagggcaaagtatcttctcgttatctactggtcagcgtagtcgcctgaacctggctgtgacattagctttacgcgatgttgcaaacctcaaggcgtcggtgcaatgtaacctgtttgtgttagatgaaatccttgagaacatgagcgagcgcggcgtacaagaatcggttgagatgctgaagcataaatttggcgggaacaatctgttcgttatcagtcaacgcgagcaggaattccaagagtatttccaacacaacatccgttatggtctgcgtaatggcatgactgaagtgattaagaaggattgatcatgggaagtctgattcgtgtttctaagatatccaagggcgctgaaaagcgcctgtacaaagccgttcgcctgatgatcctctacaagtacactgctgagaatcggtgctggcgaaagaaaacacgtgcccgatatcagttagctcagtacaatatgaggcgtcatgtcgtccggttggcgttgaagatcgcggggaattacaagtattggacttgtgaggctgttggtagccgcaaggctattgacctcataaatgaccggaatctatctttcctgatcaatctatcggattatgaggacatgtcgtgaaagtcgtccacttcaagaaggagccatatgacgtctacattgggcggcctggcaaatggggtaatgacttcgaagtcaaagaccatgggcgcggcgtttgtatcgagttattcgaagacgatttgtatgtgcgtctgattgaaggggaaatcactgaggatgaactccttgaactggacggaaagattctagggtgctggtgtaaaccacgcccctgccacggggatgtttacgtcaaagtgatcggtcgtattaaactattccgtaagttgggtaaatcttttactgaatatttgagacaaacatatgtcagaagagaaagatgaaatacagattgcttgtgatatcgctgtggaattatggaatcagcgaaatagacatcatcaggtagcattgacaccaaaacgttacctagagcaaggtgggttgatcgttgttgacgccgtcttgggtttcaacaagtacacagatatgcaaatcctccaaggggatttcatagtggcatcaaaatcagcatgggacgatcctatggagtttgtctacggctggttagagtgtattgaggaatcataccatctgatctacgaataaggtgaagggcactaaatatcaagtgccctttatcttttaaccctgtcaggaggctcaatcgagctgcaagtcgacaaacacataaggaacgccatcatgacgatgacaacgttgaaggccatggccttgtccgtgacctttaccattgccagcgggagtgtgcacgcttcccaaagtaccacccaatgtgattacgaattttctgatcagcaactttcaactatggccactgcctatcacataggcaaacagcaagaccttggtttcaccctagccgcgatatcatggagggaaagccgcgccggagaagatgtggtatccatgcggaataacctgaaatcggctaatatgggggcattccaaaatcgcgtccaaaccgttgggaaacgggagggatgcaaaacacagaagtgttacgccaacgtggcgattaaattgctggtcaatcaagagtacgcagccaatgcagcccttgatgaaatgaatttctggcttggatatcataaccaaaacatccgcaaatcgctctcctcttacaatgccggtttcagtcgtaaccaaaaatccaattcatacgctgctgatgtcgtgaagaaggctaaatatcttcaacgatgtgtgtctttcaaaggcatggcaataaacccgaaggtcgacccaggagtgatcgccatgaataagcgcacgattgagaaactcaagaggacccgataatgaatatcactccatcccagaccggatacgagcacttaatggtgtttattgcgattcagggggcaaaagaaggcatcatcagttctgaatattctggaacgatgcgcctggctgattatgttgctcccgaagcgtttatcaagaaatgtatcgacgcatggtgcctttatgctctcggcacgtatcctactcctccggcaggtgagctactgtttaccgttccgaccccagccgctggcgacacagacgcgatctttgctgatatgttggccaaaggttatttggcgtatgacagtgctcatgatgtctacgatttctcggcgttgaatcgtatcttcggatacaccataacatccgaagccaatattcccgactatggaattctggcatggggtattgacggtctcggcgcaacatatgcgtcttctgatgttggtcctgtattgattgacacaggtgtgacaatggagcaatacatgtcacaattccctcagcataatgttaccggaagcgatatcattatcgtctatcctggttccaatgcgttgtcccgcctggtggacggctggaaacctcagctgaaggacatcgttgctggaatcagatttcacatccctgtcgtcaatcccacaccttaaaataattcaataaaaggggcaaacgcccctttacttcttcaataactaccgtataattctaaacataagcagtacgacaccaacttgaaaagcaggaactatatcatgaccaaaactgaattgaacaacattctgaacgttgctgggtacaagaatattcgtcgtgggaataacaaaatcgcgcgtgaccgtagcaacaaaatcatcaaggggtttcccctgtgtaaaactggtcctctcgatatcagcaacccgtccaagcgcttgtttaaagacttattgcgtgaactcggcactaaaggcctggtgctagataaaaaggatgacctggttgactttggcatcgcggttatgaagattgccaaattcaaaataactctgtgcgtacagaccattcagacatatgactactctgaaggccgtgacccgaattacgaaacaaaatatgtcaacgtaaggattgaagggttataatacgaatatgcgggggcttgatgcccctttcttttcgctaattaaggaaaaccgcaccgatttattatcaggagaattacaatggccatccaatctttcttaaatttccttgacgagtccaaacaattggacgaagcgtttaactccgctccatacgaattgactttcggcaagaaaaatgctggtgacattttcttcacgttcgtagacgaagatgaaaaagaattccgcatccaattttataccccgcaggggctgggcaagaacgttcgtcaggtcttcataggccagaaacgtggttcaacctatccagacgcaatcggtcgtttcaaaaacccaatgcgtgtcatcgcatccatgatcgaagcgactaaacaattcatggcgacaccgctgggtaaaaccattgatgggtttgcgattaacttctccaagaaagctctagaacgcggaacgacgttactcccaaaaattattcgtcaatctggtctgaagcagaaactcaacgtcatggatctgacttataccccaatccctgaccgtgccttcgtatgggttgtgcgtaaaggcaaggatcctgctcaggtatttgatggtccgaaaatgaaaggcgtgacttgggacgatccagacaaagtcggtgacgttcctgaccaggctgctcgtgacgccgcactacaaggtgacatggatgatttgagccaatccataaatgccgattctcgctggattctgaccaaagctgatcatactcagcctatgctggtctggtctggtaaagaacgtgggcggactattaccgccaatatcgcacctcacgcgaatgaacctggtgtgtatgctggtagtatcatgaacgagaaacggatccgcgctcaatccccagacgctattgtacgccaactgggattaccgcagatccctgtgaatattctgaatgatttcaccaagcagtcgtccatcttctggaaagagatgacgggggattctggaaaaatagacatgcagactttgccaaaaacaggatctgtggatgacttacgaaatcagggttatattgctcgccttctggggattgtagacacatatgctccacaacaggtcggtatgtatgcaggtgagaaactaacacgaatcggcgcgggcaaatatcgtacttcgggtggtctggacattcaggtttatttctggggaattgtcaacggaaaactttctgtagaagtggtttctgatttatctggcaatgaacagctgatgctggaaggcgacgtgaaaaacgctgcttttgaaatcgtcagtgcgatatcccgtgctgctgattctcagttcaacggtgaagtggaatgtcgtgttgttccgatcaacggtgccccatacccgattcaaaaggtttcccgttctcagggttattcttatgtcgattgtatttcgtttactttgaagatcgccgatgcattgttaaatcgatttactctgggtaaggcagtcaaactcgatctgataaaagagttccctcgaacgacgtttattgttcgttatgactggcgcagcaaagatactctgatggtccagtcttctgatgggaagactatcttcggcgaaatcaacacaaattccagtgataacgctgtcatcactgcgaacctgaaagccccaagcaatgcgaagatatttggtgaaaccgttcatgggttctctgtggcttgggacttccagttccctgcgggtagccagctgcaagccgcaagattcgttaccaacgtgacttacgacttcgatcgtaataaggccaacatcacgaccatgctgacagaaaatggccgaaatgtagtcaatagcagcggatatgacattcagttatctgtcaagtctgtgcaagatgcattgaatgagacttcacaacgcctgaaagacgctgcggctcaattgagccatcttggttcatcaaactataacccgaacaagctgttcctgaatgatatcaccgtcagtaagtttggtgcaattatgttccaagacaaagatctggcacagtccaaaaaccgcacggcagtcattgatgctttggctcgggcgaaagatcaggtcgctggtggtaagaatgcaggcgatctggccacctatgccggatccattcgtgatagcgctcctaatggacgagatctggattggcaggtatacacagccagtaatggtacagttctgaatgttgcctgggatatcactttccgtcgcaatacgaccgaaggagcatatcgtgaattcaaggatcagatcaaccgtgccaaccaatacctgcaaacggtatacaacgacgcgaaatctaaaggctataatcctaccgagccaaatctcatgactcttgaacgcgctcgccaatccgacgaatgggctatgagtaatggtgatagtgcatacagtgaatatgaacaatcgctgggtggtaatcttcagattagtctgaaataagtcatagccccttcgggggctaaatacatcaaaatccttaggagatccacgatgaaaccgtttatcgaatattttaaagaacaagctcaacaaccgcaaactcatggcggcatacaaggcaaaagtgtaaccatcaccaaacaggccgacggaacccaatggtgtactggtgccaccgtgacccagatcactccagataatcaggccgacatctataatctggaattgactaatggcaacaccatcaaagtgaagctaactccagaccagacccacggcattacccaaggccaagaagttgttgccgtccatgatggttttgaatttttcttcggcaagtcagctgacaaaggtcaggttactgaagcggaaggctggcgtttcgtcggtgaccgtggcgctcatattctgatgtatgacgcaaaagttggcggtgcctccactatccttcgcagtccaacagatcgaaccaaatttgttggtggtctcatcaacggtagaaaaattttgggtaattccccagaagaactggcggcaaaattaggtcttccaaaaattcctgataatttattaaaatcgttttttggtaaaaagtgattataaaatacaatccccctaaatacagggggattttctttttaggggtaaatatcatggtcaatgcgagtatattcatcaaccgtggactgcttgctatagcccactcccatgccctccacttcgttacaacgtcctatgccaagcacaaggcactcggggagttctacggggaactggaggacttgctggataccttcacagaggcatatatcggcgcgggtggtgtatatgtcccagggtttgagaatattaaactgtacaacccagatcctatatcgtatgtcaacagtgttgttatcgacgtaaacggaatttatggtcaatgcgacagccatcttcaaaatacgcttgatgaaatcaaaactttgtgctatcaaacgatttacaaactgaagaaattgtcctaactcaataggatcatcgtcaggttataattttgcccattcgtcatcaataggatacagataacaatgggcaaaatctttataccttctcactcagaaggttgtcaaatcgctgcaaaatttctacacaaacaaatgaattgtggtgctgtgttcgtagaaccaaattcattcgataatcgtgaatcccctgacgccatcgggttcagacctgggggttgttctatattaatggaagttaaagtctcccgcgccgacttcctgacagacaaaaagaaacctcatcgcatgaatccttccataggcatgggagcatatcgtttctatgtttgcccagcagatgttattaaaattgaagacctccctcccaaatggggtttgttgtacttcacccctagaaaatctttgaagcccgttcacgttccgaatatgcaatattcctctttgtcttcaccggaacattatgcatcatatttgtctaaatctttgggcaaaagaagtcctacagatatccctccttatctattagggtacaaacagatgctggaagaatttgcccactttgatcgcaatcaggttgcggaacaaaatattctgtatggggcttggcgacagctttgcatagctcaaagtcgcggggtggattataatgtgacagaagtgttccagaggccaaatatctaatgaaattcttggacgaacaatacatcaattttctcgccccccgacttgataaattcagttgggaacgtgttggcgctgtcgccaaatttcgttgcccattatgtggggattctaagaagagtgccaacaagcgtcgtggtcacttcttttatgatcgtgatgacgatgtgtttcgtttcaagtgccacaactgtaatgaaatgagcggttgggcgtttgaattctggttgaagaagtttgacgagcgtctgtataatgaatacaaccttgaaaaattcagaatcatgggggataccagttcccgcccgttacaaaaactcaaaccactacagcgcctgacccagacagcacgtatcggttctcaggtcgccaaacgggatgaagagcaccttgggaatatgatacgccttgatcttcttgaccgtgatcatatcgcccgtcgttatgttgaaggcaggggaatgccggagagtgcgttatctcttttgtattacagcaaaaacttccgcgaagatcttttgaattttgaaacagacgttgaaaaacagaagaagatccctgaagatgaacggttggttatcccattctggactcaagacggtcgcatgaagatcgttcaggggcgcgcattcggtgataaccttcctgataatgtgttgcggtacgccactgtcaagcccaaaaacgaggatactaaaatctatggggaagagcgcatcatatggaataagaccaaattggtcgttgaaggtccaattgatagcctgttcctcccgaattgtttagcaactgcggatgctgaccttctgagcgccaagggggatatctatatccctgacaaccaatatcgaaacaaacaagtttgtgacggtatacagaagatgatagacagcggagtcaaagtcgttctgtttccaccggaaataccgtggaaggatatcaatgatatggttcgccccgacaagggaaatatcccgatacgtgatctgcttcagatcatagccaagaacgtatatcaggggatgtcggcgtccttacgattttctgacttgaggaagatatgatggatttgaaagaactgaatagtatggacgatgaagagattcttcgtgggtatctgcaagcccgtgaaggatatgtcctgtctggcttagaatctaagagtttcatccatggttggaggaacggaatggtggacttcggcggttgtcctatcactgaagaacaaatgaatttggctcgccagtatttgaaaaagaagccgacctaaaactttttaactcaaaaaccttctctctaggcctcattctacgaggccttttaaaatacttcaaaaataatttaaaaatttattgaaaaagactttactcttcaataatcatgcactattatagttcacatagggcggtacacaacaccccgccgaatgaaaagtgaaacgacccaactacattatgaggaattacgtcatggctactaccaaaactctgatcaccaacggcaccatctcttttgaactgaacaccgaagttgcaaaagttgaaatgttccgcatcgctcaggcagctggcttcactggtggcaaaacttccttcatgaatctgctgaacggcaaagtgaaagcaaccaacggtttcaccctggtagagcaggttgtcgtagacaaagccgtggttgccaaaactgctgacaaggttggcatgctgaaagacctgggccatgacatccacgttgtcgaagcgaataccgaaacttacggcaccatcgtagtcggtaaaggtcgcattcagctgaacccgctgaacaacggaaccttctccgtgatggtattcccgaaaaagggttatgacaacagtgatatcgtgaaagccgctggcggtgaagcgaaatcccagtatgtcaaaatgggtaaactgagcgccgacgcagtagaaactctggtgaacaaactggcttaaccgcaagggggaaacctccccctttcaaagttttggtaggagaatgagacatgagtagagcaattatccaagcctctcctggcgctctatgggttgccgaccgaattcccaacgcgcactttcctcagttccaagaggaactggaacaagcaatgttgaaaatatttgaaaaatatgggtatgattccgaagttaggacttccttcaaagaaatcatgcctgtcgtcgtggtcagataatcaacactaaatcctcaagttcgactttcactttaaataccttcgcatacattgcggaggtattttcttatggccattctaaaacttggcaaccgaggttctgaagtcaaagcgcttcaacaaagcctcaacaaaatcggtttctcccttacagccgatggcatatttggtaaggcaacagagaatgctgtcaaatccgttcaggcaggtgctggattggtgattgatggtattgcaggtccaaaaaccttctatgctatccgtaatgccggagacgctcatcaggaacatcttaccgaagccgacttagttgacgcagcgcgtgaacttggtgtcgaactggccagcatgaaagctgttaaccaggtcgaatctcggggaacgggtttcaccaaaacaggtaagatcaaaactctgttcgagcgccacatcatgtataagaaagtggcggctaaattcggacaagcaagagccaatgctctgtaccaactctacccaacattggttaaccccaattctggtggctatatcggcggagacgcggagttggaacgccttcagggtgcaatcgcccttgatgaggactgcgcgtacgagagtgcgtcctacggcttattccagatcatggggttcaattgccagatatgtggttatccgaacgccaaagaaatgttcacagattttctgactggtgaacgtgcacatcttctggcatttgtgaaattcatcaaagctgacgccaatatgtggaaagcattgaagaacaaaaattgggctgaattcgcccgtcggtacaatggtccagcatatgccaagaaccagtatgacaccaaactagcggcggcatacaagagtttctgttaacaaaaggccggaaacggcctttttctttatctaacatacaggtatgctgggcgcgtcgcttaattaaaggtgtcgacaacccctgtagatataggaaggttcaacatgcaatctgcgtctaaagttgtatccatgaagccagcgaagacaaaatctgctcgtaagaaagacaccatccagaaggaagaagactggatgaagttttccaaaggcgatttcaaaattgctccgttcaatggcctctcagaaaatcagaacctcgcatatcaatccgcactcaatgaacatctcactatcgctatcggtccggcaggtacaggcaaatcctattgtggtgcgtccgctgctgccaagcatctgattgacaaaaccatcaacaagattatcatcacgcgttctcctttaccgactggaaccacagcggggttccggcctggtgacacatatgaaaagctgatgccttatttgatgccgctcatacagacattcaaaaaggttctcaaaacagatacgggttccgacggtttcttcaactatctgtgggagaaacgcatcatagaaattcaagaccttgaaactgtaaagggaatgaccttcgatgattgtttcctgattatcgaagaggcgcaggaatgcgatatggagcaactgaaaaacttattgactcgtgcttcagattcttcgtacatcttcgtgaatggagatatcaaacagtctaacaagcgactgcgtgatagtgcgttacagacgtatgtagattcctttaaagacttcaacaacaagctggaaacaggttctttgcaaattgacggtgtagaaatcggagatgaatatcctgagtgggttcagccattcagcatcatcgaattcgataagtctgatcgtaatggtcgcggtaacttcacccgcctgatgttagaaattaacgacctgtacaacatttaaataccacacgaacttccccgtcgccttcaatccatggaggcgacgtataataacagcctgcattaacacaaaccgaggatcgctatgattaacatcataaagcgggacggatcttctgtcccctttgacattgaaaaactccacactgttcttgaacgtgcctgtgaaggattagaaggagtttcggtgtctgaggtggaagcagcatcgaagattcagttcacagacaatatgaaaaccgaacgcattcaggatattatcatccaagcggcggctacattgatttctgtagaaaaacccaactaccaatatgttgctgcccgtctgaaatcgtatgacctgcgcaaagtcgtctatgggcgatacaagccacctcatttgcttgatatattcgctaaaaacatcaagcagggcgtttatgatcgtgaattcctcgaactctatacgaaagaagaatttgaagaactgaacacggtcatcaatcacaaacgcgacaagaattttacttgggcggctatgggccagctcacgcaaaaatatcttttgcgcgatcgctcagcagacagcaaagtatattacgaaacgcctcaagtcatgtatatggcgatcgcgatggctctgttctctgcatgggataaagaaacccgcctgacgatggtgaagaaattttatgaatacgccagtacaggtaaattcagtcttccaacacccatcatgtccggcgttcgtaccccaacccgccaattcagttcttgtgttcttatcaaaactggtgacactttggactcaatcaatgctacagccaaaagtattgttgattatgtatccaaacgcgctgggatcggatttgatgtcggagcaattcgcggcatcggtagccctatccgaaaaggcgaaatggtgcacacaggcctggttcctttcatcaaatatctgactggggcattaaagtcttgctctcaaggcggtattcgtggcggatcagcaacttgctatatccctatctggcattatcaatttgatgatgttgtggtcctgaagaataaccgtggtcttgaagagaaccgtgaacgccgtatcgactacggtattcagatcaaccgtgtcatgttcgaacgtctggtgaacaaacaacccctgtatttgttcgatccgaaagacaaccgcgatatgtacgaagcgtttttcgcagatgtcgataagttccgtaccctgtacgacaacatggtcaaagctgctgatgctggtttggttcgtgccaagaagatgaatgcggaagaagtattccagatgttgttggatcagcgttcagatacaggccgcatctacatcgcgtttgtcgaccacatgaaccagtacagtccattcaatctagacacaatctatagttctaatctgtgtctggagattgcgcttcctactcgtgaattccaacagtacgacgatgaagaaggtcggatcgctctgtgtactttggcctcttttaacctgacggcgtttgaagacccgacagaaatggaagatgttgctttcgttctggtttcagctttggatatgttgttggaatatcaggactacccagctcgccaagcccgtttggctgtagaagaatatcgtcctctgggtatcggtatcgtcaacgtcgcgcatttcttcgccaagaacttcacggggtatggatctcctgttgggttagaactcctggacaagtggatggcgcacctgcatttctatctggtaaaggcttcgaatcgtctggccatgaagtttggggcatgtaagaaggaaaccattcatcgttctggtaagataacagccgaccttcaaccgttgccgctggatatcctccccaatggtaaaaagccggaaggtatggcatacggccttgactgggacggtttgaaagactctctgcgttgccatggtatccgcaatgctactctcttggcgatagcaccaactgaaagctcctctcaggtgctgaacgccacgaatggtatagagccacccaaaggcctcatcagtatcaaaggcagcaaggatggcgtttacaagcagatcgttccggacgttgaaacccttggtcctctatatgacctgaaatggaatctgtcttgtatcgactatttgaaaacagcggcggtcgcccagcgttgggtagaccaatccatcagcgttaatacacattatgatccagagaaattcccagacgggaagatcccgcgctctctcatgatgcaggatattctgtctttctacatgtggggtggtaaaactctgtattacaacaccaacaaagactctaaggaagatgaagagttgaaacaaattgaagatccgaatcactgtgacacctgtgttgtctgattaggtctataccacacaatcgtttaatatcaggggtgattcgtcaccccttattcatcaggaaagaaacatgaacgaacaaaagcaattctcagtattcgatccatcttcaaataatacaggactgccattctttggcgaccctgtaagtatccagcgttatgacaaagtggcttggccttttgttcaaaaatggtatgaaaaaggcctcagccaattctggcgtccggaagaagtggatgtcaccaaagacaaagcggactttgccactctatccgctgcggaacaacacatctacttcagcaacctgaaacgccaaaccatgctggactctattcagggcgctgctccgttcgaagcgtttggcccgtgggcatcaacaccagaagcgcaatttgctattctggaatggacgcgccaagaagccatccactcactatcttacacgcatatccttcgtaacacagtgaatgatcctggtatcgtttttgaccatgttctggacgttcaggagatcgtagattgcgccggacagatcagcgtatactacgacgacatggtgcgttacagcggaatgcgtatggctggacgcgcgttcacccgtgaagacatcatgaacgccaagcgtgcattctggcgtgctctgtttgctgctaactccctcgaaggcgtccgtttctatgtttcattcgcatgctcttgggcattcatgcaattcctcaacaaaatggaaggcaatgccaagattattcgccagatcgctcgtgacgagcaagatcacctcattctgacacagactttcctgaatcgtcttccgttgttggaccctgattttggtatcatccgtgaagaactgcgtggcgaaatgacccagatgtatgtggatgtggtcaaccaagagaaagagtgggcgaactatctgttcaaagacggatccatgctgggtctgaacgcaaatatcttacaccaaatggttgattggctagcaacacatcgtatgggcgcgattggccacccgtaccctgggcaagcccgtaaagacaatccggttccatggataaatgagtggctagataataaaacaatgcaatacgcattacaggaagcggaagcccctgattatctgactggtgtccttactggatcagtctccgacggtctgaaatttgtttaaaggtgagaaatgattacgatctattccaaacaaggttgtgcgcagtgtttacaagccgaaaacatctgccgcattcgcggtattgaacacaagattctgaaacttgataaagattacaagctggaagaattacagaaaatcactggtaaacagcgcatgtctatgcccgtgattgtcctggccgatcaaaccgtgaccgatgttactggactcgccgcaagtttaaaacgttgatatttcaaagcccctgatccaggggctttacttttgaaaccttcccgcctatacttcccaataattcattggcaaacgccacaaattgaacagcaggaactatatcatgaccaattctgaaatatatcatgcttctatgcattatcatgacctacgcgcccgtccaggacacgccaaacaaaaagcattctgggtggcagaactcagattacgccgtgccgtcgttaaccaccaaataatcatgaatcggaaaaacgacaaatcaccgttgttggcagattcttatcttgaactccgcacagcctatgaacaagccaaacagcacgtaataggagcataatatgtccaacaaattactcaccaacccccgccacaactttggttatatccaattaccagaagtgatagacacgttgtttgctgaagacaaggaagaagtttgttgggcaccagcgactcttattgaacgcgaaattttcaaatctgaagtagacgtgatgttccgcatcggttcatccggagaccttcattatttggggaaagtgaccgagttaaagacctgccatgctcaaagatttttgcgcattgacaccactcaatgtgacgacttttcattcagcattctgcgtgaatatgttcaggtcaaccgtcctgtgtatcaacaccgcccttatttgacgtcgatgcatatagacaattatatttccgacattcatttacgtgcatcaactatccatgaagaagattctggtatcgcggttgatacagtactcaatatgatcgtcattactttggtgccgtgatatgtacaaattcccgatattagaaacagtgtactcagttcacaaaaaggaaggtccattatttgaaaatttggatattctgttaaaggaaaatgtccgtatccctctggttctaaagggaaagcaaataggatatagccatgactgccaaataatgcgttctgaaggcatcgacgtcttacgcatccgtctaacatttgtgttgacaggtaatctggacggtatggtggttaatctgaaaccaaccaccatagtccacagaacgggtgggaaaatattatatcgtttattaactcattttgagttggtgacaaaatgctctgccacggacgttctttaatcaatttgtccgtttcaaccatatggtgtctgttatcggctctgggttgttgaccacataaaacgttatggttacacccagagaatgataatcttcagacaatgacacatcaactgattgtatctccgctctcggttcaaataaagcaatcgcgtcttcaactttgtttttaacatcaacctgaattgtgggattcgtattttcccccagcatggtatacagcccagccccgatacttgggtatgtcggccaatccccaactgttgtcatcacgatatttcgtacggattgcaacacggcatagacacctgttttctttgtgacatctttggtgaccggatgcatgccaaacttcaggtcaatgtccttgtactctttcatgttaaacccctttggatattgcctctccggcacgagagggcattatctaccctcgccagtgtaatcatgcgtctttaccaccaaagttgccgccattatcctccagttccggttttatatcaattggaccaggaccagaagtagatactggagttgccccagcgcgtgatgcagcacgaccagcaccttcagcatatttgatgttgccttcaatagtctgcgccaccgacaacttatcacaaactatttcactagccagtattttaggcacttttaaagtattggaaacttccaacatctcgcagatgatacgcatcacgttttgcgcctgaatttcggccagctgtgaaaatttcatcaacgccgtgccagcaaccacattggagtagttcgatgaatgtagatgatagacctcacctgtcttgcgctgaacttcagtcccaccaatggtcaggttgtgatcacgaccaacatagtaacgtttgtcaaacatcgtcagatcataatgatctttgactgatttattgaccacgtccccgtcaggaagcatctgcttataagaccccgaggagtgcatccaatggagacgttccccgcctggtgtgtcatcaacctccataatatgccccgaccgagatgccatgacattgttgtaaggataacgagacccgccagcagaaggaaccgacacctctcctgtgttatcaatatcttgaacacgcccagaaggaggcggagtttcctgaacttttggtgtacgatcggggattggctcagaccatttaccattttgttgcgcaggagccgccgatgctgtcgtagcaggaccaggccaacgataacccaacacagatgagcggctgaagcgactcaccttcacagaatctgattggttgcctccaatacaccaaacataattggcatcgaatttctgtacaaacgcaacgtgtccaaatgttgggttgttgccacgacggaatacaacaactgcgccgtaacggggttctgacaaaggggaaccccattgcaaatatgaacgagccaaagccgagcgcgttgaagtgtatcctgcttggatcaacacccatcccacaaatgaagcgcaccagctgacttcatcttccgaagcgcccaaagatgttgtcttgtgatattccaaaatccttgggttattattgaatttgccagaatattctttgacacctaattcaccacgagccactgtcatccatttctcaggatcataaccgtccacaggcggttgaggttctggttgtgggtcgtcttcaatcttgacaggaacattttcaacggcgttatatttctgtctttctatagattgaacaacctggcccagcgccaacgggtttgtgtctgaaccatccgttggtgtcgccgctggccaaacccaggcgatacgaatattttggtatgcttcatccaaagcaaatcccataacatccgacccgactgtaatacccgttggagaccagcctaaacctgccgaggaggcgttagatgctggcatgagcattttaccccaaggcaataattcggtaggtaagagagtggtatcttccgtgtgtacaccatagatgcgcacagcgacgcgcccgttctggtcgggatcattcacgtcttcaacgcgcccgtaaaaccaacgcaagttgtctaacatattgaaacccattagtcaattagaatcatgggttatttatggcggggatttgaaatctgtcgaatagtaatattataggaagctctgcttccgttcaactttattcttttcgtttctactcgctgaagctcgtaacaccgccttgacaggcagtcccattccaatgccatggaggcataaaggcgaggaaaatactgtacacctcagaacatgcgtagaaaaggattatttttgcttcaaaacaaattcaaaccccaatttacacaacacttacttcagcaacaacggtgttatagcaatttaaggttaaacgttcctgtcttaacctataaaacaggtaagcaaagaaaaccccacattacgtggggttggaagatttcttggacaaacgtatctttagtgtctctgttagcaaatcaagatgccaaggcatcgactcgcttatttcaaatatgctatagccatgtgcctttaattcgtcacacatcacaaagtaggagagtaaatcaatttcaaaaattaaatgaaaatttcagtgacactgttaaatttgacttcatgttctttcccacacgaaggacatttaattttcgtcgcgtaacggatacgaggaatctttttaaagaaatcgtcggatatatcttgaacaatctcagattcaatattttcccccacccatttaacaaattcatctttgacgcgttgacgttctttagcaacatcaggtgctatgccaggctctgccggattctctactttccaaacctgaccatcatcgtcatacaaacaatcaataaatgtcgcgatcatttgctccacagatgatgcttcgttgagcactgaggcgtccgagaaggacggttgacgcatcttgatgtgatagccaccagggaggtcaaatgtttccctgaagccttctggggacacgcatttcacttggttgagggggatcggtaaaacaagttcttgaccacattctttaaggccagaaactggttcttcaccttcatttgtgtcggatggaactttgttgttacatttgtatcgtatcttcatgacctcgccgatagatatacagcgcatctttaaaaatacttcttcagtaacaccgattggcagtttactaaagggaaccccagcgtcaacacaactatcaaacagttgctccagcgtcgccacacgttcactcataggagtgttcggatcagcaacctgaagtaacatggtctgttgccctgctgtgaaagcgcggtatttaatcaccgtcggccagaaatcacttttgtgtgttctctcagttttgggcaatgatggtaaattcataatgtatctccacggtatattgtaaccatatttataatgaggaccgaaaatgcacgcgttctgtttcagtaaagttggagaacaacttaccattaattatccggatacaaatacggatttctttgtattgctaacggatttcgcaaataaattggggatcaagaatatcgccatgactgtggacgatatgaaatttggtcgtctgggtgtaaaagatttattgaattacggattcaacgtaatcatcgtcgatcagacatacagcccaattgacgaggaagatgttattccagtttgggttcaacgagacataaacactctggataagcattatcccgattgtgtcatcattggcgaattaataccatattttcataaacgtcgtcggcttgaatcaattaaaagttattttcaagatagcgatgacggatataaaatggatgtccttacgagcggttgggttgtgaagaataccacaggcaatgccgaccgaattacttcccgtctttatgaatatgttaaagaacgggattatgaactggagaacaataatgccaaacgtagaacgcgctgatattcagcaggtacaagacgtcatccttcaacgcctccgcgctgtgatcacaaatgaaaagggagaaattgcgccagggtttgaaggcgcattcgacggtttcgttgccgatgatcaggggaaccccgttgtacaaacgatcgctggcatgattatgctgaatagcgaatttatggctgatggtaaaatccattattctcccaatctcagtgttgacggagaatctttggcttccgaagtggttgatttaacggttgccgttggtgagttgggttatgttttgatggtttgcaccgcgcattatgtggacgccaacggccatgtgtcatatggtgatgaagcgcggggaatcaaacgtcatgtggatactgcggcagttctgcaacagatccgccagatgcaagacaatatggattccaaaccccgcctgattttgccagatagtaaaatcgtgacaagataatccggtctaaatacccataacacaatgaaacttcgttatgggtatttttatgaaattttgcggaatcgactattcttatggctgtcctgccatgtgcttctgggatgataaagatcctttggattttgaccatcttcatttctatgcgcatcatactgtagaaaaacattgtcgtcaggtacggcataacatcctcattttacgccagccgaaatatgaatccccagaagaacgcttctacaatatctccaaatgggccgaagctgttcttctaaccgagaagcctgatttcatcacccttgaggggtacgcaatgggcaactcgaagaattccaataacatatgccagactgctgaaaatacatctttattgaagcaagcaatgcgccggaataatatggaattccagatcgttactccttctttcgttaaaaagcatttctgtggtaaaggaaacgccgataaattggtaatgatcgatcatttcgagaagctgttcaacgttaagatgcgtggtataatggacatgttggacgtgaaggatccaaagcctatcgatgatcttgtagactcattcgcaaacatggtgtctggaccttacttcatagaaaattatcccgatttcaatagaggtgtaagaaatgattaattactggctgttggcagatatactgcttttcgccttgctgttgataacggcatttatttgggttaagggatttctgacattcctgcactcgttgagcgccatgatatctttctatgctgtgtccccccatagtgacgtcaatgtacgagcggcaaatcaatctgacatgttggctgaatacgtcatgatgcgtgataaagcaaccaggatcgtcctgttttctacactggcgggggcggttattatattcatacaacacgtcttggaggcaactcatgccgttttatgattatgcctgtacaggttgtggtaattctttcagtaccagaaggtcttgtgctgaacgccatacgcctgaattagatccatgcgctgaatgtggcggtgaaatcaaaatggtcattggagcaccaaagatcgtatctggtgtccgtggcccacaatccgcacctgatggcttcaaagacgtattgcgtcatattaagaagcagtctgggaaggggaatacaatcgatgtctgaccaacaaatagcacaagtccttacacctgattcattctcagaattggtgttgatgcgagcgagccaacgcaaggagtctattcttgaaacgatggccagtgtttgtgaagagtacgacatcgaagaggcgaaagttaagaaactgattacaccacctctcctgtcaaggctaacagctgaatgttctgatgctcggttgttgaagggtgaattgaaatctaaaaaactcatttaaggttgtaacatgagcaaagagcaaatttataaattgttgaaggcgcaagagtatctgccaggttctattcgctggcgtcatggtagcctcaacgaacatgctgatgatatggatcgcgttcgtttcacaacgccggaaggtaaatcctatgtcatcgagtaccatacattcttggaaggtcataaaaccttctcggatgtgtacgatattattgagatagacccagcaaaacaaatgattgcgggttaactcaatagcaaaaggcagtataattacatcgtcggaataatagacaaatacatttatagaatagcccgaaggggcggattatagaggaatataaaatgggtaacttatttgatcgtcttaaacaatctcgtggccaacaagccgaagccatgcaacagcgtctcgctcagcaaggtcagcgtgttggtggcggtcgtgacccgcgcatctggaagtggacttggaatgaaaaaggcacttccgaaaacattattcgtttcctgcctatccctttagtggacatgaaggctcaagaagaaggcaccatccctgaagatgctgtgttaactccatgcgccatgatcatgaaacacgctttccagggtgctggcggctggtatatcgaaaactcacctcagactttcggtaacgatgatccggttcgtgaccatgatcgtcctctgtgggcacaacagaaagaaacgaacgatgaaaaactgaagacggtcctgaaaaaacgtctgccggacaccaaatactacgccaacattctagtgatcaaagatggcaataatccggaaaataacggcaaggtcttcttgctggaattcggtaatgccgtcaagaaaatcctggattcggctcagaatcctaagttctcaaccgacccgaaattcgacccgttcgatatgtgggaaggtgcgaatctgattcttaacctgttcggcgaagagaaagagttcggaaactggaaaggcctggttgccaacttcaccaacgtgaagtgggatagtccggcaccgctgggaaccgacgaatacattgaagagatctgggaaaaagagcacagcttgtttgagttctttaatccggccaacttcaaatcgtacgaagatctggaaaaacgtttgcgcaaagttctggccatccctgataatcagcctctggttgaaggtggtgcttctactatggcacatgcgcctacccagtctcaggaaccccagcgcccgacagcgcaggaaagcctgaaccaacaacaatctcagccttctaatgcacagcaatctgttccagccaataatggtggtgcgggtgccgatgcgaaacagacggcttccattgatgagttcgagcagttcctgaagcaagactaatttcactaaagccccttcgggggctttttagtttttaacgacgtcttgtaacaaaccgagtaaactactcttctcagacgccgtcaggttatcattgcctaatatatccctctggatgccgatagagatcctttcgaagtctgccaggctgatacccgccaaattatttacaagctgatcgagttgcttgtaggccagtagggcttcacctgtaaagttcccttccttaatctttttatacagctgtccggcttttgacgctgcggtttcccaatctccggaagcaagagcatcagcaatgcccaaaggaagaaagtcgctggcgaggctgcgcgtttcatattcagtctccgataatactttattgtaagagaacgatatattgtactggttaaattggtcggcggcacttttgtcgaggtcgatggaactgaagttaataggatgagcctccgtcacataaacacggtgcaccacctggtcttctgtatccatttgctctatacaaatatcagtcacgaaatcttcgtaataacccatcttggtcgtatatgggtcgaatatcaaattcttccatttatccatgaccgacttttcataatagtcgttggcaaggagaaatgataactccagatcaacgtttgttttgttgttcggcatcttaatatggttgccgttatttgtcattggcgttgtatcaatcccagtaccaggcaaagaagcaaccatacacatcatttgtaaggaacgagacgtctggtttgtccctccaaagaaagcgtttacaatacgggcgctttgtttgaacagatccccaaatgatgaagatgaaggatatgcgttgccgtcattggatagagtcgcattagaatcgaaaataccaggcggcaacgggattgtaacacgaaatctgttcttgcgggaaatcccccgctgaagcagttgcgttagaaaattgcgatagtcttccacgatgaggactccatttaaataatatatctgctgatatttataaggagttgacaaatggccaagaacgctatgggtgaagaagacccgttgttgcttcctgccgagatggacgccccagaattggtcaagcgttacatccgcaaatatcgtcaacattttggaccagaagcgaaacgtaatatccgtcggtctcacgtgtggtttatggaacgcgtatcgaaagatgccaatttatcccccaaccacatgatgaaggcgtttgctgaaaataaacgtcctgttcagggtgtgcgctatattgttggtcgcatgtactatttcaaatatgatgcgctgaccaaagatgaactcccgtattgggatatgtatcctcttgtattcttcttcaattttgtgaagggtgacggggtaaaatttggggaacgtggtgtgacctatctttatggtctcaaccttcattatttgccaccaaagctcagattattagtgtttgaagatcttatcaagttgaggaacgaaagagcgtatcgttctaaaactcgtttgagacttacttgggatgccctgaaacggttcgccaatcaccctctgtacaaccattgtgtgaaactgtaccgcgctgatcaatttcgttcacagctttatgagattgagcctcaatactgggaagttgttttgtttatgcgcactgctcgattccagaaacagagccagatgtctgtttggaaagacgctcgtcgtaaacacaaaaatggttagttctaaaagccgttctatgggataattcgcgaacggctttctataaacatgaggttccatatatgtcatatctacacagaatttttggtattgaattcagttccaacagcgttcgttatgaccgcactcacaaagactccggcggcaagactggttacgaacttggatctcgtttggggtatactgaagagcgccgcctgagtgaacaaactcttttcacagctatgggtattgaatttgaatccgaggctgctttagatctgggggatgaggttcgttttggtaagcctgaagtcttagaatttgttgaagacttcgtgacaggttatacccatgccgctaacaatcgtcgcgccgccaatgatgctgttggtaaatttcattttgctgtgaaataccttgccgacaagcgtattcaaatggaagccgaattcagtgaagaaaacttgaaagcagttttcggagagactggcgatttcctattgacagaagaatccggtacttcttccgctcgtcatttctttagtcaaccgtttatcgtgaagggatgtaaaattctgattgatgaagaacatgtttttggtattcatccggatgttgtatacgatgaagagacgtgtctgatcatttccagattgtttaacagcctggcgtacacttcatattcattgatcgaacgctgtatggaagaagagcatcgccaagttgttgtcaacaaaattcgcttacatgttgagcagaattttaatggattccattgcgcgtctggtgagattccaaacttctacatgtaaagatatcaggggggctgtatgaagaaatatgttcgtctgaacactgtacttgaatcagttatagaatatttcataatgcagcacctcagagcagaaaatgaacacgtgagcgacgctctgacttatgatcaccggatagtggttcattccgtcgaaatagtggatgacgaattgttgattgccaatgttgagcatgctctctgggatgaagtgttagatcagtacgttcaaccgaagttcacacagatcgagttatctgccatgtggccacctgaaacagttttcaatattgatatacaaagttaatattcacgtcagtccgcgctataccagcgttccgttgtctaacacagaagtgttaaccaacttttcaataaaaagtggtttacatcttctaaaaaggcagtatattaatgttccagacgcacagttgaacattaaactgccaaagaaggttactacacaaaagggtttttattatgtcctatattcttcatatcgagtccggcctgaaatttgaaattgatggtaaaaccgctgctgatcttcaagcagaaatcaaaggtgcagaccttatcatcggcaacgtcacgttacgccgtatgatggaaggcgtcctccagtcagccaacggctttgaactcattgaaggtctgagccaggaagagaaagaagagactctcgaagctcttgaccgcgctgaagccgaaaaagttaaaccgaccgaagatgcgggggttaaacagggcgacggtgaatcaaatccggtgattgacgccgaagaaacggttgggcaacctgtcggtgatcaaaattcccagaccgtcgaagacggttctcttgaagtcaacaaagacggctccatttccctggtagttgaaggcgaagaagttcttgacgaag

(19) United States
(12) Patent Application Publication
(10) Pub. No.: US 2025/0160335 A1
(43) Pub. Date: May 22, 2025

(54) FORMULATION OF A LYTIC BACTERIOPHAGE COCKTAIL FOR BIOCONTROL OF SHIGA TOXIN-PRODUCING ESCHERICHIA COLI STRAINS

(71) Applicant: The United States of America, as Represented by the Secretary of Agriculture, Washington, DC (US)

(72) Inventors: VIVIAN C. WU, BURLINGAME, CA (US); YEN-TE LIAO, CONCORD, CA (US)

(21) Appl. No.: 18/948,160

(22) Filed: Nov. 14, 2024

Related U.S. Application Data
(60) Provisional application No. 63/599,751, filed on Nov. 16, 2023.

(51) Int. Cl. A01N 63/40; A01P 1/00; C12N 7/00

(52) U.S. Cl. CPC A01N 63/40; A01P 1/00; C12N 7/00; C12N 2795/00031

(57) ABSTRACT

The present disclosure provides for a combination of bacteriophages from various environmental sources combined to produce a phage mixture targeting shiga-toxin-producing Escherichia coli strains. Techniques and methods of production and use of the phage combinations are also provided.

### CROSS-REFERENCE

The present application claims priority to U.S. Provisional Patent Application Ser. No. 63/599,751 filed Nov. 16, 2024, the content of which is expressly incorporated herein by reference.

### SEQUENCE LISTING

The instant application contains a Sequence Listing XML required by 37 C.F.R. § 1.831(a) which has been submitted in XML file format via the USPTO patent electronic filing system and is hereby incorporated by reference in its entirety. The XML file was created on 11/08/2024 is named Sequence_Listing-002023.xml and has 434 KB bytes.

### BACKGROUND OF THE INVENTION

### Field of Invention

The present disclosure provides for a combination of bacteriophages from various environmental sources combined to produce a phage mixture targeting Shiga-toxin-producing Escherichia coli strains. Techniques and methods of production and use of the phage combinations are also provided.

### Background

Shiga toxin-producing E. coli (STEC) strains—commensal to the gastrointestinal tract of ruminant animals, particularly cattle—have been linked to numerous foodborne outbreaks, causing approximately 265,000 illnesses, 3,600 hospitalizations, and 30 deaths every year in the United States (National Enteric Disease Surveillance: Shiga toxin-producing E. coli (STEC) Annual Report, 2017. Atlanta, Georgia: US Department of Health and Human Services, CDC). More than 30% of these outbreaks are associated with various produce—alfalfa sprouts, iceberg lettuce, and melon—contributing to over 350 identified cases in the United States between 2018 and 2021 (Glaize et al., Int'l J. Food Microbiol., (2021); 347:109196). Besides meat products, global epidemiologic data showed an estimated 147,000 cases of produce-related foodborne outbreaks reported from 1988 to 2020 contributed to 214 hospitalization and 58 deaths, primarily from the infection of STEC 0157 serogroup (Miyahira & Antunes, Int'l J. Food Microbiol., (2021): 352:109266). In 2018, a massive foodborne outbreak involving 22 states in the United States was linked to consuming E. coli O157:H7-contaminated romaine lettuce, causing 46 hospitalizations and 10 cases of the hemolytic uremic syndrome (CDC, 2018. Multistate Outbreaks of E. coli O157:H7 Infections Linked to Romaine Lettuce (Final Update)).

The use of antimicrobial intervention is a common practice regulated by regulatory agencies to ensure food safety during production and upon consumers′ consumption. Chemical-based antimicrobial treatment, such as chlorination, is commonly used in the produce industry because of its easy access, easy installation, and low cost. However, the major drawback is that frequent application results in the development of bacterial resistance, thus compromising the efficacy of these chemical antimicrobials. Additionally, chemical residuals may also negatively influence the produce quality (Fong et al., Front. Microbiol., (2017); 8:2193) and result in an environmental burden (Kraemer et al., Microorganisms, (2019); 7:180). Therefore, alternative interventional methods are critically needed to ensure microbiological safety in the produce industry with the minimum adverse impact (Mir & Kudva, Zonoses Pub. Health, (2019); 66:1-13).

Phages are bacteria-infecting viruses and are highly diverse and ubiquitous in the ecosystem where their bacterial hosts exist (O'Sullivan et al., Ann. Rev. Food Sci. Tech., (2019); 10:151-72). Phages bind specifically to the receptor proteins on the membrane of the target bacterial cells to initiate the infection (Milho et al., Scientific Rep., (2019); 9:18183; Pinto et al., Crit. Rev. Biotech., (2020); 40:1081-97). Due to the nature of the lytic cycle, phage infection always leads to the lysis of bacterial cells and the release of phage progenies for subsequent infection (Clokie et al., Bacteriophage, (2011); 1:31-45). Moreover, lytic phages can evolve with the constant changing of their bacterial hosts to combat the potential development of phage resistance. Consequently, we sought to examine a phage-based intervention technology to specifically address the challenge of STEC contamination using biological, genomic characterization, and in vitro antimicrobial tests to formulate a phage cocktail that can effectively control E. coli O157:H7 using environmentally isolated phages.

### SUMMARY OF THE INVENTION

Provided herein are bacteriophage cocktails, wherein the cocktail comprises at least two isolated bacteriophages that target Escherichia coli selected from the group consisting of: (1) a bacteriophage having a genome sequence at least 95% identical to G1571w (PTA-127634)(SEQ ID NO:1); (2) a bacteriophage having a genome sequence at least 95% identical to Sa1571w (PTA-127635))(SEQ ID NO:2); and (3) a bacteriophage having a genome sequence at least 95% identical to Elw (PTA-127636)(SEQ ID NO:3). In a particular embodiment, the bacteriophage cocktail comprises a bacteriophage having a genome sequence at least 95% identical to G1571w (PTA-127634) and a bacteriophage having a genome sequence at least 95% identical to Elw (PTA-127636). In another particular embodiment, the bacteriophage cocktail comprises a bacteriophage having a genome sequence at least 95% identical to G1571w (PTA-127634)(SEQ ID NO:1), a bacteriophage having a genome sequence at least 95% identical to Sa1571w (PTA-127635)(SEQ ID NO:2), and a bacteriophage having a genome sequence at least 95% identical to Elw (PTA-127636)(SEQ ID NO:3). In a specific embodiment, the bacteriophage cocktail contains bacteriophage G1571w (PTA-127634), bacteriophage Sa1571w (PTA-127635); and bacteriophage Elw (PTA-127636).

A method of treating a surface, comprising administering any of the bacteriophage cocktails recited above to the surface. In some embodiments, the surface is a plant or plant part, such as a seed, pre-harvest crop or leafy vegetable (e.g., lettuce or spinach). In other embodiments, the surface is a food contact surface, such as food packaging or a food processing surface.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides transmission electron microscopic images for the lytic phages used in this study. Phages Sa1571w, G1571w, and Ro1571w show that the morphology belongs to the family Myoviridae (long and contractile tail). Phages Elw and UDF1571w belong to the family Siphoviridae (long and non-contractile tail).

FIG. 2 provides a graphic representation of phylogenetic analysis of whole-genome sequences of Sa1571w and closely related reference phages belonging to the Ackermannviridae family at the nucleotide sequence level using VICTOR (formula d0). Annotations, including family, genus, and species cluster proposed by VICTOR, genomic G+C content, and sequence length, are given to the right-hand side of the tree.

FIG. 3 provides a graphic representation of phylogenetic analysis of whole-genome sequences of G1571w and closely related reference phages belonging to the Straboviridae family at the nucleotide sequence level using VICTOR (formula d0). Annotations, including family, genus, and species cluster proposed by VICTOR, genomic G+C content, and sequence length, are given to the right-hand side of the tree.

FIG. 4 provides a graphic representation of phylogenetic analysis of whole-genome sequences of Elw and closely related reference phages belonging to the Demerecviridae family at the nucleotide sequence level using VICTOR (formula d0). Annotations, including family, genus, and species cluster proposed by VICTOR, genomic G+C content, and sequence length, are given to the right-hand side of the tree.

FIG. 5 provides a representation of the recovery of E. coli O157:H7 RM9799 cells from 48-h biofilm treated with different phage cocktails (G1571w+Sa1571w+Elw vs. commercial phage cocktail) at 8 log PFU/mL for 1 hour at 22° C. Peptone water was used for the control group. The number of asterisks varies, indicating a significant difference at P<0.05. The error bars show the standard deviation of the mean (SEM).

FIG. 6 provides a depiction of bacterial reduction of the mung beans spiked with a four-strain E. coli O157:H7 cocktail by the treatment of different phage cocktails (G1571w+Sa1571w+Elw vs. commercial phage cocktail) at 8 log PFU/mL for 1 hour at room temperature (˜20° C.). The inoculation levels of the control group were ˜3.5 log CFU/g. The asterisk indicates a significant difference at P<0.05 between the two treatments. The error bars show the SEM.

### STATEMENT OF DEPOSIT

Strains representative of the inventions disclosed herein were deposited on Sep. 13, 2023 under the terms of the Budapest Treaty with the American Type Culture Collection (ATCC). A representative bacteriophage G1571w (Tequatrovirus genus under the Straboviridae family) was deposited under ATCC Reference No. PTA-127634. A representative bacteriophage Sa1571w (Kuttervirus genus under the Ackermannviridae family) was deposited under ATCC Reference No. PTA-127635. A representative bacteriophage Elw (Tequintavirus genus under the Demerecviridae family) was deposited under ATCC Reference No. PTA-127636. The microorganism deposits were made under the provisions of the “Budapest Treaty on the International Recognition of the Deposit of Microorganisms for the Purposes of Patent Procedure”. All restrictions on the availability to the public of these deposited microorganisms will be irrevocably removed upon issuance of a United States patent based on this application. For the purposes of this invention, any bacteriophage strains having the identifying characteristics of PTA-127634, PTA-127635, or PTA-127636, including subcultures and variants thereof which have the identifying characteristics and activity as described herein are included.

### DETAILED DESCRIPTION OF THE INVENTION

Lytic phages are innovative antimicrobials that can target bacterial pathogens without killing background flora (Mohammad et al., Int'l J. Food Microbiol., (2019), 289:57-63). Most importantly, they do not contribute to the development of antibiotic resistance in the target bacteria, commonly seen from current chemical interventions (Moye et al., J. Food Safety, (2020), 40:e12763). These phages can combat antibiotic-resistant strains in the agricultural field (Kahn et al., Ann. New York Acad. Sci., (2019), 1441:31-9) and minimize any environmental burden on subsequent terrestrial surroundings (O'Sullivan, et al., supra). Furthermore, FDA has authorized several commercial phage products as Generally Recognized As Safe (GRAS) since 2006 to ensure safe application directly on ready-to-eat food or during food production (Vikram et al., J. Food Protection, (2020), 83:668-76). Therefore, phage-based technology sheds new light on developing a green and novel antimicrobial intervention measurement. It also helps provide the resolution of antibiotic resistance in the food industry.

The instant disclosure focuses on the formulation of phage cocktails, including the three-phage cocktail of G1571w+Sa1571w+Elw, from five different lytic phages isolated from agricultural-associated environments. During the formulation process, we used whole-genome sequencing technology to ensure the diversity of phages and the novelty compared to the reference phages published in the National Center for Biotechnology Information (NCBI). Additionally, sequencing technology is used to thoroughly screen the unwanted genes, such as toxin, lysogenic, and antibiotic-resistant genes, in the phage genomes that could be potential threats to human health and jeopardize the phage application.

As detailed herein, phage G1571w is included in each version of the cocktails, as it showed the most significant antimicrobial activity against E. coli O157:H7 as a single phage and the largest burst size among all the phages in the cocktail. Phage cocktails of the present disclosure show a wide host range capable of infecting other top 6 non-O157 STEC and provide additional mitigating effects against bacterial pathogens other than E. coli O157:H7. G1571w was isolated from the compost sample of food scraps and yard trimming, and phages Sa1571w and Elw were isolated from produce pre-harvest surface water and municipal sewage water samples, respectively. Geographical differences in the source of isolation ensure the natural diversity of the phages besides the genomic variety. Many studies showed that lytic phages were highly associated with the presence of their bacterial host (Liao et al., PLoS One, (2018), 13:e0190534; Wang et al., Can. J. Microbiol., (2015), 61:467-75). Therefore, the samples with high animal contaminations, such as animal feces and sewages, are usually favorable for isolating phages specific to foodborne pathogens, like E. coli O157:H7. However, two (G1571w and Sa1571w) of three phages in this phage cocktail were from the environmental samples with low animal contamination, making our phages, particularly G1571w, unique for developing a phage cocktail.

The instant disclosure provides in vitro antimicrobial activities of various phage combinations to determine whether combining different phages had facilitating, neutral or antagonistic effects compared to that of fewer phages combined. As a result, a cocktail comprising phages G1571w and Elw was an effective combination among all possible two-phage combinations in reducing single E. coli O157:H7, contributing to non-detectable bacterial level, with 7 log reduction (99.99999% reduction) at a 6-h time point. Furthermore, a three-phage cocktail, G1571w+Sa1571w+Elw, demonstrated stronger in vitro antimicrobial effects compared to the two-phage cocktail (G1571w+Elw) at 24-h treatment, with 1.1 log more reduction (92.3% reduction). Surprisingly, the three-phage cocktail was able to mitigate a single E. coli O157:H7 strain and a four-strain E. coli O157:H7 cocktail more than a commercial phage cocktail in vitro after 24-h treatment by 6 log (99.9999% reduction) and 3.4 log reduction (99.96% reduction), respectively.

Further disclosed herein are methods of treating various known contamination sources, such as agricultural irrigation water, commonly susceptible to external contamination and associated with various foodborne outbreaks (Lacombe et al., Front. Food Sci. Tech., (2022), 2:1068690), was used to determine the efficacy of our phage cocktail. The phage cocktail, G1571w+Sa1571w+Elw, formulated in this study continued to mitigate a four-strain E. coli O157:H7 spiked in agricultural well water throughout 24-h treatment, with a maximum 1.3 log reduction (95% reduction) at a 6-h time point. Moreover, the efficacy of the phage cocktail, G1571w+Sa1571w+Elw, was superior to a commercial phage cocktail product in controlling the pathogenic bacteria. Furthermore, a previous study showed that the single phage Sa1571w reduced E. coli O157:H7 on mung bean seeds by 1.1 log (92.3% reduction). Based on the current in vitro results, the phage cocktails of the instant disclosure provide surprisingly better antimicrobial efficacy than Sa1571w alone. Phage cocktails of the instant disclosure may have significant versatile application values with the potential GRAS approval for the application in animal feeds, directly on food, food-processing environments, and food packaging, in addition to the pre-harvest agricultural environment, such as in-farm application and irrigation treatment.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for producing bacteriophage cocktails to target STEC.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “consisting essentially of” excludes additional method (or process) steps or composition components that substantially interfere with the intended activity of the method (or process) or composition and can be readily determined by those skilled in the art (for example, from a consideration of this specification or practice of the invention disclosed herein).

The term “antimicrobial activity”, and grammatical variations thereof, refers to the ability of a composition of the present invention to impede growth of a microorganism, or kill a microorganism, when present in an effective amount. “Antibacterial” refers specifically to the capability of a composition to impede growth of, or kill, bacteria when present in an effective amount.

The term “controlling microbial growth”, as used herein, denotes any activity for completely inhibiting or at least reducing the growth of bacteria (such as E. coli) in a given environment. The term “inhibiting”, as used herein, is to be understood as not only to include the prevention of further growth of but also to killing of any given bacteria. The term “reducing”, as used herein, denotes any decrease in a bacteria's growth (or growth rate), for example, a decrease of at least 5%, 10%, 15%, 20%, 25%, 30%, 40%, 50%, 60%, 70%, 80%, 85%, 90% or 95% as compared to control conditions (i.e. in the absence of antimicrobial agents according to the present invention). Levels of reduction or inhibition of bacterial growth can be indicated by log reduction, for example 1 log reduction=90% reduction (e.g. bacterial counts dropped from 100 CFU to 10 CFU); 1.1 log reduction=92.3% reduction (e.g. bacterial Bacterial counts dropped from 13 CFU to 1 CFU); 1.3 log reduction=95% reduction (e.g. bacterial counts dropped from 20 CFU to 1 CFU); 2 log reduction=99% reduction (e.g. bacterial counts dropped from 100 CFU to 1 CFU); 3.4 log reduction=99.96% reduction (e.g. bacterial counts dropped from 2,500 CFU to 1 CFU); 6 log reduction=99.9999% reduction (e.g. bacterial counts dropped from 1,000,000 CFU to 1 CFU); 7 log reduction=99.99999% reduction (e.g. bacterial counts dropped from 10,000,000 CFU to 1 CFU).

The term “effective amount” of a bacteriophage mixture composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “fresh produce” as used herein refers generally to farm-produced fruits and vegetable crops including, but not limited to fruit and vegetable crops such as e.g., lettuce, spinach, cilantro, cabbage, almonds, cucumbers, cantaloupes, etc.

A “phage cocktail” as used herein refers to a. composition comprising two or more bacteriophages combined according to the methods of the instant invention which can provide a control for at least one strain of E.coli when administered to an organic or inorganic surface.

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seeds (including embryo, endosperm, and seed coat) and fruit (the mature ovary), plant tissue (e.g., vascular tissue, ground tissue, and the like) and cells (e.g., guard cells, egg cells, trichomes and the like).

The terms “G1571w” and “PTA-127634” refer to bacteriophage of the Tequatrovirus genus under the Straboviridae family having the biological and genetic characteristics of the virus deposited under ATCC Reference No. PTA-127634. The terms include genetically modified versions of this virus. A representative genome is available as GenBank accession #OK331996 and this sequence is incorporated in its entirety (SEQ ID NO: 1).

The terms “Sa1571w” and “PTA-127635” refer to bacteriophage of the Kuttervirus genus under the Ackermannviridae family having the biological and genetic characteristics of the virus deposited under ATCC Reference No. PTA-127635. The terms also include genetically modified versions of this virus. A representative genome is available as GenBank accession #OK322699 and this sequence is incorporated in its entirety (SEQ ID NO:2).

The terms “Elw” and “PTA-127636” refer to bacteriophage of the Tequintavirus genus under the Demerecviridae family having the biological and genetic characteristics of the virus deposited under ATCC Reference No. PTA-127636. The terms also include genetically modified versions of this virus. A representative genome is available as GenBank accession #OR621298 and this sequence is incorporated in its entirety (SEQ ID NO:3).

As disclosed herein, a plurality of individually isolated phage was obtained from diverse environmental sources. Members of the same bacteriophage species detailed herein (e g., PTA-127634, PTA-127635, and PTA-127636) have not been shown to reside together in nature, but can be collected from diverse environmental locations, including soil, water treatment plants, raw sewage, sea water, lakes, rivers, streams, standing cesspools, animal fecal matter, organic substrates, biofilms, or medical/hospital sources. The present disclosure includes phage mixtures produced by combining phage of the same species as reported herein, or phages with genomes having 95.0%, 95.1%, 95.2%, 95.3, 95.4%, 95.5%, 95.6%, 95.7%, 95.8%, 95.9%, 96.0%, 96.1%, 96.2%, 96.3%, 96.4%, 96.5%, 96.6, 96.7%, 96.8%, 96.9, 97.0%, 97.1%, 97.2% 97.3%, 97.4%, 97.5%, 97.6%, 97.7, 97.8% 97.9%, 98.0%, 98.1%, 98.2%, 98.3%, 98.4, 98.5%, 98.6%, 98.7%, 98.8%, 98.9%, 99.1%, 99.2%, 99.3% 99.4%, 99.5%, 99.6%, 99.7%, 99.8%, 99.9%, or 100% genomic identity to SEQ ID NO:1, SEQ ID NO:2, and/or SEQ ID NO:3.

Individual phage utilized in the instant disclosure can be purified by any purification technique known in the art, including, but not limited to cesium chloride gradient ultracentrifugation, removing cesium chloride, differentiation filtration, column chromatography and phase partition chromatography.

Any of these compositions described herein can be used to treat bacterial contamination in an environment, or on specific surfaces. Exemplary environments and surfaces that can be treated include, but are not limited to, meat processing facilities, fresh produce, unharvested crops (e.g., lettuce), sprayed or dipped leafy greens (such as mung bean sprouts and lettuce), irrigation water, ready-to-eat foods, seeds (such as mung bean seeds), controlled environment agriculture systems, biofilms, food-contact surfaces, and water troughs.

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

Isolation and characterization of candidate bacteriophages

Escherichia phage “Ro1571w”, “G1571w”, “UDF1571w”, “Sa1571w”, and “Elw” were previously isolated with E. coli O157:H7 (RM18959, ATCC 43888, or ATCC 35150) from various environmental samples, such as non-fecal compost (Ro1571w and G1571w), bovine feces (UDF1571w), surface water in a produce-growing area (Sa1571w), and sewage water (Elw). Phage propagation was performed by mixing 50 μL phage lysate (˜10⁹ PFU/mL) with 45 mL of the log-phase bacterial host culture in tryptic soy broth (TSB; Becton Dickinson, Sparks, MD) and CaCl₂ at 10 mM for incubation at 37° C. for 20 hours. The propagated phages were filtered through a 0.22-μm filter membrane, following centrifugation at 8,000×g for 10 minutes before downstream analysis. A proprietary commercial phage, ECOSHIELD, was used to compare with our developed phage cocktails.

A collection of the top 6 non-O157 STEC—the serogroups O26, O45, O103, O111, O121, O145-E. coli O157:H7 and generic E. coli strains were obtained from the Produce Safety and Microbiology (PSM) Research Unit at the U.S. Department of Agriculture (USDA), Agricultural Research Service (ARS), Western Regional Research Center, Albany, CA, USA for this study. E. coli O157:H7 (ATCC 43888) was used for propagation and quantification of Elw, Sa1571w, and G1571w, E. coli O157:H7 (RM 18959) was used for Ro1571w, and E. coli O157:H7 (ATCC 35150) was used for UDF1571w. Fresh bacterial culture was prepared by inoculating 10 mL TSB with 1 μL loopful of each strain for overnight incubation at 37° C. before use.

The phage cocktail formulation started with the determination of phage varieties based on morphological and genomic characterizations. Additionally, the whole-genome sequencing data of individual phages was used to ensure no toxin genes or lysogenic genes were encoded in each phage genome for the safety of the application. Later, the bacterial host range and productive infection of individual phages against the top six non-O157 and 0157 serogroups of STEC were obtained as a preliminary antimicrobial criterium. The phages with a wide host range were promising for cocktail development; therefore, phage G1571w was selected as the core phage for phage cocktail development.

All selected phages were purified through a CsCl gradient and subsequently subjected to DNA extraction and DNA library preparation prior to sequencing as previously described (Liao et al., Fronts. Microbiol., (2022) 13:1053583). Later, the samples were loaded to a MiSeq Reagent Kit v2 (500-cycle) and sequenced on the MiSeq platform (Illumina, San Deigo, CA, USA). The raw 2×250-bp paired-end reads were subjected to quality control via FASTQC and Trimmomatic with the setting of Q30. De novo assembly was conducted on the resulting quality reads using Unicycler v0.5.0, followed by annotation via the Prokka pipeline Galaxy 1.13 (Seeman, T., Bioinformatics, (2014); 30:2068-69) with default settings. Subsequently, the annotation was manually curated with Universal Protein Resource (UniProt) database (Bairoch et al., Nucl. Acids Res., (2005); 33: D154-D159) using Geneious (v11.0.3, Biomatters, New Zealand). tRNAscan-SE (v2.0) server was used to confirm the predicted tRNAs in the phage genome (Lowe & Chan, Nucl. Acids Res., (2016); 44:W54-W57). The final annotated sequences were deposited in National Center for Biotechnology Information (NCBI) database. The presence of antibiotic-resistance genes and virulence genes in the phage genome was identified using the ResFinder 4.1 webserver (Florensa et al., Microbial Genomics, (2022); 8:000748) and VirulenceFinder (Malberg Tetzchner et al., J. Clin. Microbiol., (2020); 58:e01269-01220).

Purified phages were purified using a CsCl gradient and subsequently subjected to negative staining as previously described (Liao et al., 2022 I, supra). The specimens were then examined in a transmitted electron microscope (FEI Tecnai G₂).

The host range tests for the phages included in this study were conducted against non-pathogenic E. coli, E. coli O157:H7, top six non-0157 STEC, and various Salmonella strains using a spot test assay as previously described (Liao et al., Antibiotics, (2019), 8:74). Briefly, each bacterial culture with 100 μL was mixed with 50% molten TSA and then poured into a Petri dish containing 10-mL bottom TSA. After solidifying, 5 μL of each phage at approximately 7 log PFU/mL was spotted on the plate, followed by incubation at 37° C. The phage-sensitive strains were subsequently tested with productive infection of each phage using the efficiency of plating (EOP) assay described in a previous study (Liao et al., J. Virology, (2011), 85:6567-78). Briefly, fresh cultures were prepared in TSB at 37° C. overnight and used for quantification of Sa1571w using double-layer plaque assay with diluted phage lysate with four successive dilutions (10-3 to 10-7). The plates were incubated at 37° C. for 18 h. The experiment was conducted in 3 replications. Generally, a high phage-producing efficiency had EOP of 0.5 or more; a medium-producing efficiency had EOP above 0.1 but below 0.5; a low-producing efficiency had EOP between 0.001 and 0.1; inefficient phage production was any value lower than 0.001.

Growth factors of the phages included in the phage cocktail were determined using E. coli O157:H7 (ATCC 43888) based on the previous method with subtle modification (Liao et al., Microbiol. Spectrum, (2022 II), 10:e02220-02221). Briefly, 0.2 mL of fresh overnight culture in TSB was sub-cultured in sterile 19.8 mL of TSB and incubated for 2 h at 37° C. to reach the log phase of bacterial growth. Later, a single phage was added to the log-phase bacterial solution (MOI of 0.01) with CaCl₂ at 10 mM and incubated at 37° C. for 10 min, allowing phage adsorption onto the bacterial membranes. The phage-bacterial mixture was centrifuged at 10,000 xg for 5 min at 4° C. to discard the supernatant. After washing, the bacterial pellet was resuspended with 20 mL of fresh TSB before further 10-fold dilution (0.3 mL of resuspension in 29.7 mL of TSB). The sample was then incubated at 37° C. for a specific period (varied by different phages). Meanwhile, the phage-infected bacterial cells were quantified before incubation (time 0) by mixing 50 μL of the 30-mL phage-bacterial mixture (no filtration) with 100 μL of fresh overnight bacterial culture for a double-layer plaque assay. Subsequently, a 1 mL phage-bacterial mixture was obtained from the selected time points and filtered using a 0.22-μm filter membrane. The phage titers at each time point were determined using the double-layer plaque assay. The experiment was conducted in three replications to estimate the latent period and burst size of the selected phages (G1571w & Elw).

A quick antimicrobial activity screening on various two-phage combinations from the first included phages G1571w, Sa1571w, UDF1571w, and Ro1571w was conducted using a spectrophotometer (Elw phage was isolated later to increase the morphological variety). Detailed information regarding the experiment and assay used in the formulation step is described below.

### Results and Discussion

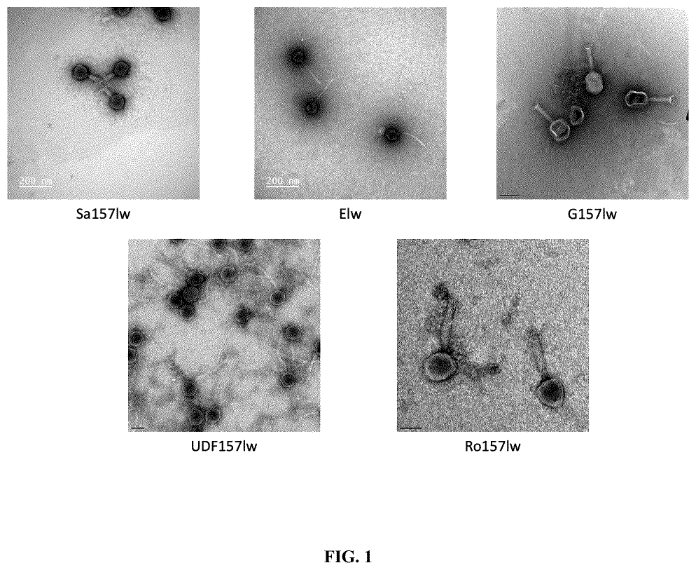

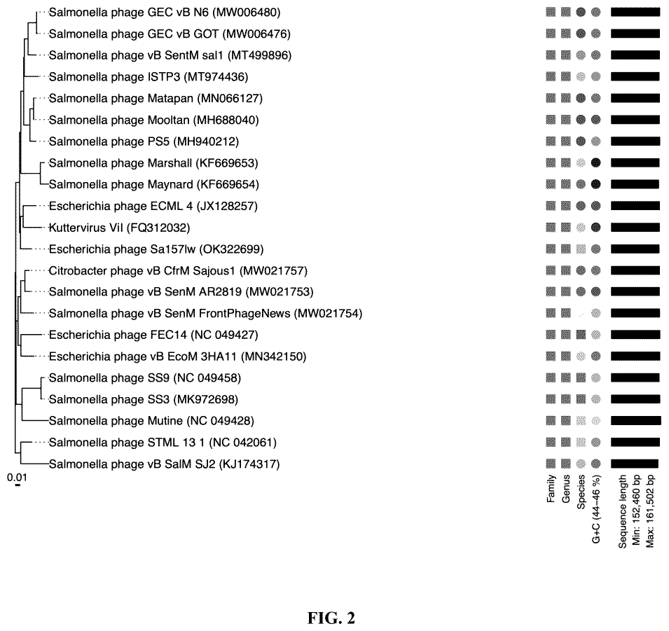

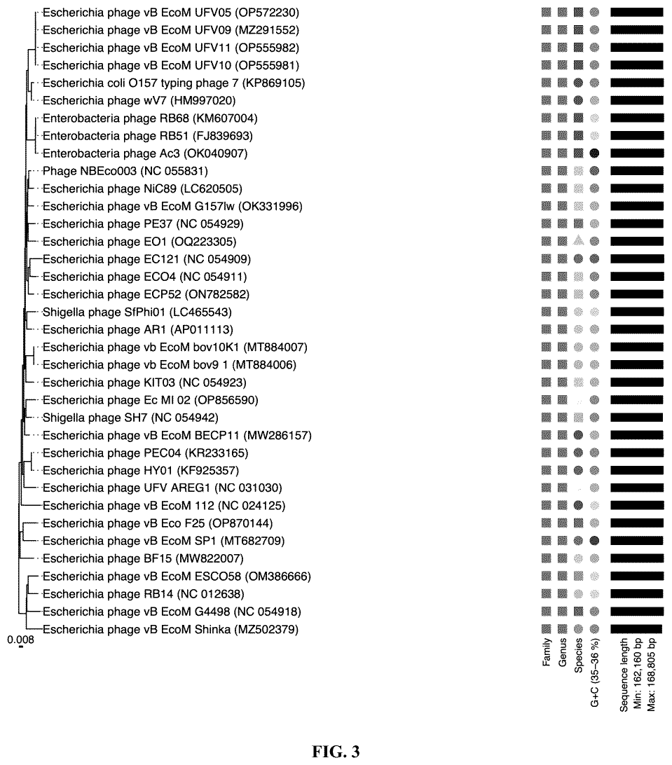

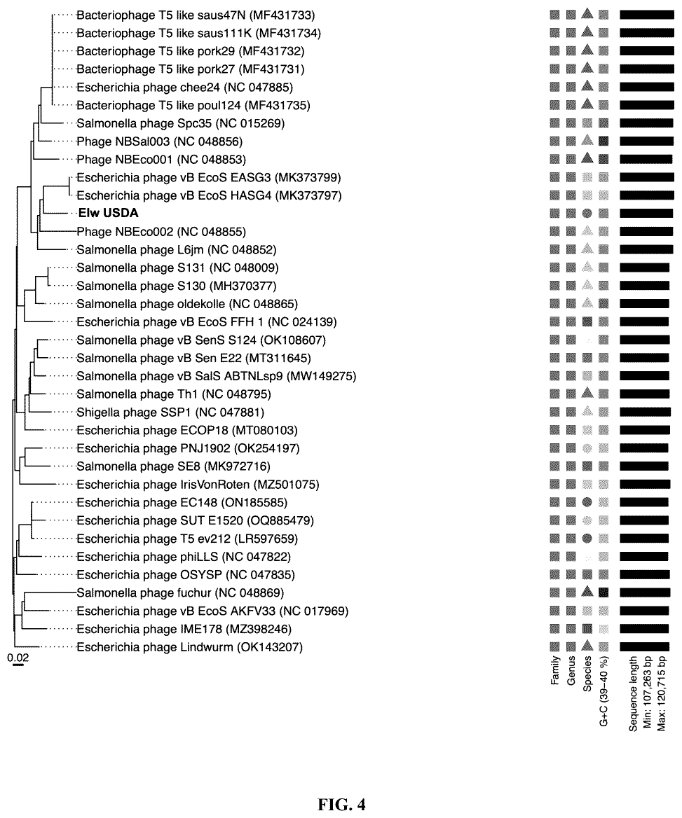

Five lytic phages isolated from various environmental samples are in genetic variation (Table 1) and morphological differences (FIG. 1). Although all phages Ro1571w, Sa1571w, and G1571w have a long and contractile tail showing the Myoviridae morphology, their genomic taxonomies vary. The sequencing results show that G1571w and Sa1571w have larger genome sizes, classified into the Tequatrovirus genus under the Straboviridae family and the Kuttervirus genus under the Ackermannviridae family, respectively. Ro1571w belongs to the Wifcevirus genus, a new phage classification without an assigned family. UDF1571w is genomically classified in the Rogunavirus genus under the Drexlerviridae family and contains a long, non-contractile tail demonstrating the Siphoviridae morphology. Elw also has the Siphoviridae morphology and the taxonomy belonging to the Tequintavirus genus under the Demerecviridae family. The phylogenetic analyses show that phages Sa1571w, G1571w, and Elw are at their own leaf (FIG. 2, FIG. 3, FIG. 4), indicating the nucleotide sequence novelty of these three phages among the close-related reference phages.

TABLE 1
Genomic classification of lytic phages isolated
from different environmental samples.

| | Isolation | Genome | | | |
|---|---|---|---|---|---|
| Phage ID | sources | size | | Family | Genus |
| Elw | Sewage | 118,119 | bp | Demerecviridae | Tequintavirus |
| Ro157lw | Non-fecal | 72,179 | bp | not classified | Wifcevirus |
| | compost | | | | |
| UDF157lw | Bovine | 46,604 | bp | Drexlerviridae | Rogunavirus |
| | feces | | | | |
| Sa157lw | Surface | 155,887 | bp | Ackermann- | Kuttervirus |
| | water | | | viridae | |
| G157lw | Non-fecal | 167,170 | bp | Straboviridae | Tequatrovirus |
| | compost | | | | |

Among all five phages, G1571w has a broader host range targeting different STEC serogroups, including 045, 0103, and 0145, than the other phages in addition to infecting E. coli O157:H7 strain (Table 2; H=host strain used for the phage isolation and R=the bacterial strain is resistant to the phage). For Table 2, the EOP was calculated by the ratio of phage titer on a test bacterium versus the primary bacterial host. High production efficiency is EOP≥0.5, medium production efficiency is 0.5>EOP≥0.1, low production efficiency is 0.1>EOP>0.001, and the inefficiency of phage production is EOP 0.001. H means the host strain used for the phage isolation, and R means the bacterial strain is resistant to the phage infection. Additionally, these phages express a variety of productive infections against different E. coli O157:H7 strains. The phenomenon is favorable as the development of a phage cocktail. Phage Sa1571w was a polyvalent phage able to infect various Salmonella enterica serovars: Heidelberg, Saintpaul, Agona, Typhimurium, and Enteritidis (Liao et al., 2022 I, supra). However, these phages show a wide infection capability against generic E. coli strain. Growth factors—the latent period and burst size—were examined on the three phages included in the cocktail. The results show that phages G1571w and Sa1571w have similar latent periods of 30 min, whereas Elw has around 40 min (Table 3). Additionally, G1571w has the largest burst size of approximately 1,220 PFU/CFU among all the phages.

TABLE 2
Host range and efficiency of plating (EOP*) for
phages against various top 6 non-O157, E. coli O157:H7,
and non-pathogenic E. coli strains.

| | Tested phages | | | | |
|---|---|---|---|---|---|
| Strain Ref. No. | Elw | Ro157lw | UDF157lw | Sa157lw | G157lw |
| STEC O26, | R | R | R | R | R |
| O111, and O121 | | | | | |
| E. coli O45:H16 | R | R | R | R | 0.1 |
| E. coli O103:H2 | R | R | R | R | 0.04 |
| E. coli O145:H28 | R | R | R | R | 0.14 |
| (RM13514) | | | | | |
| E. coli O157:H7 | 0.88 | H | 1.1 | 0.8 | 0.2 |
| (RM18959) | | | | | |
| E. coli O157:H7 | 1.84 | 0.9 | H | 0.82 | 0.2 |
| (ATCC 35150) | | | | | |
| E. coli O157:H7 | H | 0.8 | <0.001 | H | H |
| (ATCC 43888) | | | | | |
| Generic E. coli | 0.52 | <0.001 | 0.06 | R | 0.04 |
| ATCC 13706 | | | | | |

TABLE 3
One-step growth curve for phages on
E. coli O157:H7 (ATCC 43888).

| | Bacterial | Growth factors | |
|---|---|---|---|
| Phage ID | strain | Latent period (min) | Burst size (PFU/CFU) |
| G157lw | ATCC 43888 | 30 | 1220 |
| Sa157lw | ATCC 43888 | 30 | 130 |
| Elw | ATCC 43888 | 40 | 55 |

### Example 2

### Development of Phage Combinations

A bacterial challenge assay was performed to measure the effects of phages Ro1571w, UDF1571w, and G1571w with different MOIs on the cocktail of two E. coli O157:H7 (RM 18959 and E. coli O157:H7 (RM 35150) based on bacterial optical density at a wavelength of 600 nm (OD₆₀₀) as previously described with minor change (Zhang et al., Microorganisms, (2021), 9:1527). In brief, fresh bacterial cultures of E. coli O157:H7 (ATCC 35150) and E. coli O157:H7 (RM18959) were prepared in TSB at 37° C. overnight and further diluted in TSB to 1×10⁶ CFU/mL. An aliquot of 200 μL diluted bacterial solution containing both bacterial cultures per well was added to a 96-well plate. Later, 10 μL of individual phage with different titers was added to the wells to reach MOIs of 1, 10, and 100; the control group contained only bacterial solution without phage. The reading of OD₆₀₀ was measured using a spectrophotometer (Promega, Madison, WI, USA) at 25° C. every 30 min for 12 h. The experiment was conducted in 3 replications.

A culture of E. coli O157:H7 (RM9995) was used to evaluate the antimicrobial activity of an individual and different combinations of phages using the method as previously described with subtle modifications (Liao et al., 2022 II, supra). In brief, the bacterial culture was prepared in 10 ml TSB at 37° C. for 18 h, followed by dilution in LB broth (Invitrogen, Carlsbad, CA, USA) to reach the final concentration at approximately 4 log CFU/ml for the experiment. Single phages or multi-phage cocktails in SM buffer was added to 4-ml bacterial solution at an MOI of 1. For the control, SM buffer, with the same volume as the phage used in the treatment, was also added to 4-ml bacterial solution. Both control and treatment groups were incubated at 25° C. or 30° C., and the bacterial counts were quantified at various time points (0, 3, 4, 6, 8 or 24 hours) during the incubation. Bacterial counts were quantified on Sorbitol MacConkey agar (SMAC; BD, Franklin Lakes, NJ) overlayered thin TSA (Thin Agar Layer Method, TAL) (Wu, V., Food Microbiol, (2008), 25:735-744).

Agricultural water from a produce-growing area was collected and used to evaluate the efficacy of a phage cocktail formulated in this study versus a commercial phage cocktail in reducing a four-strain E. coli O157:H7 cocktail (RM9995, RM18419, RM6416, and ATCC35150). The water sample was filtered through a 0.22-mm membrane before use. Each bacterial culture was prepared in 10 ml TSB at 37° C. overnight. An aliquot of 0.1 ml per bacterial strain was added to a sterile tube to prepare the bacterial cocktail used to inoculate 60-ml water sample at the final concentration of 5 log CFU/ml. The E. coli O157:H7-inoculated water was evenly dispensed into three conical tubes (20 ml per tube) and treated separately with a three-phage cocktail and the commercial phages at an MOI=50. The SM butter, with the same volume used for the phage cocktails, was added to a 20-ml bacterial cocktail as the control group. The control and treatment tubes were incubated at 25° C. for 24 h with shaking. Bacterial counts were evaluated for control and treatment groups by plating on duplicated SMAC TAL at 0, 4, 6, and 24 h. The plates were incubated at 24° C. overnight for bacterial quantification.

A quick screening assay was used to determine antimicrobial suppression regarding two-phage combinations of G1571w, Sa1571w, and Ro1571w against a cocktail of E. coli O157:H7. The results showed that the order of antimicrobial activities for the combination groups from high to low is G1571w+Sa1571w or G1571w+UDF1571w and G1571w+Ro1571w (results not shown). As a result, the combination with Ro1571w had the worst antimicrobial effect among all and thus was removed. Therefore, phage Ro1571w was excluded from the subsequent antimicrobial activity test in LB broth.

The remaining phages and phage Elw were subjected to in vitro antimicrobial activity tests on two-phage, three-phage, and four-phage combinations to determine “facilitating” (greater efficacy than the most effective constituent phages acting alone), “neutral” (no better or worse than the most effective constituent phages acting alone) or “antagonistic” (lower efficacy than the most effective constituent phages acting alone) effects among different phages used in the same cocktail. First, antimicrobial effects of individual phages of G1571w, Sa1571w, and UDF1571w were tested against E. coli O157:H7 (RM11781) at 30° C. for 3 h. The results showed that phage G1571w was the best among the three phages, resulting in more than 6 log bacterial reduction (99.9999% reduction) in 3 h (Table 4; *numbers represent the viable cell counts of bacteria (log₁₀ CFU/ml)). Later, G1571w was used as a core phage in combination with Sa1571w or UDF1571w to test a two-phage cocktail against the same bacterial strain. The results showed that the phage combination of G1571w and Sa1571w had an enhanced antimicrobial effect in mitigating E. coli O157:H7 (RM11781) in comparison to phages G1571w and UDF1571w combined (Table 5; *numbers represent the viable cell counts of bacteria (log₁₀ CFU/ml)). The other two-phage combination (G1571w and UDF1571w) had a similar bacterial reduction level compared to the single phage G1571w. Furthermore, phage Elw was tested in combination with G1571w to compare with the two-phage cocktail of G1571w and Sa1571w. The results showed that combining phage Elw greatly increased the antimicrobial activity of G1571w alone at 6 h and 24 h of the treatment at 25° C. (Table 6; *numbers represent the viable cell counts of bacteria (log₁₀ CFU/ml); ^{α}O represents a non-detectable bacterial count). Additionally, the combination of G1571w and Elw had better antimicrobial activity than G1571w and Sa1571w combined in LB broth.

TABLE 4
In vitro antimicrobial activities of individual phages.

Phage treatment

| Time | Control | G157lw | Sa157lw | UDF157lw |
|---|---|---|---|---|
| 3 h | 8.7* | 2.4 | 3.9 | 4.0 |

TABLE 5
In vitro antimicrobial activities of two-phage combinations.

Phage treatment

| Time | Control | G157lw + Sa157lw | G157lw + UDF157lw |
|---|---|---|---|
| 3 h | 8.8* | 2.2 | 2.3 |

TABLE 6
In vitro antimicrobial activities of two-phage combinations.

| | | Treatment groups | | | |
|---|---|---|---|---|---|
| Time | | Control | G157lw | G157lw + Elw | G157lw + Sa157lw |
| 0 | h | 7.0* | 7.0 | 7.0 | 7.0 |
| 4 | h | 7.5 | 1.3 | 0.5 | 0.7 |
| 6 | h | 8.0 | 1.3 | 0.0^{a} | 1.5 |
| 24 | h | 8.6 | 6.5 | 1.7 | 5.4 |

Next, phage Elw was added to the combination of G1571w and Sa1571w against E. coli O157:H7 (ATCC 35150) at 25° C. for 6 h. The results showed that the three-phage cocktail combination containing Elw reduced more bacterial cells of E. coli O157:H7 at 4-h and 6-h time points than the two-phage cocktail (G1571w and Sa1571w), with 1.3 and 1.1 log reduction, respectively (Table 7, *numbers represent the viable cell counts of bacteria (log₁₀ CFU/ml); ^{α}0 represents a non-detectable bacterial count). Subsequently, the phage cocktail of G1571w, Sa1571w, and Elw was combined with UDF1571w to combat E. coli O157:H7 for 24 h; however, no synergistic effect was observed (results not shown). Subsequently, a three-phage cocktail, containing G1571w, Sa1571w, and Elw, was used to compare with ECOSHIELD, the sole commercial phage product tested herein, to mitigate single E. coli O157:H7 strain (ATCC 35150) or a four-strain E. coli O157:H7 cocktail (ATCC 35150, RM6416, RM9995, and RM18419) at 25° C. for 24 h. The results showed that the three-phage cocktail in this study reduced single E. coli O157:H7 to the non-detectable level after 6 h to 24 h of the phage treatment (Table 8, *numbers represent the viable cell counts of bacteria (log₁₀ CFU/ml); _{α}0 represents a non-detectable bacterial count). For the commercial phage product, the bacterial growth rebounded starting from 6 h. For the antimicrobial effect against a four-bacteria cocktail, our phage cocktail was able to reduce the bacterial levels from 6.5 log CFU/ml to below 1 log CFU/ml up to 8 h (Table 9, *numbers represent the viable cell counts of bacteria (log₁₀ CFU/ml)). Although bacteria grew back after 24 h of treatment, the bacterial levels mitigated by our three-phage cocktail were significantly lower than that caused by the commercial phage product by more than 3 log.

TABLE 7
In vitro antimicrobial activities of two-
phage and three-phage combinations.

| | Phage treatment | | |
|---|---|---|---|
| Time | Control | G157lw + Sa157lw | G157lw + Sa157lw + Elw |
| 0 h | 6.4* | 6.4 | 6.4 |
| 4 h | 7.3 | 1.6 | 0.3 |
| 6 h | 8.1 | 1.1 | 0^{a} |

TABLE 8
In vitro antimicrobial activities of a three-phage
combination and a commercial phage product (ECOSHIELD)
against a single E. coli O157:H7 strain.

| | | Phage treatment | | |
|---|---|---|---|---|
| Time | | Control | G157lw + Sa157lw + Elw | ECOSHIELD |
| 0 | h | 6.4* | 6.4 | 6.4 |
| 4 | h | 6.6 | 2.2 | 1.7 |
| 6 | h | 6.8 | 0^{a} | 1.9 |
| 24 | h | 8.5 | 0^{a} | 6 |

TABLE 9
In vitro antimicrobial activities of a three-phage combination
and a commercial phage product against a four-strain E. coli
O157:H7 cocktail (ATCC 35150, RM18419, RM6416, and RM9995).

| | | Phage treatment | | |
|---|---|---|---|---|
| Time | | Control | G157lw + Sa157lw + Elw | Commercial phage |
| 0 | h | 6.5* | 6.5 | 6.5 |
| 4 | h | 7.0 | 3.1 | 2.8 |
| 6 | h | 7.6 | 0.9 | 2.3 |
| 8 | h | 8.1 | 0.9 | 2.5 |
| 24 | h | 8.6 | 3.2 | 6.6 |

The efficacy of phage application was determined in treating agricultural well water inoculated with a four-strain E. coli O157:H7 cocktail. The results showed that using the MOI of 50, the 3-phage cocktail continued to mitigate the bacterial levels examined at 4 and 6 h time points at 25° C., with a maximum reduction of 1.3 log compared to the control group. Although the E. coli O157:H7 bounced back at 24 h, the treatment with the 3-phage combination kept the bacterial level lower than the control group by 0.6 log and that treated with a commercial phage. Besides the antimicrobial activities, some nutrients might be present in the commercial phage product to facilitate bacterial growth in the water sample (Table 10, *numbers represent the viable cell counts of bacteria (log₁₀ CFU/ml)).

TABLE 10
Phage application of the 3-phage cocktail and a commercial
phage product against a four-strain E. coli O157:H7
cocktail (ATCC 35150, RM18419, RM6416, and RM9995) spiked
in agricultural water at 25° C. for 24 h.

| Time | | Control | G157lw + Sa157lw + Elw | Commercial phage |
|---|---|---|---|---|
| 0 | h | 5.0* | 5.0 | 5.0 |
| 4 | h | 5.0 | 4.0 | 5.0 |
| 6 | h | 5.1 | 3.8 | 4.4 |
| 24 | h | 5.7 | 5.1 | 6.8 |

Experiments subjected to statistical analysis were conducted in three individual replications. The quantification of bacteria and phages was calculated as CFU/g and PFU/mL, respectively, with logarithmical conversion for statistical analysis.

### Example 3

Antimicrobial Activity of Phage Cocktails Against Pathogenic E. coli Other than STEC

The susceptibility of the tested strains, such as EPEC, ETEC, and EIEC, to phage cocktails, was determined using a spot test assay. Later, the susceptible strains with strong lysis were used to determine EOP. If the tested strains showed medium to high EOP with the 3-phage cocktail, further experiments would be used for an in vitro antimicrobial activity test.

The results showed that the 3-phage cocktail (G1571w+Sa1571w+Elw) did not infect EIEC or EPEC and rendered weak antimicrobial activity against two ETEC strains of the serogroups 025 and 0117, with low efficiency and inefficiency, respectively (Table 11; the EOP was calculated by the ratio of phage titer on a test bacterium versus the primary bacterial host. E. coli O157:H7 ATCC43888 was used as host strain for EOP. High production efficiency is EOP≥0.5, medium production efficiency is 0.5>EOP≥0.1, low production efficiency is 0.1>EOP>0.001, and the inefficiency of phage production is EOP≤0.001. R means the phage cocktail did not infect and lyze the bacterial strains). The results suggested that the 3-phage cocktail is specific to STEC strains and has little mitigating effects on the pathogenic E. coli other than STEC O157:H7.

TABLE 11
Susceptibility test and efficiency of plating (EOP) of the
three-phage cocktail of G157lw + Sa157lw + Elw to
the selected pathogenic E. coli strains other than STEC.

| Number | Pathotypes | Description | Spot test/EOP |
|---|---|---|---|
| 1 | EIEC | O21:NM | R |
| 2 | EIEC | O29:NM | R |
| 3 | EPEC | O55:H7 | R |
| 4 | EPEC | O119:NM | R |
| 5 | EPEC | O142:H6 | R |
| 6 | ETEC | O19 | R |
| 7 | ETEC | O117 | inefficiency |
| 8 | ETEC | O25 | low efficiency |
| 9 | ETEC | O159 | R |

### Example 4

Application of Phage Cocktails on Mung Bean Seeds Contaminated with E. coli O157:H7

Mung beans were disinfected with 2% of NaClO for 15 min and then rinsed with sterile water five times to remove NaClO residue. The sterilized seeds were air-dried in a biosafety hood. Before the experiment, the sterilized mung beans were homogenized with 20 mL sterile 0.1% peptone water for 2 min and plated on Sorbitol MacConkey (SMAC) with a thin TSA overlay to check the beans were free of background flora.

Mung beans (100 g) were submerged in 140 mL bacterial solution containing a four-strain E. coli O157:H7 cocktail (RM9995, RM6416, RM18419, and ATCC35150) at approximately 5.5 log CFU/mL for 1 h at room temperature (˜20° C.). After air drying under a biosafety hood, inoculated beans were stored in a sterile zip-loc bag at 4° C. overnight before the experiment. Later, three groups of ten grams of inoculated mung beans were measured and subjected to the control, a test 3-phage cocktail (G1571w+Sa1571w+Elw), and commercial phage treatments, respectively. For the phage cocktail treatment, mung beans were submerged in 50 mL phage solution with ˜8 log PFU/mL for 1 h at room temperature (˜20° C.). For the control, mung beans were mixed with 50 mL 0.1% peptone water under the same condition. After removing the phage solution and peptone water, the control and treated beans were dried on a separate sterile aluminum foil sheet under the hood for 2 h. Subsequently, the bacterial levels were quantified by plating on SMAC with thin TSA overlay plates.

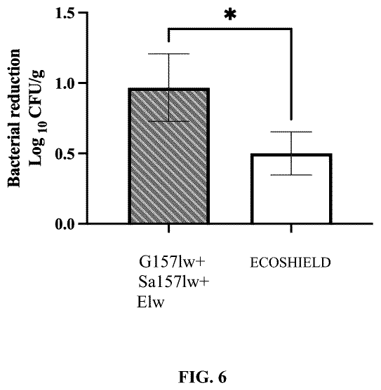

The results showed that the 3-phage cocktail (G1571w+Sa1571w+Elw) reduced the four-strain E. coli O157:H7 cocktail on the contaminated mung beans by 1 log after phage treatment for 1 hour (FIG. 6). Moreover, the 3-phage cocktail caused a significant reduction compared to the commercial phage product by 0.5 log. The findings reveal that this 3-phage cocktail is a promising and alternative antimicrobial agent in preventing E. coli O157:H7 contamination on seeds and other plant tissues.

### Example 5

### Antimicrobial Activity of Phage Cocktails Against E. Coli O157:H7 Biofilm.

E. coli O157:H7 (RM9995) culture was prepared in 10 mL TSB and incubated at 37° C. for 20 h. Later, an aliquot of 0.1 ml of the overnight culture was placed on each sterile stainless coupon (n=9), put and sealed in a sterile 12-well plate to grow the biofilm at 22° C. for 48 h without shaking. After incubation, the planktonic cells were removed from the stainless surface by washing with 1 mL 0.1% peptone water twice. Later, three coupons per group were individually subjected to 0.1% peptone water, a 3-phage cocktail (G1571w+Sa1571w+Elw), and ECOSHIELD (commercial phage product) for 1 h at 22° C. (room temperature). In brief, each coupon was submerged in 3 mL peptone water or phage solutions at ˜8 log PFU/mL for 1 h. After treatment, each coupon was washed with peptone water, followed by biofilm cells being removed from the surface into 10 mL peptone water using ultrasonication before bacterial quantification on SMAC TAL plates.

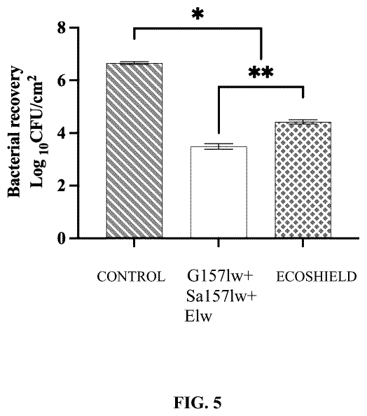

The results showed that both phage cocktails with ˜8 log PFU/mL significantly mitigated 48-h E. coli O157:H7 biofilm on the stainless surface (FIG. 5). Additionally, the 3-phage cocktail rendered much stronger anti-biofilm activity (P<0.05) in reducing the biofilm with 0.9 log more than commercial ECOSHIELD. The findings indicate that the 3-phage cocktail reported herein is superior to the commercial phage product used in this study as an anti-biofilm agent.

### Example 6

### Additional Phage Combination Testing In Vitro and in Agricultural Water

Additional phage cocktail experiments in bacterial culture and additional experiments in agricultural water were carried out substantially as described in Example 2 above, including bacterial culture preparation, single phage or multi-phage cocktails, agricultural water handling, incubation times and media utilized.

### Additional Bacterial Culture Analysis

The results from these experiments showed that G157+Elw had the best antimicrobial activity within 6-h treatment at 25° C. (Table 12; *numbers represent the viable cell counts of bacteria (log₁₀ CFU/ml)). However, Elw+Sa1571w had further reduced bacterial levels to 1.2 log CFU/ml, with the highest reduction among all treatment groups. We selected G157+Elw as the two-phage cocktail for the following experiment because this two-phage combination reached the maximum bacterial reduction faster than Elw+Sa1571w.

TABLE 12
In vitro antimicrobial activities of two-phage combinations with MOI
of 0.1 against E. coli O157:H7 (ATCC35150) at 25° C. for 24 h.

| | Phage treatment | | | | |
|---|---|---|---|---|---|
| | | | G157lw + | G157lw + | Elw + |
| Time | Control | G157lw | Elw | Sa157lw | Sa157lw |
| 0 | 6.6* | 6.6 | 6.6 | 6.6 | 6.6 |
| 4 | 7.0 | 6.8 | 6.7 | 6.7 | 6.7 |
| 6 | 7.8 | 3.5 | 1.7 | 2.2 | 4.2 |
| 24 | 8.8 | 5.5 | 5.1 | 5.5 | 1.2 |

The results also showed that both two-phage and three-phage cocktails are better than the commercial phage product over a 24-h treatment period, even though the commercial product (ECOSHIELD) reduced the bacteria to the minimum levels (in 4 h) faster than both of our phage cocktails (in 6 h) (Table 13; *numbers represent the viable cell counts of bacteria (log₁₀ CFU/ml)). Additionally, the three-phage cocktail (G1571w+Sa1571w+Elw) continued to reduce the bacterial population to the non-detectable levels at the 24-h timepoint; however, the bacteria grew back at 24 h from the treatment of the two-phage cocktail and the commercial phage cocktail.

TABLE 13
In vitro comparison of antimicrobial activities of various phage cocktails
against E. coli O157:H7 (ATCC35150) at 25° C. for 24 h.

| | Phage treatment | | | |
|---|---|---|---|---|
| | | | G157lw + | |
| Time | Control | G157lw + Elw | Sa157lw + Elw | ECOSHIELD |
| 0 | 6.4* | 6.4 | 6.4 | 6.4 |
| 4 | 7.0 | 6.7 | 5.9 | 1.8 |
| 6 | 7.6 | 0.6 | 0.5 | 2.0 |
| 24 | 8.4 | 3.2 | 0.0 | 5.5 |

### Additional Agricultural Water Analysis

The results showed that both two-phage and three-phage cocktails are better than the tested commercial phage product (ECOSHIELD) upon application in reducing a four-strain E. coli O157:H7 cocktail (ATCC 35150, RM18419, RM6416, and RM9995) in agricultural water (Table 14; *numbers represent the viable cell counts of bacteria (log₁₀ CFU/ml)). Additionally, the three-phage cocktail (G1571w+Sa1571w+Elw) continued to reduce the bacterial population over a 24-h treatment period, contributing to a 1.6 log reduction compared to the control. However, the bacterial levels were suppressed by the commercial phage product until the 6-h timepoint but grew back at 24 h.

TABLE 14
Effects of a two-phage cocktail, a 3-phage cocktail, and a commercial
phage product with MOI of 50 against a four-strain E. coli O157:H7
cocktail spiked in agricultural water at 25° C. for 24 h.

| | Phage treatment | | | |
|---|---|---|---|---|
| | | | G157lw + | |
| Time | Control | G157lw + Elw | Sa157lw + Elw | ECOSHIELD |
| 0 | 4.8* | 4.8 | 4.8 | 4.8 |
| 4 | 5.1 | 4.7 | 4.6 | 4.8 |
| 6 | 5.3 | 4.7 | 4.6 | 4.8 |
| 24 | 5.6 | 4.7 | 4.0 | 5.8 |

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A composition comprising a bacteriophage cocktail, wherein the cocktail comprises at least two isolated bacteriophages that target Escherichia coli selected from the group consisting of: (1) a bacteriophage having a genome sequence at least 95% identical to G1571w (PTA-127634)(SEQ ID NO:1); (2) a bacteriophage having a genome sequence at least 95% identical to Sa1571w (PTA-127635)(SEQ ID NO:2); and (3) a bacteriophage having a genome sequence at least 95% identical to Elw (PTA-127636)(SEQ ID NO:3).

2. The composition of claim 1, wherein the bacteriophage cocktail comprises a bacteriophage having a genome sequence at least 95% identical to G1571w (PTA-127634) and a bacteriophage having a genome sequence at least 95% identical to Elw (PTA-127636).

3. The composition of claim 1, wherein the bacteriophage cocktail comprises a bacteriophage having a genome sequence at least 95% identical to G1571w (PTA-127634)(SEQ ID NO:1), a bacteriophage having a genome sequence at least 95% identical to Sa1571w (PTA-127635)(SEQ ID NO:2), and (3) a bacteriophage having a genome sequence at least 95% identical to Elw (PTA-127636)(SEQ ID NO:3).

4. The composition of claim 1, wherein the bacteriophage cocktail comprises bacteriophage G1571w (PTA-127634), bacteriophage Sa1571w (PTA-127635); and bacteriophage Elw (PTA-127636).

5. A method of treating a surface, comprising administering the composition of claim 1 to said surface.

6. The method of claim 5, wherein the surface is a plant or plant part.

7. The method of claim 6, wherein plant part is a seed.

8. The method of claim 5, wherein the plant is a pre-harvest crop.

9. The method of claim 5, wherein the surface is a plant.

10. The method of claim 9, wherein the leafy vegetable is lettuce or spinach.

11. The method of claim 5, wherein the surface is a food contact surface.

12. The method of claim 11, wherein the food contact surface is food packaging surface.

13. The method of claim 11, wherein the food contact surface is a food processing surface.
